(19) United States
(12) Patent Application Publication
(10) Pub. No.: US 2003/0171562 A1
(43) Pub. Date: Sep. 11, 2003

(54) Genes and proteins for the biosynthesis of polyketides

(76) Inventors: Chris M. Farnet, Outremont (CA); Emmanuel Zazopoulos, Montreal (CA); Alfredo Staffa, Saint-Laurent (CA); Xianshu Yang, Montreal (CA)

Correspondence Address: Ywe J. Looper, ECOPIA BIOSCIENCES INC., 7290 Frederick-Banting, Saint-Laurent, QC, H4S 2A1, CA

(21) Appl. No.: 10/132,134

(22) Filed: Apr. 26, 2002

Related U.S. Application Data
(60) Provisional application No. 60/286,346, filed on Apr. 26, 2001.

(51) Int. Cl. C07H 21/02; C07H 21/04

(52) U.S. Cl. 536/23.1

(57) ABSTRACT

Genes and proteins involved in the biosynthesis of polyketides by microorganisms, including the genes and proteins forming the biosynthetic loci for the polyketide dorrigocin from Streptomyces platensis subsp. rosaceus and the polyketide lactimidomycin from Streptomyces amphibiosporus. The genes and proteins allow direct manipulation of dorrigocin, lactimidomycin and related chemical structures via chemical engineering of the enzymes involved in the biosynthesis of dorrigocin and lactimidomycin.

### CROSS-REFERENCING TO RELATED APPLICATION:

This application claims benefit under 35 USC §119 of provisional application U.S. Ser. No. 60/286,346 filed on Apr. 26, 2001 which is hereby incorporated by reference in its entirety for all purposes.

### FIELD OF INVENTION

The present invention relates to nucleic acids molecules which encode proteins that direct the synthesis of polyketides, particularly dorrigocin, migrastatin and lactimidomycin polyketides. The present invention also is directed to use of DNA to produce compounds exhibiting antibiotic activity based on the dorrigocin, migrastatin and lactimidomycin structures.

### BACKGROUND

Dorrigocins, migrastatins and lactimidomycins are polyketides. Polyketides occur in many types of organisms including fungi and bacteria, in particular, the actinomycetes. The structure of two dorrigocins, designated as dorrigocin A and dorrigocin B, is described in Hochlowski et al, J. Antibiotics 47:870 (1994) and U.S. Pat. No. 5,484,799. Dorrigocins have been reported to have antifungal and antitumor activity (Karwowski et al., J. Antibiotics 47:862 (1994); U.S. Pat. No. 5,589,485). Biological properties of dorrigocins are also discussed in Kadam and McAlpine, J. Antibiotics 47:875 (1994). The structure of migrastatin is described in Nakae et al, J. of Antibiotics 53: 1228 (2000). Migrastatin has been reported to inhibit tumor cell migration (Nakae et al, J. of Antibiotics 53:1130 (2000). A related compound, referred to as isomigrastin was described in Woo et al., J. Antibiotics, Vol 55, pp.141-146 (2002).

Polyketides are a class of compounds formed of 2-carbon units through a series of condensations and subsequent modifications. Polyketides are synthesized in nature by polyketide synthase (PKS) enzymes. Given the difficulty in producing polyketide compounds by traditional chemical methodology, and the typically low production of polyketides in wild-type cells, there has been considerable interest in finding improved or alternate means to produce polyketide compounds.

Polyketide synthase (PKS) enzymes are complexes of multiple large proteins. PKSs catalyse the biosynthesis of polyketides through repeated, decarboxylative Claisen condensations between acylthioester building blocks. PKS enzymes are generally classified into Type I or “modular” PKSs and Type II or “iterative” PKSs according to the type of polyketide synthetized and the mode by which the polyketide is synthesized. Type I PKSs are responsible for producing a large number of 12-, 14- and 16- membered macrolide antibiotics.

Type I or modular PKS enzymes are formed by a set of separate catalytic active sites for each cycle of carbon chain elongation and modification in the polyketide synthesis pathway. Each active site is termed a domain. A set of active sites is termed a module. The typical modular PKS multienzyme system is composed of several large polypeptides, which can be segregated from amino to carboxy termini into a loading module, multiple extender modules, and a releasing module that frequently contains a thioesterase domain.

Generally, the loading module is responsible for binding the first building block used to synthesize the polyketide and transferring it to the first extender module. The loading molecule recognizes a particular acyl-CoA (usually acetyl or propionyl but sometimes butyryl or other acyl-CoA) and transfers it as a thiol ester to the ACP of the loading module.

The AT on each of the extender modules recognizes a particular extender-CoA and transfers it to the ACP of that extender module to form a thioester. Each extender module is responsible for accepting a compound from a prior module, binding a building block, attaching the building block to the compound from the prior module, optionally performing one or more additional functions, and transferring the resulting compound to the next module.

Each extender module of all modular PKS reported to date contains a KS, AT, ACP, and zero, one, two or three domains that modify the beta-carbon of the growing polyketide chain. A typical (non-loading) minimal Type I PKS extender may contain a KS domain, an AT domain, and an ACP domain. Such domains are sufficient to activate a 2-carbon extender unit and attach it to the growing polyketide molecule. The next extender module, in turn, is responsible for attaching the next building block and transferring the growing compound to the next extender module until synthesis is complete.

Once the PKS is primed with acyl-ACPs, the acyl group of the loading module is transferred to form a thiol ester (trans-esterification) at the KS of the first extender module; at this stage, extender module one possesses an acyl-KS and a malonyl- (or substituted malonyl- ) ACP. The acyl group derived from the loading module is then covalently attached to the alpha-carbon of the malonyl group to form a carbon-carbon bond, driven by concomitant decarboxylation, and generating a new acyl-ACP that has a backbone two carbons longer than the loading building block (elongation or extension).

The polyketide chain, growing by two carbons with each extender module, is sequentially passed as covalently bound thiol esters from extender module to extender module, in an assembly line-like process. The carbon chain produced by this process alone would possess a ketone at every other carbon atom, producing a polyketone, from which the name polyketide arises. Most commonly, however, additional enzymatic activities modify the beta keto group of each two carbon unit just after it has been added to the growing polyketide chain but before it is transferred to the next module.

In addition to the typical KS, AT, and ACP domains necessary to form the carbon-carbon bond, a module may contain other domains that modify the beta-carbonyl moiety. For example, modules may contain a ketoreductase (KR) domain that reduces the keto group to an alcohol. Modules may also contain a KR domain plus a dehydratase (DH) domain that dehydrates the alcohol to a double bond. Modules may also contain a KR domain, a DH domain, and an enoylreductase (ER) domain that converts the double bond product to a saturated single bond. An extender module can also contain other enzymatic activities, such as, for example, a methylase or dimethylase activity.

After traversing the final extender module, the polyketide encounters a releasing domain that cleaves the polyketide from the PKS and typically cyclizes the polyketide. The polyketide can be further modified by tailoring enzymes; these enzymes add carbohydrate groups or methyl groups, or make other modifications, i.e. oxidation or reduction, on the polyketide core molecule.

In type I PKS polypeptides, the order of catalytic domains has been conserved in all type I PKSs reported to date. Thus, when all beta-keto processing domains are present in a module, the order of domains in that module from N-to-C-terminus has always been found to be KS, AT, DH, ER, KR, and ACP. Some or all of the beta-keto processing domains may be missing in particular modules, but the order of the domains present in a module has remained the same in all reported cases.

Engineering of these enzymes is achieved by modifying, adding, or deleting domains, or replacing them with those taken from other type I PKS enzymes. It is also achieved by deleting, replacing, or adding entire modules with those taken from other sources. A genetically engineered PKS complex should of course have the ability to catalyze the synthesis of the product predicted from the genetic alterations made.

Between the catalytic domains and at the N- and C-termini of individual polypeptides there are linker regions. The sequences of these linker regions are less well conserved than are those for the catalytic domains. Linker regions can be important for proper association between domains and between the individual polypeptides that comprise the PKS complex. One can thus view the linkers and domains together as creating a scaffold on which the domains and modules are positioned in the correct orientation to be active. This organization and positioning, if retained, permits PKS domains of different or identical substrate specificities to be substituted (usually at the DNA level) between PKS enzymes by various available methodologies. In selecting the boundaries of, for example, an AT replacement, one can thus make the replacement so as to retain the linkers of the recipient PKS or to replace them with the linkers of the donor PKS AT domain, or, preferably, make both constructs to ensure that the correct linker regions between the KS and AT domains have been included in at least one of the engineering enzymes. Thus, there is considerable flexibility in the design of new PKS enzymes with the result that known polyketides can be produced more effectively, and novel polyketides can be made.

Although large numbers of therapeutically important polyketides have been identified, there remains a need to obtain novel polyketides that have enhanced properties such as better pharmacokinetic profile and metabolism and fewer side effects. In addition there is a need to obtain novel polyketides that possess completely novel bioactivities. The complex polyketides produced by modular type I PKSs are particularly valuable, in that they include compounds with known utility as antihelminthics, insecticides, immunosuppressants, antifungal or antibacterial agents. Because of their structural complexity, such novel polyketides are not readily obtainable by total chemical synthesis, or by chemical modifications of known polyketides.

### SUMMARY OF THE INVENTION

The present invention advantageously provides genes and proteins involved in the production of polyketides. Specific embodiments of the genes and proteins are provided in the accompanying sequence listing. SEQ ID NOS: 1, 3, 5, 7, 9, 11, 13, 15, 17, 19, 21, 22 and 24 provide nucleic acids responsible for biosynthesis of the polyketide dorrigocin. SEQ ID NOS: 2, 4, 6, 8, 10, 12, 14, 16, 18, 20 and 23 provide amino acid sequences for proteins responsible for biosynthesis of the polyketide dorrigocin. SEQ ID NOS: 25, 27, 29, 31, 33, 35, 37, 39, 41 and 32 provide nucleic acid sequences for genes responsible for biosynthetisis of the polyketide lactimidomycin. SEQ ID NOS: 26, 28, 30, 32, 34, 36, 38, 41 and 42 provide amino acid sequences for proteins responsible for biosynthesis of the polyketide lactimidomycin. The genes and proteins of the invention provide the machinery for producing novel polyketide-related compounds based on dorrigocin and lactimidomycin compounds

The invention discloses polyketide synthase (PKS) genes (SEQ ID NOS: 11, 13, 15, 33, 35 and 37) and proteins (SEQ ID NOS: 10, 12, 14, 32, 34 and 36) that can be used to produce a variety of polyketides, some of which are now produced only by fermentation, others of which are now produced by fermentation and chemical modification, and still others of which are novel polyketides which are now not produced either by fermentation or chemical modification. The invention allows direct manipulation of dorrigocin, lactimidomycin and related chemical structures via chemical engineering of the enzymes involved in the biosynthesis of dorrigocin and lactimidomycin, modifications which are presently not possible by chemical methodology because of complexity of the structures.

The invention can also be used to introduce “chemical handles” into normally inert positions that permit subsequence chemical modifications. Several general approaches to achieve the development of novel polyketides are facilitated by the methods and reagents of the present invention. For example, molecular modeling can be used to predict optimal structures. Various polyketide structures can be generated by genetic manipulation of the dorrigocin gene cluster or the lactimidomycin gene cluster in accordance with the methods of the invention. The invention can be used to generate a focused library of analogs around a polyketide lead candidate to fine-tune the compound for optimal properties. Genetic engineering methods of the invention can be directed to modify positions of the molecule previously inert to chemical modifications. Known techniques allow one to manipulate a known PKS gene cluster either to produce the polyketide synthesized by that PKS at higher levels than occur in nature or in hosts that otherwise do not produce the polyketide. Known techniques allow one to produce molecules that are structurally related to, but distinct from the polyketides produces from known PKS gene clusters. See, for example, PCT publications WO 93/3663; 95/08548; 96/40968; 97/02358; 98/49315; U.S. Pat. Nos. 4,874,748; 5,063,155; 5,098,837; 5,149,639; 5,672,491; 5,712,146; 5,830,750; and 5,843,718.

Thus, in a first aspect the invention provides an isolated, purified nucleic acid or enriched comprising a sequence selected from the group consisting of SEQ ID NOS: 1, 22 and 25; the sequences complementary to SEQ ID NOS: 1, 22 and 25; fragments comprising at least 10, 15, 20, 25, 30, 35, 40, 50, 75, 100, 200 or 500 consecutive nucleotides of SEQ ID NO: 1, 22 and 25; and fragments comprising at least 10, 15, 20, 25, 30, 35, 40, 50, 75, 100, 200 or 500 consecutive nucleotides of the sequences complementary to SEQ ID NOS: 1, 22 and 25. Preferred embodiments of this aspect include isolated, purified or enriched nucleic acids capable of hybridizing to the above sequences under conditions of moderate or high stringency; isolated, purified or enriched nucleic acid comprising at least 10, 15, 20, 25, 30, 35, 40, 50, 75, 100, 200 or 500 consecutive bases of the above sequences; and isolated, purified or enriched nucleic acid having at least 70%, 75%, 80%, 85%, 90%, 95%, 97% or 99% homology to the above sequences as determined by analysis with BLASTN version 2.0 with the default parameters.

More preferred embodiments of this aspect of the invention include an isolated, purified or enriched nucleic acid comprising a sequence selected from the group consisting of SEQ ID NOS: 3, 5, 7, 9, 11, 13, 15, 17, 19, 21, 24, 27, 29, 31, 33, 35, 37, 39, 41, 43 and the sequences complementary thereto; an isolated, purified or enriched nucleic acid comprising at least 10, 15, 20, 25, 30, 35, 40, 50, 75, 100, 200 or 500 consecutive bases of a sequence selected from the group consisting of SEQ ID NOS: 3, 5, 7, 9, 11, 13, 15, 17, 19, 21, 24, 27, 29, 31, 33, 35, 37, 39, 41, 43 and the sequences complementary thereto; and an isolated, purified or enriched nucleic acid capable of hybridizing to the above listed nucleic acids under conditions of moderate or high stringency, and isolated, purified or enriched nucleic acid having at least 70%, 75%, 80%, 85%, 90%, 95%, 97% or 99% homology to the nucleic acid of claim 6 as determined by analysis with BLASTN version 2.0 with the default parameters.

Still more preferred embodiments of this aspect of the invention include an isolated nucleic acid that encodes a domains of the PKSs of SEQ ID NOS: 10, 12, 14, 32, 34 and 36; isolated nucleic acid that encodes all or part of one or more modules of the PKSs of SEQ ID NOS: 10, 12, 14, 32, 34 and 36. These nucleic acids can be readily used, alone or in combination with nucleic acids encoding other PKS domains or modules as intermediates in the construction of recombinant vectors. In another aspect, the invention provides an isolated nucleic acid that encodes all or a part of a PKS that contains at least one module in which at least one of the domains in the module is a domain from a non-dorrigocin PKS and non-lactimidomycin PKS and at least one domain is from a dorrigocin or lactimidomycin PKS.

In a second embodiment, the invention provides an isolated or purified polypeptide comprising a sequence selected from the group consisting of SEQ ID NOS: 2, 4, 6, 8, 10, 12, 14, 16, 18, 20, 23, 26, 28, 30, 32, 34, 36, 38, 40 and 42; an isolated or purified polypeptide comprising at least 10, 15, 20, 25, 30, 35, 40, 50, 75, 100, 200 or 500 consecutive amino acids of the polypeptides of SEQ ID NOS: 2, 4, 6, 8, 10, 12, 14, 16, 18, 20, 23, 26, 28, 30, 32, 34, 36, 38, 40 and 42; and an isolated or purified polypeptide having at least 70%, 75%, 80%, 85%, 90%, 95%, 97%, or 99% homology to the polypeptide of SEQ ID NOS: 2, 4, 6, 8, 10, 12, 14, 16, 18, 20, 23, 26, 28, 30, 32, 34, 36, 38, 40 and 42 as determined by analysis with BLASTP version 2.2.2 with the default parameters. In a further aspect, the invention provides a polypeptide comprising one or two or three or five or more or the above polypeptide sequences.

The invention also provides recombinant DNA expression vectors containing the above nucleic acids. These genes and the methods of the invention enable one skilled in the art to create recombinant host cells with the ability to produce polyketides. Thus, the invention provides a method of preparing a polyketide, said method comprising transforming a heterologous host cell with a recombinant DNA vector that encodes at least one of the above nucleic acids, and culturing said host cell under conditions such that a PKS is produced, which PKS catalyzes synthesis of a polyketide. In one aspect, the method is practiced with a Streptomyces host cell. In another aspect, the polyketide produced is dorrigocin or lactimidomycin. In another aspect, the polyketide produced is a polyketide related in structure to dorrigocin or lactimidomycin. One embodiment of this aspect of the invention is a method of expressing a dorrigocin biosynthetic gene product comprising culturing a host cell under conditions that permit expression of the dorrigocin biosynthetic gene product. A second embodiment of this aspect of the invention is a method of expressing a lactimidomycin biosynthetic gene product comprising culturing a host cell under conditions that permit expression of the lactimidomycin biosynthetic gene product.

The invention also encompasses a reagent comprising a probe of the invention for detecting and/or isolating putative polyketide-producing microorganisms; and a method for detecting and/or isolating putative polyketide-producing microorganisms using a probe of the invention such that hybridization is detected. Cloning, analysis, and manipulation by recombinant DNA technology of genes that encode PKS enzymes can be performed according to known techniques.

### BRIEF DESCRIPTION OF THE DRAWINGS

The present invention will be further understood from the following description with reference to the following figures:

FIG. 1 shows a diagram of the dorrigocin biosynthetic gene cluster of S. platensis highlighting the deduced domain architecture of the unusual PKS components.

FIG. 2 shows one proposed biosynthetic pathway for dorrigocins and migrastatin.

FIG. 3 illustrates the structures of the dorrigocins, migrastatin, and isomigrastatin.

FIG. 4 shows a diagram comparing the lactimidomycin biosynthetic gene cluster of S. amphibiosporus and the dorrigocin biosynthetic gene cluster of S. platensis. The deduced domain architecture of the unusual PKS components is highlighted.

FIG. 5 shows one proposed biosynthetic pathway for lactimidomycin.

FIG. 6 shows an amino acid alignment comparing DORR ORF 2 (SEQ ID NO: 4) to its lactimidomycin homologue, LACT ORF 1 (SEQ ID NO: 26), both of which are fusions of an acyltransferase and a thioesterase designated as AYTT.

FIG. 7 shows an amino acid alignment comparing DORR ORF 3 (SEQ ID NO: 6) to its lactimidomycin homologue, LACT ORF 2 (SEQ ID NO: 28), both of which are acyl carrier proteins designated as ACPI.

FIG. 8 shows an amino acid alignment comparing DORR ORF 4 (SEQ ID NO: 8) to its lactimidomycin homologue, LACT ORF 3 (SEQ ID NO: 30), both of which are amidotransferases similar to bacterial asparagine synthetases designated as AOTF.

FIGS. 9A to 9D shows an amino acid alignment comparing DORR ORF 5 (SEQ ID NO: 10) to its lactimidomycin homologue, LACT ORF 4 (SEQ ID NO: 32), both of which are unusual modular PKSs devoid of AT domains designated as PKUN.

FIGS. 10A to 10J show an amino acid alignment comparing DORR ORF 6 (SEQ ID NO: 12) to its lactimidomycin homologue, LACT ORF 5 (SEQ ID NO: 34), both of which are unusual modular PKSs devoid of AT domains designated as PKUN.

FIGS. 11A to 11C show an amino acid alignment comparing DORR ORF 7 (SEQ ID NO: 14) to its lactimidomycin homologue, LACT ORF 6 (SEQ ID NO: 12), both of which are unusual modular PKSs devoid of AT domains designated as PKUN.

FIG. 12 shows an amino acid alignment comparing DORR ORF 8 (SEQ ID NO: 16) to its lactimidomycin homologue, LACT ORF 7 (SEQ ID NO: 38), both of which are fusions of an acyltransferase and an oxidoreductase designated as AYOA.

FIG. 13 shows an amino acid alignment comparing DORR ORF 11 (SEQ ID NO: 23) to its lactimidomycin homologue, LACT ORF 8 (SEQ ID NO: 40), both of which are cytochrome P450 monooxygenases designated as OXRC.

### DETAILED DESCRIPTION OF THE INVENTION:

Some authors have distinguished between dorrigocin and migrastatin molecules. Throughout the specification reference to dorrigocin is intended to encompass the molecules referred to by some authors as migrastatin and isomigrastatin. Likewise reference to the biosynthetic locus for dorrigocin is intended to encompass the biosynthetic locus that directs the synthesis of the molecules some authors have referred to as migrastatin and isomigrastatin.

Throughout the description and the figures, the biosynthetic locus for dorrigocin from Streptomyces platensis subsp. rosaceus NRRL 18993 is sometimes referred to as DORR and the biosynthetic locus for lactimidomycin from Streptomyces amphibiosporus ATCC 53964 is sometimes referred to as LACT. The ORFs in DORR and LACT are assigned a putative function and grouped together in families based on homology to known proteins. To correlate structure and function, the protein families are given a four-letter designation used throughout the description and figures as indicated in Table I.

TABLE 1

| Families | Function |
|---|---|
| REBP | regulator, multidomain; fusion of a pathway specific activator-type regulator with a protein |
| | containing domain homology to LuxR family regulators |
| AYTT | acyltransferase-thioesterase fusion; N-terminus shows strong homology to malonyl |
| | CoA:ACP transacylases; C-terminal region shows strong homology to thioesterases |
| ACPI | acyl carrier protein; similar to proteins that may carry aminoacyl groups; similar to |
| | undecylprodigiosin RedO and coumermycin ProC PCPs that tether prolyl groups that |
| | may serve as substrates for oxidation while tethered |
| AOTF | amidotransferase, ATP-dependent, asparaginase; asparagine synthetases class B |
| | (glutamine-hydrolyzing); glutamine amidotransferase/asparagine synthase; asparagine |
| | synthetases (glutamine amidotransferases); catalyze the transfer of the carboxamide |
| | amino group of glutamine to the carboxylate group of aspartate. |
| PKUN | unusual polyketide synthase; devoid of AT domains; strong homology to B. subtilis Pks K |
| | and Pks M proteins found in an unknown polyketide locus |
| AYOA | acyltransferase-oxidoreductase fusion; strong homology to B. subtilis PksE fusion protein |
| | found in unknown polyketide locus; N-terminus shows strong homology to malonyl |
| | CoA:ACP transacylases; C-terminal region shows strong homology to 2-nitropopane |
| | dioxygenase-like enzymes found in loci required for polyunsaturated fatty acid |
| | (eicosapentaenoic acid) or polyketide biosynthesis |
| OXRY | oxidoreductase; zinc-binding, NADP-dependent dehydrogenase; similar to quinone |
| | oxidoreductases |
| MTFA | methyltransferase, SAM-dependent; includes O-methyltransferases, N,N- |
| | dimethyltransferases (e.g. spinosyn SpnS N-dimethyltransferase), C-methyltransferases |
| OXRC | oxidoreductase; cytP450 monooxygenase, hydroxylase; includes PikC, DoxA, FkbD |
| PPTF | phosphopantetheinyl transferases, required for activation of both PKSs and NRPSs from |
| | inactive apo forms to active holo forms; homology to B. subtilis Sfp, Anabaena Hetl, E. |
| | coli EntD and AcpS |

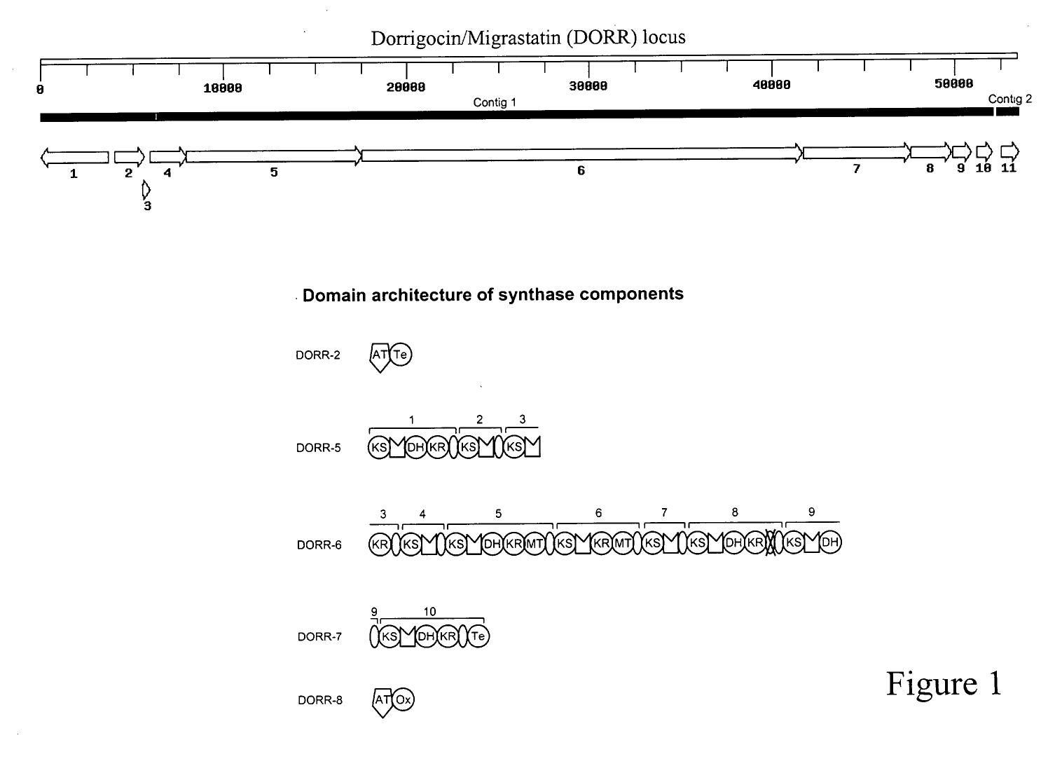

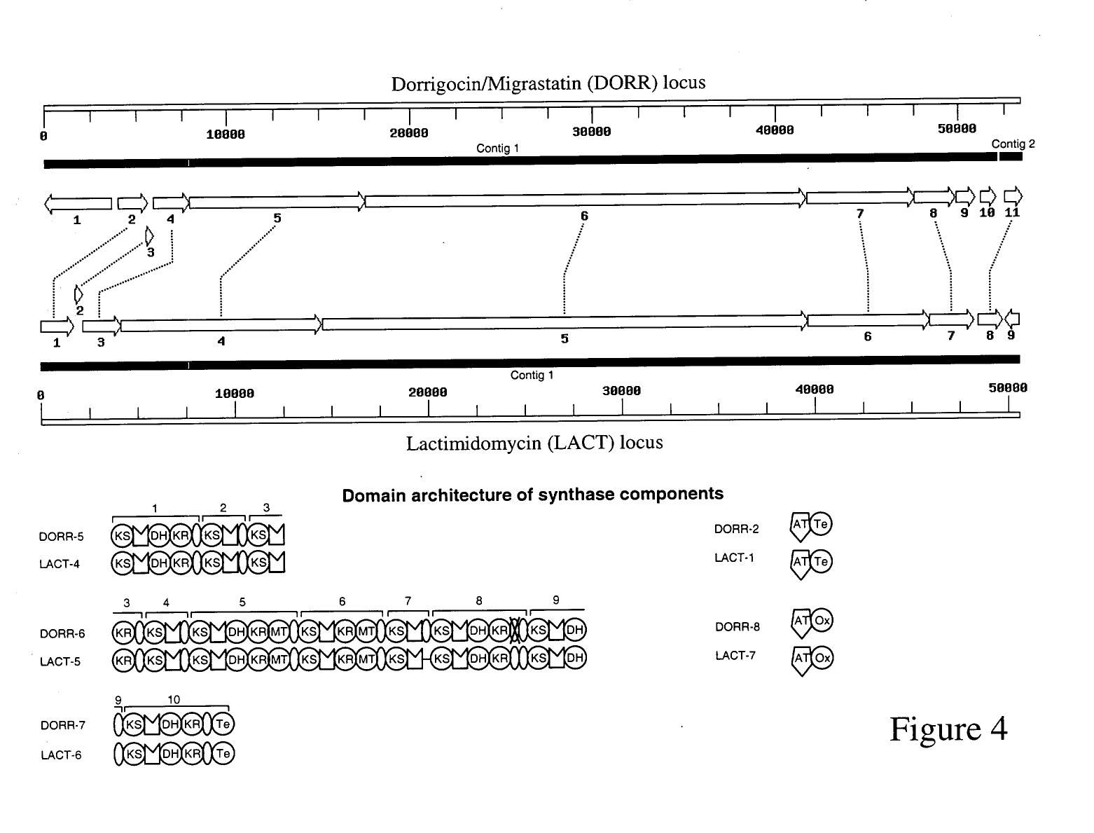

The term dorrigocin biosynthetic gene product refers to any enzyme involved in the biosynthesis of dorrigocin, migrastatin or isomigrastatin. These genes are located in the dorrigocin biosynthetic locus from Streptomyces platensis subsp. rosaceus. This locus is depicted in FIGS. 1 and 4. For the sake of particularity the dorrigocin biosynthetic pathway is associated with Streptomyces platensis subsp. rosaceus. However, it should be understood that this term encompasses dorrigocin biosynthetic enzymes (and genes encoding such enzymes) isolated from any microorganism of the genus Streptomyces, and furthermore that these genes may have novel homologues in related actinomycete microorganisms that fall within the scope of the claims here. In specific embodiments, the genes are listed in SEQ ID NOS: 2, 4, 6, 8, 10, 12, 14, 16, 18, 20 and 23.

The term lactimidomycin biosynthetic gene product refers to any enzyme involved in the biosynthesis of lactimidomycin. These genes are located in the lactimidomycin biosynthetic locus from Streptomyces amphibiosporus. This locus is depicted in FIG. 4. For the sake of particularity the lactimidomycin biosynthetic pathway is associated with Streptomyces amphibiosporus. However, it should be understood that this term encompasses lactimidomycin biosynthetic enzymes (and genes encoding such enzymes) isolated from any microorganism of the genus Streptomyces, and furthermore that these genes may have novel homologues in related actinomycete microorganisms that fall within the scope of the claims here. In specific embodiments, the genes are listed in SEQ ID NOS: 26, 28, 30, 32, 34, 36, 38, 40 and 42.

The term “isolated” means that the material is removed from its original environment, e.g. the natural environment if it is naturally occurring. For example, a naturally-occurring polynucleotide or polypeptide present in a living organism is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

The term “purified” does not require absolute purity; rather, it is intended as a relative definition. Individual nucleic acids obtained from a library have been conventionally purified to electrophoretic homogeneity. The sequences obtained from these clones could not be obtained directly from a large insert library, such as a cosmid library, or from total organism DNA. The purified nucleic acids of the present invention have been purified from the remainder of the genomic DNA in the organism by at least 10⁴ to 10⁶ fold. However, the term “purified” also includes nucleic acids which have been purified from the remainder of the genomic DNA or from other sequences in a library or other environment by at least one order of magnitude, preferably two or three orders of magnitude, and more preferably four or five orders of magnitude.

“Recombinant” means that the nucleic acid is adjacent to “backbone” nucleic acid to which it is not adjacent in its natural environment. “Enriched” nucleic acids represent 5% or more of the number of nucleic acid inserts in a population of nucleic acid backbone molecules. “Backbone” molecules include nucleic acids such as expression vectors, self-replicating nucleic acids, viruses, integrating nucleic acids, and other vectors or nucleic acids used to maintain or manipulate a nucleic acid of interest. Preferably, the enriched nucleic acids represent 15% or more, more preferably 50% or more, and most preferably 90% or more, of the number of nucleic acid inserts in the population of recombinant backbone molecules.

“Recombinant” polypeptides or proteins refers to polypeptides or proteins produced by recombinant DNA techniques, i.e. produced from cells transformed by an exogenous DNA construct encoding the desired polypeptide or protein. “Synthetic”polypeptides or proteins are those prepared by chemical synthesis.

The term “gene” means the segment of DNA involved in producing a polypeptide chain; it includes regions preceding and following the coding region (leader and trailer) as well as, where applicable, intervening regions (introns) between individual coding segments (exons).

A DNA or nucleotide “coding sequence” or “sequence encoding” a particular polypeptide or protein, is a DNA sequence which is transcribed and translated into a polypeptide or protein when placed under the control of appropriate regulatory sequences.

“Oligonucleotide” refers to a nucleic acid, generally of at least 10, preferably 15 and more preferably at least 20 nucleotides, preferably no more than 100 nucleotides, that are hybridizable to a genomic DNA molecule, a cDNA molecule, or an mRNA molecule encoding a gene, mRNA, cDNA or other nucleic acid of interest.

A promoter sequence is “operably linked to” a coding sequence recognized by RNA polymerase which initiates transcription at the promoter and transcribes the coding sequence into mRNA.

“Plasmids” are designated by a lower case p preceded or followed by capital letters and/or numbers. The starting plasmids herein are commercially available, publicly available on an unrestricted basis, or can be constructed from available plasmids in accord with published procedures. In addition, equivalent plasmids to those described herein are known in the art and will be apparent to the skilled artisan.

“Digestion” of DNA refers to enzymatic cleavage of the DNA with a restriction enzyme that acts only at certain sequences in the DNA. The various restriction enzymes used herein are commercially available and their reaction conditions, cofactors and other requirements were used as would be known to the ordinary skilled artisan. For analytical purposes, typically 1 μg of plasmid or DNA fragment is used with about 2 units of enzyme in about 20 μl of buffer solution. For the purpose of isolating DNA fragments for plasmid construction, typically 5 to 50 μg of DNA are digested with 20 to 250 units of enzyme in a larger volume. Appropriate buffers and substrate amounts for particular enzymes are specified by the manufacturer. Incubation times of about 1 hour at 37° C. are ordinarily used, but may vary in accordance with the supplier's instructions. After digestion the gel electrophoresis may be performed to isolate the desired fragment.

We have now discovered the genes and proteins involved in the biosynthesis of the polyketides dorrigocin and lactimidomycin. Nucleic acid sequences encoding proteins involved in the biosynthesis of dorrigocin are provided in the accompanying sequence listing as SEQ ID NOS: 3, 5, 7, 9, 11, 13, 15, 17, 19, 21 and 24. Polypeptides involved in the biosynthesis of dorrigocin are provided in the accompanying sequence listing as SEQ ID NOS: 2, 4, 6, 8, 10, 12, 14, 16, 18, 20 and 23. Nucleic acid sequences encoding proteins involved in the biosynthesis of lactimidomycin are provided in the accompanying sequence listing as SEQ ID NOS: 27, 29, 31, 33, 35, 37, 41 and 43. Polypeptides involved in the biosynthesis of lactimidomycin are provided in the accompanying sequence listing as SEQ ID NOS: 26, 28, 30, 32, 34, 36, 38, 40 and 42.

One aspect of the present invention is an isolated, purified, or enriched nucleic acid comprising one of the sequences of SEQ ID NOS: 3, 5, 7, 9, 11, 13, 15, 17, 19, 21, 24, 27, 29, 31, 33, 35, 37, 39 and 41, the sequences complementary thereto, or a fragment comprising at least 10, 15, 20, 25, 30, 35, 40, 50, 75, 100, 150, 200, 300, 400 or 500 consecutive bases of one of the sequences of SEQ ID NOS: 3, 5, 7, 9, 11, 13, 15, 17, 19, 21, 24, 27, 29, 31, 33, 35, 37, 39 and 41 or the sequences complementary thereto. The isolated, purified or enriched nucleic acids may comprise DNA, including cDNA, genomic DNA, and synthetic DNA. The DNA may be double stranded or single stranded, and if single stranded may be the coding or non-coding (anti-sense) strand. Alternatively, the isolated, purified or enriched nucleic acids may comprise RNA.

As discussed in more detail below, the isolated, purified or enriched nucleic acids of one of SEQ ID NOS: may be used to prepare one of the polypeptides of SEQ ID NOS: 2, 4, 6, 8, 10, 12, 14, 16, 18, 20, 23, 26, 28, 30, 32, 34, 36, 38, 40 and 42 or fragments comprising at least 5, 10, 15, 20, 25, 30, 35, 40, 50, 75, 100 or 100 consecutive amino acids of one of the polypeptides of SEQ ID NO: 2, 4, 6, 8, 10, 12, 14, 16, 18, 20, 23, 26, 28, 30, 32, 34, 36, 38, 40 and 42.

Accordingly, another aspect of the present invention is an isolated, purified or enriched nucleic acid which encodes one of the polypeptides of SEQ ID NOS: 2, 4, 6, 8, 10, 12, 14, 16, 18, 20, 23, 26, 28, 30, 32, 34, 36, 38, 40 and 42, or fragments comprising at least 5, 10, 15, 20, 25, 30, 35, 40, 50, 75, 100 or 150 consecutive amino acids of one of the polypeptides of SEQ ID NOS: 2, 4, 6, 8, 10, 12, 14, 16, 18, 20, 23, 26, 28, 30, 32, 34, 36, 38, 40 and 42. The coding sequences of these nucleic acids may be identical to one of the coding sequences of one of the nucleic acids of SEQ ID NOS: 3, 5, 7, 9, 11, 13, 15, 17, 19, 21, 24, 27, 29, 31, 33, 35, 37, 39 and 41 or a fragment thereof or may be different coding sequences which encode one of the polypeptides of SEQ ID NOS: 2, 4, 6, 8, 10, 12, 14, 16, 18, 20, 23, 26, 28, 30, 32, 34, 36, 38, 40 and 42, or fragments comprising at least 5, 10, 15, 20, 25, 30, 35, 40, 50, 75, 100 or 150 consecutive amino acids of one of the polypeptides of SEQ ID NOS: 2, 4, 6, 8, 10, 12, 14, 16, 18, 20, 23, 26, 28, 30, 32, 34, 36, 38, 40 and 42 as a result of the redundancy or degeneracy of the genetic code. The genetic code is well known to those of skill in the art and can be obtained, for example, from Stryer, Biochemistry, 3^{rd} edition, W. H. Freeman & Co., New York.

The isolated, purified or enriched nucleic acid which encodes one of the polypeptides of SEQ ID NOS: 2, 4, 6, 8, 10, 12, 14, 16, 18, 20, 23, 26, 28, 30, 32, 34, 36, 38, 40 and 42, may include, but is not limited to: (1) only the coding sequences of one of SEQ ID NOS: 3, 5, 7, 9, 11, 13, 15, 17, 19, 21, 24, 27, 29, 31, 33, 35, 37, 39 and 41; (2) the coding sequences of SEQ ID NOS: 3, 5, 7, 9, 11, 13, 15, 17, 19, 21, 24, 27, 29, 31, 33, 35, 37, 39 and 41 and additional coding sequences, such as leader sequences or proprotein; and (3) the coding sequences of SEQ ID NOS: 3, 5, 7, 9, 11, 13, 15, 17, 19, 21, 24, 27, 29, 31, 33, 35, 37, 39 and 41 and non-coding sequences, such as introns or non-coding sequences 5′ and/or 3′ of the coding sequence. Thus, as used herein, the term “polynucleotide encoding a polypeptide” encompasses a polynucleotide which includes only coding sequence for the polypeptide as well as a polynucleotide which includes additional coding and/or non-coding sequence.

The invention relates to polynucleotides based on SEQ ID NOS: 3, 5, 7, 9, 11, 13, 15, 17, 19, 21, 24, 27, 29, 31, 33, 35, 37, 39 and 41 but having polynucleotide changes that are “silent”, for example changes which do not alter the amino acid sequence encoded by the polynucleotides of SEQ ID NOS: 3, 5, 7, 9, 11, 13, 15, 17, 19, 21, 24, 27, 29, 31, 33, 35, 37, 39 and 41. The invention also relates to polynucleotides which have nucleotide changes which result in amino acid substitutions, additions, deletions, fusions and truncations of the polypeptides of SEQ ID NOS: 2, 4, 6, 8, 10, 12, 14, 16, 18, 20, 23, 26, 28, 30, 32, 34, 36, 38, 40 and 42. Such nucleotide changes may be introduced using techniques such as site directed mutagenesis, random chemical mutagenesis, exonuclease III deletion, and other recombinant DNA techniques.

The isolated, purified or enriched nucleic acids of SEQ ID NOS: 3, 5, 7, 9, 11, 13, 15, 17, 19, 21, 24, 27, 29, 31, 33, 35, 37, 39 and 41, the sequences complementary thereto, or a fragment comprising at least 10, 15, 20, 25, 30, 35, 40, 50, 75, 100, 150, 200, 300, 400 or 500 consecutive bases of one of the sequence of SEQ ID NOS: 3, 5, 7, 9, 11, 13, 15, 17, 19, 21, 24, 27, 29, 31, 33, 35, 37, 39 and 41, or the sequences complementary thereto may be used as probes to identify and isolate DNAs encoding the polypeptides of SEQ ID NOS: 2, 4, 6, 8, 10, 12, 14, 16, 18, 20, 23, 26, 28, 30, 32, 34, 36, 38, 40 and 42 respectively. In such procedures, a genomic DNA library is constructed from a sample microorganism or a sample containing a microorganism capable of producing a polyketide. The genomic DNA library is then contacted with a probe comprising a coding sequence or a fragment of the coding sequence, encoding one of the polypeptides of SEQ ID NOS: 2, 4, 6, 8, 10, 12, 14, 16, 18, 20, 23, 26, 28, 30, 32, 34, 36, 38, 40 and 42, or a fragment thereof under conditions which permit the probe to specifically hybridize to sequences complementary thereto. In a preferred embodiment, the probe is an oligonucleotide of about 10 to about 30 nucleotides in length designed based on a nucleic acid of SEQ ID NOS: 3, 5, 7, 9, 11, 13, 15, 17, 19, 21, 24, 27, 29, 31, 33, 35, 37, 39 and 41. Genomic DNA clones which hybridize to the probe are then detected and isolated. Procedures for preparing and identifying DNA clones of interest are disclosed in Ausubel et al., Current Protocols in Molecular Biology, John Wiley 503 Sons, Inc. 1997; and Sambrook et al., Molecular Cloning: A Laboratory Manual 2d Ed., Cold Spring Harbor Laboratory Press, 1989. In another embodiment, the probe is a restriction fragments or a PCT amplified nucleic acid derived from SEQ ID NOS: 3, 5, 7, 9, 11, 13, 15, 17, 19, 21, 24, 27, 29, 31, 33, 35, 37, 39 and 41.

The isolated, purified or enriched nucleic acids of SEQ ID NOS: 3, 5, 7, 9, 11, 13, 15, 17, 19, 21, 24, 27, 29, 31, 33, 35, 37, 39 and 41, the sequences complementary thereto, or a fragment comprising at least 10, 15, 20, 25, 30, 35, 40, 50, 75, 100, 150, 200, 300, 400 or 500 consecutive bases of one of the sequences of SEQ ID NOS: 3, 5, 7, 9, 11, 13, 15, 17, 19, 21, 24, 27, 29, 31, 33, 35, 37, 39 and 41, or the sequences complementary thereto may be used as probes to identify and isolate related nucleic acids. In some embodiments, the related nucleic acids may be genomic DNAs (or cDNAs) from potential polyketide producers. In a preferred embodiment isolated, purified or enriched nucleic acids of SEQ ID NOS: 11, 13, 15, 33, 35 and 37 the sequences complementary thereto, or a fragment comprising at least 10, 15, 20, 25, 30, 35, 40, 50, 75, 100, 150, 200, 300, 400 or 500 consecutive bases of one of the sequences of SEQ ID NOS: 11, 13, 15, 33, 35 and 37, or the sequences complementary thereto may be used as probes to identify and isolate related nucleic acids. In such procedures, a nucleic acid sample containing nucleic acids from a potential polyketide-producer is contacted with the probe under conditions which permit the probe to specifically hybridize to related sequences. The nucleic acid sample may be a genomic DNA (or cDNA) library from the potential polyketide-producer. Hybridization of the probe to nucleic acids is then detected using any of the methods described above.

Hybridization may be carried out under conditions of low stringency, moderate stringency or high stringency. As an example of nucleic acid hybridization, a polymer membrane containing immobilized denatured nucleic acids is first prehybridized for 30 minutes at 45° C. in a solution consisting of 0.9M NaCl, 50 mM NaH₂PO₄, pH 7.0, 5.0 mM Na₂EDTA, 0.5% SDS, 10X Denhardt's, and 0.5 mg/ml polyriboadenylic acid. Approximately 2×10⁷ cpm (specific activity 4-9×10⁸ cpm/ug) of ³²p end-labeled oligonucleotide probe are then added to the solution. After 12-16 hours of incubation, the membrane is washed for 30 minutes at room temperature in 1X SET (150 mM NaCl, 20 mM Tris hydrochloride, pH 7.8, 1 mM Na₂EDTA) containing 0.5% SDS, followed by a 30 minute wash in fresh 1X SET at Tm-10 C for the oligonucleotide probe where Tm is the melting temperature. The membrane is then exposed to auto-radiographic film for detection of hybridization signals.

By varying the stringency of the hybridization conditions used to identify nucleic acids, such as genomic DNAs or cDNAs, which hybridize to the detectable probe, nucleic acids having different levels of homology to the probe can be identified and isolated. Stringency may be varied by conducting the hybridization at varying temperatures below the melting temperatures of the probes. The melting temperature of the probe may be calculated using the following formulas:

For oligonucleotide probes between 14 and 70 nucleotides in length the melting temperature (Tm) in degrees Celcius may be calculated using the formula:

Tm=81.5+16.6(log[Na+])+0.41(fraction G+C)−(600/N)

where N is the length of the oligonucleotide.

If the hybridization is carried out in a solution containing formamide, the melting temperature may be calculated using the equation Tm=81.5+16.6(log[Na +])+0.41 (fraction G+C)−(0.63% formamide)−(600/N) where N is the length of the probe.

Prehybridization may be carried out in 6X SSC, 5X Denhardt's reagent, 0.5% SDS, 0.1 mg/ml denatured fragmented salmon sperm DNA or 6X SSC, 5X Denhardt's reagent, 0.5% SDS, 0.1 mg/ml denatured fragmented salmon sperm DNA, 50% formamide. The composition of the SSC and Denhardt's solutions are listed in Sambrook et al., supra.

Hybridization is conducted by adding the detectable probe to the hybridization solutions listed above. Where the probe comprises double stranded DNA, it is denatured by incubating at elevated temperatures and quickly cooling before addition to the hybridization solution. It may also be desirable to similarly denature single stranded probes to eliminate or diminish formation of secondary structures or oligomerization. The filter is contacted with the hybridization solution for a sufficient period of time to allow the probe to hybridize to cDNAs or genomic DNAs containing sequences complementary thereto or homologous thereto. For probes over 200 nucleotides in length, the hybridization may be carried out at 15-25° C. below the Tm. For shorter probes, such as oligonucleotide probes, the hybridization may be conducted at 5-10° C. below the Tm. Preferably, the hybridization is conducted in 6X SSC, for shorter probes. Preferably, the hybridization is conducted in 50% formamide containing solutions, for longer probes.

All the foregoing hybridizations would be considered to be examples of hybridization performed under conditions of high stringency.

Following hybridization, the filter is washed for at least 15 minutes in 2X SSC, 0.1% SDS at room temperature or higher, depending on the desired stringency. The filter is then washed with 0.1X SSC, 0.5% SDS at room temperature (again) for 30 minutes to 1 hour.

Nucleic acids which have hybridized to the probe are identified by autoradiography or other conventional techniques.

The above procedure may be modified to identify nucleic acids having decreasing levels of homology to the probe sequence. For example, to obtain nucleic acids of decreasing homology to the detectable probe, less stringent conditions may be used. For example, the hybridization temperature may be decreased in increments of 5° C. from 68° C. to 42° C. in a hybridization buffer having a Na+ concentration of approximately 1M. Following hybridization, the filter may be washed with 2X SSC, 0.5% SDS at the temperature of hybridization. These conditions are considered to be “moderate stringency” conditions above 50° C. and “low stringency” conditions below 50° C. A specific example of “moderate stringency” hybridization conditions is when the above hybridization is conducted at 55° C. A specific example of “low stringency” hybridization conditions is when the above hybridization is conducted at 45° C.

Alternatively, the hybridization may be carried out in buffers, such as 6X SSC, containing formamide at a temperature of 42° C. In this case, the concentration of formamide in the hybridization buffer may be reduced in 5% increments from 50% to 0% to identify clones having decreasing levels of homology to the probe. Following hybridization, the filter may be washed with 6X SSC, 0.5% SDS at 50° C. These conditions are considered to be “moderate stringency” conditions above 25% formamide and “low stringency” conditions below 25% formamide. A specific example of “moderate stringency” hybridization conditions is when the above hybridization is conducted at 30% formamide. A specific example of “low stringency” hybridization conditions is when the above hybridization is conducted at 10% formamide.

Nucleic acids which have hybridized to the probe are identified by autoradiography or other conventional techniques.

For example, the preceding methods may be used to isolate nucleic acids having a sequence with at least 97%, at least 95%, at least 90%, at least 85%, at least 80%, or at least 70% homology to a nucleic acid sequence selected from the group consisting of the sequences of SEQ ID NOS: 3, 5, 7, 9, 11, 13, 15, 17, 19, 21, 24, 27, 29, 31, 33, 35, 37, 39 and 41, fragments comprising at least 10, 15, 20, 25, 30, 35, 40, 50, 75, 100, 150, 200, 300, 400, or 500 consecutive bases thereof, and the sequences complementary thereto. Homology may be measured using BLASTN version 2.0 with the default parameters. For example, the homologous polynucleotides may have a coding sequence which is a naturally occurring allelic variant of one of the coding sequences described herein. Such allelic variant may have a substitution, deletion or addition of one or more nucleotides when compared to the nucleic acids of SEQ ID NOS: 3, 5, 7, 9, 11, 13, 15, 17, 19, 21, 24, 27, 29, 31, 33, 35, 37, 39 and 41, or the sequences complementary thereto.

Additionally, the above procedures may be used to isolate nucleic acids which encode polypeptides having at least 99%, 95%, at least 90%, at least 85%, at least 80%, or at least 70% homology to a polypeptide having the sequence of one of SEQ ID NOS: 2, 4, 6, 8, 10, 12, 14, 16, 18, 20, 23, 26, 28, 30, 32, 34, 36, 38, 40 and 42, or fragments comprising at least 5, 10, 15, 20, 25, 30, 35, 40, 50, 75, 100, or 150 consecutive amino acids thereof as determined using the BLASTP version 2.2.2 algorithm with default parameters.

Another aspect of the present invention is an isolated or purified polypeptide comprising the sequence of one of SEQ ID NOS: 2, 4, 6, 8, 10, 12, 14, 16, 18, 20, 23, 26, 28, 30, 32, 34, 36, 38, 40 and 42 or fragments comprising at least 5, 10, 15, 20, 25, 30, 35, 40, 50, 75, 100, or 150 consecutive amino acids thereof. As discussed above, such polypeptides may be obtained by inserting a nucleic acid encoding the polypeptide into a vector such that the coding sequence is operably linked to a sequence capable of driving the expression of the encoded polypeptide in a suitable host cell. For example, the expression vector may comprise a promoter, a ribosome binding site for translation initiation and a transcription terminator. The vector may also include appropriate sequences for modulating expression levels, an origin of replication and a selectable marker.

Promoters suitable for expressing the polypeptide or fragment thereof in bacteria include the E.coli lac or trp promoters, the lad promoter, the lacZ promoter, the T3 promoter, the T7 promoter, the gpt promoter, the lambda PR promoter, the lambda PL promoter, promoters from operons encoding glycolytic enzymes such as 3-phosphoglycerate kinase (PGK), and the acid phosphatase promoter. Fungal promoters include the a factor promoter. Eukaryotic promoters include the CMV immediate early promoter, the HSV thymidine kinase promoter, heat shock promoters, the early and late SV40 promoter, LTRs from retroviruses, and the mouse metallothionein-I promoter. Other promoters known to control expression of genes in prokaryotic or eukaryotic cells or their viruses may also be used.

Mammalian expression vectors may also comprise an origin of replication, any necessary ribosome binding sites, a polyadenylation site, splice donors and acceptor sites, transcriptional termination sequences, and 5′ flanking nontranscribed sequences. In some embodiments, DNA sequences derived from the SV40 splice and polyadenylation sites may be used to provide the required nontranscribed genetic elements.

Vectors for expressing the polypeptide or fragment thereof in eukaryotic cells may also contain enhancers to increase expression levels. Enhancers are cis-acting elements of DNA, usually from about 10 to about 300 bp in length that act on a promoter to increase its transcription. Examples include the SV40 enhancer on the late side of the replication origin bp 100 to 270, the cytomegalovirus early promoter enhancer, the polyoma enhancer on the late side of the replication origin, and the adenovirus enhancers.

In addition, the expression vectors preferably contain one or more selectable marker genes to permit selection of host cells containing the vector. Examples of selectable markers that may be used include genes encoding dihydrofolate reductase or genes conferring neomycin resistance for eukaryotic cell culture, genes conferring tetracycline or ampicillin resistance in E. coli, and the S. cerevisiae TRP1 gene.

In some embodiments, the nucleic acid encoding one of the polypeptides of SEQ ID NOS: 2, 4, 6, 8, 10, 12, 14, 16, 18, 20, 23, 26, 28, 30, 32, 34, 36, 38, 40 and 42, or fragments comprising at least 5, 10, 15, 20, 25, 30, 35, 40, 50, 75, 100, or 150 consecutive amino acids thereof is assembled in appropriate phase with a leader sequence capable of directing secretion of the translated polypeptides or fragments thereof. Optionally, the nucleic acid can encode a fusion polypeptide in which one of the polypeptide of SEQ ID NOS: 2, 4, 6, 8, 10, 12, 14, 16, 18, 20, 23, 26, 28, 30, 32, 34, 36, 38, 40 and 42 or fragments comprising at least 5, 10, 15, 20, 25, 30, 35, 40, 50, 75, 100, or 150 consecutive amino acids thereof is fused to heterologous peptides or polypeptides, such as N-terminal identification peptides which impart desired characteristics such as increased stability or simplified purification or detection.

The appropriate DNA sequence may be inserted into the vector by a variety of procedures. In general, the DNA sequence is ligated to the desired position in the vector following digestion of the insert and the vector with appropriate restriction endonucleases. Alternatively, appropriate restriction enzyme sites can be engineered into a DNA sequence by PCR. A variety of cloning techniques are disclosed in Ausbel et al. Current Protocols in Molecular Biology, John Wiley 503 Sons, Inc. 1997 and Sambrook et al., Molecular Cloning: A Laboratory Manual 2d Ed., Cold Spring Harbour Laboratory Press, 1989. Such procedures and others are deemed to be within the scope of those skilled in the art.

The vector may be, for example, in the form of a plasmid, a viral particle, or a phage. Other vectors include derivatives of chromosomal, nonchromosomal and synthetic DNA sequences, viruses, bacterial plasmids, phage DNA, baculovirus, yeast plasmids, vectors derived from combinations of plasmids and phage DNA, viral DNA such as vaccinia, adenovirus, fowl pox virus, and pseudorabies. A variety of cloning and expression vectors for use with prokaryotic and eukaryotic hosts are described by Sambrook, et al., Molecular Cloning: A Laboratory Manual, Second Edition, Cold Spring Harbor, N.Y., (1989).

Particular bacterial vectors which may be used include the commercially available plasmids comprising genetic elements of the well known cloning vector pBR322 (ATCC 37017), pKK223-3 (Pharmacia Fine Chemicals, Uppsala, Sweden), GEM1 (Promega Biotec, Madison, Wis., U.S.A.) pQE70, pQE60, pQE-9 (Qiagen), pD10, psiX174 pBluescript II KS, pNH8A, pNH16a, pNH18A, pNH46A (Stratagene), ptrc99a, pKK223-3; pKK233-3, pDR540, pRIT5 (Pharmacia), pKK232-8 and pCM7. Particular eukaryotic vectors include pSV2CAT, pOG44, pXT1, pSG (Stratagene) pSVK3, pBPV, pMSG, and pSVL (Pharmacia). However, any other vector may be used as long as it is replicable and stable in the host cell.

The host cell may be any of the host cells familiar to those skilled in the art, including prokaryotic cells or eukaryotic cells. As representative examples of appropriate hosts, there may be mentioned: bacteria cells, such as E. coli, Streptomyces, Bacillus subtilis, Salmonella typhimurium and various species within the genera Pseudomonas, Streptomyces, and Staphylococcus, fungal cells, such as yeast, insect cells such as Drosophila S2 and Spodoptera Sf9, animal cells such as CHO, COS or Bowes melanoma, and adenoviruses. The selection of an appropriate host is within the abilities of those skilled in the art.

The vector may be introduced into the host cells using any of a variety of techniques, including electroporation transformation, transfection, transduction, viral infection, gene guns, or Ti-mediated gene transfer. Where appropriate, the engineered host cells can be cultured in conventional nutrient media modified as appropriate for activating promoters, selecting transformants or amplifying the genes of the present invention. Following transformation of a suitable host strain and growth of the host strain to an appropriate cell density, the selected promoter may be induced by appropriate means (e.g., temperature shift or chemical induction) and the cells may be cultured for an additional period to allow them to produce the desired polypeptide or fragment thereof.

Cells are typically harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment thereof can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

Various mammalian cell culture systems can also be employed to express recombinant protein. Examples of mammalian expression systems include the COS-7 lines of monkey kidney fibroblasts (described by Gluzman, Cell, 23:175(1981), and other cell lines capable of expressing proteins from a compatible vector, such as the C127, 3T3, CHO, HeLa and BHK cell lines.

The constructs in host cells can be used in a conventional manner to produce the gene product encoded by the recombinant sequence. Depending upon the host employed in a recombinant production procedure, the polypeptide produced by host cells containing the vector may be glycosylated or may be non-glycosylated. Polypeptides of the invention may or may not also include an initial methionine amino acid residue.

Alternatively, the polypeptides of SEQ ID NOs: 2, 4, 6, 8, 10, 12, 14, 16, 18, 20, 23, 26, 28, 30, 32, 34, 36, 38, 40 and 42, or fragments comprising at least 5, 10, 15, 20, 25, 30, 35, 40, 50, 75, 100, or 150 consecutive amino acids thereof can be synthetically produced by conventional peptide synthesizers. In other embodiments, fragments or portions of the polynucleotides may be employed for producing the corresponding full-length polypeptide by peptide synthesis; therefore, the fragments may be employed as intermediates for producing the full-length polypeptides.

Cell-free translation systems can also be employed to produce one of the polypeptides of SEQ ID NOS: 2, 4, 6, 8, 10, 12, 14, 16, 18, 20, 23, 26, 28, 30, 32, 34, 36, 38, 40 and 42, or fragments comprising at least 5, 10, 15, 20, 25, 30, 35, 40, 50, 75, 100, or 150 consecutive amino acids thereof using mRNAs transcribed form a DNA construct comprising a promoter operably linked to a nucleic acid encoding the polypeptide or fragment therof. In some embodiments, the DNA construct may be linearized prior to conducting an in vitro transcription reaction. The transcribed mRNA is then incubated with an appropriate cell-free translation extract, such as a rabbit reticulocyte extract, to produce the desired polypeptide or fragment thereof.

The present invention also relates to variants of the polypeptides of SEQ ID NOS: 2, 4, 6, 8, 10, 12, 14, 16, 18, 20, 23, 26, 28, 30, 32, 34, 36, 38, 40 and 42, or fragments comprising at least 5, 10, 15, 20, 25, 30, 35, 40, 50, 75, 100, or 150 consecutive amino acids thereof. The term “variant” includes derivatives or analogs of these polypeptides. In particular, the variants may differ in amino acid sequence from the polypeptides of SEQ ID NOS: 2, 4, 6, 8, 10, 12, 14, 16, 18, 20, 23, 26, 28, 30, 32, 34, 36, 38, 40 and 42, by one or more substitutions, additions, deletions, fusions and truncations, which may be present in any combination.

The variants may be naturally occurring or created in vitro. In particular, such variants may be created using genetic engineering techniques such as site directed mutagenesis, random chemical mutagenesis, Exonuclease III deletion procedures, and standard cloning techniques. Alternatively, such variants, fragments, analogs, or derivatives may be created using chemical synthesis or modification procedures.

Other methods of making variants are also familiar to those skilled in the art. These include procedures in which nucleic acid sequences obtained from natural isolates are modified to generate nucleic acids which encode polypeptides having characteristics which enhance their value in industrial or laboratory applications. In such procedures, a large number of variant sequences having one or more nucleotide differences with respect to the sequence obtained from the natural isolate are generated and characterized. Preferably, these nucleotide differences result in amino acid changes with respect to the polypeptides encoded by the nucleic acids from the natural isolates.

For example, variants may be created using error prone PCR. In error prone PCR, DNA amplification is performed under conditions where the fidelity of the DNA polymerase is low, such that a high rate of point mutation is obtained along the entire length of the PCR product. Error prone PCR is described in Leung, D. W., et al., Technique, 1:11-15 (19 89) and Caldwell, R. C. & Joyce G. F., PCR Methods Applic., 2:28-33 (1992). Variants may also be created using site directed mutagenesis to generate site-specific mutations in any cloned DNA segment of interest. Oligonucleotide mutagenesis is described in Reidhaar-Olson, J. F. & Sauer, R. T., et al., Science, 241:53-57 (1988). The variants of the polypeptides of SEQ ID NOS: 2, 4, 6, 8, 10, 12, 14, 16, 18, 20, 23, 26, 28, 30, 32, 34, 36, 38, 40 and 42, may be (i) variants in which one or more of the amino acid residues of the polypeptides of SEQ ID NOS: 2, 4, 6, 8, 10, 12, 14, 16, 18, 20, 23, 26, 28, 30, 32, 34, 36, 38, 40 and 42, are substituted with a conserved or non-conserved amino acid residue (preferably a conserved amino acid residue) and such substituted amino acid residue may or may not be one encoded by the genetic code.

Conservative substitutions are those that substitute a given amino acid in a polypeptide by another amino acid of like characteristics. Typically seen as conservative substitutions are the following replacements: replacements of an aliphatic amino acid such as Ala, Val, Leu and lie with another aliphatic amino acid; replacement of a Ser with a Thr or vice versa; replacement of an acidic residue such as Asp or Glu with another acidic residue; replacement of a residue bearing an amide group, such as Asn or Gln, with another residue bearing an amide group; exchange of a basic residue such as Lys or Arg with another basic residue; and replacement of an aromatic residue such as Phe or Tyr with another aromatic residue.

Other variants are those in which one or more of the amino acid residues of the polypeptides of SEQ ID Nos: 2, 4, 6, 8, 10, 12, 14, 16, 18, 20, 23, 26, 28, 30, 32, 34, 36, 38, 40 and 42 includes a substituent group.

Still other variants are those in which the polypeptide is associated with another compound, such as a compound to increase the half-life of the polypeptide (for example, polyethylene glycol).

Additional variants are those in which additional amino acids are fused to the polypeptide, such as leader sequence, a secretory sequence, a proprotein sequence or a sequence which facilitates purification, enrichment, or stabilization of the polypeptide.

In some embodiments, the fragments, derivatives and analogs retain the same biological function or activity as the polypeptides of SEQ ID NOS: 2, 4, 6, 8, 10, 12, 14, 16, 18, 20, 23, 26, 28, 30, 32, 34, 36, 38, 40 and 42. In other embodiments, the fragment, derivative or analogue includes a fused herterologous sequence which facilitates purification, enrichment, detection, stabilization or secretion of the polypeptide that can be enzymatically cleaved, in whole or in part, away from the fragment, derivative or analogue.

Another aspect of the present invention are polypeptides or fragments thereof which have at least 70%, at least 80%, at least 85%, at least 90%, or more than 95% homology to one of the polypeptides of SEQ ID NOS: 2, 4, 6, 8, 10, 12, 14, 16, 18, 20, 23, 26, 28, 30, 32, 34, 36, 38, 40 and 42, or a fragment comprising at least 5, 10, 15, 20, 25, 30, 35, 40, 50, 75, 100, or 150 consecutive amino acids thereof. Homology may be determined using a program, such as BLASTP version 2.2.2 with the default parameters, which aligns the polypeptides or fragments being compared and determines the extent of amino acid identity or similarity between them. It will be appreciated that amino acid “homology” includes conservative substitutions such as those described above.

The polypeptides or fragments having homology to one of the polypeptides of SEQ ID NOS: 2, 4, 6, 8, 10, 12, 14, 16, 18, 20, 23, 26, 28, 30, 32, 34, 36, 38, 40 and 42, or a fragment comprising at least 5, 10, 15, 20, 25, 30, 35, 40, 50, 75, 100, or 150 consecutive amino acids thereof may be obtained by isolating the nucleic acids encoding them using the techniques described above.

Alternatively, the homologous polypeptides or fragments may be obtained through biochemical enrichment or purification procedures. The sequence of potentially homologous polypeptides or fragments may be determined by proteolytic digestion, gel electrophoresis and/or microsequencing. The sequence of the prospective homologous polypeptide or fragment can be compared to one of the polypeptides of SEQ ID NOS: 2, 4, 6, 8, 10, 12, 14, 16, 18, 20, 23, 26, 28, 30, 32, 34, 36, 38, 40 and 42, or a fragment comprising at least 5, 10, 15, 20, 25, 30, 35, 40, 50, 75, 100, or 150 consecutive amino acids thereof using a program such as BLASTP version 2.2.2 with the default parameters.

The polypeptides of SEQ ID NOS: 2, 4, 6, 8, 10, 12, 14, 16, 18, 20, 23, 26, 28, 30, 32, 34, 36, 38, 40 and 42, or fragments, derivatives or analogs thereof comprising at least 5, 10, 15, 20, 25, 30, 35, 40, 50, 75, 100, or 150 consecutive amino acids thereof invention may be used in a variety of application. For example, the polypeptides or fragments, derivatives or analogs thereof may be used to biocatalyze biochemical reactions. In particular, the polypeptides of the AYTT family, namely SEQ ID NOS: 4 and 26 or fragments, derivatives or analogs thereof; the ACPI family, namely SEQ ID NOS: 6 and 28 or fragments, derivatives or analogs thereof; the AOTF family, namely SEQ ID NOS: 8 and 30 or fragments, derivatives or analogs thereof; the PKUN family namely SEQ ID NOS: 10, 12, 14, 32, 34 and 36 or fragments, derivatives or analogs thereof; the AYOA family namely SEQ ID NOS: 16 and 38 or fragments, derivatives or analogs thereof may be used in any combination, in vitro or in vivo, to direct the synthesis or modification of a polyketide or a substructure thereof. Polypeptides of the OXRY family, namely SEQ ID NO: 18 or fragments, derivatives or analogs thereof may be used, in vitro or in vivo, to catalyze oxidoreduction reactions that modify compounds that are either endogenously produced by the host, supplemented to the growth medium, or are added to a cell-free, purified or enriched preparation of OXRY polypeptide. Polypeptides of the MTFA family, namely SEQ ID NO: 20 or fragments, derivatives or analogs thereof may be used, in vitro or in vivo, to catalyze methylation reactions that modify compounds that are either endogenously produced by the host, supplemented to the growth medium, or are added to a cell-free, purified or enriched preparation of MTFA polypeptide. Polypeptides of the OXRC family, namely SEQ ID NOS: 23 and 40 or fragments, derivatives or analogs thereof may be used, in vitro or in vivo, to catalyze oxidation reactions that modify compounds that are either endogenously produced by the host, supplemented to the growth medium, or are added to a cell-free, purified or enriched preparation of OXRC polypeptide. Polypeptides of the PPTF family, namely SEQ ID NO: 42 or fragments, derivatives or analogs thereof may be used, in vitro or in vivo, to catalyze the phosphopanteteinylation of either acyl carrier proteins or domains; of thiolation protein or domains; or of peptidyl carrier proteins or domains.

The polypeptides of SEQ ID NOS: 2, 4, 6, 8, 10, 12, 14, 16, 18, 20, 23, 26, 28, 30, 32, 34, 36, 38, 40 and 42, or fragments, derivatives or analogues thereof comprising at least 5, 10, 15, 20, 25, 30, 35, 40, 50, 75, 100, or 150 consecutive amino acids thereof, may also be used to generate antibodies which bind specifically to the polypeptides or fragments, derivatives or analogues. The antibodies generated from SEQ ID NOS: 2, 4, 6, 8, 10, 12, 14, 16, 18, 20, and 23, may be used to determine whether a biological sample contains Streptomyces platensis subsp. rosaceus or a related microorganism. The antibodies generated from SEQ ID NOS: 26, 28, 30, 32, 34, 36, 38, 40 and 42, may be used to determine whether a biological sample contains Streptomyces amphibiosporus or a related microorganism. In such procedures, a biological sample is contacted with an antibody capable of specifically binding to one of the polypeptides of SEQ ID NOS: 2, 4, 6, 8, 10, 12, 14, 16, 18, 20, 23, 26, 28, 30, 32, 34, 36, 38, 40 and 42, or fragments comprising at least 5, 10, 15, 20, 25, 30, 35, 40, 50, 75, 100, or 150 consecutive amino acids thereof. The ability of the biological sample to bind to the antibody is then determined. For example, binding may be determined by labeling the antibody with a detectable label such as a fluorescent agent, an enzymatic label, or a radioisotope. Alternatively, binding of the antibody to the sample may be detected using a secondary antibody having such a detectable label thereon. A variety of assay protocols which may be used to detect the presence of Streptomyces platensis subsp. rosaceus or Streptomyces amphibiosporus or of polypeptides related to SEQ ID NOS: 2, 4, 6, 8, 10, 12, 14, 16, 18, 20, 23, 26, 28, 30, 32, 34, 36, 38, 40 and 42, in a sample are familiar to those skilled in the art. Particular assays include ELISA assays, sandwich assays, radioimmunoassays, and Western Blots. Alternatively, antibodies generated from SEQ ID NOS: 2, 4, 6, 8, 10, 12, 14, 16, 18, 20, 23, 26, 28, 30, 32, 34, 36, 38, 40 and 42, may be used to determine whether a biological sample contains related polypeptides that may be involved in the biosynthesis of natural products of the polyketide class or other classes that are characteristically partly polyketide in nature.

Polyclonal antibodies generated against the polypeptides of SEQ ID NOS: 2, 4, 6, 8, 10, 12, 14, 16, 18, 20, 23, 26, 28, 30, 32, 34, 36, 38, 40 and 42, or fragments comprising at least 5, 10, 15, 20, 25, 30, 35, 40, 50, 75, 100, or 150 consecutive amino acids thereof can be obtained by direct injection of the polypeptides into an animal or by administering the polypeptides to an animal, preferably a nonhuman. The antibody so obtained will then bind the polypeptide itself. In this manner, even a sequence encoding only a fragment of the polypeptide can be used to generate antibodies which may bind to the whole native polypeptide. Such antibodies can then be used to isolate the polypeptide from cells expressing that polypeptide.

For preparation of monoclonal antibodies, any technique which provides antibodies produced by continuous cell line cultures can be used. Examples include the hybridoma technique (Kholer and Milstein, 1975, Nature, 256:495-497), the trioma technique, the human B-cell hybridoma technique (Kozbor et al., 1983, Immunology Today 4:72), and the EBV-hybridoma technique (Cole, et al., 1985, in Monoclonal Antibodies and Cancer Therapy, Alan R. Liss, Inc., pp. 77-96).

Techniques described for the production of single chain antibodies (U.S. Pat. No. 4,946,778) can be adapted to produce single chain antibodies to the polypeptides of SEQ ID NOS: 2, 4, 6, 8, 10, 12, 14, 16, 18, 20, 23, 26, 28, 30, 32, 34, 36, 38, 40 and 42, or fragments comprising at least 5, 10, 15, 20, 25, 30, 35, 40, 50, 75, 100, or 150 consecutive amino acids thereof. Alternatively, transgenic mice may be used to express humanized antibodies to these polypeptides or fragments thereof.

Antibodies generated against the polypeptides of SEQ ID NOS: 2, 4, 6, 8, 10, 12, 14, 16, 18, 20, 23, 26, 28, 30, 32, 34, 36, 38, 40 and 42, or fragments comprising at least 5, 10, 15, 20, 25, 30, 35, 40, 50, 75, 100, or 150 consecutive amino acids thereof may be used in screening for similar polypeptides from a sample containing organisms or cell-free extracts thereof. In such techniques, polypeptides from the sample is contacted with the antibodies and those polypeptides which specifically bind the antibody are detected. Any of the procedures described above may be used to detect antibody binding. One such screening assay is described in “Methods for measuring Cellulase Activities”, Methods in Enzymology, Vol 160, pp. 87-116.

As used herein, the term “nucleic acid codes of SEQ ID NOS: 3, 5, 7, 9, 11, 13, 15, 17, 19, 21, 24, 27, 29, 31, 33, 35, 37, 39 and 41 ” encompass the nucleotide sequences of SEQ ID NOS: 3, 5, 7, 9, 11, 13, 15, 17, 19, 21, 24, 27, 29, 31, 33, 35, 37, 39 and 41, fragments of SEQ ID NOS: 3, 5, 7, 9, 11, 13, 15, 17, 19, 21, 24, 27, 29, 31, 33, 35, 37, 39 and 41, nucleotide sequences homologous to SEQ ID NOS: 3, 5, 7, 9, 11, 13, 15, 17, 19, 21, 24, 27, 29, 31, 33, 35, 37, 39 and 41, or homologous to fragments of SEQ ID NOS: 3, 5, 7, 9, 11, 13, 15, 17, 19, 21, 24, 27, 29, 31, 33, 35, 37, 39 and 41, and sequences complementary to all of the preceding sequences. The fragments include portions of SEQ ID NOS: 3, 5, 7, 9, 11, 13, 15, 17, 19, 21, 24, 27, 29, 31, 33, 35, 37, 39 and 41, comprising at least 10, 15, 20, 25, 30, 35, 40, 50, 75, 100, 150, 200, 300, 400 or 500 consecutive nucleotides of SEQ ID NOS: 3, 5, 7, 9, 11, 13, 15, 17, 19, 21, 24, 27, 29, 31, 33, 35, 37, 39 and 41. Preferably, the fragments are novel fragments. Homologous sequences and fragments of SEQ ID NOS: 3, 5, 7, 9, 11, 13, 15, 17, 19, 21, 24, 27, 29, 31, 33, 35, 37, 39 and 41 refer to a sequence having at least 99%, 98%, 97%, 96%, 95%, 90%, 80%, 75% or 70% homology to these sequences. Homology may be determined using any of the computer programs and parameters described herein, including BLASTN and TBLASTX with the default parameters. Homologous sequences also include RNA sequences in which uridines replace the thymines in the nucleic acid codes of SEQ ID NOS: 3, 5, 7, 9, 11, 13, 15, 17, 19, 21, 24, 27, 29, 31, 33, 35, 37, 39 and 41. The homologous sequences may be obtained using any of the procedures described herein or may result from the correction of a sequencing error. It will be appreciated that the nucleic acid codes of SEQ ID NOS: 3, 5, 7, 9, 11, 13, 15, 17, 19, 21, 24, 27, 29, 31, 33, 35, 37, 39 and 41 can be represented in the traditional single character format in which G, A, T and C denote the guanine, adenine, thymine and cytosine bases of the deoxyribonucleic acid (DNA) sequence respectively, or in which G, A, U and C denote the guanine adenine, uracil and cytosine bases of the ribonucleic acid (RNA) sequence (see the inside back cover of Stryer, Biochemistry, 3^{rd} edition, W. H. Freeman & Co., New York) or in any other format which records the identity of the nucleotides in a sequence.

“Polypeptide codes of SEQ ID NOS: 2, 4, 6, 8, 10, 12, 14, 16, 18, 20, 23, 26, 28, 30, 32, 34, 36, 38, 40 and 42” encompass the polypeptide sequences of SEQ ID NOS: 2, 4, 6, 8, 10, 12, 14, 16, 18, 20, 23, 26, 28, 30, 32, 34, 36, 38, 40 and 42 which are encoded by the cDNAs of SEQ ID NOS: 3, 5, 7, 9, 11, 13, 15, 17, 19, 21, 24, 27, 29, 31, 33, 35, 37, 39 and 41, polypeptide sequences homologous to the polypeptides of SEQ ID NOS: 2, 4, 6, 8, 10, 12, 14, 16, 18, 20, 23, 26, 28, 30, 32, 34, 36, 38, 40 and 42, or fragments of any of the preceding sequences. Homologous polypeptide sequences refer to a polypeptide sequence having at least 99%, 98%, 97%, 96%, 95%, 90%, 85%, 80%, 75% or 70% homology to one of the polypeptide sequences of SEQ ID NOS: 2, 4, 6, 8, 10, 12, 14, 16, 18, 20, 23, 26, 28, 30, 32, 34, 36, 38, 40 and 42. Polypeptide sequence homology may be determined using any of the computer programs and parameters described herein, including BLASTP version 2.2.2 with the default parameters or with any user-specified parameters. The homologous sequences may be obtained using any of the procedures described herein or may result from the correction of a sequencing error. The polypeptide fragments comprise at least 5, 10, 15, 20, 25, 30, 35, 40, 50, 75, 100 or 150 consecutive amino acids of the polypeptides of SEQ ID NOS: 2, 4, 6, 8, 10, 12, 14, 16, 18, 20, 23, 26, 28, 30, 32, 34, 36, 38, 40 and 42. Preferably the fragments are novel fragments. It will be appreciated that the polypeptide codes of the SEQ ID NOS: 3, 5, 7, 9, 11, 13, 15, 17, 19, 21, 24, 27, 29, 31, 33, 35, 37, 39 and 41 can be represented in the traditional single character format or three letter format (see the inside back cover of Stryer, Biochemistry, 3^{rd} edition, W. H. Freeman & Co., New York) or in any other format which relates the identity of the polypeptides in a sequence.

It will be readily appreciated by those skilled in the art that the nucleic acid codes of SEQ ID NOS: 3, 5, 7, 9, 11, 13, 15, 17, 19, 21, 24, 27, 29, 31, 33, 35, 37, 39 and 41 and polypeptides codes of SEQ ID NOS: 2, 4, 6, 8, 10, 12, 14, 16, 18, 20, 23, 26, 28, 30, 32, 34, 36, 38, 40 and 42 can be stored, recorded and manipulated on any medium which can be read and accessed by a computer. As used herein, the words “recorded” and “stored” refer to a process for storing information on a computer medium. A skilled artisan can readily adopt any of the presently known methods for recording information on a computer readable medium to generate manufactures comprising one or more of the nucleic acid codes of SEQ ID NOS: 3, 5, 7, 9, 11, 13, 15, 17, 19, 21, 24, 27, 29, 31, 33, 35, 37, 39 and 41, one or more of the polypeptide codes of SEQ ID NOS: 2, 4, 6, 8, 10, 12, 14, 16, 18, 20, 23, 26, 28, 30, 32, 34, 36, 38, 40 and 42.

Another embodiment of the present invention is a computer readable medium having stored thereon a sequence selected from the group consisting of a nucleic acid code of SEQ ID NOS: 3, 5, 7, 9, 11, 13, 15, 17, 19, 21, 24, 27, 29, 31, 33, 35, 37, 39 and 41 and a polypeptide code of SEQ ID NOS: 2, 4, 6, 8, 10, 12, 14, 16, 18, 20, 23, 26, 28, 30, 32, 34, 36, 38, 40 and 42. Another aspect of the present invention is a computer readable medium having recorded thereon one or more nucleic acid codes of SEQ ID NOS: 3, 5, 7, 9, 11, 13, 15, 17, 19, 21, 24, 27, 29, 31, 33, 35, 37, 39 and 41, preferably at least 2, 5, 10, 15, or 20 nucleic acid codes of SEQ ID NOS: 3, 5, 7, 9, 11, 13, 15, 17, 19, 21, 24, 27, 29, 31, 33, 35, 37, 39 and 41. Another aspect of the invention is a computer readable medium having recorded thereon one or more of the polypeptide codes of SEQ ID NOS: 2, 4, 6, 8, 10, 12, 14, 16, 18, 20, 23, 26, 28, 30, 32, 34, 36, 38, 40 and 42, preferably at least 2, 5, 10, 15 or 20 polypeptide codes of SEQ ID NOS: 2, 4, 6, 8, 10, 12, 14, 16, 18, 20, 23, 26, 28, 30, 32, 34, 36, 38, 40 and 42.

Another embodiment of the present invention is a computer system comprising a processor and a data storage device wherein said data storage device has stored thereon a reference sequence selected from the group consisting of a nucleic acid code of SEQ ID NOS: 3, 5, 7, 9, 11, 13, 15, 17, 19, 21, 24, 27, 29, 31, 33, 35, 37, 39 and 41 and a polypeptide code of SEQ ID NOS: 2, 4, 6, 8, 10, 12, 14, 16, 18, 20, 23, 26, 28, 30, 32, 34, 36, 38, 40 and 42.

Computer readable media include magnetically readable media, optically readable media, electronically readable media and magnetic/optical media. For example, the computer readable media may be a hard disk, a floppy disk, a magnetic tape, CD-ROM, Digital Versatile Disk (DVD), Random Access Memory (RAM), or Read Only Memory (ROM) as well as other types of media known to those skilled in the art.

The present invention will be further described with reference to the following examples; however, it is to be understood that the present invention is not limited to such examples.

### EXAMPLE 1

### Identification and sequencing of the dorrigocin biosynthetic gene cluster

Streptomyces platensis subsp. rosaceus strain AB1981F-75 (NRRL 18993) was obtained from the Agricultural Research Service collection (National Center for Agricultural Utilization Research, 1815 N. University Street, Peoria, Ill. 61604) and cultured using standard microbiological techniques (Kieser et al., supra). This organism was propagated on oatmeal agar medium at 28 degrees Celsius for several days. For isolation of high molecular weight genomic DNA, cell mass from three freshly grown, near confluent 100 mm petri dishes was used. The cell mass was collected by gentle scraping with a plastic spatula. Residual agar medium was removed by repeated washes with STE buffer (75 mM NaCl; 20 mM Tris-HCl, pH 8.0; 25 mM EDTA). High molecular weight DNA was isolated by established protocols (Kieser et al. supra) and its integrity was verified by field inversion gel electrophoresis (FIGE) using the preset program number 6 of the FIGE MAPPER™ power supply (BIORAD). This high molecular weight genomic DNA serves for the preparation of a small size fragment genomic sampling library (GSL), i.e., the small insert library, as well as a large size fragment cluster identification library (CIL), i.e., the large insert library. Both libraries contained randomly generated S. platensis genomic DNA fragments and, therefore, are representative of the entire genome of this organism.

For the generation of the S. platensis GSL library, genomic DNA was randomly sheared by sonication. DNA fragments having a size range between 1.5 and 3 kb were fractionated on a agarose gel and isolated using standard molecular biology techniques (Sambrook et al., supra). The ends of the obtained DNA fragments were repaired using T4 DNA polymerase (Roche) as described by the supplier. This enzyme creates DNA fragments with blunt ends that can be subsequently cloned into an appropriate vector. The repaired DNA fragments were subcloned into a derivative of pBluescript SK+ vector (Stratagene) which does not allow transcription of cloned DNA fragments. This vector was selected as it contains a convenient polylinker region surrounded by sequences corresponding to universal sequencing primers such as T3, T7, SK, and KS (Stratagene). The unique EcoRV restriction site found in the polylinker region was used as it allows insertion of blunt-end DNA fragments. Ligation of the inserts, use of the ligation products to transform E. coli DH10B (Invitrogen) host and selection for recombinant clones were performed as previously described (Sambrook et al., supra). Plasmid DNA carrying the S. platensis genomic DNA fragments was extracted by the alkaline lysis method (Sambrook et al., supra) and the insert size of 1.5 to 3 kb was confirmed by electrophoresis on agarose gels. Using this procedure, a library of small size random genomic DNA fragments is generated that covers the entire genome of the studied microorganism. The number of individual clones that can be generated is infinite but only a small number is further analyzed to sample the microorganism's genome.

A CIL library was constructed from the S. platensis high molecular weight genomic DNA using the SuperCos-1 cosmid vector (Stratagene™). The cosmid arms were prepared as specified by the manufacturer. The high molecular weight DNA was subjected to partial digestion at 37 degrees Celsius with approximately one unit of Sau3AI restriction enzyme (New England Biolabs) per 100 micrograms of DNA in the buffer supplied by the manufacturer. This enzyme generates random fragments of DNA ranging from the initial undigested size of the DNA to short fragments of which the length is dependent upon the frequency of the enzyme DNA recognition site in the genome and the extent of the DNA digestion. At various timepoints, aliquots of the digestion were transferred to new microfuge tubes and the enzyme was inactivated by adding a final concentration of 10 mM EDTA and 0.1% SDS. Aliquots judged by FIGE analysis to contain a significant fraction of DNA in the desired size range (30-50 kb) were pooled, extracted with phenol/chloroform (1:1 vol:vol), and pelletted by ethanol precipitation. The 5′ ends of Sau3AI DNA fragments were dephosphorylated using alkaline phosphatase (Roche) according to the manufacturer's specifications at 37 degrees Celcius for 30 min. The phosphatase was heat inactivated at 70 degrees Celcius for 10 min and the DNA was extracted with phenol/chloroform (1:1 vol:vol), pelletted by ethanol precipitation, and resuspended in sterile water. The dephosphorylated Sau3AI DNA fragments were then ligated overnight at room temperature to the SuperCos-1 cosmid arms in a reaction containing approximately four-fold molar excess SuperCos-1 cosmid arms. The ligation products were packaged using Gigapack® III XL packaging extracts (Stratagene™) according to the manufacturer's specifications. The CIL library consisted of 864 isolated cosmid clones in E. coli DH10B (Invitrogen). These clones were picked and inoculated into nine 96-well microtiter plates containing LB broth (per liter of water: 10.0 g NaCl; 10.0 g tryptone; 5.0 g yeast extract) which were grown overnight and then adjusted to contain a final concentration of 25% glycerol. These microtiter plates were stored at −80 degrees Celcius and served as glycerol stocks of the CIL library. Duplicate microtiter plates were arrayed onto nylon membranes as follows. Cultures grown on microtiter plates were concentrated by pelleting and resuspending in a small volume of LB broth. A 3×3 96-pin grid was spotted onto nylon membranes. These membranes representing the complete CIL library were then layered onto LB agar and incubated ovenight at 37 degrees Celcius to allow the colonies to grow. The membranes were layered onto filter paper pre-soaked with 0.5N NaOH/1.5M NaCl for 10 min to denature the DNA and then neutralized by transferring onto filter paper pre-soaked with 0.5M Tris (pH 8)/1.5M NaCl for 10 min. Cell debris was gently scraped off with a plastic spatula and the DNA was crosslinked onto the membranes by UV irradiation using a GS GENE LINKER™ UV Chamber (BIORAD). Considering an average size of 8 Mb for an actinomycete genome and an average size of 35 kb of genomic insert in the CIL library, this library represents roughly a 4-fold coverage of the microorganism's entire genome.

The GSL library was analyzed by sequence determination of the cloned genomic DNA inserts. The universal primers KS or T7, referred to as forward (F) primers, were used to initiate polymerization of labeled DNA. Extension of at least 700 bp from the priming site can be routinely achieved using the TF, BDT v2.0 sequencing kit as specified by the supplier (Applied Biosystems). Sequence analysis of the small genomic DNA fragments (Genomic Sequence Tags, GSTs) was performed using a 3700 ABI capillary electrophoresis DNA sequencer (Applied Biosystems). The average length of the DNA sequence reads was ˜700 bp. Further analysis of the obtained GSTs was performed by sequence homology comparison to various protein sequence databases. The DNA sequences of the obtained GSTs were translated into amino acid sequences and compared to the National Center for Biotechnology Information (NCBI) nonredundant protein database and the proprietary Ecopia natural product biosynthetic gene Decipher™ database using previously described algorithms (Altschul et al., supra). Sequence similarity with known proteins of defined function in the database enables one to make predictions on the function of the partial protein that is encoded by the translated GST.

A total of 1536 S. platensis GSTs were generated and analyzed by sequence comparison using the Blast algorithm (Altschul et al., supra). Sequence alignments displaying an E value of at least e-5 were considered as significantly homologous and retained for further evaluation. GSTs showing similarity to a gene of interest can be at this point selected and used to identify larger segments of genomic DNA from the CIL library that include the gene(s) of interest. As dorrigocins and migrastatin are polyketides, several S. platensis GSTs that were clearly portions of type I PKS genes were pursued. Using these type I PKS GSTs, we indeed identified a type I PKS locus in S. platensis, however, the PKS domain order and number of modules of this type I PKS was inconsistent with the structures of dorrigocins and migrastatin (data not shown). In addition to the GSTs that were clearly portions of type I PKS genes, we also identified GSTs that were somewhat related to type I PKS genes. When the latter were used as probes to screen the CIL library and the resulting cosmid clones were sequenced, an unusual PKS gene cluster was identified which proved to be the dorrigocin biosynthetic locus.

Hybridization oligonucleotide probes were radiolabeled with P³² using T4 polynucleotide kinase (New England Biolabs) in 15 microliter reactions containing 5 picomoles of oligonucleotide and 6.6 picomoles of [γ-P³²]ATP in the kinase reaction buffer supplied by the manufacturer. After 1 hour at 37 degrees Celcius, the kinase reaction was terminated by the addition of EDTA to a final concentration of 5 mM. The specific activity of the radiolabeled oligonucleotide probes was estimated using a Model 3 Geiger counter (Ludlum Measurements Inc., Sweetwater, Tex.) with a built-in integrator feature. The radiolabeled oligonucleotide probes were heat-denatured by incubation at 85 degrees Celcius for 10 minutes and quick-cooled in an ice bath immediately prior to use.

The S. platensis CIL library membranes were pretreated by incubation for at least 2 hours at 42 degrees Celcius in Prehyb Solution (6X SSC; 20 mM NaH₂PO₄; 5X Denhardt's; 0.4% SDS; 0.1 mg/ml sonicated, denatured salmon sperm DNA) using a hybridization oven with gentle rotation. The membranes were then placed in Hyb Solution (6X SSC; 20 mM NaH₂PO₄; 0.4% SDS; 0.1 mg/ml sonicated, denatured salmon sperm DNA) containing 1×10⁶ cpm/ml of radiolabeled oligonucleotide probe and incubated overnight at 42 degrees Celcius using a hybridization oven with gentle rotation. The next day, the membranes were washed with Wash Buffer (6X SSC, 0.1% SDS) for 45 minutes each at 46, 48, and 50 degrees Celcius using a hybridization oven with gentle rotation. The S. platensis CIL membranes were then exposed to X-ray film to visualize and identify the positive cosmid clones. Positive clones were identified, cosmid DNA was extracted from 30 ml cultures using the alkaline lysis method (Sambrook et al., supra) and the inserts were entirely sequenced using a shotgun sequencing approach (Fleischmann et al., Science, 269:496-512).

Sequencing reads were assembled using the Phred-Phrap™ algorithm (University of Washington, Seattle, U.S.A.) recreating the entire DNA sequence of the cosmid insert. Reiterations of hybridizations of the CIL library with probes derived from the ends of the original cosmid allow indefinite extension of sequence information on both sides of the original cosmid sequence until the complete sought-after gene cluster is obtained. The structure of dorrigocin suggests that it would be synthesized by a modular type I polyketide synthases (PKSs) containing 10 modules. Three overlapping cosmid clones that were detected by the oligonucleotide probe derived from the GSTs remotely related to type I PKSs have been completely sequenced to provide approximately 54 Kb of DNA comprising the dorrigocin biosynthetic locus (FIG. 1).

### EXAMPLE 2

### Genes and proteins involved in biosynthesis of dorrigocin

The dorrigocin locus encodes 11 proteins and spans approximately 53,800 base pairs of DNA that is contiguous except for one gap beginning after base pair 52,101. More than 15 kilobases of DNA sequence were analyzed on each side of the dorrigocin locus and these regions contain primary metabolic genes. The order and relative position of the 11 open reading frames representing the proteins of the biosynthetic locus for dorrigocin (DORR ORFs) are provided in FIG. 1. The top line in FIG. 1 provides a scale in kilobase pairs. The black bars depict the two DNA contigs separated by a small gap (<100 bp) in the sequencing. The arrows represent the 11 open reading frames of the dorrigocin biosynthetic locus.

Thus, the complete locus of genes regulating the biosynthesis of dorrigocin is formed by two DNA contiguous sequences (SEQ ID NOS: 1 and 22). The contiguous nucleotide sequences are arranged such that, as found within the dorrigocin biosynthetic locus, the 52101 base pairs of DNA contig 1 (SEQ ID NO: 1) is found adjacent to the 5′ end of DNA contig 2 (SEQ ID NO: 22). The contiguous nucleotide sequence of SEQ ID NO: 1 contains the 10 open reading frames (ORFs) listed in SEQ ID NOS: 2, 4, 6, 8, 10, 12, 14, 16, 18, 20. DORR ORF 1 (SEQ ID NO: 2) is the 1217 amino acids deduced from the nucleic acid sequence of SEQ ID NO: 3 which is drawn from residues 3720 to 67 (anti sense strand) of contig 1 (SEQ ID NO: 1). DORR ORF 2 (SEQ ID NO: 4) is the 529 amino acids deduced from the nucleic acid sequence of SEQ ID NO: 5 which is drawn from residues 4092 to 5681 (sense strand) of contig 1 (SEQ ID NO: 1). DORR ORF 3 (SEQ ID NO: 6) is the 83 amino acids deduced from the nucleic acid sequence of SEQ ID NO: 7 which is drawn from residues 5767 to 6018 (sense strand) of contig 1 (SEQ ID NO: 1). DORR ORF 4 (SEQ ID NO: 8) is the 656 amino acids deduced from the nucleic acid sequence of SEQ ID NO: 9 which is drawn from residues 6023 to 7993 (sense strand) of contig 1 (SEQ ID NO: 1). DORR ORF 5 (SEQ ID NO: 10) is the 3192 amino acids deduced from the nucleic acid sequence of SEQ ID NO: 11 which is drawn from residues 8009 to 17587 (sense strand) of contig 1 (SEQ ID NO: 1). DORR ORF 6 (SEQ ID NO: 12) is the 8026 amino acids deduced from the nucleic acid sequence of SEQ ID NO: 13 which is drawn from residues 17634 to 41714 (sense strand) of contig 1 (SEQ ID NO: 1). DORR ORF 7 (SEQ ID NO: 14) is the 1953 amino acids deduced from the nucleic acid sequence of SEQ ID NO: 15 which is drawn from residues 41772 to 47633 (sense strand) of contig 1 (SEQ ID NO: 1). DORR ORF 8 (SEQ ID NO: 16) is the 751 amino acids deduced from the nucleic acid sequence of SEQ ID NO: 17 which is drawn from residues 47635 to 49890 (sense strand) of contig 1 (SEQ ID NO: 1). DORR ORF 9 (SEQ ID NO: 18) is the 338 amino acids deduced from the nucleic acid sequence of SEQ ID NO: 19 which is drawn from residues 49922 to 50938 (sense strand) of contig 1 (SEQ ID NO: 1). DORR ORF 10 (SEQ ID NO: 20) is the 281 amino acids deduced from the nucleic acid sequence of SEQ ID NO: 21 which is drawn from residues 51234 to 52079 (sense strand) of contig 1 (SEQ ID NO: 1). The contiguous nucleotide sequence of SEQ ID NO: 22 (1700 base pair) contains DORR ORF 11 (SEQ ID NO: 23). DORR ORF 11 (SEQ ID NO: 23) is the 328 amino acids representing the C-terminus of the expected polypeptide and deduced from the nucleic acid sequence of SEQ ID NO: 24 which is drawn from residues 163 to 1149 (sense strand) of contig 2 (SEQ ID NO: 22).

Two deposits, namely E. coli DH10B (088CF) strain and E. coli DH10B (088CX) strain each harbouring a cosmid clone of a partial biosynthetic locus for dorrigocin have been deposited with the International Depositary Authority of Canada, Bureau of Microbiology, Health Canada, 1015 Arlington Street, Winnipeg, Manitoba, Canada R3E 3R2 on Feb. 27, 2001 and were assigned deposit accession number IDAC270201-3 and 270101-4 respectively. The E. coli strain deposits are referred to herein as “the deposited strains”.

The deposited strains comprise the complete biosynthetic locus for dorrigocin. The sequence of the polynucleotides comprised in the deposited strains, as well as the amino acid sequence of any polypeptide encoded thereby are controlling in the event of any conflict with any description of sequences herein.

The deposit of the deposited strains has been made under the terms of the Budapest Treaty on the International Recognition of the Deposit of Micro-organisms for Purposes of Patent Procedure. The deposited strains will be irrevocably and without restriction or condition released to the public upon the issuance of a patent. The deposited strains are provided merely as convenience to those skilled in the art and are not an admission that a deposit is required for enablement, such as that required under 35 U.S.C. §112. A license may be required to make, use or sell the deposited strains, and compounds derived therefrom, and no such license is hereby granted.

In order to identify the function of the genes in the dorrigocin biosynthetic locus, DORR ORFs 1 to 11 (SEQ ID NOS: 2, 4, 6, 8, 10, 12, 14, 16, 18, 20 and 23) were compared, using the BLASTP version 2.2.2 algorithm with the default parameters, to sequences in the National Center for Biotechnology Information (NCBI) nonredundant protein database and the DECIPHER™ database of microbial genes, pathways and natural products (available on a subscription basis from Ecopia BioSciences Inc. St.-Laurent, QC, Canada).

The accession numbers of the top GenBank hits of this Blast analysis are presented in Table 2 along with the corresponding E value. The E value relates the expected number of chance alignments with an alignment score at least equal to the observed alignment score. An E value of 0.00 indicates a perfect homolog. The E values are calculated as described in Altschul et al. J. Mol. Biol., Oct. 5; 215(3) 403-10, the teachings of which is incorporated herein by reference. The E value assists in the determination of whether two sequences display sufficient similarity to justify an inference of homology.

TABLE 2

| | | | GenBank | | % | % | proposed function of |
|---|---|---|---|---|---|---|---|
| ORF | Family | #aa | homology | probability | identity | similarity | GenBank match |
| 1 | REBP | 1217 | BAB69312.1, | 0.0 | 502/784 | 554/784 | putative regulatory |
| | | | 1094aa | | (64.03%) | (70.66%) | protein, |
| | | | | | | | Streptomyces avermitilis |
| | | | CAC20917.1, | 0.0 | 456/685 | 502/685 | hypothetical protein, |
| | | | 694aa | | (66.57%) | (73.28%) | Streptomyces natalensis |
| | | | AAF73451.1, | 1e−31 | 89/250 | 123/250 | putative activator AknO, |
| | | | 272aa | | (35.6%) | (49.2%) | Streptomyces galilaeus |
| 2 | AYTT | 529 | NP_389591.1, | 3e−68 | 143/278 | 187/278 | pksC, Bacillus subtilis |
| | | | 288aa | | (51.44%) | (67.27%) | |
| | | | NP_405051.1, | 8e−50 | 120/280 | 163/280 | putative acyl transferase, |
| | | | 282aa | | (42.86%) | (58.21%) | Yersinia pestis |
| | | | NP_484284.1, | 3e−35 | 103/279 | 147/279 | malonyl co-A acyl |
| | | | 292aa | | (36.92%) | (52.69%) | carrier protein |
| | | | | | | | transacylase, Nostoc sp. |
| 3 | ACPI | 83 | NP_437899.1, | 0.002 | 23/76 | 46/76 | hypothetical protein, |
| | | | 88aa | | (30.26%) | (60.53%) | Sinorhizobium meliloti |
| | | | AAC05776.1, | 0.032 | 21/51 | 32/51 | D-alanyl carrier protein, |
| | | | 79aa | | (41.18%) | (62.75%) | Streptococcus mutans |
| 4 | AOTF | 656 | NP_437900.1, | 1e−100 | 248/655 | 346/655 | putative asparagine |
| | | | 645aa | | (37.86%) | (52.82%) | synthetase, |
| | | | | | | | Sinorhizobium meliloti |
| | | | NP_107193.1, | 1e−100 | 244/650 | 342/650 | asparagine synthetase, |
| | | | 675aa | | (37.54%) | (52.62%) | Mesorhizobium loti |
| | | | AAF34252.1, | 1e−64 | 206/623 | 297/623 | putative asparagine |
| | | | 643aa | | (33.07%) | (47.67%) | synthetase, |
| | | | | | | | Desulfovibrio gigas |
| 5 | PKUN | 3192 | NP_389600.1, | 0.0 | 714/2193 | 1044/2193 | polyketide synthase |
| | | | 4427aa | | (32.56%) | (47.61%) | of type I, |
| | | | | | | | Bacillus subtilis |
| | | | NP_389603.1, | 0.0 | 678/2479 | 1064/2479 | polyketide synthase |
| | | | 4930aa | | (27.35%) | (42.92%) | of type I, |
| | | | | | | | Bacillus subtilis |
| | | | CAA84505.1, | 1e−117 | 289/924 | 438/924 | putative polyketide |
| | | | 1763aa | | (31.28%) | (47.4%) | synthase, |
| | | | | | | | Bacillus subtilis |
| 6 | PKUN | 8026 | NP_389601.1, | 0.0 | 904/2663 | 1327/2663 | polyketide synthase, |
| | | | 4273aa | | (33.95%) | (49.83%) | Bacillus subtilis |
| | | | NP_389599.1, | 0.0 | 805/2198 | 1150/2198 | polyketide synthase |
| | | | 4447aa | | (36.62%) | (52.32%) | of type I, |
| | | | | | | | Bacillus subtilis |
| | | | AAK15074.1, | 0.0 | 877/2674 | 1215/2674 | albicidin PKS-NRPS, |
| | | | 4801aa | | (32.8%) | (45.44%) | Xanthomonas albilineans |
| 7 | PKUN | 1953 | NP_389603.1, | 0.0 | 574/1546 | 837/1546 | polyketide synthase |
| | | | 4930aa | | (37.13%) | (54.14%) | of type I, |
| | | | | | | | Bacillus subtilis |
| | | | NP_389600.1, | 0.0 | 483/1452 | 716/1452 | polyketide synthase |
| | | | 4427aa | | (33.26%) | (49.31%) | of type I, |
| | | | | | | | Bacillus subtilis |
| | | | CAA84505.1, | 1e−154 | 332/1013 | 498/1013 | putative polyketide |
| | | | 1763aa | | (32.77%) | (49.16%) | synthase, |
| | | | | | | | Bacillus subtilis |
| 8 | AYOA | 751 | NP_389593.1, | 1e−148 | 285/650 | 391/650 | pksE, Bacillus subtilis |
| | | | 650aa | | (43.85%) | (60.15%) | |
| | | | T37055, | 6e−97 | 197/450 | 263/450 | probable oxidoreductase, |
| | | | 527aa | | (43.78%) | (58.44%) | Streptomyces coelicolor |
| | | | T30186, | 9e−93 | 177/434 | 261/434 | hypothetical protein, |
| | | | 543aa | | (40.78%) | (60.14%) | Shewanella sp |
| 9 | OXRY | 338 | CAB62729.1, | 2e−97 | 180/334 | 222/334 | putative oxidoreductase, |
| | | | 364aa | | (53.89%) | (66.47%) | Streptomyces coelicolor |
| | | | NP_420823.1, | 5e−77 | 158/341 | 200/341 | alcohol dehydrogenase, |
| | | | 341aa | | (46.33%) | (58.65%) | Caulobacter crescentus |
| | | | NP_279793.1, | 6e−73 | 153/334 | 198/334 | quinone oxidoreductase, |
| | | | 380aa | | (45.81%) | (59.28%) | Halobacterium sp |
| 10 | MTFA | 281 | AAD28459.1, | 2e−73 | 138/262 | 178/262 | MitM, |
| | | | 283aa | | (52.67%) | (67.94%) | Streptomyces lavendulae |
| | | | AAG42853.1, | 3e−42 | 109/262 | 143/262 | SnogM, |
| | | | 278aa | | (41.6%) | (54.58%) | Streptomyces nogalater |
| | | | T44579, | 2e−39 | 103/268 | 143/268 | C5-O-methyltransferase, |
| | | | 283aa | | (38.43%) | (53.36%) | Streptomyces avermitilis |
| 11 | OXRC | 328 | CAB46536.1, | 2e−86 | 165/313 | 218/313 | NikF protein, |
| | | | 410aa | | (52.72%) | (69.65%) | Streptomyces tendae |
| | | | AAL85695.1, | 1e−83 | 162/313 | 213/313 | cytochrome P450, |
| | | | 410aa | | (51.76%) | (68.05%) | Streptomyces anso- |
| | | | | | | | chromogenes |
| | | | AAF71771.1, | 2e−79 | 159/310 | 202/310 | NysN, |
| | | | 398aa | | (51.29%) | (65.16%) | Streptomyces noursei |

### EXAMPLE 3

### Identification and sequencing of the lactimidomycin biosynthetic gene cluster

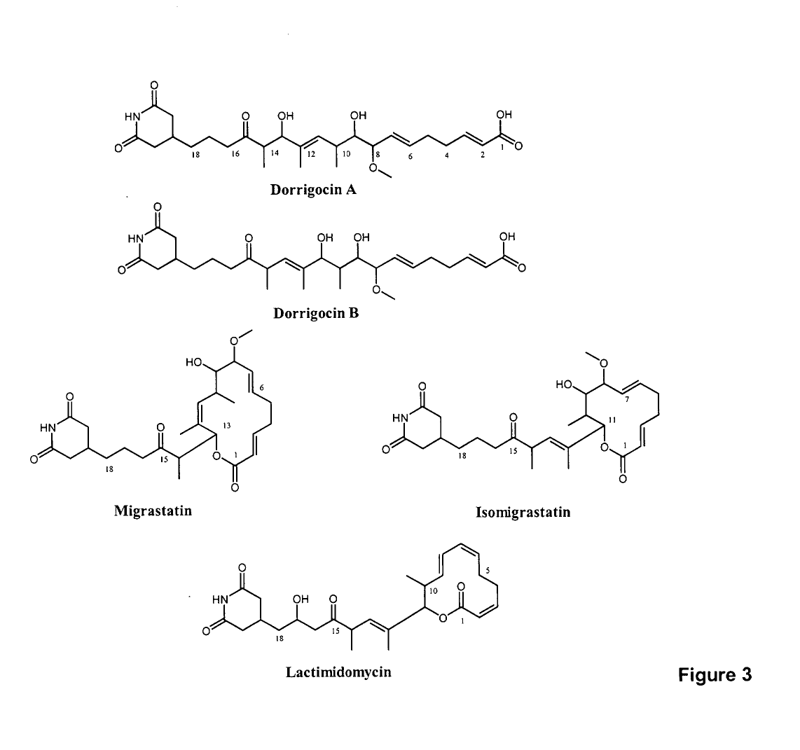

Given the structural similarities between migrastatin and lactimidomycin (FIG. 3), it is expected that their biosynthetic loci are equally similar. With the dorrigocin biosynthetic locus in hand, we set out to identify and sequence the lactimidomycin biosynthetic locus from Streptomyces amphibiosporus ATCC 53964. The genomic sampling method described in Example 1 was applied to genomic DNA from S. amphibiosporus. A total of 480 GSL clones were sequenced with the forward primer and analyzed by sequence comparison using the Blast algorithm (Altschul et al., supra) to identify those clones that contained inserts related to the dorrigocin biosynthetic genes. Several such GST clones were identified and were used to isolate cosmid clones from a S. amphibiosporus CIL library. For example, the GST clone (insert size approximately 2.5 kb) from which one oligonucleotide probe was derived was clearly a portion of a gene from the S. amphibiosporus genome that encoded a homologue of the dorrigocin ORF 7. The forward read of this GST encodes a polypeptide of at least 58% identity and 68% similarity to amino acids 1112 to 1354, corresponding to the N-terminal portion of the KR domain of module 10 of the dorrigocin synthase followed by the C-terminal portion of the DH of module 10 of the dorrigocin synthase. The reverse read of this GST encodes a polypeptide of at least 54% identity and 64% similarity to amino acids 545 to 768, corresponding to the C-terminal portion of the KS domain of module 10 of the dorrigocin synthase followed by the N-terminal portion of the interaction domain of module 10 of the dorrigocin synthase. Therefore, the 2.5 kb insert of this GST clone was oriented such that the open reading frame was in the same direction as the T3 primer of the cloning vector. Sequencing of overlapping cosmid clones provided over 50 Kb of DNA comprising the lactimidomycin biosynthetic locus (FIG. 4).

### EXAMPLE 4

### Genes and proteins involved in the biosynthesis of lactimidomycin

The lactimidomycin locus encodes 9 proteins and spans approximately 50500 base pairs of DNA disclosed in a single contiguous DNA sequence (SEQ ID NO: 25). The order and relative position of the 9 open reading frames representing the proteins of the biosynthetic locus for lactimidomycin (LACT ORFs) are provided in FIG. 4. The top line in FIG. 4 provides a scale in kilobase pairs. The arrows represent the 9 open reading frames of the lactimidomycin biosynthetic locus.

Thus, LACT ORF 1 (SEQ ID NO: 26) is the 565 amino acids deduced from the nucleic acid sequence of SEQ ID NO: 27 which is drawn from residues 1 to 1698 (sense strand) of SEQ ID NO: 25. LACT ORF 2 (SEQ ID NO: 28) is the 84 amino acids deduced from the nucleic acid sequence of SEQ ID NO: 29 which is drawn from residues 1908 to 2162 (sense strand) of SEQ ID NO: 25. LACT ORF 3 (SEQ ID NO: 30) is the 656 amino acids deduced from the nucleic acid sequence of SEQ ID NO: 31 which is drawn from residues 2166 to 4136 (sense strand) of SEQ ID NO: 25. LACT ORF 4 (SEQ ID NO: 32) is the 3436 amino acids deduced from the nucleic acid sequence of SEQ ID NO: 33 which is drawn from residues 4152 to 14462 (sense strand) of SEQ ID NO: 25. LACT ORF 5 (SEQ ID NO: 34) is the 8360 amino acids deduced from the nucleic acid sequence of SEQ ID NO: 35 which is drawn from residues 14549 to 39631 (sense strand) of SEQ ID NO: 25. LACT ORF 6 (SEQ ID NO: 36) is the 2098 amino acids deduced from the nucleic acid sequence of SEQ ID NO: 37 which is drawn from residues 39628 to 45924 (sense strand) of SEQ ID NO: 25. LACT ORF 7 (SEQ ID NO: 38) is the 768 amino acids deduced from the nucleic acid sequence of SEQ ID NO: 39 which is drawn from residues 45926 to 48232 (sense strand) of SEQ ID NO: 25. LACT ORF 8 (SEQ ID NO: 40) is the 418 amino acids deduced from the nucleic acid sequence of SEQ ID NO: 41 which is drawn from residues 48441 to 49697 (sense strand) of SEQ ID NO: 25. LACT ORF 9 (SEQ ID NO: 42) is the 247 amino acids deduced from the nucleic acid sequence of SEQ ID NO: 43 which is drawn from residues 50543 to 49800 (anti sense strand) of SEQ ID NO: 25.

In order to identify the function of the genes in the lactimidomycin biosynthetic locus, LACT ORFs 1 to 9 (SEQ ID NOS: 26, 28, 30, 32, 34, 36, 38, 40 and 42) were compared, using the BLASTP version 2.2.2 algorithm with the default parameters, to sequences in the National Center for Biotechnology Information (NCBI) nonredundant protein database and the DECIPHER™ database of microbial genes, pathways and natural products (available on a subscription basis from Ecopia BioSciences Inc. St.-Laurent, QC, Canada). The accession numbers of the top GenBank hits of this Blast analysis are presented in Table 3 along with the corresponding E value.

TABLE 3

| | | | GenBank | | % | % | proposed function of |
|---|---|---|---|---|---|---|---|
| ORF | Family | #aa | homology | probability | identity | similarity | GenBank match |
| 1 | AYTT | 600 | NP_389591.1, | 1e−65 | 135/276 | 180/276 | pksC, Bacillus subtilis |
| | | | 288aa | | (48.91%) | (65.22%) | |
| | | | NP_405051.1, | 1e−52 | 119/276 | 168/276 | putative acyl transferase, |
| | | | 282aa | | (43.12%) | (60.87%) | Yersinia pestis |
| | | | NP_484284.1, | 3e−38 | 107/277 | 151/277 | malonyl CO-A acyl carrier |
| | | | 292aa | | (38.63%) | (54.51%) | protein transacylase, |
| | | | | | | | Nostoc sp. |
| 2 | ACPI | 120 | NP_346588.1, | 0.054 | 19/52 | 31/52 | D-alanyl carrier protein, |
| | | | 79aa | | (36.54%) | (59.62%) | Streptococcus pneumoniae |
| 3 | AOTF | 657 | NP_107193.1, | 1e−108 | 252/651 | 359/651 | asparagine synthetase, |
| | | | 675aa | | (38.71%) | (55.15%) | Mesorhizobium loti |
| | | | NP_437900.1, | 3e−98 | 238/638 | 324/638 | putative asparagine |
| | | | 645aa | | (37.3%) | (50.78%) | synthetase, |
| | | | | | | | Sinorhizobium meliloti |
| | | | AAF34252.1, | 4e−67 | 210/605 | 297/605 | putative asparagine |
| | | | 643aa | | (34.71%) | (49.09%) | synthetase, |
| | | | | | | | Desulfovibrio gigas |
| 4 | PKUN | 3437 | NP_389600.1, | 1e−129 | 254/619 | 368/619 | polyketide synthase |
| | | | 4427aa | | (41.03%) | (59.45%) | of type I, |
| | | | | | | | Bacillus subtilis |
| | | | NP_389601.1, | 1e−123 | 249/614 | 357/614 | polyketide synthase, |
| | | | 4273aa | | (40.55%) | (58.14%) | Bacillus subtilis |
| | | | CAA84505.1, | 1e−109 | 250/671 | 348/671 | putative polyketide |
| | | | 1763aa | | (37.26%) | (51.86%) | synthase, |
| | | | | | | | Bacillus subtilis |
| 5 | PKUN | 8361 | NP_389601.1, | 0.0 | 968/2747 | 1382/2747 | polyketide synthase, |
| | | | 4273aa | | (35.24%) | (50.31%) | Bacillus subtilis |
| | | | AAK15074.1, | 0.0 | 927/2698 | 1261/2698 | albicidin PKS-NRPS, |
| | | | 4801aa | | (34.36%) | (46.74%) | Xanthomonas albilineans |
| | | | CAA84505.1, | 0.0 | 462/1528 | 717/1528 | putative polyketide |
| | | | 1763aa | | (30.24%) | (46.92%) | synthase, |
| | | | | | | | Bacillus subtilis |
| 6 | PKUN | 2099 | NP_389603.1, | 0.0 | 577/1497 | 823/1497 | polyketide synthase |
| | | | 4930aa | | (38.54%) | (54.98%) | of type I, |
| | | | | | | | Bacillus subtilis |
| | | | NP_389600.1, | 0.0 | 483/1493 | 729/1493 | polyketide synthase |
| | | | 4427aa | | (32.35%) | (48.83%) | of type I, |
| | | | | | | | Bacillus subtilis |
| | | | CAA84505.1, | 1e−158 | 329/961 | 521/961 | putative polyketide |
| | | | 1763aa | | (34.24%) | (54.21%) | synthase, |
| | | | | | | | Bacillus subtilis |
| 7 | AYOA | 769 | NP_389593.1, | 1e−146 | 286/651 | 393/651 | pksE, Bacillus subtilis |
| | | | 650aa | | (43.93%) | (60.37%) | |
| | | | AAL01063.1, | 5e−94 | 179/433 | 257/433 | omega-3 polyunsaturated |
| | | | 544aa | | (41.34%) | (59.35%) | fatty acid synthase, |
| | | | | | | | Photobacterium profundum |
| | | | BAA89385.1, | 4e−93 | 180/433 | 263/433 | ORF11, Moritella marina |
| | | | 538aa | | (41.57%) | (60.74%) | |
| 8 | OXRC | 419 | T36526, | 3e−64 | 153/389 | 213/389 | probable cytochrome P450 |
| | | | 411aa | | (39.33%) | (54.76%) | hydroxylase, Streptomyces |
| | | | | | | | coelicolor |
| | | | NP_390897.1, | 6e−57 | 134/394 | 212/394 | cytochrome P450-like |
| | | | 395aa | | (34.01%) | (53.81%) | enzyme, Bacillus subtilis |
| | | | NP_252021.1, | 2e−56 | 150/383 | 198/383 | cytochrome P450, |
| | | | 418aa | | (39.16%) | (51.7%) | Pseudomonas aeruginosa |
| 9 | PPTF | 315 | AAG43513.1, | 2e−81 | 149/233 | 173/233 | phosphopantetheinyl |
| | | | 246aa | | (63.95%) | (74.25%) | transferase PptA, Strepto- |
| | | | | | | | mycesverticillus |
| | | | T35172, | 4e−61 | 127/219 | 149/219 | hypothetical protein, |
| | | | 226aa | | (57.99%) | (68.04%) | Streptomyces coelicolor |
| | | | AAF71762.1, | 9e−61 | 126/219 | 144/219 | NysF, Streptomyces noursei |
| | | | 245aa | | (57.53%) | (65.75%) | |

### EXAMPLE 5

### Unusual two component PKS system involved in biosynthesis of dorrigocins and lactimidomycin

The dorrigocin locus encodes three PKSs that contain KS, KR, ACP and unusual DH domains in unusual arrangements. The three PKSs in this locus encode a total of 10 ketosynthase (KS) domains, sufficient to produce a polyketide chain the length of dorrigocin. The three PKSs share some features of typical type I PKSs, namely that the synthases contain multiple fused domains. However, the dorrigocin PKSs are distinct from type I PKSs in that they do not contain AT domains that are physically attached to the PKS. Instead, the AT function is provided in trans by distinct components. Therefore the dorrigocin PKS system represents a new, two component PKS system.

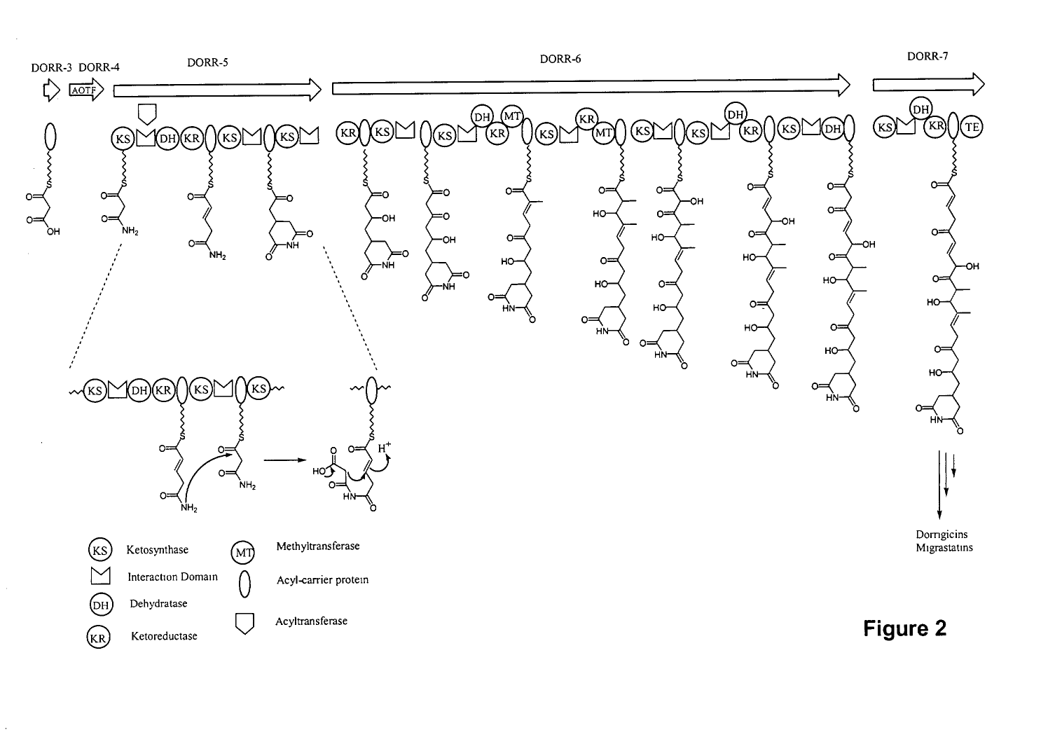

Without intending to be limited to any particular mechanism of action or biosynthetic scheme, the proteins of the invention can explain the formation of dorrigocin. FIG. 2 shows disposition of the 10 modules that act in a stepwise fashion to synthesize the polyketide backbone. Referring to FIG. 2, DORR acyl carrier protein ACPI (SEQ ID NO: 6) and DORR amidotransferase AOTF (SEQ ID NO: 8) are translationally coupled to the first PKS of the DORR locus (SEQ ID NO: 10). The ACPI (SEQ ID NO: 6) shows most significant similarity to proteins that transfer amino-substituted acyl groups. ACPI (SEQ ID NO: 6) and AOTF (SEQ ID NO: 8) cooperate to generate the starter unit for polyketide chain extension.

Unlike typical type I PKS modules which contain an AT domain downstream of the KS domain, the KS domains in each of the PKS modules in the dorrigocin locus (SEQ ID NOS: 10, 12 and 14) are not followed by an AT domain. Nonetheless, the unusual dorrigocin PKSs contain a small conserved domain downstream of the KS domains. This conserved domain is postulated to act as a docking site for the malonyl-CoA:ACP malonyltransferase activity. The malonyl-CoA:ACP malonyltransferase activity may be provided by the AYTT DORR ORF 2 (SEQ ID NO: 4), AYOA DORR ORF 8 (SEQ ID NO: 16), or by the primary metabolic fatty acid malonyl-CoA:ACP malonyltransferase.

Module 1 carries a bound malonyl extender unit and catalyzes one round of elongation of the starter unit, followed by ketoreductase and dehydration.

Module 2 is acylated by the independent AT-thioesterase fusion protein AYTT (SEQ ID NO: 4). This protein consists of a malonyl CoA:ACP malonyltransferase fused to a thioesterase. Module 2 catalyzes the formation of an imide bond between the acyl chains tethered to modules 1 and 2. The formation of an imide bond requires an unusual “backward” step in the elongation cycle, a maneuver that is facilitated by the thioesterase activity associated with the AYTT protein (SEQ ID NO: 4). The KS domain of module 1 is used again, this time to catalyze the Claisen condensation reaction that generates the cyclic glutarimide group.

The nascent polyketide chain now skips from the ACP of module 1 to the KS of module 3 for the next elongation step. Malonyl extender units are used by modules 3 to 6. Beta-ketoreduction occurs at modules 5 and 6. Methyl side chains are added by the MT domains of modules 5 and 6.

Module 7 uses a hydroxymalonyl extender. The hydroxymalonyl extender unit is generated by the independent AT-oxidoreductase fusion protein AYOA (SEQ ID NO: 16) and is transferred to module 7. The hydroxyl side chain is methylated by the MTFA O-methyltransferase (SEQ ID NO: 20).

Modules 8 to 10 use malonyl extender units. Ketoreductation and dehydration occur at modules 8 and 10. Module 9 is notable in that it contains a DH domain, but no KR domain.

The general design rules for the biosynthesis of conventional type I polyketide are applicable to the biosynthesis of the intermediate polyketide backbone structure to dorrigocin A, dorrigocin B and migrastatin molecules, as shown in FIG. 2. The intermediate differs from dorrigocin B in the state of beta-carbonyl reduction and the absence of a methyl side chain at C-14. Dorrigocin PKSs (SEQ ID NOS: 10, 12, 14) recruit ketoreductase, dehydratase and enoylreductase from the primary fatty acid synthase as needed to achieve the proper oxidation states at C-5, C-9 and C-1 7. The two modules that require interaction with enoylreductases correspond to the two modules that span separate PKS peptides. An MT domain was not found in the module that incorporates C-14, suggesting that methylation at C-14 is catalyzed by a primary methyltransferase or the MT domain in the adjacent module.

The oxidoreductases encoded by the OXRC and OXRY proteins (SEQ ID NOS: 23 and 18) provide the necessary activities to catalyze the interconversion of dorrigocin A and dorrigocin B.

The lactimidomycin biosynthetic locus consists of 9 ORFs (SEQ ID NOS: 26, 28, 30, 32, 34, 36, 38, 40 and 42), eight of which are highly homologous to a corresponding ORF of the dorrigocin biosynthetic locus. LACT ORF 1 (SEQ ID NO: 26) is homologous to DORR ORF 2 (SEQ ID NO: 4), both of which are fusions of an acyltransferase and a thioesterase designated as AYTT. LACT ORF 2 (SEQ ID NO: 28) is homologous to DORR ORF 3 (SEQ ID NO: 6), both of which are acyl carrier proteins designated as ACPI. LACT ORF 3 (SEQ ID NO: 30) is homologous to DORR ORF 4 (SEQ ID NO: 8), both of which are amidotransferases similar to bacterial asparagine synthetases designated as AOTF. LACT ORFs 4, 5 and 6 (SEQ ID NOS: 32, 34 and 36) are homologous to DORR ORFs 5, 6, and 7 (SEQ ID NOS: 10, 12 and 14), respectively, all of which are unusual modular PKSs devoid of AT domains designated as PKUN. LACT ORF 7 (SEQ ID NO: 38) is homologous to DORR ORF 8 (SEQ ID NO: 16), both of which are fusions of an acyltransferase and an oxidoreductase designated as AYOA. Finally, LACT ORF 8 (SEQ ID NO: 40) is homologous to DORR ORF 11 (SEQ ID NO: 23), both of which are cytochrome P450 monooxygenases designated as OXRC.

LACT ORF 9 (SEQ ID NO: 42) is a phosphopantetheinyl transferase designated as PPTF for which there is no counterpart in the dorrigocin locus. This phosphopantetheinyl transferase is involved in the covalent attachment of the phosphopantetheinyl prosthetic arm to the acyl carrier proteins of the lactimidomycin synthase complex. In contrast, the acyl carrier proteins of the dorrigocin may be phosphopantetheinylated by a phosphopantetheinyl transferase encoded by a gene outside of the dorrigocin biosynthetic locus.

The dorrigocin biosynthetic locus contains three ORFs that have no counterpart in the lactimidomycin locus. DORR ORF 1 (SEQ ID NO: 2) which is a regulator designated as REBP, DORR ORF 9 (SEQ ID NO: 18) which is an oxidoreductase designated as OXRY, and DORR ORF 10 (SEQ ID NO: 20) which is an O-methyltransferase designated as MTFA. The absence of an OXRY in the lactimidomycin locus is significant as this DORR ORF 9 (SEQ ID NO: 18) is implicated in the interconversion of dorrigocins A and B involving an isomerization of a double bond. No analogous isomerization event is known to occur in the case of lactimidomycin biosynthesis, presumably due to the absence of an OXRY homologue. The absence of an MTFA in the lactimidomycin locus is significant as, unlike dorrigocins and migrastatin, lactimidomycin does not contain any O-methyl groups.

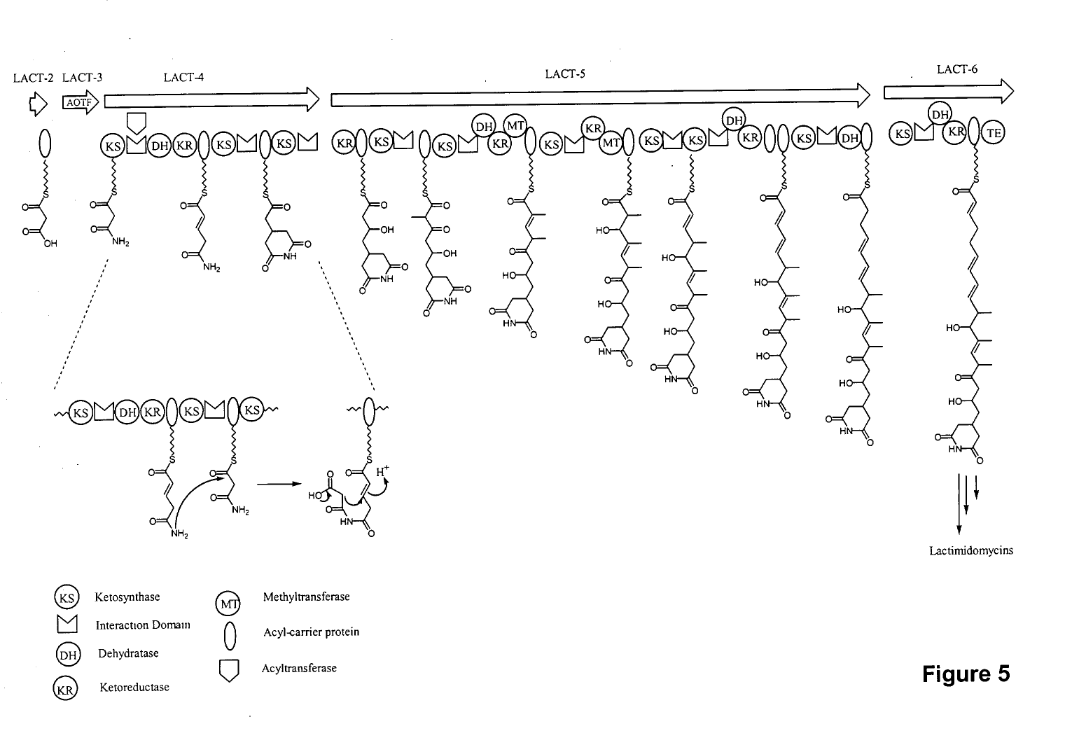

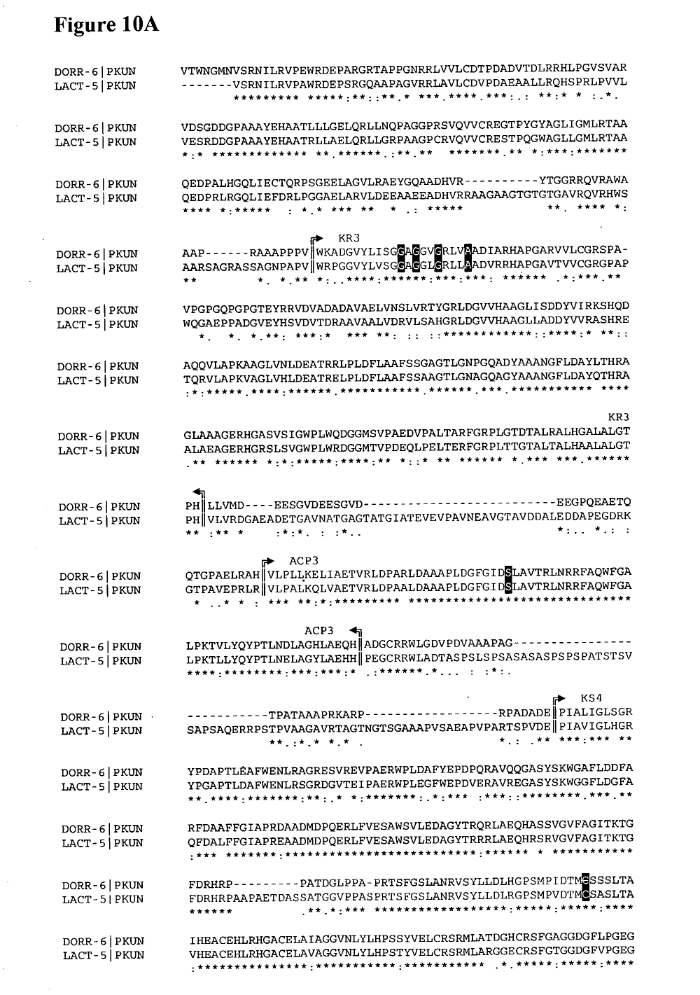

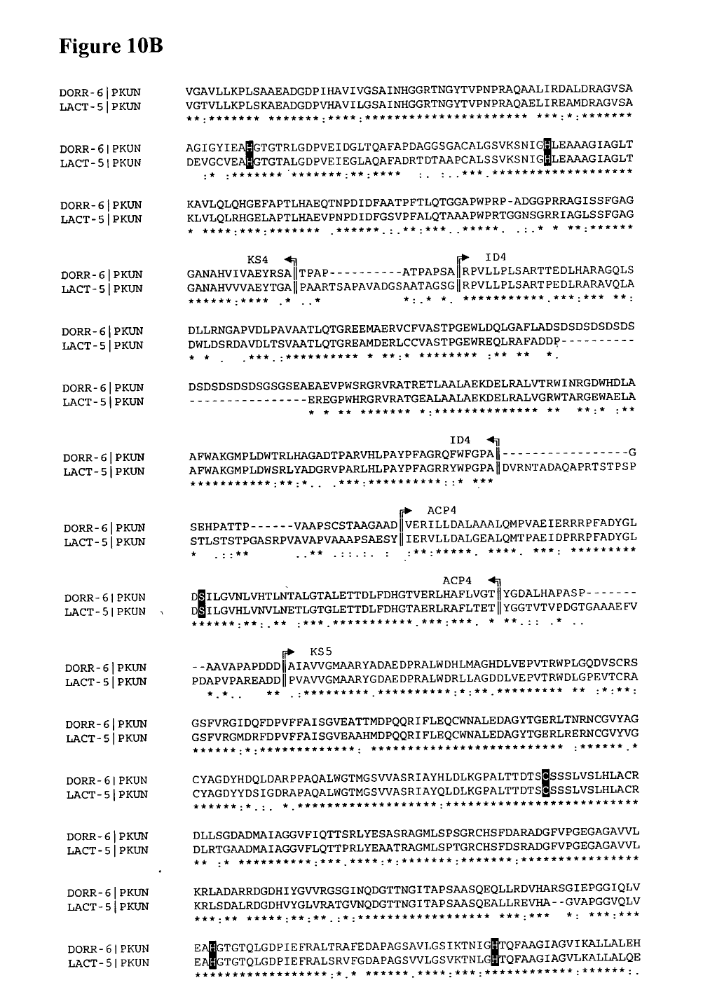

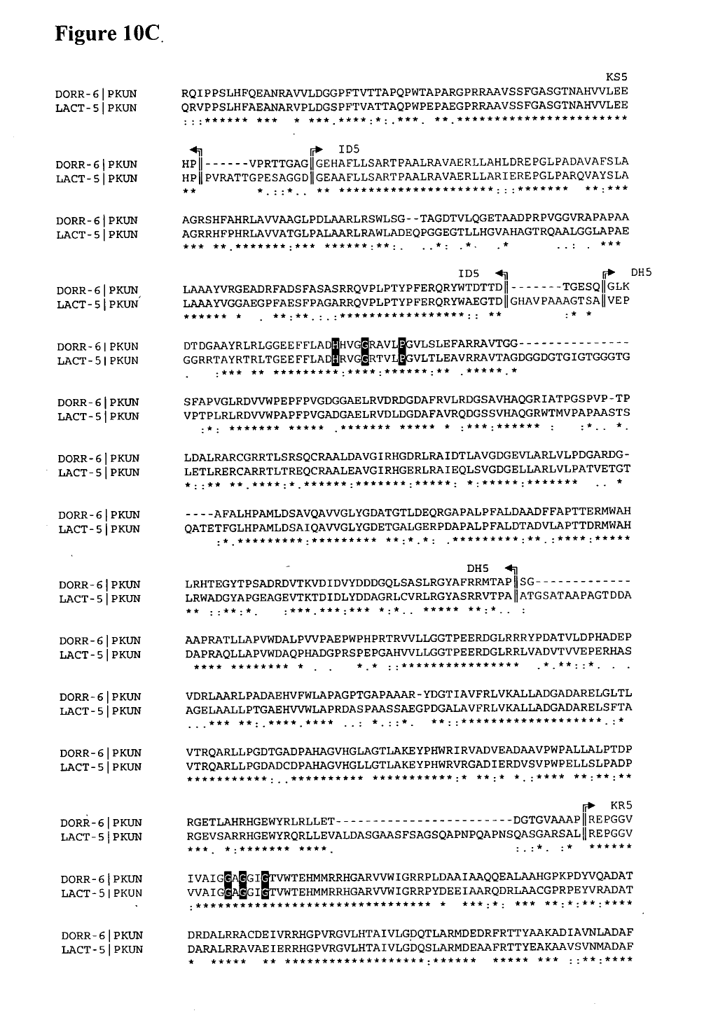

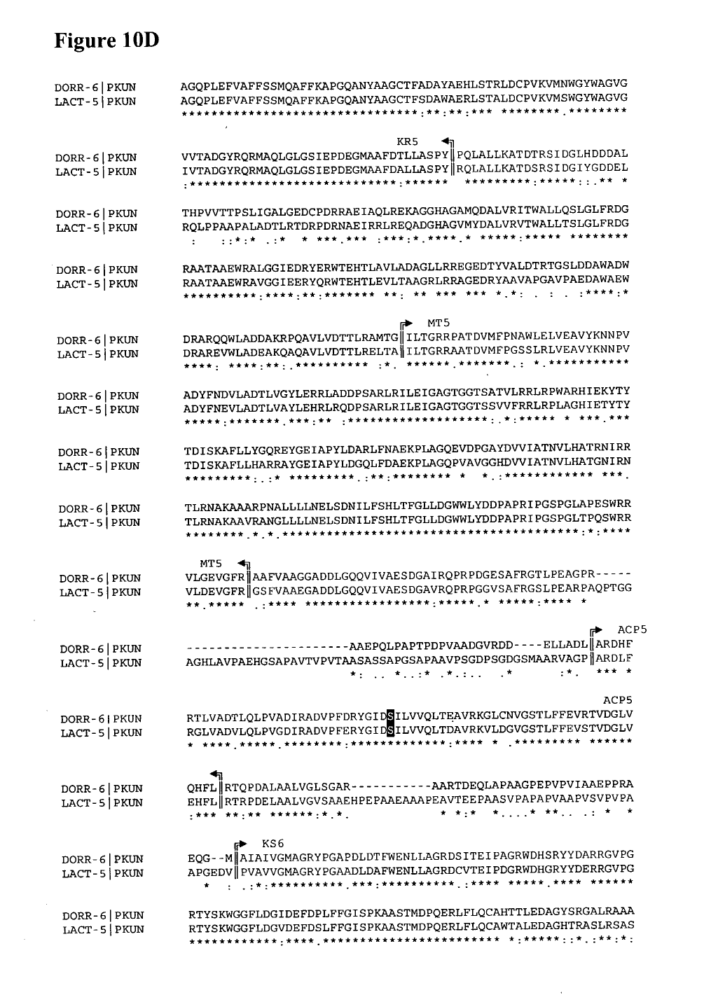

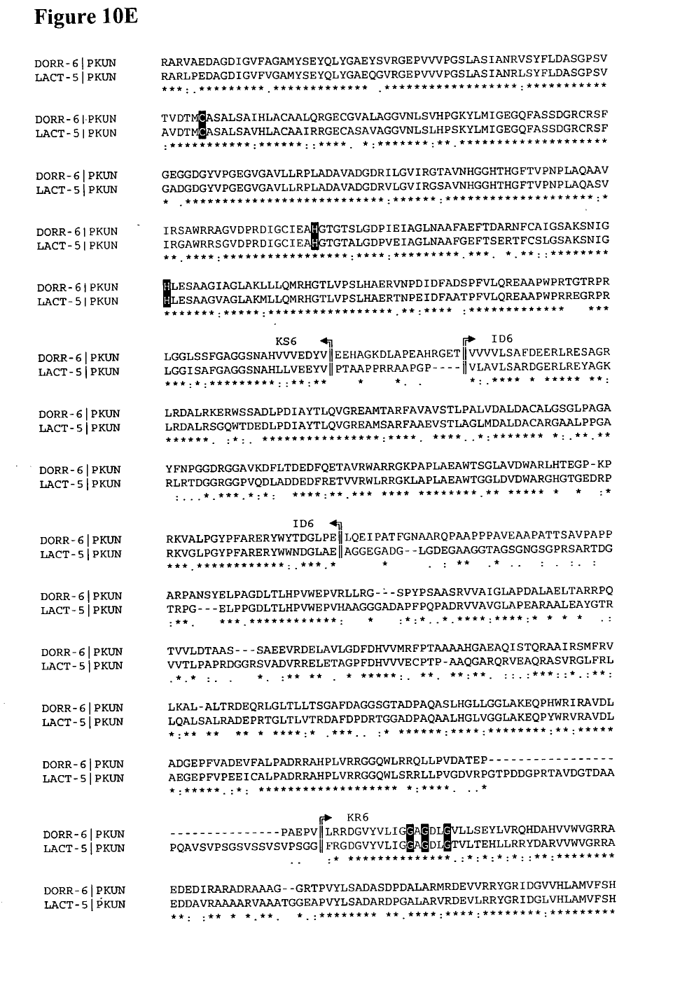

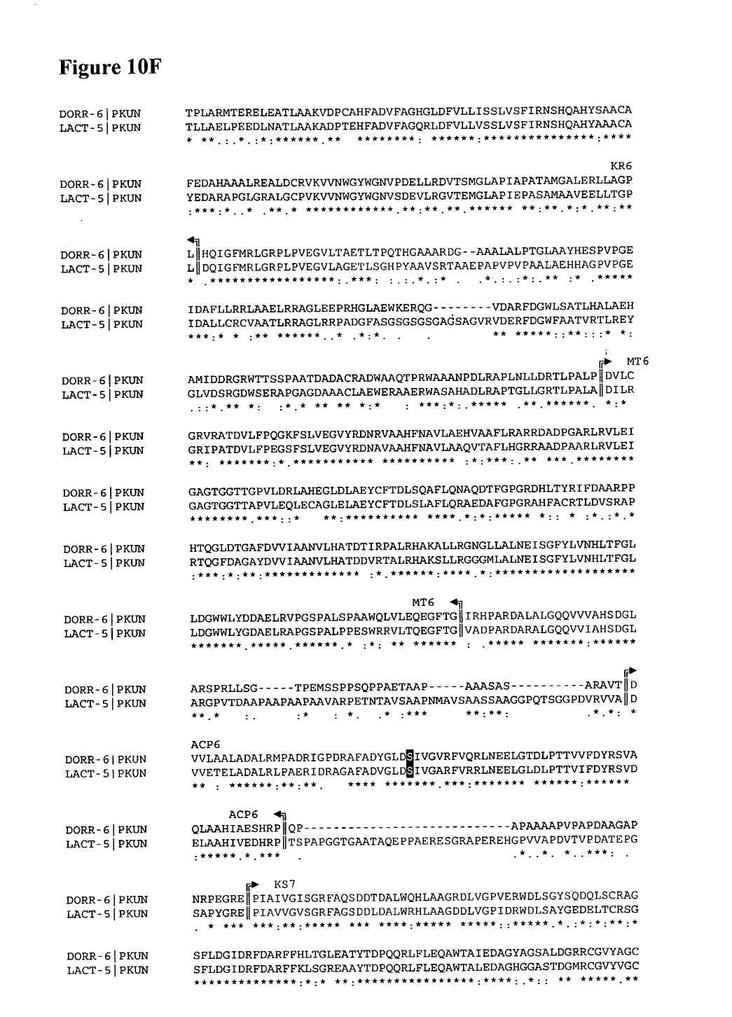

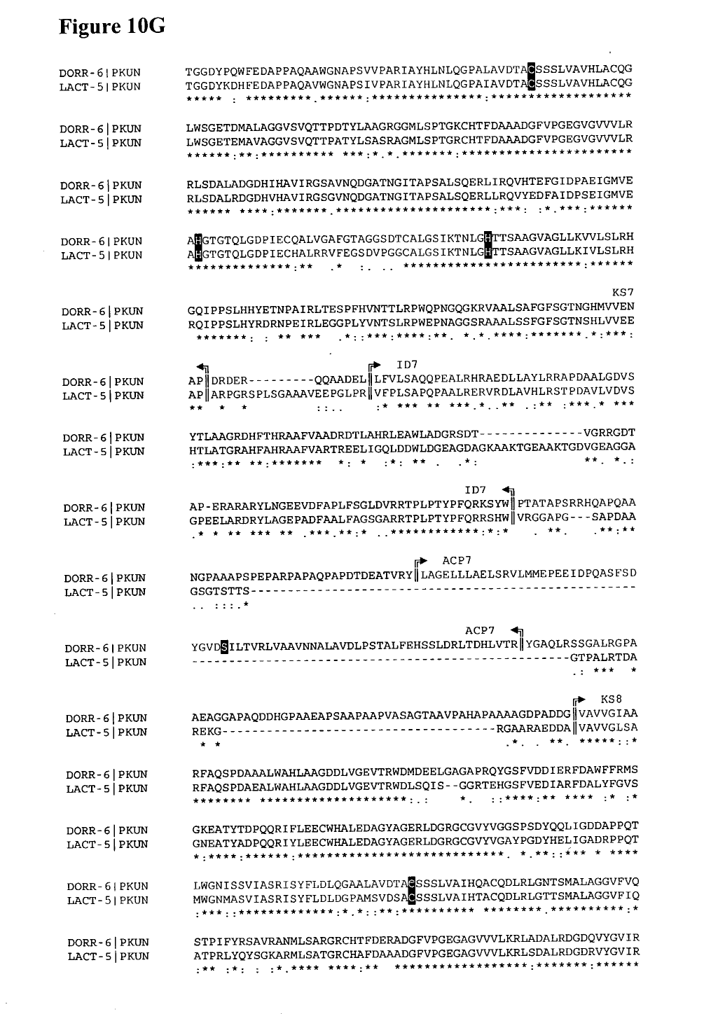

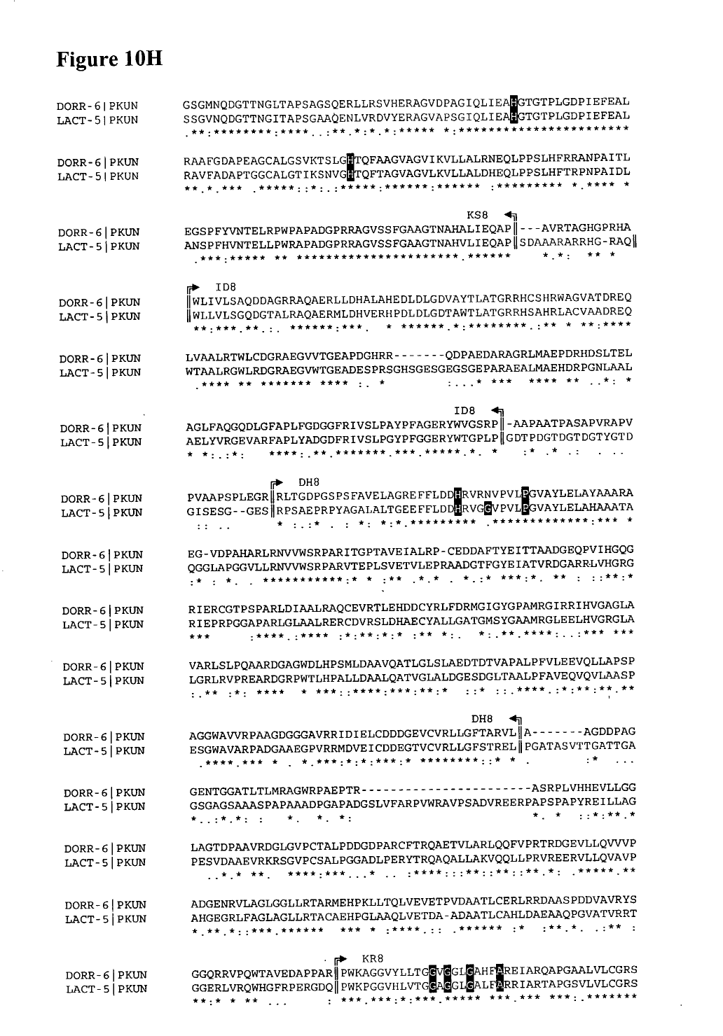

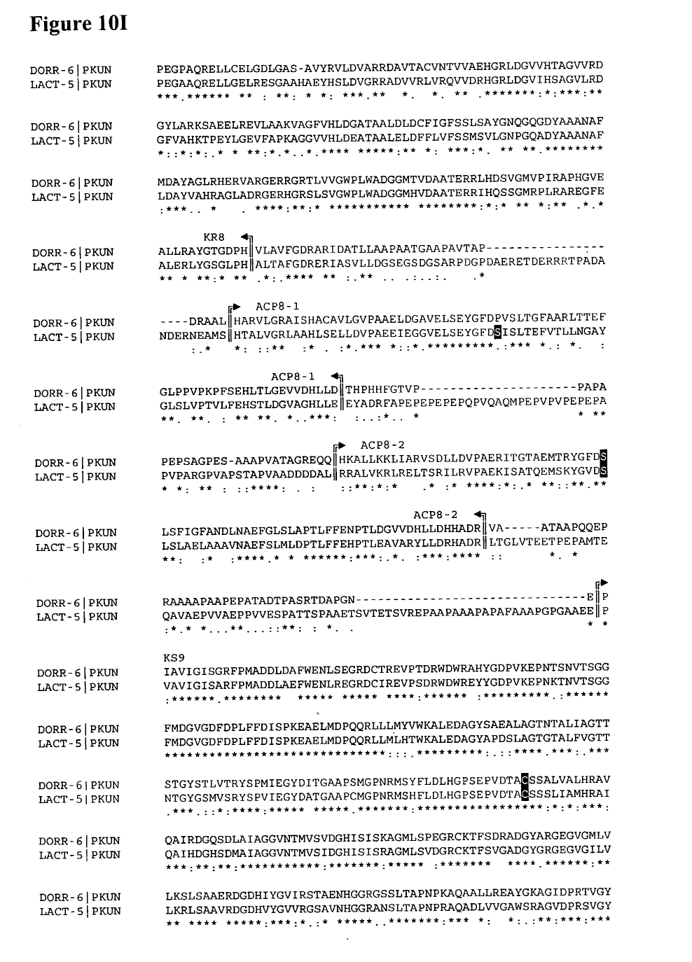

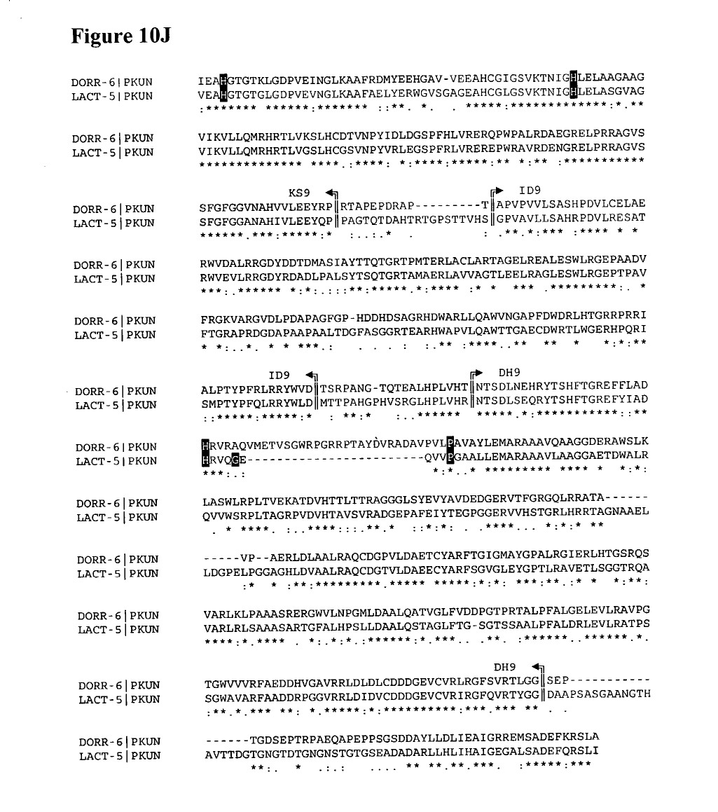

Without intending to be limited to any particular mechanism of action or biosynthetic scheme, the LACT proteins can explain the biosynthesis of lactimidomycin (FIG. 5) in a manner analogous to the biosynthetic pathway for dorrigocins and migrastatin (FIG. 2). The lactimidomycin and dorrigocin PKS systems differ in modules 7 and 8 of the respective PCK systems (FIG. 4, 10). Module 7 in the dorrigocin PKS system comprises a KS domain, an interaction domain, and an ACP domain that, together with a trans-acting AT domain, are involved in the incorporation of a methoxymalonyl extender unit (or a hydroxymalonyl extender unit that is subsequently O-methylated). Conversely, module 7 in the lactimidomycin PKS system comprises only a KS domain and an interaction domain; it lacks an ACP domain. As such, it is predicted that this module cannot carry out polyketide chain elongation. Consistent with this prediction, lactimidomycin does not contain a hydroxymethyl substitution on C-8. Module 8 in the dorrigocin PKS system comprises a KS domain, an interaction domain, a DH domain, a KR domain, and two tandem ACP domains. However, the first of these ACP domains is predicted to be inactive (indicated by the ‘X’ in FIG. 4) as the conserved serine residue that normally serves as the phosphopantetheine attachment site has been substituted by a proline residue (FIG. 10). Conversely, both ACP domains contain the active site serine residues in module 8 in the lactimidomycin PKS system. Therefore, we propose that both of these ACPs are loaded with malonyl-CoA and either the KS from module 7 or the KS from module 8 catalyzes two rounds of polyketide chain elongation or, alternatively, the KS domains from module 7 and 8 each catalyze one round of polyketide chain elongation.

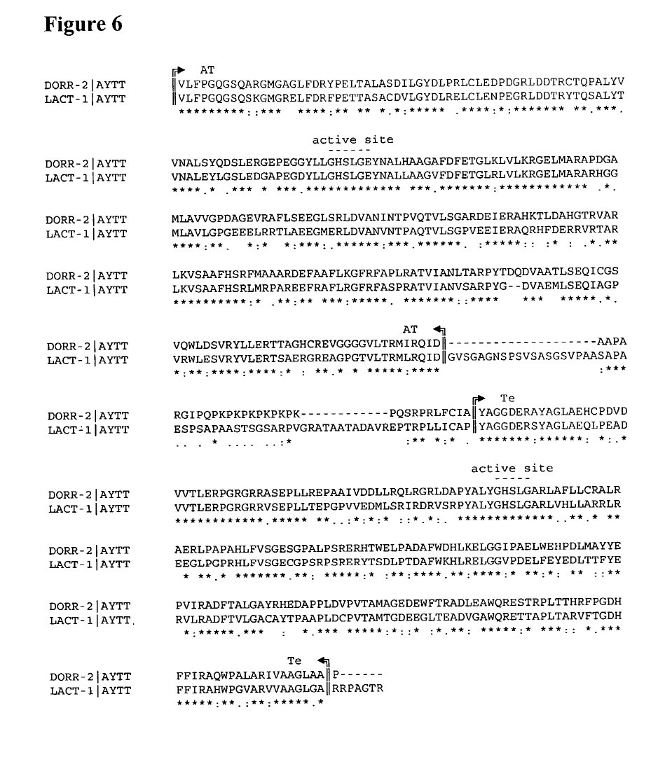

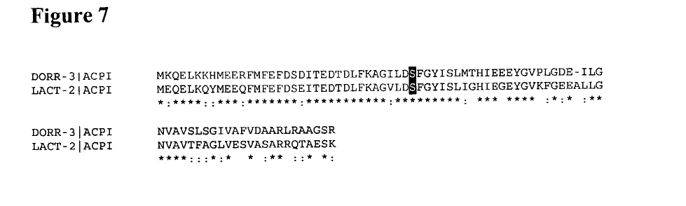

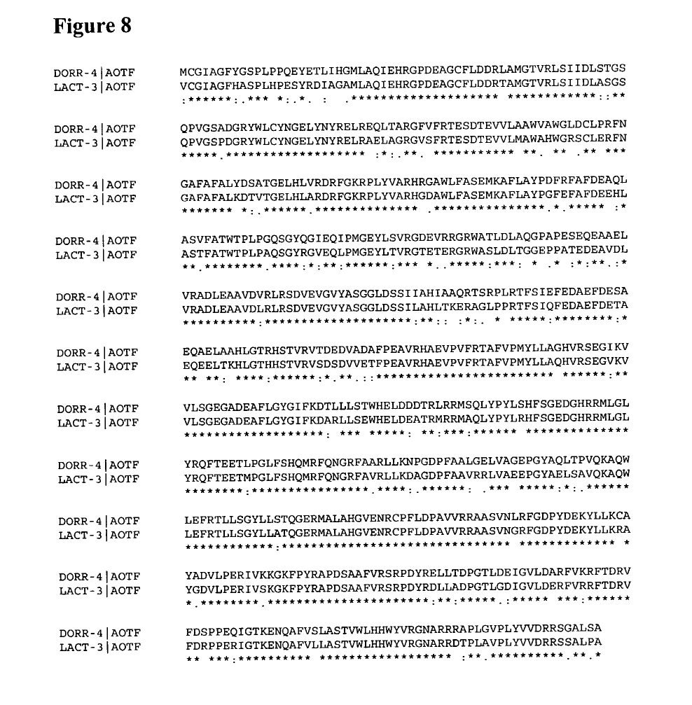

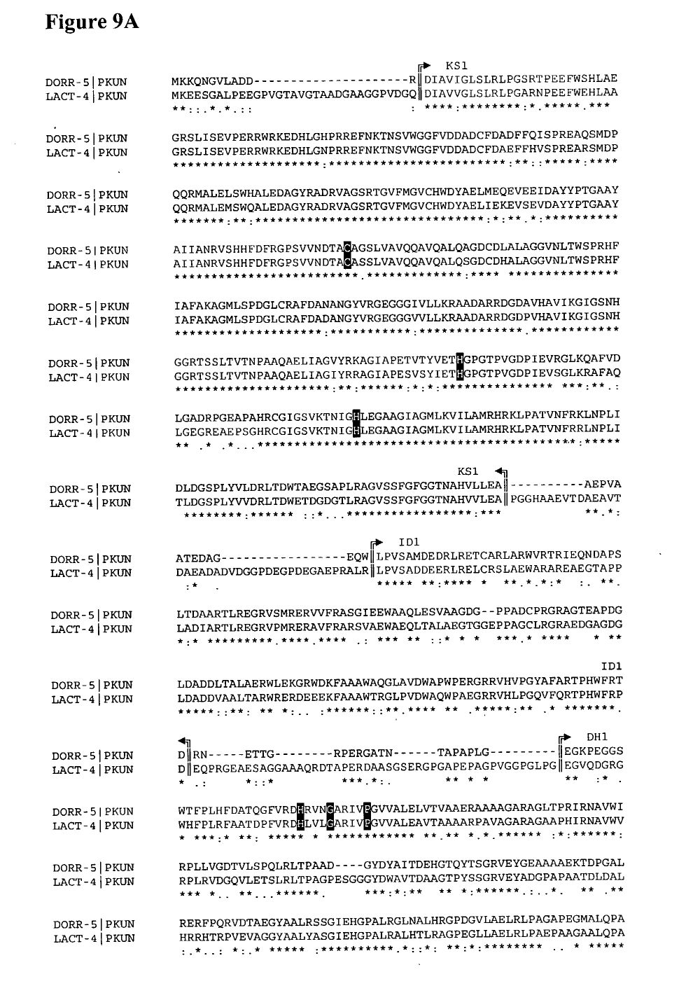

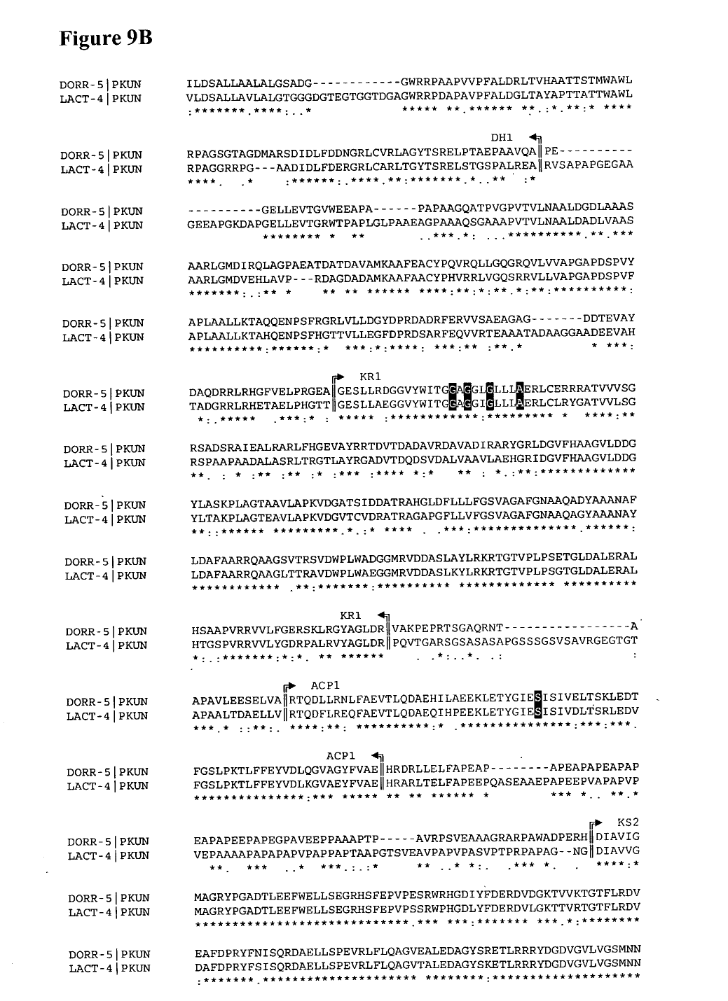

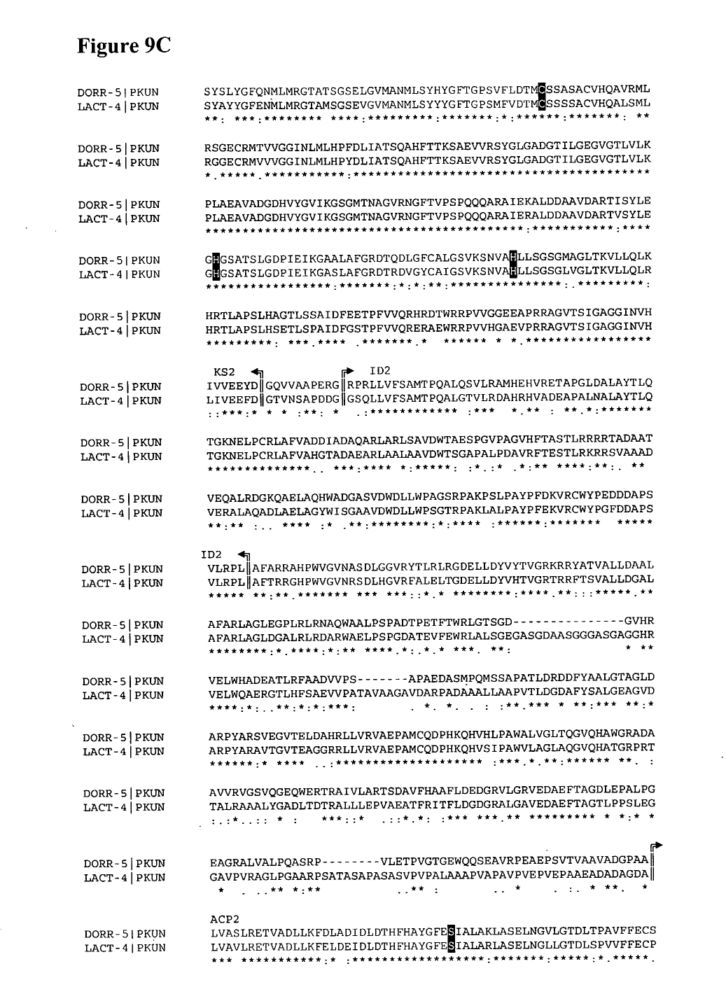

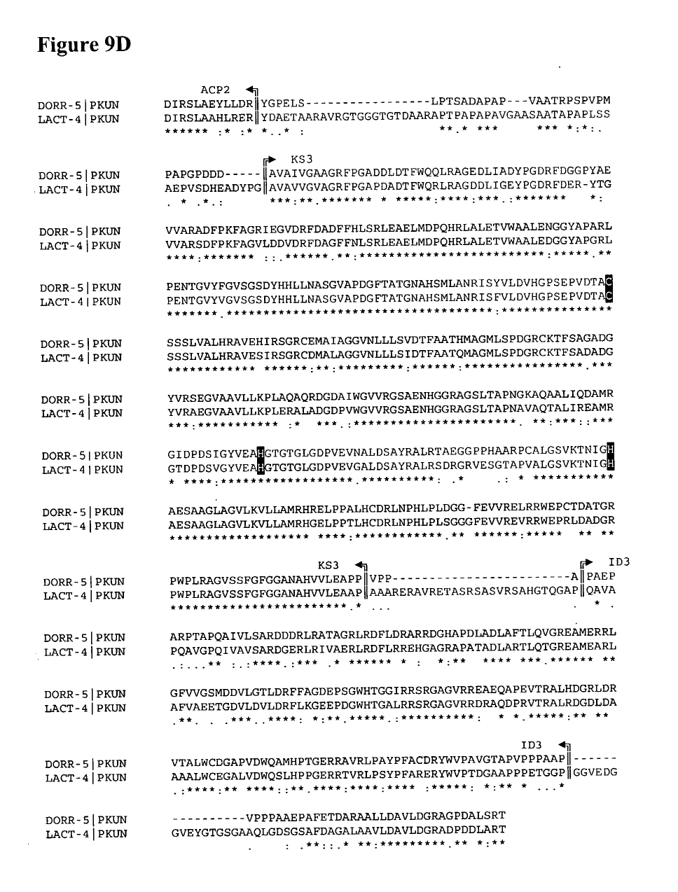

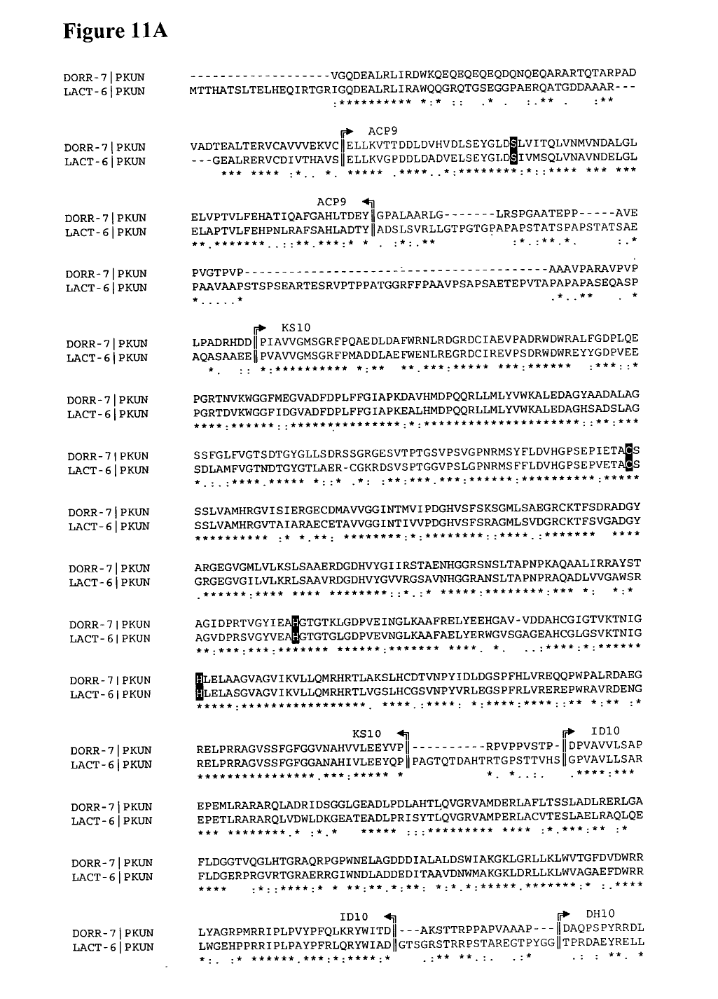

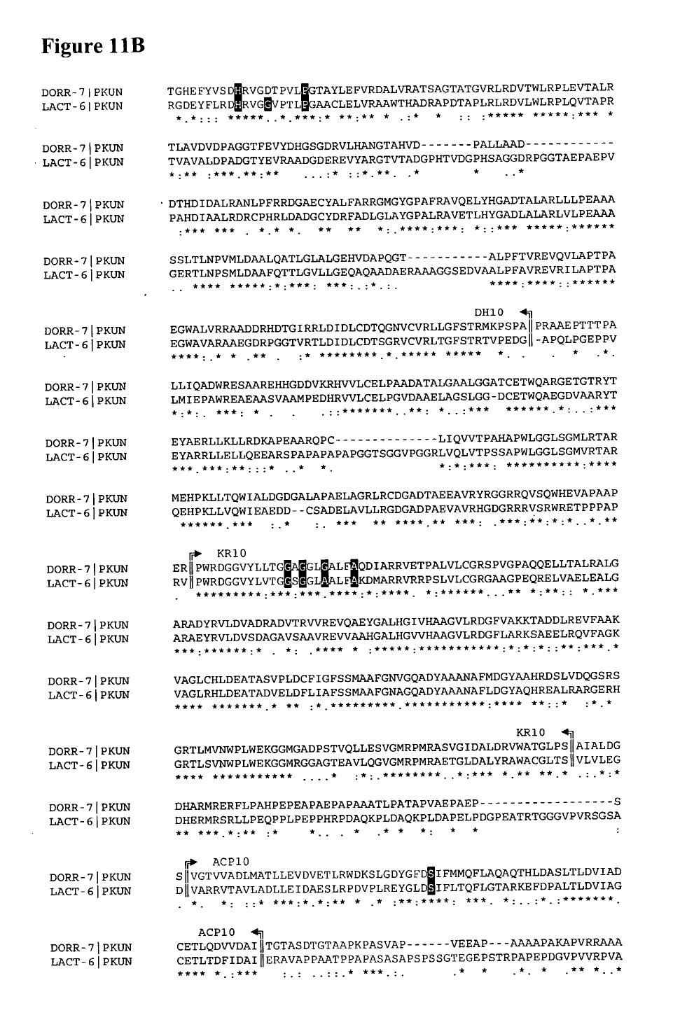

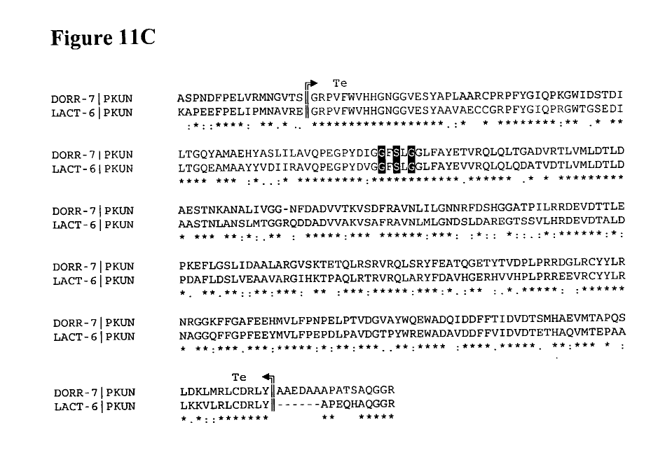

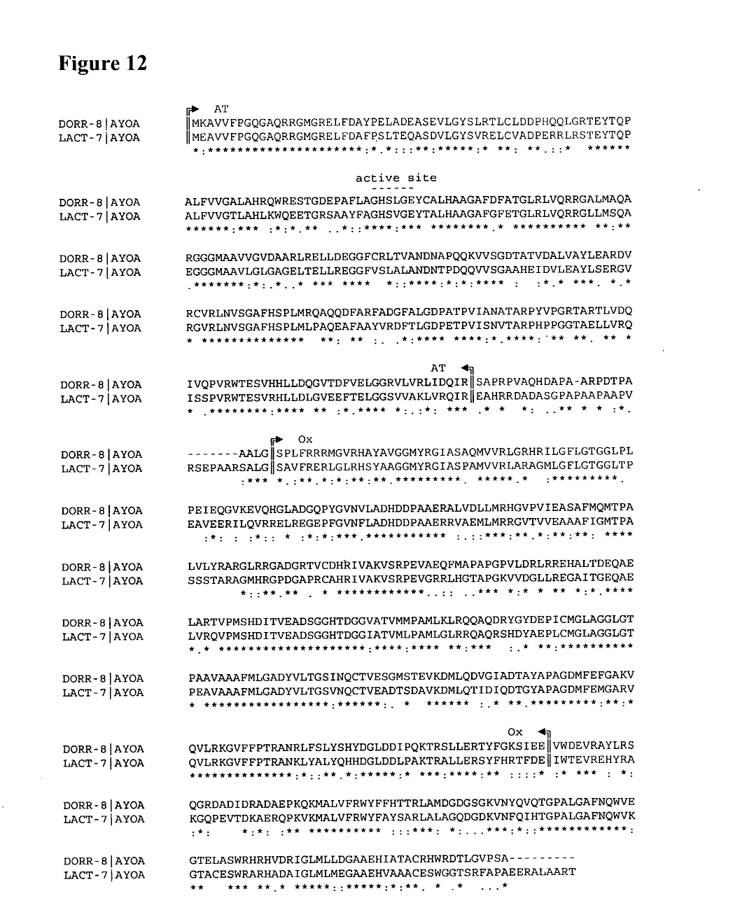

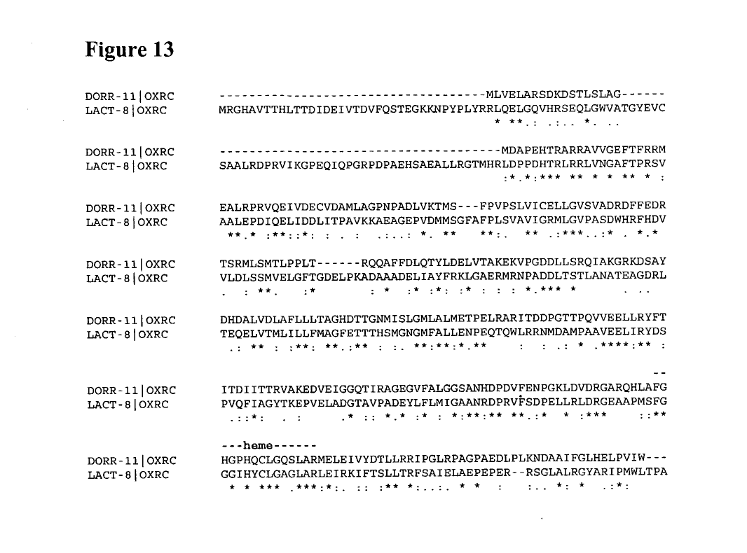

FIGS. 6 to 13 are amino acid alignments comparing the various ORFs that are common to both the dorrigocin biosynthetic locus and the lactimidomycin biosynthetic locus. Where applicable, key active site residues and motifs for the various polyketide synthase domains as described in Kakavas et al. (1997) J. Bacteriol. Vol 179 pp. 7515-7522 are indicated in FIGS. 6 to 13.

Identification of domains and assignment of their boundaries is based on the literature pertaining to type I PKSs. Given that the two component PKS systems described in this invention are quite divergent from type I PKS systems, is possible that boundaries may be slightly incorrect or that novel domains that are unique to the two component PKS systems may have been inadvertently missed. Tables 3 and 4 list the approximate amino acid coordinates of the various domains of the polyketide synthase components involved in the biosynthesis of dorrigocins, migrastatin, and isomigrastatin (Table 4) and in the biosynthesis of lactimidomycin (Table 5). The expression of the DORR locus results in the production of both linear polyketides (the dorrigocins) as well as cyclic polyketides (migrastatin and isomigrastatin). Accordingly, it is to be expected that the expression of the LACT locus results in the production of a linear polyketide product in addition to the cyclic polyketide lactimidomycin. To date, a linear of lactimidomycin has not been described either because it is produced at very low levels or it is unstable.

TABLE 4

| | | Amino acid | | |
|---|---|---|---|---|
| ORF no. | Accession no. | coordinates | Homology | Module no. |
| 2 | 088CEP_01 | 1-276 | acyl transferase domain (AT) | NA |
| | | 311-529 | thioesterase domain (Te) | |
| 3 | 088CEP_02 | NA | acyl carrier protein | NA |
| 4 | 088CEP_03 | NA | amidotransferase | NA |
| 5 | 088CEP_04 | 14-444 | ketosynthase domain (KS) | 1 |
| | | 456-604 | interaction domain (ID) | |
| | | 631-901 | dehydratase domain (DH) | |
| | | 1091-1312 | ketoreductase domain (KR) | |
| | | 1361-1432 | acyl carrier protein domain (ACP) | |
| | | 1508-1939 | ketosynthase domain (KS) | 2 |
| | | 1950-2107 | interaction domain (ID) | |
| | | 2439-2510 | acyl carrier protein domain (ACP) | |
| | | 2547-2976 | ketosynthase domain (KS) | 3 |
| | | 2989-3156 | interaction domain (ID) | |
| 6 | 088CEP_05 | 182-404 | ketoreductase domain (KR) | 3 |
| | | 446-512 | acyl carrier protein domain (ACP) | |
| | | 555-984 | ketosynthase domain (KS) | 4 |
| | | 998-1190 | interaction domain (ID) | |
| | | 1205-1276 | acyl carrier protein domain (ACP) | |
| | | 1299-1706 | ketosynthase domain (KS) | 5 |
| | | 1718-1852 | interaction domain (ID) | |
| | | 1863-2128 | dehydratase domain (DH) | |
| | | 2341-2562 | ketoreductase domain (KR) | |
| | | 2733-2949 | methyltransferase domain (MT) | |
| | | 3025-3093 | acyl carrier protein domain (ACP) | |
| | | 3143-3576 | ketosynthase domain (KS) | 6 |
| | | 3592-3761 | interaction domain (ID) | |
| | | 4012-4228 | ketoreductase domain (KR) | |
| | | 4394-4610 | methyltransferase domain (MT) | |
| | | 4677-4743 | acyl carrier protein domain (ACP) | |
| | | 4774-5186 | ketosynthase domain (KS) | 7 |
| | | 5199-5321 | interaction domain (ID) | |
| | | 5368-5440 | acyl carrier protein domain (ACP) | |
| | | 5507-5918 | ketosynthase domain (KS) | 8 |
| | | 5929-6093 | interaction domain (ID) | |
| | | 6113-6384 | dehydratase domain (DH) | |
| | | 6567-6796 | ketoreductase domain (KR) | |
| | | 6852-6907 | inactive acyl carrier protein domain | |
| | | 6943-7017 | acyl carrier protein domain (ACP) | |
| 6 | 088CEP_05 | 7061-7496 | ketosynthase domain (KS) | 9 |
| | | 7509-7666 | interaction domain (ID) | |
| | | 7667-7803 | dehydratase domain (DH) | |
| 7 | 088CEP_06 | 62-123 | acyl carrier protein domain (ACP) | 9 |
| | | 176-611 | ketosynthase domain (KS) | 10 |
| | | 622-777 | interaction domain (ID) | |
| | | 790-1060 | dehydratase domain (DH) | |
| | | 1242-1470 | ketoreductase domain (KR) | |
| | | 1533-1589 | acyl carrier protein domain (ACP) | |
| | | 1653-1943 | thioesterase domain (Te) | |
| 8 | 088CFP_01 | 1-277 | acyl transferase domain (AT) | NA |
| | | 302-637 | oxidoreductase domain (Ox) | |

NA Not Applicable

TABLE 5

| | | Amino acid | | |
|---|---|---|---|---|
| ORF no. | Accession no. | coordinates | Homology | Module no. |
| 1 | 133CBP_37 | 1-274 | acyl transferase domain (AT) | NA |
| | | 341-565 | thioesterase domain (Te) | |
| 2 | 133CBP_24 | NA | acyl carrier protein | NA |
| 3 | 133CBP_25 | NA | amidotransferase | NA |
| 4 | 133CBP_26 | 35-465 | ketosynthase domain (KS) | 1 |
| | | 501-657 | interaction domain (ID) | |
| | | 709-992 | dehydratase domain (DH) | |
| | | 1212-1433 | ketoreductase domain (KR) | |
| | | 1499-1570 | acyl carrier protein domain (ACP) | |
| | | 1657-2088 | ketosynthase domain (KS) | 2 |
| | | 2099-2253 | interaction domain (ID) | |
| | | 2618-2689 | acyl carrier protein domain (ACP) | |
| | | 2751-3180 | ketosynthase domain (KS) | 3 |
| | | 3201-3436 | interaction domain (ID) | |
| 5 | 133CBP_55 | 191-414 | ketoreductase domain (KR) | 3 |
| | | 482-552 | acyl carrier protein domain (ACP) | |
| | | 640-1080 | ketosynthase domain (KS) | 4 |
| | | 1104-1271 | interaction domain (ID) | |
| | | 1308-1379 | acyl carrier protein domain (ACP) | |
| | | 1411-1816 | ketosynthase domain (KS) | 5 |
| | | 1831-1984 | interaction domain (ID) | |
| | | 1988-2274 | dehydratase domain (DH) | |
| | | 2522-2743 | ketoreductase domain (KR) | |
| | | 2917-3133 | methyltransferase domain (MT) | |
| | | 3240-3308 | acyl carrier protein domain (ACP) | |
| | | 3371-3804 | ketosynthase domain (KS) | 6 |
| | | 3816-3986 | interaction domain (ID) | |
| | | 4270-4491 | ketoreductase domain (KR) | |
| | | 4664-4880 | methyltransferase domain (MT) | |
| | | 4966-5033 | acyl carrier protein domain (ACP) | |
| | | 5092-5504 | ketosynthase domain (KS) | 7 |
| | | 5523-5677 | interaction domain (ID) | |
| | | 5708-6117 | ketosynthase domain (KS) | 8 |
| | | 6130-6290 | interaction domain (ID) | |
| | | 6318-6593 | dehydratase domain (DH) | |
| | | 6805-7035 | ketoreductase domain (KR) | |
| | | 7095-7166 | acyl carrier protein domain (ACP) | |
| | | 7227-7296 | acyl carrier protein domain (ACP) | |
| 5 | 133CBP_55 | 7378-7814 | ketosynthase domain (KS) | 9 |
| | | 7836-7991 | interaction domain (ID) | |
| | | 8011-8294 | dehydratase domain (DH) | |
| 6 | 133CBP_06 | 67-136 | acyl carrier protein domain (ACP) | 9 |
| | | 242-677 | ketosynthase domain (KS) | 10 |
| | | 699-854 | interaction domain (ID) | |
| | | 873-1173 | dehydratase domain (DH) | |
| | | 1365-1593 | ketoreductase domain (KR) | |
| | | 1661-1730 | acyl carrier protein domain (ACP) | |
| | | 1803-2094 | thioesterase domain (Te) | |
| 7 | 133CBP_56 | 1-277 | acyl transferase domain (AT) | NA |
| | | 310-645 | oxidoreductase domain (Ox) | |

NA Not Applicable

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all sizes and all molecular weight or mass values are approximate, and are provided for description.

Some open reading frames listed herein initiate with non-standard initiation codons (i.e. GTG - Valine) rather than the standard initiation codon ATG, namely DORR ORFs 2, 6, 7 (SEQ ID NOS: 4,12 and 14) and LACT ORFs 1, 3, 5 and 9 (SEQ ID Nos: 26, 30, 34 and 42. All ORFs are listed with M or V amino acids at the amino-terminal position to indicate the specificity of the first codon of the ORF. It is expected, however, that in all cases the biosynthesized protein will contain a methionine residue, and more specifically a formylmethionine residue, at the amino terminal position, in keeping with the widely accepted principle that protein synthesis in bacteria initiates with methionine (formylmethionine) even when the encoding gene specifies a non-standard initiation codon (e.g. Stryer, Biochemistry 3^{rd} edition, 1998, W. H. Freeman and Co., New York, pp. 752-754).

Patents, patent publications, procedures and publications cited throughout this application are incorporated herein in their entirety for all purposes.

1. An isolated, purified or enriched nucleic acid comprising a sequence selected from the group consisting of SEQ ID NOS: 1, 22 and 25; the sequences complementary to SEQ ID NOS: 1, 22 and 25; fragments comprising at least 10 consecutive nucleotides of SEQ ID NOS: 1, 22 and 25; and fragments comprising at least 10 consecutive nucleotides of the sequences complementary to SEQ ID NOS: 1, 22 and 25.

2. An isolated, purified or enriched nucleic acid capable of hybridizing to the nucleic acid of claim 1 under conditions of high stringency.

3. An isolated, purified or enriched nucleic acid capable of hybridizing to the nucleic acid of claim 1 under conditions of moderate stringency.

4. An isolated, purified or enriched nucleic acid having at least 70% homology to the nucleic acid of claim 1 as determined by analysis with BLASTN version 2.0 with the default parameters.

5. An isolated, purified or enriched nucleic acid having at least 99% homology to the nucleic acid of claim 1 as determined by analysis with BLASTN version 2.0 with the default parameters.

6. An isolated, purified or enriched nucleic acid comprising a sequence selected from the group consisting of SEQ ID NOS: 3, 5, 7, 9, 11, 13, 15, 17, 19, 21, 24, 27, 29, 31, 33, 35, 37, 39, 41, 43 and the sequences complementary thereto.

7. An isolated, purified or enriched nucleic acid capable of hybridizing to the nucleic acid of claim 6 under conditions of high stringency.

8. An isolated, purified or enriched nucleic acid capable of hybridizing to the nucleic acid of claim 6 under conditions of moderate stringency.

9. An isolated, purified or enriched nucleic acid having at least 70% homology to the nucleic acid of claim 6 as determined by analysis with BLASTN version 2.0 with the default parameters.

10. An isolated purified or enriched nucleic acid having at least 99% homology to the nucleic acid of claim 6 as determined by analysis with BLASTN version 2.0 with the default parameters.

11. An isolated, purified or enriched nucleic acid comprising at least 10 consecutive bases of a sequence selected from the group consisting of SEQ ID NOS: 3, 5, 7, 9, 11, 13, 15, 17, 19, 21, 24, 27, 29, 31, 33, 35, 37, 39, 41, 43 and the sequences complementary thereto.

12. An isolated, purified or enriched nucleic acid having at least 70% homology to the nucleic acid of claim 11 as determined by analysis with BLASTN version 2.0 with the default parameters.

13. An isolated or purified polypeptide comprising a sequence selected from the group consisting of SEQ ID NOS: 2, 4, 6, 8, 10, 12, 14, 16, 18, 20, 23, 26, 28, 30, 32, 34, 36, 38, 40 and 42.

14. An isolated or purified polypeptide comprising at least 20 consecutive amino acids of the polypeptides of claim 13.

15. An isolated or purified polypeptide having at least 70% homology to the polypeptide of claim 13 as determined by analysis with BLASTP version 2.2.2 with the default parameters.

16. An isolated or purified polypeptide having at least 99% homology to the polypeptide of claim 13 as determined with BLASTP version 2.2.2 with the default parameters.

17. An isolated or purified polypeptide having at least 70% homology to the polypeptide of claim 14 as determined by analysis with BLASTP version 2.2.2 with the default parameters.

18. An isolated or purified polypeptide having at least 99% homology to the polypeptide of claim 14 as determined with BLASTP version 2.2.2 with the default parameters.

19. An isolated or purified antibody capable of specifically binding to a polypeptide having a sequence selected from the group consisting of SEQ ID NOS: 2, 4, 6, 8, 10, 12, 14, 16, 18, 20, 23, 26, 28, 30, 32, 34, 36, 38, 40 and 42.

20. An isolated or purified antibody capable of specifically binding to a polypeptide comprising at least 10 consecutive amino acids of one of the polypeptides of SEQ ID NOS: 2, 4, 6, 8, 10, 12, 14, 16, 18, 20, 23, 26, 28, 30, 32, 34, 36, 38, 40 and 42.

21. A method of making a polypeptide having a sequence selected from the group consisting of SEQ ID NOS: 2, 4, 6, 8, 10, 12, 14, 16, 18, 20, 23, 26, 28, 30, 32, 34, 36, 38, 40 and 42 comprising introducing a nucleic acid encoding said polypeptide, said nucleic acid being operably linked to a promoter, into a host cell.

22. A method of making a polypeptide having at least 10 consecutive amino acids of a sequence selected from the group consisting of SEQ ID NOS: 2, 4, 6, 8, 10, 12, 14, 16, 18, 20, 23, 26, 28, 30, 32, 34, 36, 38, 40 and 42 comprising introducing a nucleic acid encoding said polypeptide, said nucleic acid being operably linked to a promoter, into a host cell.

23. A computer readable medium having stored thereon a sequence selected from the group consisting of a nucleic acid code of SEQ ID NOS: 1, 3, 5, 7, 9, 11, 13, 15, 17, 19, 21, 22, 24, 25, 27, 29, 31, 33, 35, 37, 39, 41, and 43 and a polypeptide code of SEQ ID NOS: 2, 4, 6, 8, 10, 12, 14, 16, 18, 20, 23, 26, 28, 30, 32, 34, 36, 38, 40 and 42.

24. A computer system comprising a processor and a data storage device wherein said data storage device has stored thereon a sequence selected from the group consisting of a nucleic acid code of SEQ ID NOS: 1, 3, 5, 7, 9, 11, 13, 15, 17, 19, 21, 22, 24, 25, 27, 29, 31, 33, 35, 37, 39, 41, and 43 and a polypeptide code of SEQ ID NOS: 2, 4, 6, 8, 10, 12, 14, 16, 18, 20, 23, 26, 28, 30, 32, 34, 36, 38, 40 and 42.

25. An isolated gene cluster comprising open reading frames encoding polypeptides sufficient to direct the synthesis of a dorrigocin compound or analogue.

26. The isolated gene cluster of claim 25 comprising an open reading frame selected from the group consisting of SEQ ID NOS: 2, 4, 6, 8, 10, 12, 14, 16, 18, 20, 23 and the sequences complementary thereto.

27. An isolated gene cluster comprising open reading frames encoding polypeptides sufficient to direct the synthesis of a dorrigocin compound or analogue, wherein the gene cluster comprises an open reading frame having at least 10 consecutive bases of a sequence of claim 26.

28. An isolated gene cluster comprising open reading frames encoding polypeptides sufficient to direct the synthesis of a dorrigocin compound or analogue, wherein the gene cluster comprises an open reading frame having at least 70% homology to the sequences of claims 26 as determined with BLASTP version 2.2.2 with the default parameters.

29. The isolated gene cluster of claim 28 wherein the gene cluster is present in a bacterium.

30. A method of expressing a dorrigocin biosynthetic gene product comprising culturing a bacteria of claim 29 under conditions that permit expression of the dorrigocin biosynthetic gene product.

31. The isolated gene cluster of claim 25 wherein the gene cluster is present in E. coli strains DH10B having accession nos. IDAC 270201 -1 or IDAC 270201-2.

32. An isolated gene cluster comprising open reading frames encoding polypeptides sufficient to direct the synthesis of a lactimidomycin or a lactimidomycin analogue.

33. The isolated gene cluster of claim 32 comprising an open reading frame selected from the group consisting of SEQ ID NOS: 26, 28, 30, 32, 34, 36, 38, 40, 42 and the sequences complementary thereto.

34. An isolated gene cluster comprising open reading frames encoding polypeptides sufficient to direct the synthesis of a lactimidomycin compound or analogue, wherein the gene cluster comprises an open reading frame having at least 10 consecutive bases of a sequence of claim 32.

35. An isolated gene cluster comprising open reading frames encoding polypeptides sufficient to direct the synthesis of a lactimidomycin compound or analogue, wherein the gene cluster comprises an open reading frame having at least 70% homology to the sequences of claim 32 as determined with BLASTP version 2.2.2 with the default parameters.

36. The isolated gene cluster of claim 34 wherein the gene cluster is present in a bacterium.

37. A method of expressing a lactimidomycin biosynthetic gene product comprising culturing a bacteria of claim 36 under conditions that permit expression of the lactimidomycin biosynthetic gene product.


SEQUENCE LISTING (43 sequences)

SEQ ID NO: 1 - DNA, 52101 bp, Streptomyces platensis subsp. rosaceus
ggcgcatctgatcacctctcagctcggtcctgctccccggcacgtgcaccggaaccgtcgccgccctcacccggccgccaccgcctccgcggccccacggcccaggaggtcgtggaggaggcgggtggcggggagttccggatgaaggtagacgggttcgtcgctctggccgacggctgcgaggagggcgtggaaggtggtcttcgcgtcctccgtccgtccggtccggtgctgggcctggccgaggaggcccaggtggagggggcggcgcaagtgggttctggagttgcggagttcggcgagggaggagtgcatcagggtgaggccgtcctcctgtccggagtgggtgcggccccagccctccagcacgccgagcatggccttccagtagagcagtccgtgttcgtccgccaggcggactccctcggcgccggaggagcgggccgtgcgggcgtcgccttcccaggccgccacgaccgcgtccacgtagagagcgaaggagcggtcggagggcctgctgtcgtactcggtgaggcgcagtaattcgcggcggcgtgccgtggcggtcttgcggtcgcccatgagccagtgggtgaaggtgtcgtaggagcggcaggagacgcgtgggtcgtgctggaacgtgcgggccagggagtggccctcgccggcgtactcgtccgccatggcgacgccgtgctccagttcggtgagtgccggccgcaactggccgcggatgtggaggacgatgccctcgccgtaggccgcgccgagcaccgccaccgggtggccggtccggtcggcgaggttccggagcaggcccgagaactggcgtgaggcgtcgtagcggccggtgacgatgtacgccgcgcacagggcccagagcaccgacgggtcttcgggggagtgggtggccgcgctcagggcccgtccgcgggcgagtgccgtctgggcctcggcgtggccgtagccccgggtgatggccagcacctggccgagttgaatgtgcagccgctggttcagggacacggccgaggggtcgccgggcggcagcaggccgaccaggtgcaccgcgcggcgcagccaggtctccacctgttcgtaggcgagttgctgctcggcctgctcggcggcgcgcagcaacagaggaagcgtttcttcagtcggcagtgcgctcttggcgtgccaggcgtggtgggcgatccgctcgatctcctcgtccgccacctgaccgagcgtgcgggtcgatatcccctcggcgactctcgcgtgcagccgctgccggtcctcgcgggggagctcgtcgatgagcgtctcctggaccagggcgtgggtgaagtggagccgttccgggtggtggcgatcctcgccgagcagcccggcccggatcgccaactccagtgccgcggtgaccggttcgtcttcggtggcggtgcgttccagcagatcggtgtcgacctcggtgccgatgacggcgcagagcctcagcacgcgcagcaccgtctcggggagtgcggcgaaccgctggtgcagggcctcgcggacgcccgtcgggacgcgcgtcaggagcacgtccaccgcgtcgggccgccgcaggctgcgagcgtcacccagaagggagaggagctgcatgacgaagtacgggttgccctcgctgcgccggtgcagcacctcgacgactctcgcgtcgacgccgggtccgacctgggcgacgacgagggcggcgaccgcgcggcggggcaggccgccgagccgcagggtctcggtcctggggccgcgcagcacctcggagagcatgcggcgcagcgtcgcatcggactcgatctcgtgctcacgggcggtcaggacgatgccgagcgggtggccctggctgcgggtgctcagcagtctgaggaggtccagggaggcggggtcggaccaatgcaggtcctccatgagcaggacgagtggccgctgggcggcgagggcgagcaggacttcgcagaccgcgtcgtgggtgaggaaccgggcttggccccagtcggattcggaggcgagtccgccgggccgggccgcgcgctcgggcatcagcggggcgagcagggtgccgaacggtttcgccgcctcgcggaaggcgtccgggcgggtcgtggacagtcgccggaggatctgggtccacacccagtagggcggcacgccctcgcccaggaagcagtggctccagatcacctccaggccggcgcgctcctgaccggtgccatcctcgccggcgcgggcggcttccagctgggggacgagttcgaggagcagacgggtcttgccgacgcccgcggggccgaggacgcccgcaacgtgcccgtgaccggcgagcgcgctcgacgccgcggctgtcaggcgctgtaactcctcgctgcggccggtgaacggcgactgggcgcccggaccgccgcacaccggccctgaggcctcggtgctcgtgtcgcactcctgggctttcgcatcctgcgtcggacgcgcgctctccgctgaatccgcggtggtcgaccgcgatgtcgcggtgatcgtcaccggccggcgcgcgggtcccggtgtggcggtgggcgcgtccgcctgcgccccgtccgcggcagcgcttccggcccctcccgatcccgcggacagcgtgccgtcctggggagcgtcatcaccgcagtctgctgcgaagggctcgtccggagccgctgccgcccggccgatcccaccggacgcctcggccgctgtgggccgctcgggctcctgcgccgcgcgtggcgggtgcgcatccggccctcgtgcaccgtcgccctcgtgcgccgtggccaccccgatcacaggagcccccagcgcgggcccggcaggttcggggcgaccgggtcgtggtcccgcggggggaccgcccgcacccggctcctggcgcaggatcgcggtcctgacccggcggagttcggcggccgtgcccacgccgaactcctcgttcaggtggacgcgggtccgttcgtacacttcgagcgcctcggcctgccgcccgagctgggacagggccgtcatcaagtggccgaccaggcgttcccgcgccgggtggcggcgcacctcgcggtcgaggcccgcggccacttcctgggcctcgccgagggcgagtcgggcctgcgcgcaggactccacggcggtgagccggacgtgttcgagccgcgcactctcgtcgctgagcggaggatggccgtcgaactccgcatagggcgagccccgccacagtgtcagggctgcggcgaaccggtcccgcgcggcgagcgggtcgcgttgttccagcagccgccgcccttcggcgaccaggcgctcgaagcggcaggcgtcgagctgttcgggggcgagttccaggacgtagccgggcgcgcggtggcgcagcacggtggggcggtcgagcccggacgccggctgcagtgcgcggcgcaggtgggatacgtggctctggagcgtggcgacggcctgacgcgggggctcctcgccccacagctcctcgatgagcagttccgtgggcaccacacgccccaggcgtatgaggagcagggcgagcagggccctgcggcgtggcggaccgagtgggaggtcgcggccgtcgtggcgtgccgacatggggcccagaacgctcagttggaggggagtcccgtgcggcccgtcctcggaggccggagtgaggggcggctgattcggcgtcagggatgaaggcatatcggtcccaatcgttctcgcacgtgcgatcgtgctgacgggcgggtggcgcagtgcggccggtgcccgtccaaaccggagctttgcatagagaacggggacgttccaagatcaccctggctgtcggcaagcgaatggcaaggtccctgcagggagttcggcaaggttggcggaaaaccgctacagcgaacggcaggaattcaccagtgtgaagaccaggccgctgcaaggggggtggtgcacgatgcccgtcatcggaaaccaccgctgaagggggaggtcatgaacccgccggacgactgccctgccgtcacccgagaacactccgctgcgggcccggcatgagggcttccacagcgggagtgcccgccgtgctcttccccggccaggggtcccaggcccgtggtatgggagccggcctcttcgaccggtaccccgaactgaccgcgttggccagcgatattctcggctacgacctcccgcggctgtgcctggaggacccggacgggcggctcgacgacacgcgctgcacccagcccgcgctctacgtcgtcaacgccctctcctaccaagactcgctggagcgcggcgaacccgagggcgggtacctgctgggccacagcctcggggagtacaacgccctgcacgccgccggggccttcgacttcgagaccggcctgaagctcgtcctcaagcggggcgagctcatggcccgggctccggacggggccatgctcgccgtcgtggggccggacgcgggtgaggtgcgggccttcctctcggaggagggcctgtcgcggctcgacgtcgccaacatcaacacccccgtccagaccgtgctgtccggggcccgggacgagatcgagcgcgcgcacaagacgctcgacgcgcacgggacccgggtcgcccggctgaaggtctcggccgccttccactcgcggttcatggccgctgcccgcgacgagttcgccgccttcctcaaaggcttccggttcgcgcccctgcgggccacggtgatcgccaacctcaccgcacggccgtacacggaccaggacgtcgcggcgacgctgagcgagcagatctgcggatcggtgcagtggctggacagcgtccgctacctgctggagcgcacgaccgccggccactgccgtgaggtgggcgggggaggagtcctgacccgcatgatccggcagatcgacgcggcccccgcccgcgggattccacagccgaagccgaagccgaagccgaagccgaagccgaagccgcagtcacggccacgcctgttctgcatcgcctacgccggaggcgacgagcgcgcctacgcaggactcgccgaacactgcccggatgtcgacgtcgtgacgctggaacgccccggacgcggccggcgcgcctccgagccgctgctgcgcgaacccgccgcgatcgtcgacgatctgctccggcagctgcggggccggctcgacgccccgtacgcgctctacggccacagcctcggagcccggctggcattcctgctctgtcgggcgctgcgcgccgagcggctgcccgcaccggcccacctgttcgtctccggggagagcggaccggccctcccgagccgggaacgccacacctgggagctgccggccgacgccttctgggaccacctcaaggagctcggcggaatcccggcggagctgtgggagcaccccgacctgatggcgtattacgagccggtcatacgggccgacttcaccgcgctgggcgcctaccggcacgaggacgctccgccgctggacgtgccggtcaccgccatggccggcgaggacgagtggttcacccgggccgacctggaggcctggcaacgcgagagcacccggcccctgaccacacaccggttccccggtgatcacttcttcatccgggcccagtggcccgcgctggcccggatcgtcgctgccgggctcgcggccccctgacgccgcgccggaacagcgaagagctcacgcgcgccggccaccggcacgcgccccgaatcgaccatcccgagggtgacggacagccatgaagcaggaactcaagaagcacatggaagagcggttcatgttcgagttcgactcggacatcaccgaggacaccgacctgttcaaggcgggcatcctcgactcgttcggttatatctcgctgatgacgcacatcgaggaggagtacggcgtgccgctcggcgacgagatcctcggcaacgtcgcggtctcgctgtccggcatcgtcgcgttcgtcgacgccgcccgcctgcgggccgccgggagccggtgacccgatgtgcggcatcgccggcttctacggaagccccctgccaccgcaggaatacgagaccctgatccacggcatgctcgcccagatcgagcaccgcggcccggacgaggcgggctgcttcctcgacgaccgcctggccatgggcacggtacgactgagcatcatcgacctgtccaccggctcgcagccggtcggcagcgccgacggccgctactggctctgctacaacggcgagctgtacaactaccgggagctgcgtgagcagctgaccgcccgcggcttcgtcttccgcaccgagtccgataccgaggtcgtgctggccgcctgggtcgcctggggcctggactgcctgccccgcttcaacggtgccttcgcctttgccctttacgacagtgccaccggcgaactgcacctggtgcgcgaccggttcggcaagcggccgctgtacgtggcgcggcaccgcggcgcgtggctgttcgcctccgagatgaaggcgttcctggcctaccccgacttcaggttcgccttcgacgaggcacagctggcgtcggtcttcgccacctggaccccgctgcccggccagagcggataccaagggatcgagcagatccccatgggcgagtatctgtccgtacgcggcgacgaggtccggcgcggccgctgggccacgctcgacctggcccaaggcccggctccggagagcgagcaggaggccgccgagcttgtccgcgcggacctcgaagccgcggtcgacgtgcgcctgcgcagcgatgtcgaggtcggcgtctacgcctccggcggcctggactcctcgatcatcgcgcacatcgccgcgcagcggacgagccgcccgctgcggacgttctcgatcgagttcgaggacgcagagttcgacgaatcggccgaacaggccgagctggccgcacacctgggcacccgccactccaccgtgcgcgtgaccgacgaggacgtcgccgacgccttccccgaagccgtccggcacgccgaggtgcccgtcttccgcaccgccttcgtccccatgtacctgctggcaggccacgtccgcagcgaagggatcaaggtcgtgctcagcggcgagggcgccgacgaggccttcctcggctacggcatcttcaaggacacgctgctgctctcgacctggcacgagctggacgacgacacccgtctgcgccgcatgagccagctctacccgtacctgagccacttcagcggcgaggacggccaccgccggatgctcggcctctaccggcagttcaccgaggagaccctgcccggcctcttctcccaccagatgcggttccagaacggccgcttcgccgcacgcctgctcaagaacccgggcgaccccttcgcggccctcggggaactcgtggccggtgagcccggctacgcacagctcacccccgtacagaaggcccagtggctggagttccgcacgctcctgagcggctacctgctctcgacccagggcgagcgcatggcgctggcccacggcgtggagaaccgctgccccttcctcgatcccgccgtggtccgccgcgccgcatcggtgaacctgcggttcggcgacccctacgacgagaagtacctgctcaagtgcgcctatgccgatgtgctgccggaacggatcgtcaagaaggggaagttcccctaccgcgccccggacagcgccgcgttcgtccgctcccgcccggactaccgcgagctgctgaccgaccccggcaccctcgacgagatcggcgtcctcgatgcgcgcttcgtgaagcggttcaccgaccgcgtcttcgacagcccgcctgagcagatcggcacgaaggagaaccaggccttcgtctctttggcgtcgacggtctggctgcaccactggtacgtgcgcggcaacgcccgccgccgggctccgctcggggtccccctgtacgtcgtcgaccggcgcagtggcgccctgtcggcctaggacggagaacgcgccatgaagaagcagaacggcgtcctcgccgacgaccgggacatcgccgtcatcggcctgtccctgcggttgcccggctcgcgcacgcccgaggagttctggagccacctggccgagggccgctcgctcatcagcgaagtcccggagcgccgatggcgaaaggaggaccacctcggtcacccgcgccgcgaattcaacaagaccaacagcgtctggggcggcttcgtcgacgacgccgactgcttcgacgccgatttcttccagatctccccgcgcgaggcgcagtccatggacccgcagcagcggatggccctggagctgagctggcacgccctggaggacgccggctaccgggccgaccgcgtggcgggctcccgcaccggtgtcttcatgggcgtctgccactgggactacgccgagctgatggagcaggaagtcgaggagatcgacgcctactacccgaccggcgccgcgtacgcgatcatcgccaaccgggtctcccaccacttcgacttccgcggaccgagcgtcgtcaacgacacggcctgcgcgggctcgctcgtggccgtgcagcaggcggtgcaggccctccaggccggcgactgcgacctcgcgctcgccggcggcgtcaatctgacctggtcgccgcggcacttcatcgccttcgccaaggcgggcatgctctcgcccgacggcctgtgccgggcgttcgacgcgaatgccaacggctatgtgcgcggcgagggcggcggcatcgtgctgctgaagcgggccgcggacgcccgccgcgacggcgacgccgtgcacgccgtgatcaagggcatcggcagcaaccacggcgggcgcaccagttcgctgaccgttaccaacccggccgcacaggccgaactgatcgcgggtgtctaccgcaaggccggcatcgcacccgagaccgtcacctacgtggagacccacggccccggcacaccggtcggggaccccatcgaggtccgcggcctcaagcaggccttcgtcgacctgggcgcagaccggcccggggaggctccggcccaccggtgcggcatcggctccgtgaagaccaacatcggccacctggaaggggccgccggcatcgcgggcatgctcaaggtcatcctcgccatgcgccaccgcaagctgcccgcgacggtcaacttccgcaagctcaaccccctcatcgacctggacggcagcccgctgtacgtcctcgaccgcctcaccgactggaccgccgaagggtccgcaccgctgcgcgccggcgtcagctccttcggattcggcgggaccaacgcccacgtcctgctggaagccgcggagccggtggccgccaccgaggacgccggcgaacagtggctgccggtgtccgccatggacgaggaccggcttcgcgagacgtgcgcccggctcgcccgctgggtccggacccggatcgagcagaacgatgctccgtccctgaccgatgccgcccgcacgctgcgcgaaggccgggtgtccatgcgcgagcgcgtggtgttccgcgcaagcggcatcgaggagtgggcggcacagctggagagcgtcgccgcgggggacggcccgcccgcggactgcccgcgcggccgggccggaaccgaagcccccgacggcctggacgcggacgacctgacggccctggccgagcgctggctggagaagggccggtgggacaagttcgcggccgcctgggcccagggcctggccgtggactgggcaccgtggcccgagcgcggccgccgcgtgcacgtgcccggctacgcgttcgcccgcacgccgcactggttccggacggaccggaacgagacgaccggaaggccggagcgcggcgcgacgaacaccgctcccgccccgctcggcgaaggcaagccggaaggcggcagctggaccttccccctgcacttcgacgccacccagggattcgtccgcgaccaccgcgtcaacggcgcacggatcgtcccgggcgtggtggccctggaactcgtcaccgtggcagccgaacgggccgccgccgcaggtgcccgggccgggctgacgccccgcatccgcaacgcggtgtggatccgtccgctgctcgtcggcgacaccgtgctctccccgcagctccgcctgacccccgccgccgacggctacgactacgcgatcaccgacgagcacggcacgcagtacaccagcggccgggtcgagtacggcgaggctgccgcggccgagaagacggacccgggcgcgctgcgcgagcgcttcccccagcgcgtcgacaccgccgagggttacgccgcgctgcggtccagcggcatcgagcacggccccgccctgcgcggcctcaacgccctgcaccgcggtccggacggcgtgctggccgagctgcggctccccgcaggtgccccggagggcatggcgctgcagcccgcgatcctcgacagcgccctcctcgcggccctggctctcggctcggccgacggcggctggcgcaggcccgccgcccccgtggtgccgttcgcgctggaccggctcaccgtgcacgcggcgacgacctcgacgatgtgggcgtggctgcggccggccggcagcggcacggcaggtgacatggccagatccgacatcgacctgttcgacgacaacggccggctgtgcgtgcgcctggccggctacacgtcgagggaactgcccaccgcagaaccggcagcagtccaggccccggaaggggagctcctggaggtcaccggtgtgtgggaggaggcccctgccccggcgcccgcagccggtcaggccaccccggtcggcccggtgacggtgctcaacgccgcactggacggcgacctcgcagcggcgagcgccgcgcggctgggcatggacatccggcagctggccggtcccgcggaagccaccgatgccaccgatgccgtcgccatgaaggcggcgttcgaggcctgctacccgcaggtccggcaactgctcggtcagggacggcaggtgctcgtcgtcgccccgggcgccccggactcgccggtctacgccccactggcggcgctgctgaagaccgcacaacaggagaatccttccttccgggggaggctggtgctcctcgacggctacgacccccgcgacgccgaccgcttcgagcgtgtcgtcagcgcggaggcgggcgccggagacgacaccgaagtcgcctacgacgcccaggaccgccgactgcggcacggcttcgtggaacttccccggggcgaggcgggggagagcctgctgcgcgacggtggcgtctactggatcaccgggggcgccggcggactcggcctgctgctcgccgagcggctctgcgagcgccgccgcgccacggtcgtcgtcagcggccgctcggcggacagccgggccatcgaggcactgcgggcccgcctgttccacggcgaggtggcgtaccgccgcacggacgtcacggacgcggacgccgtacgggacgcggtcgcggacatccgcgcgcggtacggacggctcgacggcgtgttccacgcggccggcgtcctcgacgacggctacctcgcgagcaagcccctcgcgggcaccgccgccgtactcgcgcccaaggtggacggcgccacgtccatcgatgacgcgacgcgcgcccacggcctggacttcctcctgctgttcggctccgtggcgggcgccttcggcaacgccgcgcaggccgactacgccgccgccaacgccttcctcgacgcgttcgccgcacgacggcaggccgccggcagcgtgacccgctccgtcgactggccgctgtgggccgacggcggcatgcgcgtggacgacgccagcctcgcctacctgcgcaagcgcaccgggaccgtgccactgccgagcgagaccggcctggacgcactggagcgcgcactgcactccgccgcgccggtccgccgcgtggtgctcttcggggagcggtccaagctgcgcgggtacgcgggcctggaccgcgtcgcgaagccggagccgcgcacgtccggggcgcagcggaacacggccgcgccggccgtcctggaggagagcgaactcgtagcccgtacacaggacctgctgcggaacctgttcgccgaggtgaccctgcaggacgcggagcacatcctggccgaggagaagctggagacctacggtatcgaatcgatctccatcgtcgagctgaccagcaagctggaggacaccttcgggtcgctgcccaagacgctcttcttcgagtacgtcgatctgcagggcgtggccggctacttcgtcgccgagcaccgcgaccggctcctcgaactcttcgcccccgaagcacccgcccccgaagcacccgcccccgaagcacccgcccccgaagcacccgcgcccgaggagcccgccccggaggggcctgccgtcgaggagccgcccgcggccgcgcccaccccggccgtccggccgtccgtggaggccgccgccgggcgcgcccgcccggcctgggccgatccggagcgccacgacatcgcggtcatcggtatggcgggccggtacccgggcgccgacaccctggaggagttctgggagctgctcagcgagggccggcacagtttcgagcccgtcccggaatcgcggtggcggcacggcgacatctacttcgacgagcgtgacgtcgacggcaagaccgtcgtcaagaccggcaccttcctgcgggacgtcgaggcgttcgatccgcgctacttcaacatctcccagcgcgacgccgagctgctgtcgccggaggtccggctgttcctgcaggcgggcgtggaggccctggaggacgcgggctactcacgtgagacgctgcggcgccgctacgacggcgacgtcggtgtgctcgtcggctcgatgaacaacagctactcgctctacggcttccagaacatgctgatgcgcggcaccgcgaccagcggcagcgagctcggtgtgatggcgaacatgctgtcgtaccactacggcttcaccgggccgtccgtgttcctcgacaccatgtgctcctcggcgtcggcgtgtgtgcaccaggcggtgcgtatgctgcgcagcggcgagtgccgcatgaccgtcgtcggcggcatcaatctgatgctgcacccgttcgacctcatcgcgacctcgcaggcgcacttcaccaccaagtcggccgaagtcgtgcgcagttacggcctcggcgccgacggcacgatcctgggcgaaggcgtgggcacgctcgtgctcaagccgctggccgaagccgtcgccgacggcgaccacgtctacggcgtgatcaagggcagcggcatgaccaacgccggggtccgcaacggcttcacggtgccgagcccacagcagcaggcgcgcgccatcgagaaggcgctcgacgacgccgccgtggacgcgcgcacgatcagctacctggagggtcacggctcggcgacctccctcggcgaccccatcgagatcaagggcgccgccctcgcgttcggccgggacacccaggacctggggttctgcgcgctgggctcggtcaagtccaacgtggcgcacctgctgtccggatccggcatggccggcctgaccaaggtgctgctgcagctcaagcaccgcacgctggcgccctcgctgcacgccgggacgctcagctcagcgatcgacttcgaggagaccccgttcgtggtgcagcgccaccgcgacacgtggcggcgccccgtggtcggcggcgaggaggcgccgcgccgggcaggcgtcacgtccatcggcgcgggcggcatcaacgtgcacatcgtcgtcgaggagtacgacggccaggtcgtcgccgcaccggagcgcggtcgcccgcggctgctggtgttctccgccatgacaccccaggccctgcagtcggtgctgcgcgccatgcacgagcacgtacgggagaccgcaccgggcctggacgccctcgcgtacaccctgcagaccggcaagaacgaactgccgtgccggctggccttcgtcgcggacgacatcgcggacgcccaggcccgtctggcccggctgtccgcggtggactggacggcggagtcacccggcgtgcccgcaggcgtgcacttcacggcgagcacgctgcggcgccggcgcaccgccgacgcggcgaccgtcgaacaggccctgcgcgacgggaagcaggcggagctggcgcagcactgggcggacggcgcgagcgtcgactgggacctgctgtggccggcgggaagccgtccggccaagccgtcgctgcccgcctaccccttcgacaaggtgcgctgctggtaccccgaggacgacgacgcgcccagcgtgctgcggccgctcgccttcgcccggcgcgcgcacccctgggtcggcgtcaacgcctcggacctgggcggggtgcgctacaccctccggctgcgcggcgacgaactcctcgactacgtctacaccgtaggacgcaagcgccgttacgccaccgtggcgctgctggacgcggcactggcgttcgcgcggctcgccgggctggaagggccgctgcggttgcggaacgcgcagtgggccgcactcccgtcgcccgcggacacccccgagacgttcacctggcggctcggcacgtccggcgacggcgtgcatcgcgtcgagctgtggcacgccgatgaggccacgctccggttcgccgccgacgtcgtaccgtccgcgcctgccgaagacgcatcgatgccgcagatgagcagcgcgcccgcgaccctcgaccgggacgacttctacgccgcgctcggcaccgcgggcctcgacgcccggccgtacgcgcgcagcgtcgaaggggtcaccgaactcgacgcccaccggctgctcgtacgggtcgccgaaccggccatgtgccaggacccgcacaagcagcacgtgcatctcccggcctgggcgctcgtcgggctgacccagggtgttcagcacgcgtggggccgggccgacgccgccgtggtgcgggtcggatccgtgcagggcgagcagtgggagcgcacccgggcgatcgtgctggcgcggacgtccgacgccgtcttccatgcggctttcctcgacgaggacggccgcgtgctgggccgggtcgaggacgccgagttcaccgcgggcgacctggagccggcactccccggtgaggccggacgcgcactcgtggcactgccgcaggcgtcgcgtccggtgctggagacgccggttggtacgggggagtggcagcagtcggaggccgtgcggccggaggccgagccgtccgtgaccgttgcggcggtcgcggacgggccggcggcgctcgtcgcgtcgctgcgcgagaccgtcgccgacctgctcaagttcgacctggcggacatcgacctcgacacgcacttccacgcgtacggcttcgagtccatcgcgctggccaaactggcctcggaactcaacggcgtcctcggcacggacctcacccccgccgtcttcttcgagtgctccgacatccgcagcctcgccgagtacctgctcgaccgctacggccccgagctgagcctccccacgagcgccgacgcccccgcgccggtcgccgccacccggccgtccccagtgccgatgccggcacccgggccggacgacgacgcggtggccatcgtcggcgctgccggacggttccccggcgcggacgacctggacaccttctggcagcagctgcgcgcgggcgaggacctgatcgccgactaccccggcgaccgcttcgacgggggcccctacgcggaggtcgtcgcgcgggcggacttcccgaagtttgccggccggatcgagggcgtggaccgcttcgacgcggacttcttccacctgtcgcggctggaggcggagctgatggacccgcagcaccggctggccctggagaccgtgtgggccgcgctggagaacggcggctacgccccggcgcgcctccccgagaacaccggggtctacttcggcgtctccggcagcgactaccaccacctgctcaacgccagtggcgtggcacccgacggcttcaccgccaccggcaacgcccactcgatgctggccaaccggatctcctacgtcctggacgtgcacgggccgagcgaacccgtcgacacggcctgctccagctcgctcgtcgcgctgcaccgcgccgtcgagcacatccggtcgggccgatgcgagatggccatcgcgggcggtgtcaacctgctgctgagcgtggacaccttcgccgcgacgcacatggcgggcatgctcagccccgacggccgctgcaagaccttctccgccggcgcggacggctacgtccgctccgagggcgtcgccgcggtgctgctcaagccgctcgcccaggcgcagcgggacggcgacgccatctggggcgtcgtccggggcagcgccgagaaccacggcggccgcgccggttcgctgaccgcccccaacggcaaggcgcaggccgccctgatccaggacgccatgcgcggcatcgacccggacagcatcggctacgtcgaggcgcacggcacgggcaccggcctgggcgacccggtcgaggtcaacgccctcgacagcgcctaccgcgccctgcgcaccgccgagggcgggccgccgcacgcggcccggccgtgcgcgctcggctcggtgaagaccaacatcggccacgcggagtcggccgcgggcctggccggagtgctgaaggtgctgctcgccatgcgtcaccgcgagctgccgccggccttgcactgcgaccggctcaacccgcacctgccgctcgacggcggattcgaggtcgtacgcgaactgcgccgctgggaaccgtgcaccgacgccaccgggcggccgtggcccctgcgggccggagtgagcagcttcggcttcggcggcgccaacgcccatgtcgtcctcgaagcaccgcccgtaccgcccgcaccggcggagccggcccgcccgaccgccccccaggccatcgtgctgtccgcccgcgacgacgaccggctgcgtgccacggccggacgactgcgggacttcctcgaccgggcgcgccgcgacggacacgccccggacctggcggacctggcgttcaccctccaggtaggccgggaggccatggaacggcgcctgggcttcgtcgtcggaagcatggacgacgtgctcggtacgctggaccggttcttcgcgggcgacgagccctccggctggcacaccggcggcatcaggcggtcgcgtggcgccggagtgcggcgcgaggcggagcaggcccccgaggtgacccgggccctccacgacggacggctcgaccgggtgacggccctgtggtgcgacggcgccccggtcgactggcaggcgatgcatcccacaggcgagcgccgcgccgtgcggctgcccgcgtaccccttcgcctgcgaccgctactgggtgcccgcggtcggcacagcccccgtcccgccgcccgcggcacccgtcccgccccccgcggccgagcccgcgttcgagaccgatgcccgtgcggcgctgctcgacgcggtcctcgacggccgtgccggcccggacgccctgagcaggacctgacgccttccccctgcctcctccccggaccgggcggctcggccgccccgtgacctggaacggaatgaacgtgagcagaaacatccttcgtgtgccggaatggcgcgacgaaccggcgcgagggcgcaccgcgccccccggaaaccggcggctggtcgtgctgtgcgacacccccgacgcggacgtgaccgacctgcgccggcacctgcccggcgtgtccgtcgcccgcgtggacagcggtgacgacgggcccgctgccgcctacgagcacgcggcgaccctgctgctcggcgagctccagcggctgctgaaccagccggccggcggcccccgttccgtgcaggtcgtgtgccgggaggggactccgtacggctacgccggtctgatcggcatgctgcgcaccgccgcgcaggaggacccggcgctgcacggccagctgatcgagtgcacgcagcggccgtcgggcgaggaactcgccggcgtgctgcgggcggagtacgggcaggcggcggatcacgtgcgctacaccggcggccgccgccaggtccgcgcctgggcagcggccccgcgtgcggcggcacccccgccggtgtggaaggccgacggcgtctacctgatcagcgggggagcgggcggcgtcggccggctggtcgccgccgacatcgcacggcacgcccccggcgcccgggtcgtcctgtgcggacgctcgccggcggtccccgggcccggtcagccgggcccggggaccgagtaccgccgggtggacgtcgccgacgccgacgccgtggcggagctcgtcaactcccttgtgcgcacgtacggcaggctcgacggggtcgtgcacgcggcgggcctgatcagcgacgactacgtgatccgcaagtcccaccaggacgcccagcaggtcctggcgccgaaagccgctgggctggtgaacctcgacgaggccacccgccgcctgccgttggacttcctcgcggcgttctcctccggcgcggggacgctgggcaaccccgggcaggccgactacgccgccgcgaacgggttcctcgacgcctacctgacccaccgcgccggcctggccgccgcgggcgagcgccacggcgcgagcgtctcgatcggctggccgctgtggcaggacggcgggatgagcgtcccggccgaggacgtgcccgcgctcaccgcccgcttcgggcgccccctgggaacggacacggcactgcgggccctgcacggcgcactggcgctcggcacaccacacctactggtcatggacgaggagagcggagtggacgaagagagcggagtggacgaggaaggtccgcaggaggcggagacgcagcagacggggccggcggaactgcgggcacatgtgctgcccctgctgaaggagttgatcgccgagacggtgcggctcgaccccgcccggctggacgccgccgctccgctcgacggcttcggcatcgactcgctggccgtgacccggctcaaccgccggttcgcgcagtggttcggcgcgctgcccaagacggtgctctaccagtacccgacgctgaacgacctggccgggcacctggcggagcagcacgcggacggctgccgccgctggctcggcgacgtcccggacgtggccgccgccccggccgggactccggcgacggcggccgcgccgcggaaggcgcggccccgtccggccgacgcggacgagccgatcgccctcatcggcctgagcgggcgctatccggacgccccgaccctggaggcgttctgggagaacctgcgcgcgggccgcgagagcgtccgcgaggtccccgccgagcgctggccgctggacgccttctacgaaccggacccgcagcgggccgtgcagcagggcgccagctacagcaagtggggcgcgttcctcgacgacttcgcccgcttcgacgccgcgttcttcgggatcgcgccgcgcgacgccgccgacatggacccgcaggaacggctgttcgtcgagagcgcgtggtcggtgctggaggacgcgggctatacgcggcagcgcctcgccgagcagcacgcatcgtcggtcggcgtcttcgccgggatcaccaagaccggcttcgaccgccaccgcccgccggcgaccgacggactgccgcccgcgccgcgcacgtccttcggatcgctggccaaccgggtgtcgtacctgctggacctgcacggcccgagcatgccgatcgacaccatgtgctcgtcgtcgctgaccgcgattcacgaggcatgcgagcacctgcgccacggcgcgtgcgagctggccatcgccggcggtgtcaacctctacctgcacccctcctcgtacgtcgagttgtgccgttcccggatgctcgccaccgacgggcactgccgcagcttcggcgcgggcggcgacgggttcctgcccggcgagggcgtcggcgcggtgctgttgaagccgctgtccgcggccgaggccgacggcgaccccatccacgcggtgatcgtcggctccgcgatcaaccatggtgggcgcaccaacggttacaccgtgcccaacccgcgcgcacaggccgcgctgatccgcgacgcgctggaccgcgccggtgtgtccgcggccggcatcggctacatcgaggcgcacggcaccggcacccggctcggcgaccccgtcgagatcgacggcctgacccaggccttcgctcctgacgccggcgggagcggtgcgtgcgccctcggctcggtcaagtcgaacatcgggcacctggaggccgctgcgggtatcgcgggcctgaccaaggccgtactgcaactgcagcacggcgagttcgcgcccaccctgcatgccgagcagaccaacccggacatcgacttcgcggccaccccgttcaccctgcagaccggcggggccccttggccgcggcccgcggacggcggcccgcggagggcaggcatctcctcgttcggcgcgggcggcgccaacgcccatgtcatcgtcgccgagtaccggagcgcgacgcccgcacccgccacgcccgccccgtccgcgcggccggtgctgctgccgctgtccgcccggaccaccgaggacctgcacgcacgggccggccaactgtccgacctgctccgcaacggcgcccccgtggacctgcccgccgtcgcggccaccctccagaccggccgcgaggagatggcggagcgggtgtgcttcgtcgcgagcacacccggggaatggctcgaccagctcggcgccttcctcgccgactccgactccgactccgactccgactccgactccgactccgactccgactccgactccgactccggctccggctccgaggccgaggccgaggtcccgtggtcccgcggccgggtcagggccacccgcgagaccctggcagccctggcggagaaggacgaactgcgcgcactcgtcacccgctggatcaaccgcggcgactggcacgacctggccgccttctgggccaagggcatgccgctcgactggacccgcctgcacgccggtgcggacacgcccgcacgggtccacctgcccgcctaccccttcgccggacggcagttctggttcggcccggccggcagcgagcacccggcaacgacgccggtggccgccccgtcctgctcgacggcagccggtgccgccgacgtcgagcgcatcctgctcgacgcactggcagcggccctgcagatgccggtcgccgagatcgagcgccgccgccccttcgccgactacggcctggactccatcctcggcgtgaacctggtccacacgctcaacacggccctcggcaccgcgctggagaccaccgatctgttcgaccacggcaccgtcgagcgcctgcacgcgttcctcgtcggtacctacggtgacgcactgcacgcaccggcctccccggcagccgtcgccccggcgccagacgacgacgccatcgccgtcgtcgggatggccgcccgctacgccgacgccgaggacccgcgcgcgctctgggaccacttgatggccggccacgacctcgtcgaaccggtgacccgctggccgctcggccaggacgtgagctgccgctccggcagcttcgtccgcggcatcgaccagttcgacccggtgttcttcgcgatctccggtgtcgaggccaccaccatggaccctcagcagcgcatcttcctcgaacagtgctggaacgccctggaggacgccggctacaccggcgaacgcctgaccaaccgcaactgtggcgtctacgccggctgctacgccggcgactaccacgaccagctggacgcccggccgccggcgcaggcgctgtggggcaccatgggctcggtcgtcgcctcccggatcgcctaccacctcgacctcaagggccctgccctcaccaccgacacctcctgctccagctcactcgtctccctgcacctggcctgccgcgacctgctctccggggacgccgacatggcgatcgcgggcggggtgttcatccagaccacgtcgcggctgtacgagtcggcgtcgcgcgcgggcatgctctcgcccagcggccgctgccacagcttcgacgcccgcgccgacggcttcgtcccgggcgagggcgcgggcgcagtcgtcctcaaacggctcgccgacgcccggcgcgacggcgaccacatctacggcgtcgtccgcggctccggcatcaaccaggacggcaccaccaacggcatcaccgccccgagcgcggcctcgcaggaacagctcctgcgcgacgtccacgcccgcagcggcatcgagccgggcggcatccagctcgtcgaggcgcacggcacgggtacccagctcggcgacccgatcgaattccgcgcgctcacccgcgcgttcgaggacgccccggccgggagcgccgtgctgggatcgatcaagaccaacatcgggcacacgcagttcgccgccggcatcgcgggcgtcatcaaggcgctgctggccctggagcaccggcagatcccgccgtcgctccacttccaagaggccaaccgggccgtcgtgctcgacggcggcccgttcaccgtcaccaccgccccgcagccctggacggcgcctgcccgcggcccgcgccgggcggccgtgagttccttcggggccagcggcaccaacgcgcatgtcgtgctggaggagcacccggtcccccggacgaccggcgcgggcggggaacacgcctttctgctgtcggcccgcacaccggccgctctccgtgccgtcgccgaacggctgctcgcccacctcgaccgcgaacccgggctgcccgccgacgccgtcgccttcagcctggccgcgggacgcagccacttcgcgcaccggctggccgtcgtcgccgccggcctgcccgacctggcggcacgcctgcgctcctggctgtccggcaccgccggtgacacggtgctgcagggggagaccgccgcggacccccgccccgtcggcggtgtgcgcgcgccggccccggccgcgctggccgcagcgtacgtacggggcgaggccgaccggttcgccgacagcttcgcgtccgcctcgcgccgccaggtgccgctgccgacctacccgttcgagcggcagcgctactggaccgacacgaccgacaccggggaaagccaggggctcaaggacacggacggggccgcgtaccgcctccggctcggcggcgaggagttcttcctggccgaccaccacgtgggcggccgggccgtgctgcccggcgtgctctcgctggagttcgcacgccgtgccgtgaccggcggttccttcgcgccggtcggcctgcgcgatgtcgtatggccggagccgttccccgtcggggacggcggcgccgaactacgagtcgatcgggacggcgacgccttccgcgtcctgcgcgacggctcggccgtacacgcccagggccggatcgccacgcccggctcgcccgtccccacgccgttggacgccctgcgggcccgctgcggccgccgcaccctgtcgcggagccagtgccgtgcggccctcgacgccgtcggcatccgccacggagaccgcctgcgcgccatcgacaccctggccgtcggtgacggcgaggtcctggcccggctcgtcctgcccgacggcgcccgcgacggcgcgttcgcgctgcaccccgcgatgctcgacagcgccgtgcaggccgtcgtcggcctctacggcgacgccaccggcacgctcgacgagcaacgcggcgcgcccgcactgcccttcgccctggacgccgccgacttcttcgcccccaccaccgaacgcatgtgggcccacctgcgccacaccgagggctacaccccctcggccgaccgggacgtgacgaaagtggacatcgacgtgtacgacgacgacggacagctctccgcgagcctgcgcggctacgcgttccgccgcatgaccgccccgtccggcgcggccccgcgtgccacgctgctggcaccggtgtgggacgccctgcccgtcgtgcccgccgagccgtggccccacccgcggacccgcgtcgtgctgctgggcggcacccccgaggaacgggacgggctccgccgccgctaccccgacgccaccgtcctggacccccacgccgacgaaccggtcgaccggctggccgcgcggctgcccgccgacgccgagcacgtcttctggctcgccccggccggccccaccggcgccccggccgccgcgcggtacgacggcacgatcgccgtattccgactggtcaaggcgctcctggccgacggcgcggacgcccgtgaactgggcctgaccctggtcacccggcaggcgcgcctgctaccgggcgacaccggtgccgaccccgcccacgccggtgtgcacggcctcgccggcaccctggccaaggagtacccgcactggcggatccgcgtcgccgacgtcgaggcggacgccgccgtgccctggccggctctgctggctctgcccaccgacccccgcggcgagaccctggcccaccggcacggcgagtggtaccgcctgcgcctgctggagacggacgggaccggcgtcgcggccgccccgcgcgagcccggcggcgtgatcgtggccatcggcggcgccggcggcatcggcaccgtgtggaccgagcacatgatgcgccgtcacggcgcccgggtcgtctggatcggacgccgcccgctggacgccgccatcgccgctcagcaggaagccctggcagcccacggccccaagccggactacgtgcaggccgacgcgaccgaccgcgacgccctgcgccgcgcctgcgacgagatcgtgcggcggcacggccccgtgcgcggcgtcctgcacaccgcgatcgtcctcggcgaccagaccctcgcccggatggacgaggaccggttccgcacgacctacgccgccaaggccgacatcgccgtgaacctcgccgacgccttcgccggccagccgctggaattcgtcgcgttcttctcctccatgcaggccttcttcaaggcccccggccaggccaactacgcggcgggctgcaccttcgccgacgcctacgccgagcacctgtccacccggctcgactgcccggtcaaggtcatgaactggggttactgggccggcgtcggcgtcgtcaccgccgacggctaccggcagcggatggcacagctgggcctgggctcgatcgaaccggacgagggcatggccgccttcgacaccctgctggcctccccgtacccgcagctcgcactcctcaaggccacggacacccgcagcatcgacggcctccacgacgacgacgccctcacgcacccggtcgtcaccaccccctccctgatcggcgccctgggcgaggactgccccgaccgccgcgccgagatcgcgcagctgcgtgagaaggcgggcgggcacgccggagccatgcaggacgcgctcgtccgcatcacctgggcgctgctgcagtccctgggcctgttccgcgacggccgcgcggccaccgccgccgagtggcgcgccctcggcggcatcgaggaccgctacgagcgctggaccgagcacaccctcgccgtactcgccgacgcaggcctcctgcgccgcgagggcgaggacacgtacgtggccctcgacacccgtaccggatccctcgacgacgcctgggccgactgggaccgggcgcggcagcagtggctggccgacgacgccaagcgtccccaggcggtcctcgtcgacacgacgctgcgcgccatgaccggcatcctcaccggccgccgcccggccaccgacgtgatgttcccgaacgcctggctcgaactcgtcgaggccgtgtacaagaacaaccccgtcgccgactacttcaacgacgtgctcgccgacaccctcgtcggctacctcgaacggcggctggcggacgacccgtccgcccgcctgcgcatcctggagatcggcgccggcaccggcggtaccagcgccacggtcctgcgcaggctgcggccgtgggcccggcacatcgagaagtacacctacaccgacatctccaaggcgttcttgctgtacgggcagcgggagtacggcgagatcgccccgtacctggacgcacggctcttcaatgccgagaagccgctggcaggccaggaggtggaccccggcgcgtacgacgtcgtgatcgccaccaacgtgctgcacgcgacccgcaacatccgcaggacgctgcgcaacgccaaggccgccgcgcgcccgaacgccctgctgctgctcaacgagctcagcgacaacatcctcttcagccacctcacgttcggcctcctggacggctggtggctctacgacgacccggcgccgcgtatccccggttctccgggcctggcgccggagagctggcggcgggtcctcggcgaggtcggcttccgcgcggcgttcgtcgccgccgggggcgccgacgacctcggccagcaggtgatcgtcgccgagagcgacggcgcgatccgccagccgcgcccggacggggagtccgctttccgcggcaccctcccggaggccgggccgcgggccgccgagcctcaactgcccgccccgacaccggatccggtcgccgccgacggcgtacgtgacgacgagctcctggcggacctggcccgcgaccacttccgcaccctggtcgcggacaccttgcaactgccggtcgccgacatccgcgccgatgtgcccttcgaccgctacggcatcgactcgatcctggtcgtccagctgacggaagcggtccgcaaggggctctgcaacgtcggcagcacgctgttcttcgaagtacggacggtcgacgggctcgtccagcacttcctgcgcacccagcccgacgcgctcgcggcactggtcggcctgagcggcgcgcgggcagcgcgcacggacgagcagctcgcgccggccgccgggccggagccggtccccgtcatcgccgccgaaccgccccgcgccgagcagggcatggccatcgcgatcgtcggcatggcaggccgctaccccggcgcacccgacctggacaccttctgggagaacctgctcgccggccgggacagcatcaccgagatcccggccgggcgctgggaccacagccgctactacgacgcgcgtcgcggcgtgcccggcaggacgtacagcaagtggggcggcttcctcgacgggatcgacgagttcgacccgctgttcttcgggatctcgccgaaggcggcgtccacgatggacccgcaggagcggctgttcctgcagtgcgcccacaccacgctggaggacgccggctactcgcgcggcgccctgcgcgccgccgcccgcgcccgggtggcggaggacgccggcgacatcggggtgttcgccggcgcgatgtactccgagtaccagctctacggcgccgagtacagcgtgcgcggtgagccggtcgtggtgccggggagcctggcgtccatcgccaaccgcgtctcgtacttcctggacgcgagcggccccagcgtcaccgtcgacaccatgtgcgcctcggcgctgtccgcgatccacctcgcctgcgccgccctccagcgaggggagtgcggtgtcgccctggccggcggggtcaacctgtcggtgcacccgggcaagtacctgatgatcggggagggccagttcgcctccagcgacggccgctgccgcagcttcggcgagggcggcgacggctacgtgcccggcgagggcgtcggcgcggtgctgctgcgcccgctcgccgacgccgtcgccgacggcgaccgcatcctcggcgtgatccgcggcaccgccgtgaaccacggcggccacacgcacggattcaccgtgccgaacccgctcgcgcaggcggcggtcatccgcagcgcctggcgccgggccggagtggacccccgggacatcggctgcatcgaggcgcacggtaccggcacctcgctgggcgacccgatcgaaatcgccgggctgaacgcggccttcgccgagttcaccgacgcacggaacttctgcgccatcggctcggcgaagtcgaacatcggccacctggagtccgcggcgggtatcgcgggcctcgccaagctgctgctgcagatgcggcacggcacgctcgtgccctccctgcacgccgaacgcgtcaacccggacatcgacttcgccgacagccccttcgtcctgcagcgcgaagccgcgccctggccgaggaccggcacccgcccgcgcctcggcggcctctcctcgttcggcgcgggcggctccaacgcccacgtcgtggtcgaggactacgtcgaggagcacgccgggaaggacctcgcgcccgaggcgcaccgtggcgaaaccgtcgtcgtggtgctgtccgccttcgacgaggagcgcctgcgcgagtcggccgggcggctgcgcgacgcgctgcggaaggagcggtggagcagcgcggacctgcccgacatcgcctacacgctgcaggtcggccgcgaggcgatgaccgcacggttcgccgtggccgtcagcacgcttcccgccctggtcgacgcgctggacgcctgcgcgctcggcagcgggctgcccgcgggcgcgtatttcaaccccggcggcgaccggggcggcgcggtcaaggacttcctcaccgacgaggacttccaggagacggccgtgcgctgggcacggcgcggaaagccggcgccgctggccgaggcctggaccagcggcctggccgtcgactgggcccgcctccacaccgagggaccgaagccgcgcaaggtcgcactgcccggctacccgttcgcccgggagcgctactggtacaccgacggacttccggaactccaggaaatccccgccacgttcgggaacgccgcacggcagcccgccgccccgccccctgccgtggaggccgcgcctgcgacgacgtccgccgtgcccgccccgcccgcgcggccggccaactcctacgagcttcccgcgggcgacctcaccctgcaccccgtctgggagcctgtccggctgctgcgcggcagcccttacccgtccgcggcctcccgtgtggtggcgatcggcctcgcaccggacgcgctcgcggagctgaccgcccgccgcccgcagaccgtggtgctggacaccgccgcgtcatccgccgaagaggtgcgtgacgaactcgccgtcctgggcgacttcgaccacgtcgtcatgcggttcccgaccgcagccgccgcccacggcgccgaggcgcagatcagcacgcagcgcgcggcgatccggagcatgttccgggtcctcaaggcactggccctcacccgggacgagcagcggctcggactcaccctcctgaccagcggcgcgttcgacgcaggcggctcggggaccgccgacccggcgcaggcgagcctgcacggtctgctcggcggcctggccaaggagcagccgcactggcgcatccgcgcggtcgacctggccgacggcgaaccgttcgtcgccgacgaggtcttcgccctgcccgccgaccgccgcgcgcacccgctcgtccgccgcggcggccagtggctgcgccgtcagctcctgccggtggacgccaccgagccgcccgcggagcccgtgctgcgccgcgacggcgtctatgtgctcatcggcggcgcgggcgacctcggcgtgctgctcagcgagtacctcgtacggcaacacgacgcacacgtcgtatgggtcggccgccgcgccgaggacgaggacatccgggccagggcggaccgggccgcagcgggcgggcggacccccgtctacctgtccgccgacgcctccgaccccgacgcgctcgcccgcatgcgggacgaggtcgtccgccgctacggccgcatcgacggcgtggtgcacctggcgatggtgttcagtcacacgccgctcgcccggatgaccgagcgcgaactggaggccaccctcgcggccaaggtcgacccgtgcgcgcacttcgccgacgtcttcgccgggcacggcctggacttcgtcctgctgatctcctcgctggtgagcttcatccgcaactcccaccaggcgcactactcggcggcctgcgccttcgaggacgcgcacgccgccgccctgcgcgaggcgctggactgccgggtcaaggtcgtcaactggggctactggggcaacgtccccgacgagctcctgcgcgacgtgacgtccatgggactggccccgatcgccccggccacggcgatgggcgcactggagcgcctcctggccggcccgctccaccagatcggcttcatgcgcctcggccgcccgctgcccgtcgaaggggtgctcaccgcggagacgctgaccccgcagacgcacggtgcggcggcccgcgacggcgccgcggccctcgctctgcccaccggcctggccgcgtaccacgagagcccggtcccgggcgagatcgacgcgttcctgctccgccgcctcgccgccgagctgcgccgagcgggtctggaggagccgcgccacggcctggccgagtggaaggagcggcagggcgtcgacgcacggttcgacggctggctctcggccaccctgcacgcgctcgccgagcacgcgatgatcgacgaccggggccgctggaccaccagcagtccggccgccacggacgccgacgcctgccgcgccgactgggccgcgcagacaccccggtgggccgccgccaaccccgatctgcgcgcaccgctgaacctgctggaccggaccctgcccgcgctccccgacgtcctgtgcggccgggtgcgcgccaccgacgtgctcttcccccaggggaagttctccctggtcgaaggcgtctaccgcgacaaccgcgtggccgcgcacttcaacgccgtcctcgccgaacacgtggcggccttcctgcgcgcacgccgggacgccgatcccggcgcccgcctgcgcgtgctggagatcggcgcaggcaccggcggtaccaccggccccgtgctcgaccgcctcgcccacgaagggctggacctggccgagtactgcttcaccgacctgtcccaggcattcctgcagaacgcccaggacaccttcgggccgggccgcgaccacctcacctaccgcatcttcgacgcggccaggcccccgcacacccaagggctcgacaccggcgccttcgacgtcgtcatcgcggccaacgtgctgcacgccaccgacaccatccgcccggccctgcggcacgccaaggcgctcctgcgcggcaacggcctgctggctctcaacgagatcagcggcttctacctcgtcaaccacctcaccttcggcctgctcgacggctggtggctctacgacgacgccgaactgcgcgtgcccggcagccccgcgctgtcgccggcggcctggcagctcgtactggaacaggaaggcttcaccggcatccgccatccggcgcgggacgccctggcactcgggcagcaggtcgtcgtggcccacagcgacggtctcgcccgcagcccgcgcctgctctccggaacgcccgagatgagcagcccgccctcccagccgccggcggaaaccgcggctccggccgccgcctccgcttcggcccgggccgtcacggacgtggtgctggccgcgctcgccgacgcgctgcgcatgcccgccgaccggatcggcccggaccgggcgttcgccgactacggcctcgactccatcgtcggcgtccggttcgtccagcgcctcaacgaggagctgggcaccgacctgccgaccacggtcgtcttcgactaccgcagcgtggcgcagctcgccgcccacatcgccgagagccaccggccgcaaccggcccccgccgcggcggcaccggtgcccgcaccggacgccgccggggcaccgaaccgtcccgaaggacgcgagcccatcgccatcgtcgggatcagcggccgcttcgcgcagtcggacgacaccgacgccctctggcagcacctcgccgccggccgcgacctcgtgggcccggtcgaacggtgggacctctccggctacagccaggaccaactgtcctgccgcgcgggcagcttcctcgacggcatcgaccggttcgacgcacgcttcttccacctgaccggcctcgaagccacctacaccgacccccagcagcggctgttcctggaacaggcgtggacggccatcgaggatgccggctacgcgggctccgcgctggacggccgccggtgcggcgtctacgccggctgcaccggcggcgactacccccagtggttcgaggacgcgccgcccgcccaggcggcatggggcaacgcgccctcggtcgtaccggcgcgcatcgcctaccacctgaacctccagggtcccgccctcgcggtcgacacggcctgctccagctcactggtcgccgtccacctcgcctgccagggcctgtggagcggcgaaaccgacatggccctcgcaggaggcgtcagcgtccagaccaccccggacacctacctggcggccggccgcggcgggatgctctcgcccaccggcaagtgccacaccttcgacgccgccgccgacggattcgtccccggcgagggcgtgggcgtcgtggtgctccgccgcctgtccgacgcactggccgacggcgaccacatccacgccgtgatccgcggctccgccgtcaaccaggacggggcgaccaacggcatcaccgcacccagcgccctgtcgcaggaacgcctcatccgccaggtgcacaccgaattcggcatcgacccggccgagatcggcatggtcgaggcgcacggcaccggcacccagctcggcgaccccatcgaatgccaggccctggtcggcgcgttcggcacggccggcggcagcgacacctgcgcactcggctcgatcaagacgaacctcggtcacaccacctccgccgcgggcgtggccggcctgctcaaggtcgtgctctcgctgcgccacggtcagatcccgccgtccctccaccactacgagaccaaccccgcgatccgactcaccgaaagtcccttccacgtgaacaccacgctgcggccgtggcagcccaacggccagggcaagcgcgtcgccgccctgagcgcgttcggcttcagcggcaccaacggccatatggtcgtggagaacgccccggaccgtgacgagcgccaacaggccgccgacgagctgctgttcgtcctgtccgcccagcagcccgaggcgctgcgccaccgcgccgaggacctcttggcgtacctgcgccgcgcacccgacgccgcgctgggcgacgtcagctacacgctggcggcaggccgggaccacttcacccaccgcgcggcctttgtcgccgccgaccgcgacacgctcgcccaccggctggaggcctggctggccgacggacggagcgacaccgtcggccggcgcggcgacaccgcgccggagcgcgcccgggcccggtacctgaacggcgaggaggtcgacttcgcgccgctgttctccggcctcgacgtccgtcgcacgccgctgcccacctacccgttccagcgcaagagctactggccgacggccactgctccgagccggcgccaccaagccccgcaggccgcgaacggccctgccgccgccccgtcgcccgagcccgcccggccggcacccgcgcagccggcaccggacacggacgaggcgaccgtgcggtacctggccggcgaactcctgctggccgagctctcccgcgtgctcatgatggagcccgaggagatcgacccgcaggcgtccttctccgactacggcgtggactcgatcctcaccgtcaggctcgtcgcagcggtgaacaacgccctcgccgtcgacctgccgagcaccgcactgttcgaacacagctcgctcgaccggctgacggaccacctggtcacccggtacggggcgcagttgcggtcctccggtgcgctgcgcgggccggcagccgaggccggaggggctccggcgcaggacgaccacgggcccgccgccgaggctccgtccgctgctcctgctgctcctgtcgcctccgccggaactgccgccgtccccgctcacgcccccgcggccgctgcgggcgacccggccgacgacggcgtcgccgtggtgggcatcgccgcccggttcgcgcagtcgcccgacgccgccgccctgtgggcacacctcgccgcgggcgacgacctggtcggcgaggtcacccgctgggacatggacgaggagctgggcgcgggcgccccgcgccagtacggaagcttcgtcgacgacatcgagcgcttcgacgcctggttcttccggatgtccggtaaggaggccacctacaccgacccgcaacagcgcatcttcctggaggagtgctggcacgccctggaggatgcgggctacgccggtgaacggctcgacggccgcgggtgcggcgtctacgtcggcggctcacccagcgactaccagcagttgatcggcgacgacgcgccaccgcagacactgtggggcaacatctcctcggtcatcgcgtcgcggatctcctacttcctcgacctgcagggtgccgcgctggcggtcgacacggcctgctccagttcgctggtggccattcaccaggcctgccaggacctccgcctgggcaacacgtccatggcgctggcgggcggagtcttcgtccagtccacgccgatcttctaccggtccgccgtgcgggcgaacatgctgtccgcccgcgggcgctgccacaccttcgacgagcgcgccgacggcttcgtgccgggggagggcgccggcgtggtcgtgctcaagaggctcgccgacgcgctgcgcgacggcgaccaggtctacggcgtgatccgcggctccggcatgaaccaggacggcaccaccaacgggctcaccgcgcccagcgccggatcgcaggaacgcctcctgcgcagcgtccacgagcgcgccggcgtcgaccccgccggcatccagctgatcgaggcgcacgggaccggcacgccgctgggcgaccccatcgagttcgaggccctgcgcgccgcgttcggcgacgcgcccgaggcaggctgcgccctggggtccgtcaagaccagcctcgggcacacccagttcgccgcgggcgtggccggcgtcatcaaggtgctgctggcgctcaggaacgagcaactgcccccgtcgctgcacttccgccgggccaacccggcgatcacgctggagggcagccccttctacgtcaacacggaactgcgcccgtggcccgcacccgccgacggtccgcgccgcgccggcgtcagctcgttcggcgccgcaggcaccaacgcgcacgcgctgatcgaacaggcccccgccgtgcggaccgccgggcacggcccccggcatgcctggctgatcgtcctgtcggcacaggacgacgccggccgccgagcccaggccgagcgcctgctggaccacgccctcgcccacgaggacctggacctgggcgacgtggcgtacaccctggccaccggacgccgccactgcagccaccgctgggcgggcgtggccacggaccgcgagcagctcgtcgccgccctgcggacctggctgtgcgacggccgggcggagggcgtggtcaccggtgaggcgcccgacgggcaccgccgtcaggaccccgccgaggacgcccgcgccggccgcctgatggccgagcccgaccgtcacgacagcctcaccgagctggccgggctcttcgcccaggggcaagatctgggcttcgccccgctcttcggcgacggcggcttccgtatcgtctccctcccggcctatccgttcgcgggcgagcgctactgggtcggatcacgtccggcggcccccgctgcgaccccggcctccgctccggtacgcgccccggtccccgttgcggccccgtcgccgctggaaggccgccggctgaccggtgatcccggctcgccgtccttcgccgtcgagctggccggccgcgagttcttcctcgacgaccaccgggtgcgcaacgtgccggtgctccccggcgtggcctatctggagctggcgtacgcggcggcccgggccgagggcgtcgaccccgcccacgcccgcctgcgcaacgtcgtctggtcgcgccccgcacggatcaccgggccgaccgcggtcgagatcgcgctgcggccgtgcgaggacgacgccttcacctacgagatcaccacggcggccgacggcgaacagccggtgatccacgggcaaggacgcatcgagcggtgcgggacgccgtcacccgcgcgcctggacatcgccgcgctgcgcgcccagtgcgaggtgcgcactctggaacacgacgactgctaccggctcttcgaccgcatgggcatcggctacggcccggccatgcggggcatccggcggatccacgtcggcgccgggctcgccgtcgcacgcctgagcctgccgcaggccgcccgggacggcgccggctgggacctgcacccgtcgatgctcgacgccgccgtacaggccaccttgggcctgtcactggccgaggacaccgacaccgtggcgccggcactgcccttcgtcctggaggaggtgcagctgctcgcgcccagcccggccggcgggtgggccgtggtgcgacccgcagcgggcgacggcggcggagccgtacgccgcatcgacatcgaactgtgcgacgacgacggcgaggtgtgcgtacgcctgctcgggttcaccgcacgcgtcctggccgccggtgacgaccccgccggcggagagaacaccgggggcgcgacgctcaccctcatgcgtgccggctggcgcccggccgagcccacccgggcctcgcgcccgctggtgcaccacgaggtgctgctcggcggactcgcagggaccgaccccgcggcggtccgggacgggctcggtgtgccctgcaccgcattgcccgacgacggcgatccggcccggtgtttcacccgccaggccgagacggtgctggcccgcctgcagcagttcgtcccacgcacccgcgacggcgaggtcctgctgcaggtggtcgtgcccgccgacggtgagaaccgggtcctcgcgggcctgggcggcctgctgcgcacggcccgcatggagcaccccaagctgctgacccagctcgtcgaggtggagacgcccgtcgacgccgcgacgctgtgcgagcgcctgcgccgggacgcggcgagccccgacgacgtggccgtgcggtactccggcgggcagcgccgggtgccgcagtggaccgccgtcgaggacgccccgccggcccgcccctggaaagccggcggcgtctacctcctcaccgggggagtcggcgggctcggcgcacacttcgcccgcgagatcgcccggcaggcgcccggcgccgccctcgtgctctgcgggcgctcgccggagggcccggcccagcgtgaactcctgtgtgagctgggcgacttgggtgcctccgccgtctaccgggtgctggacgtcgcccggcgcgacgccgtgaccgcctgcgtgaacaccgtcgtcgccgagcacggccgcctggacggcgtcgtccacaccgccggtgtggtgcgcgacggctacctggcccgtaagagcgccgaagagctgcgggaggtcctcgccgccaaggtcgccggcttcgtccacctcgacggagcgaccgccgcgctcgacctggactgcttcatcggattctcctcactgtcggcgtacggcaaccagggccagggcgactacgcggcggccaacgccttcatggacgcctacgccggcctccgccacgagcgggtggccaggggcgagcgccgcggccgcacactggtggtcggctggcccctgtgggccgacggcggcatgacggtggacgccgccaccgaacgccgcctgcacgacagcgtcggcatggtgccgatccgcgccccgcacggtgtggaggcgctgctacgcgcctacggcaccggcgacccgcacgtcctggccgtcttcggcgaccgcgcccgcatcgacgccaccctcctggccgccccggcggccacgggcgccgcaccggcggtgaccgcacccgaccgcgccgccctgcacgcgagggtcctcggccgcgccatcagccacgcctgcgccgtgctgggcgttccggcggcggagctcgacggtgcggtggagctgagcgagtacggcttcgaccccgtctcgctcaccgggttcgccgcccgcctcaccacggagttcgggcttccgcccgtgcccaagcccttctccgaacacctcaccctgggagaggtcgtggaccacctgctcgacacccacccccatcacttcgggacggtcccgccggcccccgcgcccgagccctccgccgggcccgaaagcgccgccgcgcccgtcgcgacggccggccgggagcagcagcacaaggcgctgctgaagaagctgatcgcccgcgtgtccgacctgctggacgtgcccgccgagcggatcaccggcacggccgagatgacccgctacggcttcgactccctctcgttcatcggcttcgccaacgacctcaacgccgagttcgggctctcgctggcaccgaccctgttcttcgagaaccccaccctggacggggtcgtcgaccacctcctcgaccaccacgccgaccgcgtcgccgccaccgcggcaccgcagcaggaaccgcgcgcggcggcggcccccgccgccccagagcccgccacagccgacacccccgcgtcccgtacggatgcgcccgggaacgagccgatcgccgtcatcggcatcagcggccgcttcccgatggccgacgatctcgacgcgttctgggagaacctcagcgaaggccgcgactgcacccgtgaggtccccacggaccgctgggactggcgcgcccactacggcgaccccgtcaaagagcccaacacgtcgaacgtgacgtccggcggcttcatggacggcgtcggcgacttcgacccgcttttcttcgacatctcccccaaggaagcggagttgatggatccgcagcagcggctcctgctgatgtacgtatggaaggcgctggaggacgccgggtactcggcggaggccctcgcgggcacgaacacggccctcatcgccggcaccaccagcaccggctacagcaccctcgtcacccggtactcgccgatgatcgagggatacgacatcaccggcgcggccccctccatgggcccgaaccggatgagctacttccttgacctgcacggtccgagcgagcccgtcgacacggcctgttcgagcgcgctcgtcgccctgcaccgggccgtccaggccatccgcgacggtcagtcggacctggccatcgccggcggcgtcaacaccatggtcagcgtcgacgggcacatcagcatctccaaggcgggcatgctcagccccgaaggccgctgcaagaccttctccgaccgcgcggacggttatgcccgtggtgagggcgtgggcatgctggtgctcaagagcctgtcggcggccgagcgcgacggcgaccacatctacggggtcatccgctcgacggccgagaaccacggcggccggggcagctccctgaccgcgcccaaccccaaggcccaggccgccctcctgcgggaggcctacgggaaggccgggatcgatcctcggacggtgggctacatcgaggcccacggcaccggcaccaaactcggcgacccggtcgagatcaacgggctcaaggccgcgttccgggacatgtacgaggagcacggcgcggtggtcgaggaggcccactgcggtatcggctcggtgaagaccaatatcggtcatctcgaactggccgcgggcgccgccggcgtgatcaaggtgttgctccagatgcggcaccgcaccctggtcaagagcctgcactgcgacaccgtcaacccctacatcgacctcgacggcagcccgttccacctcgtacgcgaacggcagccctggcccgccctgcgcgatgccgaaggccgtgagctgccgcgccgggccggagtcagctccttcggcttcggcggcgtcaacgcccatgtggttcttgaggagtaccggccgcgcaccgcacccgagccggaccgggcgcccaccgcaccggtccccgtcgtcctgtcggcgagccaccccgacgtgctgtgcgaactcgccgagcgctgggtggacgcactgcgccgcggcgactacgacgacaccgacatggcgtcgatcgcctacaccacgcagaccggacgcacgcccatgaccgagcgcctcgcctgcctggcccgcacggccggcgaactgcgggaggcactggagtcctggctgcgcggcgagcccgcggccgacgtcttccgcggcaaggtcgcgcgcggcgtcgacctgccggacgcaccagccgggttcggcccgcacgacgaccacgacagcgcgggccggcacgactgggcccgcctgctccaggcatgggtgaacggcgcccccttcgactgggaccgcctccacaccgggcgccgcccgcgccggatcgccctgccgacctacccgttccgcctccggcgctactgggtcgacacctcgcgccccgcgaacggcacacaaacggaggcactgcacccgctggtgcacacgaacacctcggacctgaacgagcaccgctacacctcgcacttcaccggccgcgagttcttcctcgccgaccaccgcgtacgcgcccaggtgatggagacggtctccggctggcggcccggccgccggcccaccgcctacgacgtccgcgcggacgccgtgccggtgctgccggcggtggcctacctggagatggcgcgcgccgccgcggtccaggcggccggcggcgacgagcgcgcctggtcactgaagttggcctcctggctgcgcccgctcaccgtcgagaaggcgaccgacgtgcacaccacgctgaccacccgggccggcggcggactgagctacgaggtgtacgcggtggacgaggacggcgaacgcgtcaccttcggccgcggccagctgcggcgcgcaacagcggtgcccgccgagcggctcgacctcgcggccctgcgcgcgcagtgcgacggccccgtgctcgacgccgagacctgctacgcacgcttcaccggcatcggcatggcctacggcccggcactgcgcggcatcgagcgcctgcacaccggctcgcggcagtcggtggcgcggctgaagctgcccgccgccgcgtcccgcgagcgcggctgggtactcaacccgggcatgctcgacgccgccctccaagccacggtcggcctcttcgtcgacgaccccggcacgccgcgcacggcactgccgttcgccctgggcgagctggaggtgctgcgggcggtcccgggcaccggctgggtcgtggtccgcttcgccgaggacgaccacgtgggcgccgtgcgccgcctcgacctcgacctctgcgacgacgacggcgaggtgtgcgtacgcctgcgcggcttcagcgtccgcacgctcggcggcagcgagcccaccggtgacagcgagcccacccggcccgccgaacaggcacccgagccgccgtccgggtccgacgacgcctacctgctggacctgatcgaagccattggccgacgcgagatgagcgcggacgaattcaagaggagcctggcatgagcaccacccgcatcgcatccctggacgacctgcaccgggctcattcgcgagggacaggtgggacaggacgaagcgctccgcctgatccgcgactggaagcaggagcaggagcaggagcaggagcaggatcagaatcaggagcaggcgcgagcacggacgcagaccgcacggccggctgacgtcgccgacaccgaggccctgacggagcgggtctgcgccgtcgtggtggagaaggtctgcgagctgctcaaggtcaccacggacgacctggacgtgcatgtcgacctcagcgaatacgggctcgactccctcgtcatcactcagctggtgaacatggtgaacgacgctctgggtctggaactcgtgcccaccgtgctgttcgagcacgcgacgatccaggccttcggcgcccacctgaccgacgagtacggccctgcgctggccgcccgcctggggctgcggtcgcccggcgccgccacggagccccctgccgtcgagcccgtcggtacgcctgtgccggccgcagccgtccccgcacgggccgtacccgtcccgctgcccgccgaccggcacgacgacccgatcgcggtggtcggcatgagcggccggttcccccaggccgaggacctcgacgccttctggcgcaacctgcgcgacggccgcgactgcatcgcggaagtccccgccgaccggtgggactggcgcgccctcttcggcgacccccttcaggaaccgggccgcaccaacgtgaagtggggcgggttcatggagggcgtcgccgacttcgatccgctgttcttcggcatcgctccgaaggacgccgtccacatggacccgcagcagcgcctgctgatgctgtacgtgtggaaggcgctggaggacgccggctacgccgccgacgccctggccgggagcagcttcggcctgttcgtcggcaccagcgacaccggctacggcctgctctccgaccgcagcagcggcaggggcgagagcgtcacgcccacgggcagcgtcccctccgtcggcccgaaccggatgagctacttcctggacgtacacgggccgagcgagccgatcgagacggcctgttcgagttccctggtcgccatgcaccgcggcgtcatctcgatcgaacgcggcgagtgcgacatggccgtcgtcggcggtatcaacaccatggtgatccccgatggccacgtcagcttcagcaagtccgggatgctcagcgccgaggggcgctgcaagaccttctccgaccgcgcggacggttatgcccgtggtgagggcgtgggcatgctggtgctcaagagcctgtcggcggccgagcgcgacggcgaccacgtctacggcatcatccgctcgacggccgagaaccacggcggccgctccaactccctgaccgcgcccaaccccaaggcccaggccgccctgatccggcgcgcctacagcaccgcgggcatcgaccctcggacggtgggctacatcgaggcccacggcaccggcaccaagctcggcgacccggtcgagatcaacgggctcaaggccgcgttccgggaactgtacgaggagcacggcgcggtggtcgacgacgcccactgcggtatcggcacggtgaagaccaacatcggccacctcgaactcgcggcgggcgtcgccggcgtgatcaaggtgctgctgcagatgcggcaccgcacgctcgccaagagcctgcactgcgacaccgtcaacccctacatcgacctcgacggcagcccgttccacctcgtacgcgagcagcagccctggcccgccctgcgcgatgcggagggccgtgagctgccgcgccgggccggagtgagctccttcggcttcggcggcgtcaacgcccatgtggtgcttgaggagtacgtgccgcgccccgtaccgccggtgagcacaccggaccccgtggccgtcgtcctgtcggcccccgagcccgagatgctgcgcgcccgggcccggcagctggccgaccggatcgactcgggcgggctcggcgaggccgacctgccggacctcgcccacacgctgcaggtgggccgcgtcgcgatggacgagcgcctcgccttcctgacctcctcgctcgccgacctgcgcgagcggctgggcgccttcctcgacggcggcaccgtacagggcctccacaccggacgggcacagcgcccggggccgtggaacgagctcgccggagacgacgacatcgccctcgccctcgacagctggatagccaagggcaagctcggacgcctgctcaaactctgggtcaccggcttcgacgtggactggcggcgcctgtacgccggccggccgatgcggcgcatcccgctgcccgtctacccgttccagctgaagcgctactggatcaccgacgcgaagagcacgacccggcccccggcaccggtggccgcggcgccggacgcacaaccgtcgccctaccgccgcgacctgaccgggcacgagttctacgtgagcgaccaccgcgtgggggacacgcccgtcctgcccggcaccgcctacctcgagttcgtgcgcgacgcgctcgtccgggccacgtccgcaggcaccgccacgggcgtgcgcctgcgcgacgtgacctggctgcgtcccctggaggtgacggcactgcgcaccctcgccgtcgacgtggacccggccggtgggacattcgaggtgtacgaccacggctccggcgaccgcgtcctgcacgcgaacggcaccgcacacgtcgaccccgcactcctcgccgccgacgacacccacgacatcgacgcactgcgcgcgaacctcccgttccggcgtgacggcgccgagtgctacgcgctgttcgcgcgcaggggcatgggatacggccccgcgttccgggccgtgcaggagctgtaccacggtgcggacaccgcccttgcccgcctcctcctccccgaggcggcggcatcctcgctgacgctcaacccggtcatgctcgacgccgccctgcaggcgaccctgggactggcgctcggcgagcacgtcgacgccccgcaggggacggcacttccgttcaccgtgcgcgaggtacaggtcctggcccccaccccggccgagggctgggcgctcgtgcgccgtgccgcggacgaccgccacgacaccggcatacgccgcctggacatcgacctctgtgacacgcagggcaacgtctgcgtgcgtctgctgggcttttccacccgcatgaagccgagcccggcgccccgcgccgccgagccgaccaccacgcccgcactgctgatccaggcggactggcgcgagagcgcggcacgggagcaccacggcgacgacgtcaagcggcacgtcgtcctgtgcgaactcccggcggcggacgccaccgcgctcggcgcagcgctgggcggcgccacctgcgaaacctggcaggcccgcggcgagaccggcacccgctacaccgagtacgccgagcggttgctgaagctgctgcgcgacaaggcaccggaggccgcccggcagccgtgcctgatccaggtcgtcacccccgcgcacgcgccctggctgggcgggttgagcggcatgctccgcacggcgcgcatggagcaccccaagctgctgacgcagtggatcgctctggacggtgacggcgccctggccccggccgagctggccggacggctgcggtgcgacggcgccgacacggccgaggaggccgtgcgctaccgcggaggacgccggcaggtgtcccagtggcacgaagtcgcaccggccgcacccgaacggccctggcgcgacggcggcgtctacctgctgaccggcggcgccggaggactcggcgccctgttcgcgcaggacatcgcccggcgcgtcgagacgcccgccctggtcctgtgcggtcgcagcccggtcggcccggcacagcaggaactgcttaccgccctgcgcgcgctgggtgcccgtgccgactaccgcgtgctcgatgtcgccgaccgcgccgacgtgacccgggtcgtgcgcgaggtccaggccgagtacggcgcgctgcatggcatcgtgcacgccgccggagtgctgcgcgacggcttcgtggccaagaagaccgcggacgacctccgcgaggtgttcgcggccaaggtggccgggctgtgccacctcgacgaggcgactgcctccgtcccgctcgactgcttcatcggcttctcctccatggccgccttcggcaacgtcggacaggccgactacgccgccgccaacgccttcatggacggatacgccgcccaccgcgactccctggtggaccagggcagccggtcgggccgcaccctgatggtgaactggccgctgtgggagaagggcggcatgggcgccgacccgtcgaccgtccagctcctggagtccgtgggcatgcggccgatgcgcgcatccgtgggcatcgacgccctcgaccgcgtctgggcgaccggcctgcccagcgccatcgccctcgacggcgaccacgcccggatgcgggagcgcttcctgccggcgcaccccgagccggaggcccctgccgaacccgcgccggccgccgcgacgttgccggccaccgcaccggtcgccgagccggccgagccgtcgagcgtgggaaccgtcgtcgcggacctcatggcgacactgctggaggtcgacgtcgagaccctgcggtgggacaagtccctgggtgactacggcttcgactccatcttcatgatgcagttcctcgcccaggcgcagacgcacctcgacgcgtccctcaccctcgacgtcatcgccgactgcgaaacgctgcaggacgtcgtcgacgcgatcaccggcaccgcctctgacaccggcacggccgccccaaagcccgcttcggtggctcccgttgaggaggccccggcggccgccgcaccggcaaaggccccggtacggcgcgccgcggccgcatcgccgaacgacttccccgaactggtccgcatgaacggtgtcacctccggccggcccgtgttctgggtccaccacggcaacggcggcgtggagtcctacgccccgctggccgcacgctgcccgcgtcccttctacggcatccagccgaagggctggatcgactccaccgacatcctcaccggtcagtacgccatggccgagcactacgcgtccctcatcctcgccgtacagccggaaggcccgtacgacatcggcgggttctccctgggcggcctgttcgcgtacgagaccgtgcggcagctccagctgacgggcgccgacgtgcgcacgctggtcatgctggacacgctggacgccgaatccaccaacaaggccaacgccctcatcgtcggcgggaacttcgacgccgacgtggtcaccaaggtgagcgacttccgcgccgtcaacctgatcctcggcaacaaccgcttcgactcgcacggcggcgccaccccgatcctgcgccgcgacgaggtcgacaccaccctggaacccaaggagttcctcggctccctgatcgacgccgccctcgcccgcggcgtcagcaagaccgagacgcagctgcgctcccgcgtccggcaactgtcccgctacttcgaggccacgcagggcgagacgtacacggtggacccgctgccgcggcgcgacggactgcgctgctactacctgcgcaaccggggcggcaagttcttcggcgccttcgaggagcacatggtgctcttcccgaaccccgagctccccaccgtggacggcgtcgcgtactggcaggagtgggccgaccagatcgacgacttcttcaccatcgacgtcgacacctcgatgcatgccgaggtcatgacggccccgcagtcgctcgacaagctgatgcgcctctgcgaccggctctacgccgccgaggacgccgccgccccggcgacctccgcgcagggaggccgctgacatgaaggcagtcgtctttcccggccagggcgcccagcggcgcggcatgggacgcgagctgttcgacgcgtatcccgaactcgccgacgaagcctccgaagtcctcggctactccctccgcacgctgtgcctggacgatccgcaccagcaactgggccgcaccgagtacacgcagcccgcgctgttcgtggtcggggcgctcgcccaccggcagtggcgcgagagcaccggcgacgagccggccttcctcgccgggcacagcctgggggagtactgcgccctgcacgccgccggcgccttcgacttcgcgaccggactgcgcctcgtccagcggcgcggcgcactgatggcgcaggcacggggcggtggcatggcggccgtggtcggggtcgacgcggcacggctgcgcgaactcctcgacgaaggcggcttctgccgcctgaccgtcgccaacgacaacgcaccccagcagaaggtcgtgtccggcgacaccgcgaccgtcgacgcgctggtggcatacctcgaggcgcgcgacgtccgctgcgtgagactgaacgtgtccggcgccttccactcgcccctgatgcggcaggcacagcaggacttcgcccgcttcgccgacggattcgccctcggggacccggcgacgcccgtgatcgccaacgccacggcgcgcccgtacgtccccggccggaccgcgcgcacactcgtagaccagatcgtgcagcccgtgcgctggaccgagagcgtgcaccacctcctcgaccagggcgtcaccgacttcgtcgaactgggaggccgggtgctcgtcaggctgatcgaccagatccgctccgccccgcggccggtcgcccagcacgatgcaccggctgcccggcccgacacaccggccgccgcgctcggcagccccttgttccggcggcgcatgggcgtgcgccacgcctacgccgtgggcggcatgtaccggggaatcgcctcggcccagatggtcgtcaggctcggccgtcaccgcattctcggcttcctgggaaccggcggcctgccgctcccggagatcgagcagggggtgaaggaggtccagcacggcctggccgacgggcagccctacggcgtcaacgtactggccgaccacgacgatcccgcggccgagcgcgcgctggtcgacttgctgatgcgccacggcgtccccgtcatcgaggcgtcagccttcatgcagatgacccccgcgctcgttctctaccgggcacggggactccgccgcggtgccgacggccggacggtgtgcgaccaccgcatcgtggccaaggtctcccggccggaggtcgccgagcagttcatggcacccgcccccgggccggtcctggacaggctccgccgggagcacgccctcaccgacgaacaggcggaactcgcccggacggtgccgatgagccacgacatcacggtcgaggcggactccggagggcacacggacggcggcgtcgccacggtcatgatgcccgccatgctcaagctccggcagcaggcccaggaccggtacggctacgacgaaccgatctgcatggggctcgccggcggcctcggcacccccgcggcggtcgcggcggccttcatgctgggcgccgactacgtactcaccggatccatcaaccagtgcacggtggaatccggcatgagcaccgaggtcaaggacatgctgcaggacgtcggcatcgccgacaccgcctacgcgcccgcaggcgacatgttcgaattcggtgccaaggtgcaggtgctccgcaaaggcgtcttcttccccacgcgggccaacagactgttctccctctactcccactacgacggcctcgacgatattccgcagaaaacgcgctccctcctggagagaacctacttcggaaagagcatcgaagaggtctgggacgaagtacgcgcctatctccgttcgcaggggcgcgacgccgacatcgaccgggccgacgccgagcccaaacagaagatggcgctggtattccgctggtacttcttccacaccacgcgcctcgccatggacggcgacggctccggaaaagtgaactaccaggtccagaccggtccggcgctgggtgccttcaaccagtgggtcgaaggcaccgaactcgcctcatggcggcaccgccacgtagaccggatcggcctcatgctgctcgacggtgccgccgaacacatcgccaccgcatgccggcactggcgcgacaccctcggggtgcccagtgcgtgacggacatcccaccgcagtcgaggagaacgtgatgaccgttccgtccacgggcaccgaagtccggctcgccacccgccccgaggggtggccgaccactgagaacttctcggtcgtgcaggcggaaccgcccgcggtcaggaccggccaggtgctgatccgcaacctggtgatgagcgtcgacccgtacatgcgcgggcggatgaacaaaaccaggtcctacgttccgccgttcgccgtcggcaaggcgctcgacgggggcgccgtcggcgaggtcgtcgtctcgaagtcatcgcaactcgccgtcggtgacctggtcctgcacggcctcggctggcgggagtacgccgtcgtgggcgctgccggcgcggtcaggatcgacccggcgctcgcgccgcccggcgcgtatctcggagtgctcggcatgccggggcacgccgcctacacggggttgctcaaggccgccgaattccggcccggcgacaccgtgttcgtctccggggccgcgggcgcggtgggctccctcgtcggtcagatcgcccggctctgcggcgcggaacgcgtgatcggatcggcgggcagcgccgagaaagtcgcctatctgaccggggagctcggcttcgacgcggcattcgactacaaggacgggccggttctcgaacagctggcgaaggccgcgccgacgggcatcgacgtgtacttcgacaacgtgggcggcgaccacctggacgccgccctggtcctggccaggatgggcgcgcggttcgccctctgcggcaacatctcgcaggccaacgagaaggacccgccggccggcccacggaacctgacgcaggccatcgccaagggcatcaccctgcgcggcgtcctcgtcggaggccacgccgacctcccggacgagttcaccgcccgcatgggtggctggctggcagacgggagaatctcctaccgggagaccgtcgtcaggggactggagaacgcacccgccgccttcatcgacatgctgcgcggcgccaacaccggcaaaatgctcgtgagaatcgccgaatgaaaacaccacgccctgcggaagaaaagggtgaactccagggagtttcctgtctccacggaatttcctgtggaattgttccccattttcttcccggccggtatggtgatctccaagcgcagacgccacacagatggcgggcttcacgcctcgcgcagagaattccccacctgccccattttccctgggcattcagcctgcggtgagtagacgcttccggcgttccggccgaatccacgcgcccaccgtgcacgttccacccctttccggttcaccagacagaaaacggaggacttccatgtccagcacgtccacgaccgcccccgtctccgtccccgtctccgcccccgtccccgaagaggtcggacacctctacgaccgcctcaccgcactggacaccgaagcggccggcggcagcctccacctcggctactgggacgtcgacgacaacgacaccccgctcgtggaagcggccgaccggctcaccgacacgatgaccgaccgcctgcggatcgaccagggacagcgggtcctcgacgtcggctgcggagtcggccagccggccatgcggatcgcccggcgcaccggcgcccatgtcacgggcatcgcgatcagcaaggaccagatcgcccgcgccaccgccctcgccgagggcgccggcctgagcgaccgcgtggagttccggcacgccgacgccatggaactgcccttccccgacgactccttcgacgccgccatcgccatcgagtcgatcttccacatgcccgaccgcggacgggtcctcgccgagatccgccgcgtactgcgccccggcggccgcctggtcctcaccgacttcttcgagcgcggccccgtccccgccgagaagcagcccgcggtggaccggctcctccgcgacttcatcatgacgctggcccggcccgaggactacgtgcccatgctgcgcgacgcaggcctgcgcttcgtcgagctcctcgacatcaccgagcagagcgtgcgtcagaccttcgagcagatgagccagggctcccaggagatgcagaccgtcttcgacgacgaggcagaggaaaagttcagccccgcctccatgatcgacgtcgacgaattcggctccgttctgctgaccgcccaaaagcccctctgaccggggacgttcgaacgaggtg

SEQ ID NO: 2 - protein, 1217 aa, Streptomyces platensis subsp. rosaceus
MPSSLTPNQPPLTPASEDGPHGTPLQLSVLGPMSARHDGRDLPLGPPRRRALLALLLIRLGRVVPTELLIEELWGEEPPRQAVATLQSHVSHLRRALQPASGLDRPTVLRHRAPGYVLELAPEQLDACRFERLVAEGRRLLEQRDPLAARDRFAAALTLWRGSPYAEFDGHPPLSDESARLEHVRLTAVESCAQARLALGEAQEVAAGLDREVRRHPARERLVGHLMTALSQLGRQAEALEVYERTRVHLNEEFGVGTAAELRRVRTAILRQEPGAGGPPAGPRPGRPEPAGPALGAPVIGVATAHEGDGARGPDAHPPRAAQEPERPTAAEASGGIGRAAAAPDEPFAADCGDDAPQDGTLSAGSGGAGSAAADGAQADAPTATPGPARRPVTITATSRSTTADSAESARPTQDAKAQECDTSTEASGPVCGGPGAQSPFTGRSEELQRLTAAASSALAGHGHVAGVLGPAGVGKTRLLLELVPQLEAARAGEDGTGQERAGLEVIWSHCFLGEGVPPYWVWTQILRRLSTTRPDAFREAAKPFGTLLAPLMPERAARPGGLASESDWGQARFLTHDAVCEVLLALAAQRPLVLLMEDLHWSDPASLDLLRLLSTRSQGHPLGIVLTAREHEIESDATLRRMLSEVLRGPRTETLRLGGLPRRAVAALVVAQVGPGVDARVVEVLHRRSEGNPYFVMQLLSLLGDARSLRRPDAVDVLLTRVPTGVREALHQRFAALPETVLRVLRLCAVIGTEVDTDLLERTATEDEPVTAALELAIRAGLLGEDRHHPERLHFTHALVQETLIDELPREDRQRLHARVAEGISTRTLGQVADEEIERIAHHAWHAKSALPTEETLPLLLRAAEQAEQQLAYEQVETWLRRAVHLVGLLPPGDPSAVSLNQRLHIQLGQVLAITRGYGHAEAQTALARGRALSAATHSPEDPSVLWALCAAYIVTGRYDASRQFSGLLRNLADRTGHPVAVLGAAYGEGIVLHIRGQLRPALTELEHGVAMADEYAGEGHSLARTFQHDPRVSCRSYDTFTHWLMGDRKTATARRRELLRLTEYDSRPSDRSFALYVDAVVAAWEGDARTARSSGAEGVRLADEHGLLYWKAMLGVLEGWGRTHSGQEDGLTLMHSSLAELRNSRTHLRRPLHLGLLGQAQHRTGRTEDAKTTFHALLAAVGQSDEPVYLHPELPATRLLHDLLGRGAAEAVAAG

SEQ ID NO: 3 - DNA, 3654 bp, Streptomyces platensis subsp. rosaceus
atgatctggatgagctggcgccagttccgctggcaggccctggccggtgccgtcgccctggtgccgttggtggcctacttgatcgtcacgagcctggacatccggcgcgcccacgaccgctatcaggcgcagtgcgcgtccatcggcaactgcgccgaggcgatgctccagttccagaacgacttccgcacccgcctgctgctgctcgccatcctgctggccgcgatccccggcatcctcggggtgttctggggcgcgccgctggtggcccgcgagctcgagaccggcacgcaccgcctggtctggaaccagagcgtcacccggcgccggtggctggcggtcaaggtgctgttcgtcggtgtcgccgcgatggccgtggccacgctcgtcagcacgctgctgacctgggcgagcagcccggtcgacgcggtgtcgcaggaccggttcggcgcgctggtgttcgacgcccgcaacatcgtgccggtcgcgtacgccgccttcgccctcgtcctcggcacggtgatcggcctgctcgtgcgccgcaccatcccggccatggcgctcaccatgctcgtcttcgccgtcgtgcagttcaccgtgccggcgctggcccggccgcacctgatggcgccggagacccagacccggcagatgacgttgcaggagttcggcgaggtgcgcggcttcggcgacgagcccacggtcaacgggctgagcatccggggcgcgtgggtgaccagcaccagcccgctgctcaccgccgacgggacccggctcgacaaggccacgtaccgcaaatgcgtgaccgaccccccggccgtctcgggcggagctcccggcgtcggcggcaccgtcgcctgcctggccgacctcgatctgcacgtcgaggtggcctaccagcccaacgaccggtactggaccttccagtggatcgagtcggccctctacctggcgctcggtggactgctcctcgccgtgggcctgtggcgcatccgccgccacgtcatctgaggccgggcggcagcggctccggacgagcccttcgcagcagactgcggtgatgacgctccccaggacggcacgctgtccgcgggatcgggaggggccggaagcgctgccgcggacggggcgcaggcggacgcgcccaccgccacaccgggacccgcgcgccggccggtgacgatcaccgcgacatcgcggtcgaccaccgcggattcagcggagagcgcgcgtccgacgcaggatgcgaaagcccaggagtgcgacacgagcaccgaggcctcagggccggtgtgcggcggtccgggcgcccagtcgccgttcaccggccgcagcgaggagttacagcgcctgacagccgcggcgtcgagcgcgctcgccggtcacgggcacgttgcgggcgtcctcggccccgcgggcgtcggcaagacccgtctgctcctcgaactcgtcccccagctggaagccgcccgcgccggcgaggatggcaccggtcaggagcgcgccggcctggaggtgatctggagccactgcttcctgggcgagggcgtgccgccctactgggtgtggacccagatcctccggcgactgtccacgacccgcccggacgccttccgcgaggcggcgaaaccgttcggcaccctgctcgccccgctgatgcccgagcgcgcggcccggcccggcggactcgcctccgaatccgactggggccaagcccggttcctcacccacgacgcggtctgcgaagtcctgctcgccctcgccgcccagcggccactcgtcctgctcatggaggacctgcattggtccgaccccgcctccctggacctcctcagactgctgagcacccgcagccagggccacccgctcggcatcgtcctgaccgcccgtgagcacgagatcgagtccgatgcgacgctgcgccgcatgctctccgaggtgctgcgcggccccaggaccgagaccctgcggctcggcggcctgccccgccgcgcggtcgccgccctcgtcgtcgcccaggtcggacccggcgtcgacgcgagagtcgtcgaggtgctgcaccggcgcagcgagggcaacccgtacttcgtcatgcagctcctctcccttctgggtgacgctcgcagcctgcggcggcccgacgcggtggacgtgctcctgacgcgcgtcccgacgggcgtccgcgaggccctgcaccagcggttcgccgcactccccgagacggtgctgcgcgtgctgaggctctgcgccgtcatcggcaccgaggtcgacaccgatctgctggaacgcaccgccaccgaagacgaaccggtcaccgcggcactggagttggcgatccgggccgggctgctcggcgaggatcgccaccacccggaacggctccacttcacccacgccctggtccaggagacgctcatcgacgagctcccccgcgaggaccggcagcggctgcacgcgagagtcgccgaggggatatcgacccgcacgctcggtcaggtggcggacgaggagatcgagcggatcgcccaccacgcctggcacgccaagagcgcactgccgactgaagaaacgcttcctctgttgctgcgcgccgccgagcaggccgagcagcaactcgcctacgaacaggtggagacctggctgcgccgcgcggtgcacctggtcggcctgctgccgcccggcgacccctcggccgtgtccctgaaccagcggctgcacattcaactcggccaggtgctggccatcacccggggctacggccacgccgaggcccagacggcactcgcccgcggacgggccctgagcgcggccacccactcccccgaagacccgtcggtgctctgggccctgtgcgcggcgtacatcgtcaccggccgctacgacgcctcacgccagttctcgggcctgctccggaacctcgccgaccggaccggccacccggtggcggtgctcggcgcggcctacggcgagggcatcgtcctccacatccgcggccagttgcggccggcactcaccgaactggagcacggcgtcgccatggcggacgagtacgccggcgagggccactccctggcccgcacgttccagcacgacccacgcgtctcctgccgctcctacgacaccttcacccactggctcatgggcgaccgcaagaccgccacggcacgccgccgcgaattactgcgcctcaccgagtacgacagcaggccctccgaccgctccttcgctctctacgtggacgcggtcgtggcggcctgggaaggcgacgcccgcacggcccgctcctccggcgccgagggagtccgcctggcggacgaacacggactgctctactggaaggccatgctcggcgtgctggagggctggggccgcacccactccggacaggaggacggcctcaccctgatgcactcctccctcgccgaactccgcaactccagaacccacttgcgccgccccctccacctgggcctcctcggccaggcccagcaccggaccggacggacggaggacgcgaagaccaccttccacgccctcctcgcagccgtcggccagagcgacgaacccgtctaccttcatccggaactccccgccacccgcctcctccacgacctcctgggccgtggggccgcggaggcggtggcggccgggtga

SEQ ID NO: 4 - protein, 529 aa, Streptomyces platensis subsp. rosaceus
VLFPGQGSQARGMGAGLFDRYPELTALASDILGYDLPRLCLEDPDGRLDDTRCTQPALYVVNALSYQDSLERGEPEGGYLLGHSLGEYNALHAAGAFDFETGLKLVLKRGELMARAPDGAMLAVVGPDAGEVRAFLSEEGLSRLDVANINTPVQTVLSGARDEIERAHKTLDAHGTRVARLKVSAAFHSRFMAAARDEFAAFLKGFRFAPLRATVIANLTARPYTDQDVAATLSEQICGSVQWLDSVRYLLERTTAGHCREVGGGGVLTRMIRQIDAAPARGIPQPKPKPKPKPKPKPQSRPRLFCIAYAGGDERAYAGLAEHCPDVDVVTLERPGRGRRASEPLLREPAAIVDDLLRQLRGRLDAPYALYGHSLGARLAFLLCRALRAERLPAPAHLFVSGESGPALPSRERHTWELPADAFWDHLKELGGIPAELWEHPDLMAYYEPVIRADFTALGAYRHEDAPPLDVPVTAMAGEDEWFTRADLEAWQRESTRPLTTHRFPGDHFFIRAQWPALARIVAAGLAAP

SEQ ID NO: 5 - DNA, 1590 bp, Streptomyces platensis subsp. rosaceus
gtgctcttccccggccaggggtcccaggcccgtggtatgggagccggcctcttcgaccggtaccccgaactgaccgcgttggccagcgatattctcggctacgacctcccgcggctgtgcctggaggacccggacgggcggctcgacgacacgcgctgcacccagcccgcgctctacgtcgtcaacgccctctcctaccaagactcgctggagcgcggcgaacccgagggcgggtacctgctgggccacagcctcggggagtacaacgccctgcacgccgccggggccttcgacttcgagaccggcctgaagctcgtcctcaagcggggcgagctcatggcccgggctccggacggggccatgctcgccgtcgtggggccggacgcgggtgaggtgcgggccttcctctcggaggagggcctgtcgcggctcgacgtcgccaacatcaacacccccgtccagaccgtgctgtccggggcccgggacgagatcgagcgcgcgcacaagacgctcgacgcgcacgggacccgggtcgcccggctgaaggtctcggccgccttccactcgcggttcatggccgctgcccgcgacgagttcgccgccttcctcaaaggcttccggttcgcgcccctgcgggccacggtgatcgccaacctcaccgcacggccgtacacggaccaggacgtcgcggcgacgctgagcgagcagatctgcggatcggtgcagtggctggacagcgtccgctacctgctggagcgcacgaccgccggccactgccgtgaggtgggcgggggaggagtcctgacccgcatgatccggcagatcgacgcggcccccgcccgcgggattccacagccgaagccgaagccgaagccgaagccgaagccgaagccgcagtcacggccacgcctgttctgcatcgcctacgccggaggcgacgagcgcgcctacgcaggactcgccgaacactgcccggatgtcgacgtcgtgacgctggaacgccccggacgcggccggcgcgcctccgagccgctgctgcgcgaacccgccgcgatcgtcgacgatctgctccggcagctgcggggccggctcgacgccccgtacgcgctctacggccacagcctcggagcccggctggcattcctgctctgtcgggcgctgcgcgccgagcggctgcccgcaccggcccacctgttcgtctccggggagagcggaccggccctcccgagccgggaacgccacacctgggagctgccggccgacgccttctgggaccacctcaaggagctcggcggaatcccggcggagctgtgggagcaccccgacctgatggcgtattacgagccggtcatacgggccgacttcaccgcgctgggcgcctaccggcacgaggacgctccgccgctggacgtgccggtcaccgccatggccggcgaggacgagtggttcacccgggccgacctggaggcctggcaacgcgagagcacccggcccctgaccacacaccggttccccggtgatcacttcttcatccgggcccagtggcccgcgctggcccggatcgtcgctgccgggctcgcggccccctga

SEQ ID NO: 6 - protein, 83 aa, Streptomyces platensis subsp. rosaceus
MKQELKKHMEERFMFEFDSDITEDTDLFKAGILDSFGYISLMTHIEEEYGVPLGDEILGNVAVSLSGIVAFVDAARLRAAGSR

SEQ ID NO: 7 - DNA, 252 bp, Streptomyces platensis subsp. rosaceus
atgaagcaggaactcaagaagcacatggaagagcggttcatgttcgagttcgactcggacatcaccgaggacaccgacctgttcaaggcgggcatcctcgactcgttcggttatatctcgctgatgacgcacatcgaggaggagtacggcgtgccgctcggcgacgagatcctcggcaacgtcgcggtctcgctgtccggcatcgtcgcgttcgtcgacgccgcccgcctgcgggccgccgggagccggtga

SEQ ID NO: 8 - protein, 656 aa, Streptomyces platensis subsp. rosaceus
MCGIAGFYGSPLPPQEYETLIHGMLAQIEHRGPDEAGCFLDDRLAMGTVRLSIIDLSTGSQPVGSADGRYWLCYNGELYNYRELREQLTARGFVFRTESDTEVVLAAWVAWGLDCLPRFNGAFAFALYDSATGELHLVRDRFGKRPLYVARHRGAWLFASEMKAFLAYPDFRFAFDEAQLASVFATWTPLPGQSGYQGIEQIPMGEYLSVRGDEVRRGRWATLDLAQGPAPESEQEAAELVRADLEAAVDVRLRSDVEVGVYASGGLDSSIIAHIAAQRTSRPLRTFSIEFEDAEFDESAEQAELAAHLGTRHSTVRVTDEDVADAFPEAVRHAEVPVFRTAFVPMYLLAGHVRSEGIKVVLSGEGADEAFLGYGIFKDTLLLSTWHELDDDTRLRRMSQLYPYLSHFSGEDGHRRMLGLYRQFTEETLPGLFSHQMRFQNGRFAARLLKNPGDPFAALGELVAGEPGYAQLTPVQKAQWLEFRTLLSGYLLSTQGERMALAHGVENRCPFLDPAVVRRAASVNLRFGDPYDEKYLLKCAYADVLPERIVKKGKFPYRAPDSAAFVRSRPDYRELLTDPGTLDEIGVLDARFVKRFTDRVFDSPPEQIGTKENQAFVSLASTVWLHHWYVRGNARRRAPLGVPLYVVDRRSGALSA

SEQ ID NO: 9 - DNA, 1971 bp, Streptomyces platensis subsp. rosaceus
atgtgcggcatcgccggcttctacggaagccccctgccaccgcaggaatacgagaccctgatccacggcatgctcgcccagatcgagcaccgcggcccggacgaggcgggctgcttcctcgacgaccgcctggccatgggcacggtacgactgagcatcatcgacctgtccaccggctcgcagccggtcggcagcgccgacggccgctactggctctgctacaacggcgagctgtacaactaccgggagctgcgtgagcagctgaccgcccgcggcttcgtcttccgcaccgagtccgataccgaggtcgtgctggccgcctgggtcgcctggggcctggactgcctgccccgcttcaacggtgccttcgcctttgccctttacgacagtgccaccggcgaactgcacctggtgcgcgaccggttcggcaagcggccgctgtacgtggcgcggcaccgcggcgcgtggctgttcgcctccgagatgaaggcgttcctggcctaccccgacttcaggttcgccttcgacgaggcacagctggcgtcggtcttcgccacctggaccccgctgcccggccagagcggataccaagggatcgagcagatccccatgggcgagtatctgtccgtacgcggcgacgaggtccggcgcggccgctgggccacgctcgacctggcccaaggcccggctccggagagcgagcaggaggccgccgagcttgtccgcgcggacctcgaagccgcggtcgacgtgcgcctgcgcagcgatgtcgaggtcggcgtctacgcctccggcggcctggactcctcgatcatcgcgcacatcgccgcgcagcggacgagccgcccgctgcggacgttctcgatcgagttcgaggacgcagagttcgacgaatcggccgaacaggccgagctggccgcacacctgggcacccgccactccaccgtgcgcgtgaccgacgaggacgtcgccgacgccttccccgaagccgtccggcacgccgaggtgcccgtcttccgcaccgccttcgtccccatgtacctgctggcaggccacgtccgcagcgaagggatcaaggtcgtgctcagcggcgagggcgccgacgaggccttcctcggctacggcatcttcaaggacacgctgctgctctcgacctggcacgagctggacgacgacacccgtctgcgccgcatgagccagctctacccgtacctgagccacttcagcggcgaggacggccaccgccggatgctcggcctctaccggcagttcaccgaggagaccctgcccggcctcttctcccaccagatgcggttccagaacggccgcttcgccgcacgcctgctcaagaacccgggcgaccccttcgcggccctcggggaactcgtggccggtgagcccggctacgcacagctcacccccgtacagaaggcccagtggctggagttccgcacgctcctgagcggctacctgctctcgacccagggcgagcgcatggcgctggcccacggcgtggagaaccgctgccccttcctcgatcccgccgtggtccgccgcgccgcatcggtgaacctgcggttcggcgacccctacgacgagaagtacctgctcaagtgcgcctatgccgatgtgctgccggaacggatcgtcaagaaggggaagttcccctaccgcgccccggacagcgccgcgttcgtccgctcccgcccggactaccgcgagctgctgaccgaccccggcaccctcgacgagatcggcgtcctcgatgcgcgcttcgtgaagcggttcaccgaccgcgtcttcgacagcccgcctgagcagatcggcacgaaggagaaccaggccttcgtctctttggcgtcgacggtctggctgcaccactggtacgtgcgcggcaacgcccgccgccgggctccgctcggggtccccctgtacgtcgtcgaccggcgcagtggcgccctgtcggcctag

SEQ ID NO: 10 - protein, 3192 aa, Streptomyces platensis subsp. rosaceus
MKKQNGVLADDRDIAVIGLSLRLPGSRTPEEFWSHLAEGRSLISEVPERRWRKEDHLGHPRREFNKTNSVWGGFVDDADCFDADFFQISPREAQSMDPQQRMALELSWHALEDAGYRADRVAGSRTGVFMGVCHWDYAELMEQEVEEIDAYYPTGAAYAIIANRVSHHFDFRGPSVVNDTACAGSLVAVQQAVQALQAGDCDLALAGGVNLTWSPRHFIAFAKAGMLSPDGLCRAFDANANGYVRGEGGGIVLLKRAADARRDGDAVHAVIKGIGSNHGGRTSSLTVTNPAAQAELIAGVYRKAGIAPETVTYVETHGPGTPVGDPIEVRGLKQAFVDLGADRPGEAPAHRCGIGSVKTNIGHLEGAAGIAGMLKVILAMRHRKLPATVNFRKLNPLIDLDGSPLYVLDRLTDWTAEGSAPLRAGVSSFGFGGTNAHVLLEAAEPVAATEDAGEQWLPVSAMDEDRLRETCARLARWVRTRIEQNDAPSLTDAARTLREGRVSMRERVVFRASGIEEWAAQLESVAAGDGPPADCPRGRAGTEAPDGLDADDLTALAERWLEKGRWDKFAAAWAQGLAVDWAPWPERGRRVHVPGYAFARTPHWFRTDRNETTGRPERGATNTAPAPLGEGKPEGGSWTFPLHFDATQGFVRDHRVNGARIVPGVVALELVTVAAERAAAAGARAGLTPRIRNAVWIRPLLVGDTVLSPQLRLTPAADGYDYAITDEHGTQYTSGRVEYGEAAAAEKTDPGALRERFPQRVDTAEGYAALRSSGIEHGPALRGLNALHRGPDGVLAELRLPAGAPEGMALQPAILDSALLAALALGSADGGWRRPAAPVVPFALDRLTVHAATTSTMWAWLRPAGSGTAGDMARSDIDLFDDNGRLCVRLAGYTSRELPTAEPAAVQAPEGELLEVTGVWEEAPAPAPAAGQATPVGPVTVLNAALDGDLAAASAARLGMDIRQLAGPAEATDATDAVAMKAAFEACYPQVRQLLGQGRQVLVVAPGAPDSPVYAPLAALLKTAQQENPSFRGRLVLLDGYDPRDADRFERVVSAEAGAGDDTEVAYDAQDRRLRHGFVELPRGEAGESLLRDGGVYWITGGAGGLGLLLAERLCERRRATVVVSGRSADSRAIEALRARLFHGEVAYRRTDVTDADAVRDAVADIRARYGRLDGVFHAAGVLDDGYLASKPLAGTAAVLAPKVDGATSIDDATRAHGLDFLLLFGSVAGAFGNAAQADYAAANAFLDAFAARRQAAGSVTRSVDWPLWADGGMRVDDASLAYLRKRTGTVPLPSETGLDALERALHSAAPVRRVVLFGERSKLRGYAGLDRVAKPEPRTSGAQRNTAAPAVLEESELVARTQDLLRNLFAEVTLQDAEHILAEEKLETYGIESISIVELTSKLEDTFGSLPKTLFFEYVDLQGVAGYFVAEHRDRLLELFAPEAPAPEAPAPEAPAPEAPAPEEPAPEGPAVEEPPAAAPTPAVRPSVEAAAGRARPAWADPERHDIAVIGMAGRYPGADTLEEFWELLSEGRHSFEPVPESRWRHGDIYFDERDVDGKTVVKTGTFLRDVEAFDPRYFNISQRDAELLSPEVRLFLQAGVEALEDAGYSRETLRRRYDGDVGVLVGSMNNSYSLYGFQNMLMRGTATSGSELGVMANMLSYHYGFTGPSVFLDTMCSSASACVHQAVRMLRSGECRMTVVGGINLMLHPFDLIATSQAHFTTKSAEVVRSYGLGADGTILGEGVGTLVLKPLAEAVADGDHVYGVIKGSGMTNAGVRNGFTVPSPQQQARAIEKALDDAAVDARTISYLEGHGSATSLGDPIEIKGAALAFGRDTQDLGFCALGSVKSNVAHLLSGSGMAGLTKVLLQLKHRTLAPSLHAGTLSSAIDFEETPFVVQRHRDTWRRPVVGGEEAPRRAGVTSIGAGGINVHIVVEEYDGQVVAAPERGRPRLLVFSAMTPQALQSVLRAMHEHVRETAPGLDALAYTLQTGKNELPCRLAFVADDIADAQARLARLSAVDWTAESPGVPAGVHFTASTLRRRRTADAATVEQALRDGKQAELAQHWADGASVDWDLLWPAGSRPAKPSLPAYPFDKVRCWYPEDDDAPSVLRPLAFARRAHPWVGVNASDLGGVRYTLRLRGDELLDYVYTVGRKRRYATVALLDAALAFARLAGLEGPLRLRNAQWAALPSPADTPETFTWRLGTSGDGVHRVELWHADEATLRFAADVVPSAPAEDASMPQMSSAPATLDRDDFYAALGTAGLDARPYARSVEGVTELDAHRLLVRVAEPAMCQDPHKQHVHLPAWALVGLTQGVQHAWGRADAAVVRVGSVQGEQWERTRAIVLARTSDAVFHAAFLDEDGRVLGRVEDAEFTAGDLEPALPGEAGRALVALPQASRPVLETPVGTGEWQQSEAVRPEAEPSVTVAAVADGPAALVASLRETVADLLKFDLADIDLDTHFHAYGFESIALAKLASELNGVLGTDLTPAVFFECSDIRSLAEYLLDRYGPELSLPTSADAPAPVAATRPSPVPMPAPGPDDDAVAIVGAAGRFPGADDLDTFWQQLRAGEDLIADYPGDRFDGGPYAEVVARADFPKFAGRIEGVDRFDADFFHLSRLEAELMDPQHRLALETVWAALENGGYAPARLPENTGVYFGVSGSDYHHLLNASGVAPDGFTATGNAHSMLANRISYVLDVHGPSEPVDTACSSSLVALHRAVEHIRSGRCEMAIAGGVNLLLSVDTFAATHMAGMLSPDGRCKTFSAGADGYVRSEGVAAVLLKPLAQAQRDGDAIWGVVRGSAENHGGRAGSLTAPNGKAQAALIQDAMRGIDPDSIGYVEAHGTGTGLGDPVEVNALDSAYRALRTAEGGPPHAARPCALGSVKTNIGHAESAAGLAGVLKVLLAMRHRELPPALHCDRLNPHLPLDGGFEVVRELRRWEPCTDATGRPWPLRAGVSSFGFGGANAHVVLEAPPVPPAPAEPARPTAPQAIVLSARDDDRLRATAGRLRDFLDRARRDGHAPDLADLAFTLQVGREAMERRLGFVVGSMDDVLGTLDRFFAGDEPSGWHTGGIRRSRGAGVRREAEQAPEVTRALHDGRLDRVTALWCDGAPVDWQAMHPTGERRAVRLPAYPFACDRYWVPAVGTAPVPPPAAPVPPPAAEPAFETDARAALLDAVLDGRAGPDALSRT

SEQ ID NO: 11 - DNA, 9579 bp, Streptomyces platensis subsp. rosaceus
atgaagaagcagaacggcgtcctcgccgacgaccgggacatcgccgtcatcggcctgtccctgcggttgcccggctcgcgcacgcccgaggagttctggagccacctggccgagggccgctcgctcatcagcgaagtcccggagcgccgatggcgaaaggaggaccacctcggtcacccgcgccgcgaattcaacaagaccaacagcgtctggggcggcttcgtcgacgacgccgactgcttcgacgccgatttcttccagatctccccgcgcgaggcgcagtccatggacccgcagcagcggatggccctggagctgagctggcacgccctggaggacgccggctaccgggccgaccgcgtggcgggctcccgcaccggtgtcttcatgggcgtctgccactgggactacgccgagctgatggagcaggaagtcgaggagatcgacgcctactacccgaccggcgccgcgtacgcgatcatcgccaaccgggtctcccaccacttcgacttccgcggaccgagcgtcgtcaacgacacggcctgcgcgggctcgctcgtggccgtgcagcaggcggtgcaggccctccaggccggcgactgcgacctcgcgctcgccggcggcgtcaatctgacctggtcgccgcggcacttcatcgccttcgccaaggcgggcatgctctcgcccgacggcctgtgccgggcgttcgacgcgaatgccaacggctatgtgcgcggcgagggcggcggcatcgtgctgctgaagcgggccgcggacgcccgccgcgacggcgacgccgtgcacgccgtgatcaagggcatcggcagcaaccacggcgggcgcaccagttcgctgaccgttaccaacccggccgcacaggccgaactgatcgcgggtgtctaccgcaaggccggcatcgcacccgagaccgtcacctacgtggagacccacggccccggcacaccggtcggggaccccatcgaggtccgcggcctcaagcaggccttcgtcgacctgggcgcagaccggcccggggaggctccggcccaccggtgcggcatcggctccgtgaagaccaacatcggccacctggaaggggccgccggcatcgcgggcatgctcaaggtcatcctcgccatgcgccaccgcaagctgcccgcgacggtcaacttccgcaagctcaaccccctcatcgacctggacggcagcccgctgtacgtcctcgaccgcctcaccgactggaccgccgaagggtccgcaccgctgcgcgccggcgtcagctccttcggattcggcgggaccaacgcccacgtcctgctggaagccgcggagccggtggccgccaccgaggacgccggcgaacagtggctgccggtgtccgccatggacgaggaccggcttcgcgagacgtgcgcccggctcgcccgctgggtccggacccggatcgagcagaacgatgctccgtccctgaccgatgccgcccgcacgctgcgcgaaggccgggtgtccatgcgcgagcgcgtggtgttccgcgcaagcggcatcgaggagtgggcggcacagctggagagcgtcgccgcgggggacggcccgcccgcggactgcccgcgcggccgggccggaaccgaagcccccgacggcctggacgcggacgacctgacggccctggccgagcgctggctggagaagggccggtgggacaagttcgcggccgcctgggcccagggcctggccgtggactgggcaccgtggcccgagcgcggccgccgcgtgcacgtgcccggctacgcgttcgcccgcacgccgcactggttccggacggaccggaacgagacgaccggaaggccggagcgcggcgcgacgaacaccgctcccgccccgctcggcgaaggcaagccggaaggcggcagctggaccttccccctgcacttcgacgccacccagggattcgtccgcgaccaccgcgtcaacggcgcacggatcgtcccgggcgtggtggccctggaactcgtcaccgtggcagccgaacgggccgccgccgcaggtgcccgggccgggctgacgccccgcatccgcaacgcggtgtggatccgtccgctgctcgtcggcgacaccgtgctctccccgcagctccgcctgacccccgccgccgacggctacgactacgcgatcaccgacgagcacggcacgcagtacaccagcggccgggtcgagtacggcgaggctgccgcggccgagaagacggacccgggcgcgctgcgcgagcgcttcccccagcgcgtcgacaccgccgagggttacgccgcgctgcggtccagcggcatcgagcacggccccgccctgcgcggcctcaacgccctgcaccgcggtccggacggcgtgctggccgagctgcggctccccgcaggtgccccggagggcatggcgctgcagcccgcgatcctcgacagcgccctcctcgcggccctggctctcggctcggccgacggcggctggcgcaggcccgccgcccccgtggtgccgttcgcgctggaccggctcaccgtgcacgcggcgacgacctcgacgatgtgggcgtggctgcggccggccggcagcggcacggcaggtgacatggccagatccgacatcgacctgttcgacgacaacggccggctgtgcgtgcgcctggccggctacacgtcgagggaactgcccaccgcagaaccggcagcagtccaggccccggaaggggagctcctggaggtcaccggtgtgtgggaggaggcccctgccccggcgcccgcagccggtcaggccaccccggtcggcccggtgacggtgctcaacgccgcactggacggcgacctcgcagcggcgagcgccgcgcggctgggcatggacatccggcagctggccggtcccgcggaagccaccgatgccaccgatgccgtcgccatgaaggcggcgttcgaggcctgctacccgcaggtccggcaactgctcggtcagggacggcaggtgctcgtcgtcgccccgggcgccccggactcgccggtctacgccccactggcggcgctgctgaagaccgcacaacaggagaatccttccttccgggggaggctggtgctcctcgacggctacgacccccgcgacgccgaccgcttcgagcgtgtcgtcagcgcggaggcgggcgccggagacgacaccgaagtcgcctacgacgcccaggaccgccgactgcggcacggcttcgtggaacttccccggggcgaggcgggggagagcctgctgcgcgacggtggcgtctactggatcaccgggggcgccggcggactcggcctgctgctcgccgagcggctctgcgagcgccgccgcgccacggtcgtcgtcagcggccgctcggcggacagccgggccatcgaggcactgcgggcccgcctgttccacggcgaggtggcgtaccgccgcacggacgtcacggacgcggacgccgtacgggacgcggtcgcggacatccgcgcgcggtacggacggctcgacggcgtgttccacgcggccggcgtcctcgacgacggctacctcgcgagcaagcccctcgcgggcaccgccgccgtactcgcgcccaaggtggacggcgccacgtccatcgatgacgcgacgcgcgcccacggcctggacttcctcctgctgttcggctccgtggcgggcgccttcggcaacgccgcgcaggccgactacgccgccgccaacgccttcctcgacgcgttcgccgcacgacggcaggccgccggcagcgtgacccgctccgtcgactggccgctgtgggccgacggcggcatgcgcgtggacgacgccagcctcgcctacctgcgcaagcgcaccgggaccgtgccactgccgagcgagaccggcctggacgcactggagcgcgcactgcactccgccgcgccggtccgccgcgtggtgctcttcggggagcggtccaagctgcgcgggtacgcgggcctggaccgcgtcgcgaagccggagccgcgcacgtccggggcgcagcggaacacggccgcgccggccgtcctggaggagagcgaactcgtagcccgtacacaggacctgctgcggaacctgttcgccgaggtgaccctgcaggacgcggagcacatcctggccgaggagaagctggagacctacggtatcgaatcgatctccatcgtcgagctgaccagcaagctggaggacaccttcgggtcgctgcccaagacgctcttcttcgagtacgtcgatctgcagggcgtggccggctacttcgtcgccgagcaccgcgaccggctcctcgaactcttcgcccccgaagcacccgcccccgaagcacccgcccccgaagcacccgcccccgaagcacccgcgcccgaggagcccgccccggaggggcctgccgtcgaggagccgcccgcggccgcgcccaccccggccgtccggccgtccgtggaggccgccgccgggcgcgcccgcccggcctgggccgatccggagcgccacgacatcgcggtcatcggtatggcgggccggtacccgggcgccgacaccctggaggagttctgggagctgctcagcgagggccggcacagtttcgagcccgtcccggaatcgcggtggcggcacggcgacatctacttcgacgagcgtgacgtcgacggcaagaccgtcgtcaagaccggcaccttcctgcgggacgtcgaggcgttcgatccgcgctacttcaacatctcccagcgcgacgccgagctgctgtcgccggaggtccggctgttcctgcaggcgggcgtggaggccctggaggacgcgggctactcacgtgagacgctgcggcgccgctacgacggcgacgtcggtgtgctcgtcggctcgatgaacaacagctactcgctctacggcttccagaacatgctgatgcgcggcaccgcgaccagcggcagcgagctcggtgtgatggcgaacatgctgtcgtaccactacggcttcaccgggccgtccgtgttcctcgacaccatgtgctcctcggcgtcggcgtgtgtgcaccaggcggtgcgtatgctgcgcagcggcgagtgccgcatgaccgtcgtcggcggcatcaatctgatgctgcacccgttcgacctcatcgcgacctcgcaggcgcacttcaccaccaagtcggccgaagtcgtgcgcagttacggcctcggcgccgacggcacgatcctgggcgaaggcgtgggcacgctcgtgctcaagccgctggccgaagccgtcgccgacggcgaccacgtctacggcgtgatcaagggcagcggcatgaccaacgccggggtccgcaacggcttcacggtgccgagcccacagcagcaggcgcgcgccatcgagaaggcgctcgacgacgccgccgtggacgcgcgcacgatcagctacctggagggtcacggctcggcgacctccctcggcgaccccatcgagatcaagggcgccgccctcgcgttcggccgggacacccaggacctggggttctgcgcgctgggctcggtcaagtccaacgtggcgcacctgctgtccggatccggcatggccggcctgaccaaggtgctgctgcagctcaagcaccgcacgctggcgccctcgctgcacgccgggacgctcagctcagcgatcgacttcgaggagaccccgttcgtggtgcagcgccaccgcgacacgtggcggcgccccgtggtcggcggcgaggaggcgccgcgccgggcaggcgtcacgtccatcggcgcgggcggcatcaacgtgcacatcgtcgtcgaggagtacgacggccaggtcgtcgccgcaccggagcgcggtcgcccgcggctgctggtgttctccgccatgacaccccaggccctgcagtcggtgctgcgcgccatgcacgagcacgtacgggagaccgcaccgggcctggacgccctcgcgtacaccctgcagaccggcaagaacgaactgccgtgccggctggccttcgtcgcggacgacatcgcggacgcccaggcccgtctggcccggctgtccgcggtggactggacggcggagtcacccggcgtgcccgcaggcgtgcacttcacggcgagcacgctgcggcgccggcgcaccgccgacgcggcgaccgtcgaacaggccctgcgcgacgggaagcaggcggagctggcgcagcactgggcggacggcgcgagcgtcgactgggacctgctgtggccggcgggaagccgtccggccaagccgtcgctgcccgcctaccccttcgacaaggtgcgctgctggtaccccgaggacgacgacgcgcccagcgtgctgcggccgctcgccttcgcccggcgcgcgcacccctgggtcggcgtcaacgcctcggacctgggcggggtgcgctacaccctccggctgcgcggcgacgaactcctcgactacgtctacaccgtaggacgcaagcgccgttacgccaccgtggcgctgctggacgcggcactggcgttcgcgcggctcgccgggctggaagggccgctgcggttgcggaacgcgcagtgggccgcactcccgtcgcccgcggacacccccgagacgttcacctggcggctcggcacgtccggcgacggcgtgcatcgcgtcgagctgtggcacgccgatgaggccacgctccggttcgccgccgacgtcgtaccgtccgcgcctgccgaagacgcatcgatgccgcagatgagcagcgcgcccgcgaccctcgaccgggacgacttctacgccgcgctcggcaccgcgggcctcgacgcccggccgtacgcgcgcagcgtcgaaggggtcaccgaactcgacgcccaccggctgctcgtacgggtcgccgaaccggccatgtgccaggacccgcacaagcagcacgtgcatctcccggcctgggcgctcgtcgggctgacccagggtgttcagcacgcgtggggccgggccgacgccgccgtggtgcgggtcggatccgtgcagggcgagcagtgggagcgcacccgggcgatcgtgctggcgcggacgtccgacgccgtcttccatgcggctttcctcgacgaggacggccgcgtgctgggccgggtcgaggacgccgagttcaccgcgggcgacctggagccggcactccccggtgaggccggacgcgcactcgtggcactgccgcaggcgtcgcgtccggtgctggagacgccggttggtacgggggagtggcagcagtcggaggccgtgcggccggaggccgagccgtccgtgaccgttgcggcggtcgcggacgggccggcggcgctcgtcgcgtcgctgcgcgagaccgtcgccgacctgctcaagttcgacctggcggacatcgacctcgacacgcacttccacgcgtacggcttcgagtccatcgcgctggccaaactggcctcggaactcaacggcgtcctcggcacggacctcacccccgccgtcttcttcgagtgctccgacatccgcagcctcgccgagtacctgctcgaccgctacggccccgagctgagcctccccacgagcgccgacgcccccgcgccggtcgccgccacccggccgtccccagtgccgatgccggcacccgggccggacgacgacgcggtggccatcgtcggcgctgccggacggttccccggcgcggacgacctggacaccttctggcagcagctgcgcgcgggcgaggacctgatcgccgactaccccggcgaccgcttcgacgggggcccctacgcggaggtcgtcgcgcgggcggacttcccgaagtttgccggccggatcgagggcgtggaccgcttcgacgcggacttcttccacctgtcgcggctggaggcggagctgatggacccgcagcaccggctggccctggagaccgtgtgggccgcgctggagaacggcggctacgccccggcgcgcctccccgagaacaccggggtctacttcggcgtctccggcagcgactaccaccacctgctcaacgccagtggcgtggcacccgacggcttcaccgccaccggcaacgcccactcgatgctggccaaccggatctcctacgtcctggacgtgcacgggccgagcgaacccgtcgacacggcctgctccagctcgctcgtcgcgctgcaccgcgccgtcgagcacatccggtcgggccgatgcgagatggccatcgcgggcggtgtcaacctgctgctgagcgtggacaccttcgccgcgacgcacatggcgggcatgctcagccccgacggccgctgcaagaccttctccgccggcgcggacggctacgtccgctccgagggcgtcgccgcggtgctgctcaagccgctcgcccaggcgcagcgggacggcgacgccatctggggcgtcgtccggggcagcgccgagaaccacggcggccgcgccggttcgctgaccgcccccaacggcaaggcgcaggccgccctgatccaggacgccatgcgcggcatcgacccggacagcatcggctacgtcgaggcgcacggcacgggcaccggcctgggcgacccggtcgaggtcaacgccctcgacagcgcctaccgcgccctgcgcaccgccgagggcgggccgccgcacgcggcccggccgtgcgcgctcggctcggtgaagaccaacatcggccacgcggagtcggccgcgggcctggccggagtgctgaaggtgctgctcgccatgcgtcaccgcgagctgccgccggccttgcactgcgaccggctcaacccgcacctgccgctcgacggcggattcgaggtcgtacgcgaactgcgccgctgggaaccgtgcaccgacgccaccgggcggccgtggcccctgcgggccggagtgagcagcttcggcttcggcggcgccaacgcccatgtcgtcctcgaagcaccgcccgtaccgcccgcaccggcggagccggcccgcccgaccgccccccaggccatcgtgctgtccgcccgcgacgacgaccggctgcgtgccacggccggacgactgcgggacttcctcgaccgggcgcgccgcgacggacacgccccggacctggcggacctggcgttcaccctccaggtaggccgggaggccatggaacggcgcctgggcttcgtcgtcggaagcatggacgacgtgctcggtacgctggaccggttcttcgcgggcgacgagccctccggctggcacaccggcggcatcaggcggtcgcgtggcgccggagtgcggcgcgaggcggagcaggcccccgaggtgacccgggccctccacgacggacggctcgaccgggtgacggccctgtggtgcgacggcgccccggtcgactggcaggcgatgcatcccacaggcgagcgccgcgccgtgcggctgcccgcgtaccccttcgcctgcgaccgctactgggtgcccgcggtcggcacagcccccgtcccgccgcccgcggcacccgtcccgccccccgcggccgagcccgcgttcgagaccgatgcccgtgcggcgctgctcgacgcggtcctcgacggccgtgccggcccggacgccctgagcaggacctga

SEQ ID NO: 12 - protein, 8026 aa, Streptomyces platensis subsp. rosaceus
VTWNGMNVSRNILRVPEWRDEPARGRTAPPGNRRLVVLCDTPDADVTDLRRHLPGVSVARVDSGDDGPAAAYEHAATLLLGELQRLLNQPAGGPRSVQVVCREGTPYGYAGLIGMLRTAAQEDPALHGQLIECTQRPSGEELAGVLRAEYGQAADHVRYTGGRRQVRAWAAAPRAAAPPPVWKADGVYLISGGAGGVGRLVAADIARHAPGARVVLCGRSPAVPGPGQPGPGTEYRRVDVADADAVAELVNSLVRTYGRLDGVVHAAGLISDDYVIRKSHQDAQQVLAPKAAGLVNLDEATRRLPLDFLAAFSSGAGTLGNPGQADYAAANGFLDAYLTHRAGLAAAGERHGASVSIGWPLWQDGGMSVPAEDVPALTARFGRPLGTDTALRALHGALALGTPHLLVMDEESGVDEESGVDEEGPQEAETQQTGPAELRAHVLPLLKELIAETVRLDPARLDAAAPLDGFGIDSLAVTRLNRRFAQWFGALPKTVLYQYPTLNDLAGHLAEQHADGCRRWLGDVPDVAAAPAGTPATAAAPRKARPRPADADEPIALIGLSGRYPDAPTLEAFWENLRAGRESVREVPAERWPLDAFYEPDPQRAVQQGASYSKWGAFLDDFARFDAAFFGIAPRDAADMDPQERLFVESAWSVLEDAGYTRQRLAEQHASSVGVFAGITKTGFDRHRPPATDGLPPAPRTSFGSLANRVSYLLDLHGPSMPIDTMCSSSLTAIHEACEHLRHGACELAIAGGVNLYLHPSSYVELCRSRMLATDGHCRSFGAGGDGFLPGEGVGAVLLKPLSAAEADGDPIHAVIVGSAINHGGRTNGYTVPNPRAQAALIRDALDRAGVSAAGIGYIEAHGTGTRLGDPVEIDGLTQAFAPDAGGSGACALGSVKSNIGHLEAAAGIAGLTKAVLQLQHGEFAPTLHAEQTNPDIDFAATPFTLQTGGAPWPRPADGGPRRAGISSFGAGGANAHVIVAEYRSATPAPATPAPSARPVLLPLSARTTEDLHARAGQLSDLLRNGAPVDLPAVAATLQTGREEMAERVCFVASTPGEWLDQLGAFLADSDSDSDSDSDSDSDSDSDSDSGSGSEAEAEVPWSRGRVRATRETLAALAEKDELRALVTRWINRGDWHDLAAFWAKGMPLDWTRLHAGADTPARVHLPAYPFAGRQFWFGPAGSEHPATTPVAAPSCSTAAGAADVERILLDALAAALQMPVAEIERRRPFADYGLDSILGVNLVHTLNTALGTALETTDLFDHGTVERLHAFLVGTYGDALHAPASPAAVAPAPDDDAIAVVGMAARYADAEDPRALWDHLMAGHDLVEPVTRWPLGQDVSCRSGSFVRGIDQFDPVFFAISGVEATTMDPQQRIFLEQCWNALEDAGYTGERLTNRNCGVYAGCYAGDYHDQLDARPPAQALWGTMGSVVASRIAYHLDLKGPALTTDTSCSSSLVSLHLACRDLLSGDADMAIAGGVFIQTTSRLYESASRAGMLSPSGRCHSFDARADGFVPGEGAGAVVLKRLADARRDGDHIYGVVRGSGINQDGTTNGITAPSAASQEQLLRDVHARSGIEPGGIQLVEAHGTGTQLGDPIEFRALTRAFEDAPAGSAVLGSIKTNIGHTQFAAGIAGVIKALLALEHRQIPPSLHFQEANRAVVLDGGPFTVTTAPQPWTAPARGPRRAAVSSFGASGTNAHVVLEEHPVPRTTGAGGEHAFLLSARTPAALRAVAERLLAHLDREPGLPADAVAFSLAAGRSHFAHRLAVVAAGLPDLAARLRSWLSGTAGDTVLQGETAADPRPVGGVRAPAPAALAAAYVRGEADRFADSFASASRRQVPLPTYPFERQRYWTDTTDTGESQGLKDTDGAAYRLRLGGEEFFLADHHVGGRAVLPGVLSLEFARRAVTGGSFAPVGLRDVVWPEPFPVGDGGAELRVDRDGDAFRVLRDGSAVHAQGRIATPGSPVPTPLDALRARCGRRTLSRSQCRAALDAVGIRHGDRLRAIDTLAVGDGEVLARLVLPDGARDGAFALHPAMLDSAVQAVVGLYGDATGTLDEQRGAPALPFALDAADFFAPTTERMWAHLRHTEGYTPSADRDVTKVDIDVYDDDGQLSASLRGYAFRRMTAPSGAAPRATLLAPVWDALPVVPAEPWPHPRTRVVLLGGTPEERDGLRRRYPDATVLDPHADEPVDRLAARLPADAEHVFWLAPAGPTGAPAAARYDGTIAVFRLVKALLADGADARELGLTLVTRQARLLPGDTGADPAHAGVHGLAGTLAKEYPHWRIRVADVEADAAVPWPALLALPTDPRGETLAHRHGEWYRLRLLETDGTGVAAAPREPGGVIVAIGGAGGIGTVWTEHMMRRHGARVVWIGRRPLDAAIAAQQEALAAHGPKPDYVQADATDRDALRRACDEIVRRHGPVRGVLHTAIVLGDQTLARMDEDRFRTTYAAKADIAVNLADAFAGQPLEFVAFFSSMQAFFKAPGQANYAAGCTFADAYAEHLSTRLDCPVKVMNWGYWAGVGVVTADGYRQRMAQLGLGSIEPDEGMAAFDTLLASPYPQLALLKATDTRSIDGLHDDDALTHPVVTTPSLIGALGEDCPDRRAEIAQLREKAGGHAGAMQDALVRITWALLQSLGLFRDGRAATAAEWRALGGIEDRYERWTEHTLAVLADAGLLRREGEDTYVALDTRTGSLDDAWADWDRARQQWLADDAKRPQAVLVDTTLRAMTGILTGRRPATDVMFPNAWLELVEAVYKNNPVADYFNDVLADTLVGYLERRLADDPSARLRILEIGAGTGGTSATVLRRLRPWARHIEKYTYTDISKAFLLYGQREYGEIAPYLDARLFNAEKPLAGQEVDPGAYDVVIATNVLHATRNIRRTLRNAKAAARPNALLLLNELSDNILFSHLTFGLLDGWWLYDDPAPRIPGSPGLAPESWRRVLGEVGFRAAFVAAGGADDLGQQVIVAESDGAIRQPRPDGESAFRGTLPEAGPRAAEPQLPAPTPDPVAADGVRDDELLADLARDHFRTLVADTLQLPVADIRADVPFDRYGIDSILVVQLTEAVRKGLCNVGSTLFFEVRTVDGLVQHFLRTQPDALAALVGLSGARAARTDEQLAPAAGPEPVPVIAAEPPRAEQGMAIAIVGMAGRYPGAPDLDTFWENLLAGRDSITEIPAGRWDHSRYYDARRGVPGRTYSKWGGFLDGIDEFDPLFFGISPKAASTMDPQERLFLQCAHTTLEDAGYSRGALRAAARARVAEDAGDIGVFAGAMYSEYQLYGAEYSVRGEPVVVPGSLASIANRVSYFLDASGPSVTVDTMCASALSAIHLACAALQRGECGVALAGGVNLSVHPGKYLMIGEGQFASSDGRCRSFGEGGDGYVPGEGVGAVLLRPLADAVADGDRILGVIRGTAVNHGGHTHGFTVPNPLAQAAVIRSAWRRAGVDPRDIGCIEAHGTGTSLGDPIEIAGLNAAFAEFTDARNFCAIGSAKSNIGHLESAAGIAGLAKLLLQMRHGTLVPSLHAERVNPDIDFADSPFVLQREAAPWPRTGTRPRLGGLSSFGAGGSNAHVVVEDYVEEHAGKDLAPEAHRGETVVVVLSAFDEERLRESAGRLRDALRKERWSSADLPDIAYTLQVGREAMTARFAVAVSTLPALVDALDACALGSGLPAGAYFNPGGDRGGAVKDFLTDEDFQETAVRWARRGKPAPLAEAWTSGLAVDWARLHTEGPKPRKVALPGYPFARERYWYTDGLPELQEIPATFGNAARQPAAPPPAVEAAPATTSAVPAPPARPANSYELPAGDLTLHPVWEPVRLLRGSPYPSAASRVVAIGLAPDALAELTARRPQTVVLDTAASSAEEVRDELAVLGDFDHVVMRFPTAAAAHGAEAQISTQRAAIRSMFRVLKALALTRDEQRLGLTLLTSGAFDAGGSGTADPAQASLHGLLGGLAKEQPHWRIRAVDLADGEPFVADEVFALPADRRAHPLVRRGGQWLRRQLLPVDATEPPAEPVLRRDGVYVLIGGAGDLGVLLSEYLVRQHDAHVVWVGRRAEDEDIRARADRAAAGGRTPVYLSADASDPDALARMRDEVVRRYGRIDGVVHLAMVFSHTPLARMTERELEATLAAKVDPCAHFADVFAGHGLDFVLLISSLVSFIRNSHQAHYSAACAFEDAHAAALREALDCRVKVVNWGYWGNVPDELLRDVTSMGLAPIAPATAMGALERLLAGPLHQIGFMRLGRPLPVEGVLTAETLTPQTHGAAARDGAAALALPTGLAAYHESPVPGEIDAFLLRRLAAELRRAGLEEPRHGLAEWKERQGVDARFDGWLSATLHALAEHAMIDDRGRWTTSSPAATDADACRADWAAQTPRWAAANPDLRAPLNLLDRTLPALPDVLCGRVRATDVLFPQGKFSLVEGVYRDNRVAAHFNAVLAEHVAAFLRARRDADPGARLRVLEIGAGTGGTTGPVLDRLAHEGLDLAEYCFTDLSQAFLQNAQDTFGPGRDHLTYRIFDAARPPHTQGLDTGAFDVVIAANVLHATDTIRPALRHAKALLRGNGLLALNEISGFYLVNHLTFGLLDGWWLYDDAELRVPGSPALSPAAWQLVLEQEGFTGIRHPARDALALGQQVVVAHSDGLARSPRLLSGTPEMSSPPSQPPAETAAPAAASASARAVTDVVLAALADALRMPADRIGPDRAFADYGLDSIVGVRFVQRLNEELGTDLPTTVVFDYRSVAQLAAHIAESHRPQPAPAAAAPVPAPDAAGAPNRPEGREPIAIVGISGRFAQSDDTDALWQHLAAGRDLVGPVERWDLSGYSQDQLSCRAGSFLDGIDRFDARFFHLTGLEATYTDPQQRLFLEQAWTAIEDAGYAGSALDGRRCGVYAGCTGGDYPQWFEDAPPAQAAWGNAPSVVPARIAYHLNLQGPALAVDTACSSSLVAVHLACQGLWSGETDMALAGGVSVQTTPDTYLAAGRGGMLSPTGKCHTFDAAADGFVPGEGVGVVVLRRLSDALADGDHIHAVIRGSAVNQDGATNGITAPSALSQERLIRQVHTEFGIDPAEIGMVEAHGTGTQLGDPIECQALVGAFGTAGGSDTCALGSIKTNLGHTTSAAGVAGLLKVVLSLRHGQIPPSLHHYETNPAIRLTESPFHVNTTLRPWQPNGQGKRVAALSAFGFSGTNGHMVVENAPDRDERQQAADELLFVLSAQQPEALRHRAEDLLAYLRRAPDAALGDVSYTLAAGRDHFTHRAAFVAADRDTLAHRLEAWLADGRSDTVGRRGDTAPERARARYLNGEEVDFAPLFSGLDVRRTPLPTYPFQRKSYWPTATAPSRRHQAPQAANGPAAAPSPEPARPAPAQPAPDTDEATVRYLAGELLLAELSRVLMMEPEEIDPQASFSDYGVDSILTVRLVAAVNNALAVDLPSTALFEHSSLDRLTDHLVTRYGAQLRSSGALRGPAAEAGGAPAQDDHGPAAEAPSAAPAAPVASAGTAAVPAHAPAAAAGDPADDGVAVVGIAARFAQSPDAAALWAHLAAGDDLVGEVTRWDMDEELGAGAPRQYGSFVDDIERFDAWFFRMSGKEATYTDPQQRIFLEECWHALEDAGYAGERLDGRGCGVYVGGSPSDYQQLIGDDAPPQTLWGNISSVIASRISYFLDLQGAALAVDTACSSSLVAIHQACQDLRLGNTSMALAGGVFVQSTPIFYRSAVRANMLSARGRCHTFDERADGFVPGEGAGVVVLKRLADALRDGDQVYGVIRGSGMNQDGTTNGLTAPSAGSQERLLRSVHERAGVDPAGIQLIEAHGTGTPLGDPIEFEALRAAFGDAPEAGCALGSVKTSLGHTQFAAGVAGVIKVLLALRNEQLPPSLHFRRANPAITLEGSPFYVNTELRPWPAPADGPRRAGVSSFGAAGTNAHALIEQAPAVRTAGHGPRHAWLIVLSAQDDAGRRAQAERLLDHALAHEDLDLGDVAYTLATGRRHCSHRWAGVATDREQLVAALRTWLCDGRAEGVVTGEAPDGHRRQDPAEDARAGRLMAEPDRHDSLTELAGLFAQGQDLGFAPLFGDGGFRIVSLPAYPFAGERYWVGSRPAAPAATPASAPVRAPVPVAAPSPLEGRRLTGDPGSPSFAVELAGREFFLDDHRVRNVPVLPGVAYLELAYAAARAEGVDPAHARLRNVVWSRPARITGPTAVEIALRPCEDDAFTYEITTAADGEQPVIHGQGRIERCGTPSPARLDIAALRAQCEVRTLEHDDCYRLFDRMGIGYGPAMRGIRRIHVGAGLAVARLSLPQAARDGAGWDLHPSMLDAAVQATLGLSLAEDTDTVAPALPFVLEEVQLLAPSPAGGWAVVRPAAGDGGGAVRRIDIELCDDDGEVCVRLLGFTARVLAAGDDPAGGENTGGATLTLMRAGWRPAEPTRASRPLVHHEVLLGGLAGTDPAAVRDGLGVPCTALPDDGDPARCFTRQAETVLARLQQFVPRTRDGEVLLQVVVPADGENRVLAGLGGLLRTARMEHPKLLTQLVEVETPVDAATLCERLRRDAASPDDVAVRYSGGQRRVPQWTAVEDAPPARPWKAGGVYLLTGGVGGLGAHFAREIARQAPGAALVLCGRSPEGPAQRELLCELGDLGASAVYRVLDVARRDAVTACVNTVVAEHGRLDGVVHTAGVVRDGYLARKSAEELREVLAAKVAGFVHLDGATAALDLDCFIGFSSLSAYGNQGQGDYAAANAFMDAYAGLRHERVARGERRGRTLVVGWPLWADGGMTVDAATERRLHDSVGMVPIRAPHGVEALLRAYGTGDPHVLAVFGDRARIDATLLAAPAATGAAPAVTAPDRAALHARVLGRAISHACAVLGVPAAELDGAVELSEYGFDPVSLTGFAARLTTEFGLPPVPKPFSEHLTLGEVVDHLLDTHPHHFGTVPPAPAPEPSAGPESAAAPVATAGREQQHKALLKKLIARVSDLLDVPAERITGTAEMTRYGFDSLSFIGFANDLNAEFGLSLAPTLFFENPTLDGVVDHLLDHHADRVAATAAPQQEPRAAAAPAAPEPATADTPASRTDAPGNEPIAVIGISGRFPMADDLDAFWENLSEGRDCTREVPTDRWDWRAHYGDPVKEPNTSNVTSGGFMDGVGDFDPLFFDISPKEAELMDPQQRLLLMYVWKALEDAGYSAEALAGTNTALIAGTTSTGYSTLVTRYSPMIEGYDITGAAPSMGPNRMSYFLDLHGPSEPVDTACSSALVALHRAVQAIRDGQSDLAIAGGVNTMVSVDGHISISKAGMLSPEGRCKTFSDRADGYARGEGVGMLVLKSLSAAERDGDHIYGVIRSTAENHGGRGSSLTAPNPKAQAALLREAYGKAGIDPRTVGYIEAHGTGTKLGDPVEINGLKAAFRDMYEEHGAVVEEAHCGIGSVKTNIGHLELAAGAAGVIKVLLQMRHRTLVKSLHCDTVNPYIDLDGSPFHLVRERQPWPALRDAEGRELPRRAGVSSFGFGGVNAHVVLEEYRPRTAPEPDRAPTAPVPVVLSASHPDVLCELAERWVDALRRGDYDDTDMASIAYTTQTGRTPMTERLACLARTAGELREALESWLRGEPAADVFRGKVARGVDLPDAPAGFGPHDDHDSAGRHDWARLLQAWVNGAPFDWDRLHTGRRPRRIALPTYPFRLRRYWVDTSRPANGTQTEALHPLVHTNTSDLNEHRYTSHFTGREFFLADHRVRAQVMETVSGWRPGRRPTAYDVRADAVPVLPAVAYLEMARAAAVQAAGGDERAWSLKLASWLRPLTVEKATDVHTTLTTRAGGGLSYEVYAVDEDGERVTFGRGQLRRATAVPAERLDLAALRAQCDGPVLDAETCYARFTGIGMAYGPALRGIERLHTGSRQSVARLKLPAAASRERGWVLNPGMLDAALQATVGLFVDDPGTPRTALPFALGELEVLRAVPGTGWVVVRFAEDDHVGAVRRLDLDLCDDDGEVCVRLRGFSVRTLGGSEPTGDSEPTRPAEQAPEPPSGSDDAYLLDLIEAIGRREMSADEFKRSLA

SEQ ID NO: 13 - DNA, 24081 bp, Streptomyces platensis subsp. rosaceus
gtgacctggaacggaatgaacgtgagcagaaacatccttcgtgtgccggaatggcgcgacgaaccggcgcgagggcgcaccgcgccccccggaaaccggcggctggtcgtgctgtgcgacacccccgacgcggacgtgaccgacctgcgccggcacctgcccggcgtgtccgtcgcccgcgtggacagcggtgacgacgggcccgctgccgcctacgagcacgcggcgaccctgctgctcggcgagctccagcggctgctgaaccagccggccggcggcccccgttccgtgcaggtcgtgtgccgggaggggactccgtacggctacgccggtctgatcggcatgctgcgcaccgccgcgcaggaggacccggcgctgcacggccagctgatcgagtgcacgcagcggccgtcgggcgaggaactcgccggcgtgctgcgggcggagtacgggcaggcggcggatcacgtgcgctacaccggcggccgccgccaggtccgcgcctgggcagcggccccgcgtgcggcggcacccccgccggtgtggaaggccgacggcgtctacctgatcagcgggggagcgggcggcgtcggccggctggtcgccgccgacatcgcacggcacgcccccggcgcccgggtcgtcctgtgcggacgctcgccggcggtccccgggcccggtcagccgggcccggggaccgagtaccgccgggtggacgtcgccgacgccgacgccgtggcggagctcgtcaactcccttgtgcgcacgtacggcaggctcgacggggtcgtgcacgcggcgggcctgatcagcgacgactacgtgatccgcaagtcccaccaggacgcccagcaggtcctggcgccgaaagccgctgggctggtgaacctcgacgaggccacccgccgcctgccgttggacttcctcgcggcgttctcctccggcgcggggacgctgggcaaccccgggcaggccgactacgccgccgcgaacgggttcctcgacgcctacctgacccaccgcgccggcctggccgccgcgggcgagcgccacggcgcgagcgtctcgatcggctggccgctgtggcaggacggcgggatgagcgtcccggccgaggacgtgcccgcgctcaccgcccgcttcgggcgccccctgggaacggacacggcactgcgggccctgcacggcgcactggcgctcggcacaccacacctactggtcatggacgaggagagcggagtggacgaagagagcggagtggacgaggaaggtccgcaggaggcggagacgcagcagacggggccggcggaactgcgggcacatgtgctgcccctgctgaaggagttgatcgccgagacggtgcggctcgaccccgcccggctggacgccgccgctccgctcgacggcttcggcatcgactcgctggccgtgacccggctcaaccgccggttcgcgcagtggttcggcgcgctgcccaagacggtgctctaccagtacccgacgctgaacgacctggccgggcacctggcggagcagcacgcggacggctgccgccgctggctcggcgacgtcccggacgtggccgccgccccggccgggactccggcgacggcggccgcgccgcggaaggcgcggccccgtccggccgacgcggacgagccgatcgccctcatcggcctgagcgggcgctatccggacgccccgaccctggaggcgttctgggagaacctgcgcgcgggccgcgagagcgtccgcgaggtccccgccgagcgctggccgctggacgccttctacgaaccggacccgcagcgggccgtgcagcagggcgccagctacagcaagtggggcgcgttcctcgacgacttcgcccgcttcgacgccgcgttcttcgggatcgcgccgcgcgacgccgccgacatggacccgcaggaacggctgttcgtcgagagcgcgtggtcggtgctggaggacgcgggctatacgcggcagcgcctcgccgagcagcacgcatcgtcggtcggcgtcttcgccgggatcaccaagaccggcttcgaccgccaccgcccgccggcgaccgacggactgccgcccgcgccgcgcacgtccttcggatcgctggccaaccgggtgtcgtacctgctggacctgcacggcccgagcatgccgatcgacaccatgtgctcgtcgtcgctgaccgcgattcacgaggcatgcgagcacctgcgccacggcgcgtgcgagctggccatcgccggcggtgtcaacctctacctgcacccctcctcgtacgtcgagttgtgccgttcccggatgctcgccaccgacgggcactgccgcagcttcggcgcgggcggcgacgggttcctgcccggcgagggcgtcggcgcggtgctgttgaagccgctgtccgcggccgaggccgacggcgaccccatccacgcggtgatcgtcggctccgcgatcaaccatggtgggcgcaccaacggttacaccgtgcccaacccgcgcgcacaggccgcgctgatccgcgacgcgctggaccgcgccggtgtgtccgcggccggcatcggctacatcgaggcgcacggcaccggcacccggctcggcgaccccgtcgagatcgacggcctgacccaggccttcgctcctgacgccggcgggagcggtgcgtgcgccctcggctcggtcaagtcgaacatcgggcacctggaggccgctgcgggtatcgcgggcctgaccaaggccgtactgcaactgcagcacggcgagttcgcgcccaccctgcatgccgagcagaccaacccggacatcgacttcgcggccaccccgttcaccctgcagaccggcggggccccttggccgcggcccgcggacggcggcccgcggagggcaggcatctcctcgttcggcgcgggcggcgccaacgcccatgtcatcgtcgccgagtaccggagcgcgacgcccgcacccgccacgcccgccccgtccgcgcggccggtgctgctgccgctgtccgcccggaccaccgaggacctgcacgcacgggccggccaactgtccgacctgctccgcaacggcgcccccgtggacctgcccgccgtcgcggccaccctccagaccggccgcgaggagatggcggagcgggtgtgcttcgtcgcgagcacacccggggaatggctcgaccagctcggcgccttcctcgccgactccgactccgactccgactccgactccgactccgactccgactccgactccgactccgactccggctccggctccgaggccgaggccgaggtcccgtggtcccgcggccgggtcagggccacccgcgagaccctggcagccctggcggagaaggacgaactgcgcgcactcgtcacccgctggatcaaccgcggcgactggcacgacctggccgccttctgggccaagggcatgccgctcgactggacccgcctgcacgccggtgcggacacgcccgcacgggtccacctgcccgcctaccccttcgccggacggcagttctggttcggcccggccggcagcgagcacccggcaacgacgccggtggccgccccgtcctgctcgacggcagccggtgccgccgacgtcgagcgcatcctgctcgacgcactggcagcggccctgcagatgccggtcgccgagatcgagcgccgccgccccttcgccgactacggcctggactccatcctcggcgtgaacctggtccacacgctcaacacggccctcggcaccgcgctggagaccaccgatctgttcgaccacggcaccgtcgagcgcctgcacgcgttcctcgtcggtacctacggtgacgcactgcacgcaccggcctccccggcagccgtcgccccggcgccagacgacgacgccatcgccgtcgtcgggatggccgcccgctacgccgacgccgaggacccgcgcgcgctctgggaccacttgatggccggccacgacctcgtcgaaccggtgacccgctggccgctcggccaggacgtgagctgccgctccggcagcttcgtccgcggcatcgaccagttcgacccggtgttcttcgcgatctccggtgtcgaggccaccaccatggaccctcagcagcgcatcttcctcgaacagtgctggaacgccctggaggacgccggctacaccggcgaacgcctgaccaaccgcaactgtggcgtctacgccggctgctacgccggcgactaccacgaccagctggacgcccggccgccggcgcaggcgctgtggggcaccatgggctcggtcgtcgcctcccggatcgcctaccacctcgacctcaagggccctgccctcaccaccgacacctcctgctccagctcactcgtctccctgcacctggcctgccgcgacctgctctccggggacgccgacatggcgatcgcgggcggggtgttcatccagaccacgtcgcggctgtacgagtcggcgtcgcgcgcgggcatgctctcgcccagcggccgctgccacagcttcgacgcccgcgccgacggcttcgtcccgggcgagggcgcgggcgcagtcgtcctcaaacggctcgccgacgcccggcgcgacggcgaccacatctacggcgtcgtccgcggctccggcatcaaccaggacggcaccaccaacggcatcaccgccccgagcgcggcctcgcaggaacagctcctgcgcgacgtccacgcccgcagcggcatcgagccgggcggcatccagctcgtcgaggcgcacggcacgggtacccagctcggcgacccgatcgaattccgcgcgctcacccgcgcgttcgaggacgccccggccgggagcgccgtgctgggatcgatcaagaccaacatcgggcacacgcagttcgccgccggcatcgcgggcgtcatcaaggcgctgctggccctggagcaccggcagatcccgccgtcgctccacttccaagaggccaaccgggccgtcgtgctcgacggcggcccgttcaccgtcaccaccgccccgcagccctggacggcgcctgcccgcggcccgcgccgggcggccgtgagttccttcggggccagcggcaccaacgcgcatgtcgtgctggaggagcacccggtcccccggacgaccggcgcgggcggggaacacgcctttctgctgtcggcccgcacaccggccgctctccgtgccgtcgccgaacggctgctcgcccacctcgaccgcgaacccgggctgcccgccgacgccgtcgccttcagcctggccgcgggacgcagccacttcgcgcaccggctggccgtcgtcgccgccggcctgcccgacctggcggcacgcctgcgctcctggctgtccggcaccgccggtgacacggtgctgcagggggagaccgccgcggacccccgccccgtcggcggtgtgcgcgcgccggccccggccgcgctggccgcagcgtacgtacggggcgaggccgaccggttcgccgacagcttcgcgtccgcctcgcgccgccaggtgccgctgccgacctacccgttcgagcggcagcgctactggaccgacacgaccgacaccggggaaagccaggggctcaaggacacggacggggccgcgtaccgcctccggctcggcggcgaggagttcttcctggccgaccaccacgtgggcggccgggccgtgctgcccggcgtgctctcgctggagttcgcacgccgtgccgtgaccggcggttccttcgcgccggtcggcctgcgcgatgtcgtatggccggagccgttccccgtcggggacggcggcgccgaactacgagtcgatcgggacggcgacgccttccgcgtcctgcgcgacggctcggccgtacacgcccagggccggatcgccacgcccggctcgcccgtccccacgccgttggacgccctgcgggcccgctgcggccgccgcaccctgtcgcggagccagtgccgtgcggccctcgacgccgtcggcatccgccacggagaccgcctgcgcgccatcgacaccctggccgtcggtgacggcgaggtcctggcccggctcgtcctgcccgacggcgcccgcgacggcgcgttcgcgctgcaccccgcgatgctcgacagcgccgtgcaggccgtcgtcggcctctacggcgacgccaccggcacgctcgacgagcaacgcggcgcgcccgcactgcccttcgccctggacgccgccgacttcttcgcccccaccaccgaacgcatgtgggcccacctgcgccacaccgagggctacaccccctcggccgaccgggacgtgacgaaagtggacatcgacgtgtacgacgacgacggacagctctccgcgagcctgcgcggctacgcgttccgccgcatgaccgccccgtccggcgcggccccgcgtgccacgctgctggcaccggtgtgggacgccctgcccgtcgtgcccgccgagccgtggccccacccgcggacccgcgtcgtgctgctgggcggcacccccgaggaacgggacgggctccgccgccgctaccccgacgccaccgtcctggacccccacgccgacgaaccggtcgaccggctggccgcgcggctgcccgccgacgccgagcacgtcttctggctcgccccggccggccccaccggcgccccggccgccgcgcggtacgacggcacgatcgccgtattccgactggtcaaggcgctcctggccgacggcgcggacgcccgtgaactgggcctgaccctggtcacccggcaggcgcgcctgctaccgggcgacaccggtgccgaccccgcccacgccggtgtgcacggcctcgccggcaccctggccaaggagtacccgcactggcggatccgcgtcgccgacgtcgaggcggacgccgccgtgccctggccggctctgctggctctgcccaccgacccccgcggcgagaccctggcccaccggcacggcgagtggtaccgcctgcgcctgctggagacggacgggaccggcgtcgcggccgccccgcgcgagcccggcggcgtgatcgtggccatcggcggcgccggcggcatcggcaccgtgtggaccgagcacatgatgcgccgtcacggcgcccgggtcgtctggatcggacgccgcccgctggacgccgccatcgccgctcagcaggaagccctggcagcccacggccccaagccggactacgtgcaggccgacgcgaccgaccgcgacgccctgcgccgcgcctgcgacgagatcgtgcggcggcacggccccgtgcgcggcgtcctgcacaccgcgatcgtcctcggcgaccagaccctcgcccggatggacgaggaccggttccgcacgacctacgccgccaaggccgacatcgccgtgaacctcgccgacgccttcgccggccagccgctggaattcgtcgcgttcttctcctccatgcaggccttcttcaaggcccccggccaggccaactacgcggcgggctgcaccttcgccgacgcctacgccgagcacctgtccacccggctcgactgcccggtcaaggtcatgaactggggttactgggccggcgtcggcgtcgtcaccgccgacggctaccggcagcggatggcacagctgggcctgggctcgatcgaaccggacgagggcatggccgccttcgacaccctgctggcctccccgtacccgcagctcgcactcctcaaggccacggacacccgcagcatcgacggcctccacgacgacgacgccctcacgcacccggtcgtcaccaccccctccctgatcggcgccctgggcgaggactgccccgaccgccgcgccgagatcgcgcagctgcgtgagaaggcgggcgggcacgccggagccatgcaggacgcgctcgtccgcatcacctgggcgctgctgcagtccctgggcctgttccgcgacggccgcgcggccaccgccgccgagtggcgcgccctcggcggcatcgaggaccgctacgagcgctggaccgagcacaccctcgccgtactcgccgacgcaggcctcctgcgccgcgagggcgaggacacgtacgtggccctcgacacccgtaccggatccctcgacgacgcctgggccgactgggaccgggcgcggcagcagtggctggccgacgacgccaagcgtccccaggcggtcctcgtcgacacgacgctgcgcgccatgaccggcatcctcaccggccgccgcccggccaccgacgtgatgttcccgaacgcctggctcgaactcgtcgaggccgtgtacaagaacaaccccgtcgccgactacttcaacgacgtgctcgccgacaccctcgtcggctacctcgaacggcggctggcggacgacccgtccgcccgcctgcgcatcctggagatcggcgccggcaccggcggtaccagcgccacggtcctgcgcaggctgcggccgtgggcccggcacatcgagaagtacacctacaccgacatctccaaggcgttcttgctgtacgggcagcgggagtacggcgagatcgccccgtacctggacgcacggctcttcaatgccgagaagccgctggcaggccaggaggtggaccccggcgcgtacgacgtcgtgatcgccaccaacgtgctgcacgcgacccgcaacatccgcaggacgctgcgcaacgccaaggccgccgcgcgcccgaacgccctgctgctgctcaacgagctcagcgacaacatcctcttcagccacctcacgttcggcctcctggacggctggtggctctacgacgacccggcgccgcgtatccccggttctccgggcctggcgccggagagctggcggcgggtcctcggcgaggtcggcttccgcgcggcgttcgtcgccgccgggggcgccgacgacctcggccagcaggtgatcgtcgccgagagcgacggcgcgatccgccagccgcgcccggacggggagtccgctttccgcggcaccctcccggaggccgggccgcgggccgccgagcctcaactgcccgccccgacaccggatccggtcgccgccgacggcgtacgtgacgacgagctcctggcggacctggcccgcgaccacttccgcaccctggtcgcggacaccttgcaactgccggtcgccgacatccgcgccgatgtgcccttcgaccgctacggcatcgactcgatcctggtcgtccagctgacggaagcggtccgcaaggggctctgcaacgtcggcagcacgctgttcttcgaagtacggacggtcgacgggctcgtccagcacttcctgcgcacccagcccgacgcgctcgcggcactggtcggcctgagcggcgcgcgggcagcgcgcacggacgagcagctcgcgccggccgccgggccggagccggtccccgtcatcgccgccgaaccgccccgcgccgagcagggcatggccatcgcgatcgtcggcatggcaggccgctaccccggcgcacccgacctggacaccttctgggagaacctgctcgccggccgggacagcatcaccgagatcccggccgggcgctgggaccacagccgctactacgacgcgcgtcgcggcgtgcccggcaggacgtacagcaagtggggcggcttcctcgacgggatcgacgagttcgacccgctgttcttcgggatctcgccgaaggcggcgtccacgatggacccgcaggagcggctgttcctgcagtgcgcccacaccacgctggaggacgccggctactcgcgcggcgccctgcgcgccgccgcccgcgcccgggtggcggaggacgccggcgacatcggggtgttcgccggcgcgatgtactccgagtaccagctctacggcgccgagtacagcgtgcgcggtgagccggtcgtggtgccggggagcctggcgtccatcgccaaccgcgtctcgtacttcctggacgcgagcggccccagcgtcaccgtcgacaccatgtgcgcctcggcgctgtccgcgatccacctcgcctgcgccgccctccagcgaggggagtgcggtgtcgccctggccggcggggtcaacctgtcggtgcacccgggcaagtacctgatgatcggggagggccagttcgcctccagcgacggccgctgccgcagcttcggcgagggcggcgacggctacgtgcccggcgagggcgtcggcgcggtgctgctgcgcccgctcgccgacgccgtcgccgacggcgaccgcatcctcggcgtgatccgcggcaccgccgtgaaccacggcggccacacgcacggattcaccgtgccgaacccgctcgcgcaggcggcggtcatccgcagcgcctggcgccgggccggagtggacccccgggacatcggctgcatcgaggcgcacggtaccggcacctcgctgggcgacccgatcgaaatcgccgggctgaacgcggccttcgccgagttcaccgacgcacggaacttctgcgccatcggctcggcgaagtcgaacatcggccacctggagtccgcggcgggtatcgcgggcctcgccaagctgctgctgcagatgcggcacggcacgctcgtgccctccctgcacgccgaacgcgtcaacccggacatcgacttcgccgacagccccttcgtcctgcagcgcgaagccgcgccctggccgaggaccggcacccgcccgcgcctcggcggcctctcctcgttcggcgcgggcggctccaacgcccacgtcgtggtcgaggactacgtcgaggagcacgccgggaaggacctcgcgcccgaggcgcaccgtggcgaaaccgtcgtcgtggtgctgtccgccttcgacgaggagcgcctgcgcgagtcggccgggcggctgcgcgacgcgctgcggaaggagcggtggagcagcgcggacctgcccgacatcgcctacacgctgcaggtcggccgcgaggcgatgaccgcacggttcgccgtggccgtcagcacgcttcccgccctggtcgacgcgctggacgcctgcgcgctcggcagcgggctgcccgcgggcgcgtatttcaaccccggcggcgaccggggcggcgcggtcaaggacttcctcaccgacgaggacttccaggagacggccgtgcgctgggcacggcgcggaaagccggcgccgctggccgaggcctggaccagcggcctggccgtcgactgggcccgcctccacaccgagggaccgaagccgcgcaaggtcgcactgcccggctacccgttcgcccgggagcgctactggtacaccgacggacttccggaactccaggaaatccccgccacgttcgggaacgccgcacggcagcccgccgccccgccccctgccgtggaggccgcgcctgcgacgacgtccgccgtgcccgccccgcccgcgcggccggccaactcctacgagcttcccgcgggcgacctcaccctgcaccccgtctgggagcctgtccggctgctgcgcggcagcccttacccgtccgcggcctcccgtgtggtggcgatcggcctcgcaccggacgcgctcgcggagctgaccgcccgccgcccgcagaccgtggtgctggacaccgccgcgtcatccgccgaagaggtgcgtgacgaactcgccgtcctgggcgacttcgaccacgtcgtcatgcggttcccgaccgcagccgccgcccacggcgccgaggcgcagatcagcacgcagcgcgcggcgatccggagcatgttccgggtcctcaaggcactggccctcacccgggacgagcagcggctcggactcaccctcctgaccagcggcgcgttcgacgcaggcggctcggggaccgccgacccggcgcaggcgagcctgcacggtctgctcggcggcctggccaaggagcagccgcactggcgcatccgcgcggtcgacctggccgacggcgaaccgttcgtcgccgacgaggtcttcgccctgcccgccgaccgccgcgcgcacccgctcgtccgccgcggcggccagtggctgcgccgtcagctcctgccggtggacgccaccgagccgcccgcggagcccgtgctgcgccgcgacggcgtctatgtgctcatcggcggcgcgggcgacctcggcgtgctgctcagcgagtacctcgtacggcaacacgacgcacacgtcgtatgggtcggccgccgcgccgaggacgaggacatccgggccagggcggaccgggccgcagcgggcgggcggacccccgtctacctgtccgccgacgcctccgaccccgacgcgctcgcccgcatgcgggacgaggtcgtccgccgctacggccgcatcgacggcgtggtgcacctggcgatggtgttcagtcacacgccgctcgcccggatgaccgagcgcgaactggaggccaccctcgcggccaaggtcgacccgtgcgcgcacttcgccgacgtcttcgccgggcacggcctggacttcgtcctgctgatctcctcgctggtgagcttcatccgcaactcccaccaggcgcactactcggcggcctgcgccttcgaggacgcgcacgccgccgccctgcgcgaggcgctggactgccgggtcaaggtcgtcaactggggctactggggcaacgtccccgacgagctcctgcgcgacgtgacgtccatgggactggccccgatcgccccggccacggcgatgggcgcactggagcgcctcctggccggcccgctccaccagatcggcttcatgcgcctcggccgcccgctgcccgtcgaaggggtgctcaccgcggagacgctgaccccgcagacgcacggtgcggcggcccgcgacggcgccgcggccctcgctctgcccaccggcctggccgcgtaccacgagagcccggtcccgggcgagatcgacgcgttcctgctccgccgcctcgccgccgagctgcgccgagcgggtctggaggagccgcgccacggcctggccgagtggaaggagcggcagggcgtcgacgcacggttcgacggctggctctcggccaccctgcacgcgctcgccgagcacgcgatgatcgacgaccggggccgctggaccaccagcagtccggccgccacggacgccgacgcctgccgcgccgactgggccgcgcagacaccccggtgggccgccgccaaccccgatctgcgcgcaccgctgaacctgctggaccggaccctgcccgcgctccccgacgtcctgtgcggccgggtgcgcgccaccgacgtgctcttcccccaggggaagttctccctggtcgaaggcgtctaccgcgacaaccgcgtggccgcgcacttcaacgccgtcctcgccgaacacgtggcggccttcctgcgcgcacgccgggacgccgatcccggcgcccgcctgcgcgtgctggagatcggcgcaggcaccggcggtaccaccggccccgtgctcgaccgcctcgcccacgaagggctggacctggccgagtactgcttcaccgacctgtcccaggcattcctgcagaacgcccaggacaccttcgggccgggccgcgaccacctcacctaccgcatcttcgacgcggccaggcccccgcacacccaagggctcgacaccggcgccttcgacgtcgtcatcgcggccaacgtgctgcacgccaccgacaccatccgcccggccctgcggcacgccaaggcgctcctgcgcggcaacggcctgctggctctcaacgagatcagcggcttctacctcgtcaaccacctcaccttcggcctgctcgacggctggtggctctacgacgacgccgaactgcgcgtgcccggcagccccgcgctgtcgccggcggcctggcagctcgtactggaacaggaaggcttcaccggcatccgccatccggcgcgggacgccctggcactcgggcagcaggtcgtcgtggcccacagcgacggtctcgcccgcagcccgcgcctgctctccggaacgcccgagatgagcagcccgccctcccagccgccggcggaaaccgcggctccggccgccgcctccgcttcggcccgggccgtcacggacgtggtgctggccgcgctcgccgacgcgctgcgcatgcccgccgaccggatcggcccggaccgggcgttcgccgactacggcctcgactccatcgtcggcgtccggttcgtccagcgcctcaacgaggagctgggcaccgacctgccgaccacggtcgtcttcgactaccgcagcgtggcgcagctcgccgcccacatcgccgagagccaccggccgcaaccggcccccgccgcggcggcaccggtgcccgcaccggacgccgccggggcaccgaaccgtcccgaaggacgcgagcccatcgccatcgtcgggatcagcggccgcttcgcgcagtcggacgacaccgacgccctctggcagcacctcgccgccggccgcgacctcgtgggcccggtcgaacggtgggacctctccggctacagccaggaccaactgtcctgccgcgcgggcagcttcctcgacggcatcgaccggttcgacgcacgcttcttccacctgaccggcctcgaagccacctacaccgacccccagcagcggctgttcctggaacaggcgtggacggccatcgaggatgccggctacgcgggctccgcgctggacggccgccggtgcggcgtctacgccggctgcaccggcggcgactacccccagtggttcgaggacgcgccgcccgcccaggcggcatggggcaacgcgccctcggtcgtaccggcgcgcatcgcctaccacctgaacctccagggtcccgccctcgcggtcgacacggcctgctccagctcactggtcgccgtccacctcgcctgccagggcctgtggagcggcgaaaccgacatggccctcgcaggaggcgtcagcgtccagaccaccccggacacctacctggcggccggccgcggcgggatgctctcgcccaccggcaagtgccacaccttcgacgccgccgccgacggattcgtccccggcgagggcgtgggcgtcgtggtgctccgccgcctgtccgacgcactggccgacggcgaccacatccacgccgtgatccgcggctccgccgtcaaccaggacggggcgaccaacggcatcaccgcacccagcgccctgtcgcaggaacgcctcatccgccaggtgcacaccgaattcggcatcgacccggccgagatcggcatggtcgaggcgcacggcaccggcacccagctcggcgaccccatcgaatgccaggccctggtcggcgcgttcggcacggccggcggcagcgacacctgcgcactcggctcgatcaagacgaacctcggtcacaccacctccgccgcgggcgtggccggcctgctcaaggtcgtgctctcgctgcgccacggtcagatcccgccgtccctccaccactacgagaccaaccccgcgatccgactcaccgaaagtcccttccacgtgaacaccacgctgcggccgtggcagcccaacggccagggcaagcgcgtcgccgccctgagcgcgttcggcttcagcggcaccaacggccatatggtcgtggagaacgccccggaccgtgacgagcgccaacaggccgccgacgagctgctgttcgtcctgtccgcccagcagcccgaggcgctgcgccaccgcgccgaggacctcttggcgtacctgcgccgcgcacccgacgccgcgctgggcgacgtcagctacacgctggcggcaggccgggaccacttcacccaccgcgcggcctttgtcgccgccgaccgcgacacgctcgcccaccggctggaggcctggctggccgacggacggagcgacaccgtcggccggcgcggcgacaccgcgccggagcgcgcccgggcccggtacctgaacggcgaggaggtcgacttcgcgccgctgttctccggcctcgacgtccgtcgcacgccgctgcccacctacccgttccagcgcaagagctactggccgacggccactgctccgagccggcgccaccaagccccgcaggccgcgaacggccctgccgccgccccgtcgcccgagcccgcccggccggcacccgcgcagccggcaccggacacggacgaggcgaccgtgcggtacctggccggcgaactcctgctggccgagctctcccgcgtgctcatgatggagcccgaggagatcgacccgcaggcgtccttctccgactacggcgtggactcgatcctcaccgtcaggctcgtcgcagcggtgaacaacgccctcgccgtcgacctgccgagcaccgcactgttcgaacacagctcgctcgaccggctgacggaccacctggtcacccggtacggggcgcagttgcggtcctccggtgcgctgcgcgggccggcagccgaggccggaggggctccggcgcaggacgaccacgggcccgccgccgaggctccgtccgctgctcctgctgctcctgtcgcctccgccggaactgccgccgtccccgctcacgcccccgcggccgctgcgggcgacccggccgacgacggcgtcgccgtggtgggcatcgccgcccggttcgcgcagtcgcccgacgccgccgccctgtgggcacacctcgccgcgggcgacgacctggtcggcgaggtcacccgctgggacatggacgaggagctgggcgcgggcgccccgcgccagtacggaagcttcgtcgacgacatcgagcgcttcgacgcctggttcttccggatgtccggtaaggaggccacctacaccgacccgcaacagcgcatcttcctggaggagtgctggcacgccctggaggatgcgggctacgccggtgaacggctcgacggccgcgggtgcggcgtctacgtcggcggctcacccagcgactaccagcagttgatcggcgacgacgcgccaccgcagacactgtggggcaacatctcctcggtcatcgcgtcgcggatctcctacttcctcgacctgcagggtgccgcgctggcggtcgacacggcctgctccagttcgctggtggccattcaccaggcctgccaggacctccgcctgggcaacacgtccatggcgctggcgggcggagtcttcgtccagtccacgccgatcttctaccggtccgccgtgcgggcgaacatgctgtccgcccgcgggcgctgccacaccttcgacgagcgcgccgacggcttcgtgccgggggagggcgccggcgtggtcgtgctcaagaggctcgccgacgcgctgcgcgacggcgaccaggtctacggcgtgatccgcggctccggcatgaaccaggacggcaccaccaacgggctcaccgcgcccagcgccggatcgcaggaacgcctcctgcgcagcgtccacgagcgcgccggcgtcgaccccgccggcatccagctgatcgaggcgcacgggaccggcacgccgctgggcgaccccatcgagttcgaggccctgcgcgccgcgttcggcgacgcgcccgaggcaggctgcgccctggggtccgtcaagaccagcctcgggcacacccagttcgccgcgggcgtggccggcgtcatcaaggtgctgctggcgctcaggaacgagcaactgcccccgtcgctgcacttccgccgggccaacccggcgatcacgctggagggcagccccttctacgtcaacacggaactgcgcccgtggcccgcacccgccgacggtccgcgccgcgccggcgtcagctcgttcggcgccgcaggcaccaacgcgcacgcgctgatcgaacaggcccccgccgtgcggaccgccgggcacggcccccggcatgcctggctgatcgtcctgtcggcacaggacgacgccggccgccgagcccaggccgagcgcctgctggaccacgccctcgcccacgaggacctggacctgggcgacgtggcgtacaccctggccaccggacgccgccactgcagccaccgctgggcgggcgtggccacggaccgcgagcagctcgtcgccgccctgcggacctggctgtgcgacggccgggcggagggcgtggtcaccggtgaggcgcccgacgggcaccgccgtcaggaccccgccgaggacgcccgcgccggccgcctgatggccgagcccgaccgtcacgacagcctcaccgagctggccgggctcttcgcccaggggcaagatctgggcttcgccccgctcttcggcgacggcggcttccgtatcgtctccctcccggcctatccgttcgcgggcgagcgctactgggtcggatcacgtccggcggcccccgctgcgaccccggcctccgctccggtacgcgccccggtccccgttgcggccccgtcgccgctggaaggccgccggctgaccggtgatcccggctcgccgtccttcgccgtcgagctggccggccgcgagttcttcctcgacgaccaccgggtgcgcaacgtgccggtgctccccggcgtggcctatctggagctggcgtacgcggcggcccgggccgagggcgtcgaccccgcccacgcccgcctgcgcaacgtcgtctggtcgcgccccgcacggatcaccgggccgaccgcggtcgagatcgcgctgcggccgtgcgaggacgacgccttcacctacgagatcaccacggcggccgacggcgaacagccggtgatccacgggcaaggacgcatcgagcggtgcgggacgccgtcacccgcgcgcctggacatcgccgcgctgcgcgcccagtgcgaggtgcgcactctggaacacgacgactgctaccggctcttcgaccgcatgggcatcggctacggcccggccatgcggggcatccggcggatccacgtcggcgccgggctcgccgtcgcacgcctgagcctgccgcaggccgcccgggacggcgccggctgggacctgcacccgtcgatgctcgacgccgccgtacaggccaccttgggcctgtcactggccgaggacaccgacaccgtggcgccggcactgcccttcgtcctggaggaggtgcagctgctcgcgcccagcccggccggcgggtgggccgtggtgcgacccgcagcgggcgacggcggcggagccgtacgccgcatcgacatcgaactgtgcgacgacgacggcgaggtgtgcgtacgcctgctcgggttcaccgcacgcgtcctggccgccggtgacgaccccgccggcggagagaacaccgggggcgcgacgctcaccctcatgcgtgccggctggcgcccggccgagcccacccgggcctcgcgcccgctggtgcaccacgaggtgctgctcggcggactcgcagggaccgaccccgcggcggtccgggacgggctcggtgtgccctgcaccgcattgcccgacgacggcgatccggcccggtgtttcacccgccaggccgagacggtgctggcccgcctgcagcagttcgtcccacgcacccgcgacggcgaggtcctgctgcaggtggtcgtgcccgccgacggtgagaaccgggtcctcgcgggcctgggcggcctgctgcgcacggcccgcatggagcaccccaagctgctgacccagctcgtcgaggtggagacgcccgtcgacgccgcgacgctgtgcgagcgcctgcgccgggacgcggcgagccccgacgacgtggccgtgcggtactccggcgggcagcgccgggtgccgcagtggaccgccgtcgaggacgccccgccggcccgcccctggaaagccggcggcgtctacctcctcaccgggggagtcggcgggctcggcgcacacttcgcccgcgagatcgcccggcaggcgcccggcgccgccctcgtgctctgcgggcgctcgccggagggcccggcccagcgtgaactcctgtgtgagctgggcgacttgggtgcctccgccgtctaccgggtgctggacgtcgcccggcgcgacgccgtgaccgcctgcgtgaacaccgtcgtcgccgagcacggccgcctggacggcgtcgtccacaccgccggtgtggtgcgcgacggctacctggcccgtaagagcgccgaagagctgcgggaggtcctcgccgccaaggtcgccggcttcgtccacctcgacggagcgaccgccgcgctcgacctggactgcttcatcggattctcctcactgtcggcgtacggcaaccagggccagggcgactacgcggcggccaacgccttcatggacgcctacgccggcctccgccacgagcgggtggccaggggcgagcgccgcggccgcacactggtggtcggctggcccctgtgggccgacggcggcatgacggtggacgccgccaccgaacgccgcctgcacgacagcgtcggcatggtgccgatccgcgccccgcacggtgtggaggcgctgctacgcgcctacggcaccggcgacccgcacgtcctggccgtcttcggcgaccgcgcccgcatcgacgccaccctcctggccgccccggcggccacgggcgccgcaccggcggtgaccgcacccgaccgcgccgccctgcacgcgagggtcctcggccgcgccatcagccacgcctgcgccgtgctgggcgttccggcggcggagctcgacggtgcggtggagctgagcgagtacggcttcgaccccgtctcgctcaccgggttcgccgcccgcctcaccacggagttcgggcttccgcccgtgcccaagcccttctccgaacacctcaccctgggagaggtcgtggaccacctgctcgacacccacccccatcacttcgggacggtcccgccggcccccgcgcccgagccctccgccgggcccgaaagcgccgccgcgcccgtcgcgacggccggccgggagcagcagcacaaggcgctgctgaagaagctgatcgcccgcgtgtccgacctgctggacgtgcccgccgagcggatcaccggcacggccgagatgacccgctacggcttcgactccctctcgttcatcggcttcgccaacgacctcaacgccgagttcgggctctcgctggcaccgaccctgttcttcgagaaccccaccctggacggggtcgtcgaccacctcctcgaccaccacgccgaccgcgtcgccgccaccgcggcaccgcagcaggaaccgcgcgcggcggcggcccccgccgccccagagcccgccacagccgacacccccgcgtcccgtacggatgcgcccgggaacgagccgatcgccgtcatcggcatcagcggccgcttcccgatggccgacgatctcgacgcgttctgggagaacctcagcgaaggccgcgactgcacccgtgaggtccccacggaccgctgggactggcgcgcccactacggcgaccccgtcaaagagcccaacacgtcgaacgtgacgtccggcggcttcatggacggcgtcggcgacttcgacccgcttttcttcgacatctcccccaaggaagcggagttgatggatccgcagcagcggctcctgctgatgtacgtatggaaggcgctggaggacgccgggtactcggcggaggccctcgcgggcacgaacacggccctcatcgccggcaccaccagcaccggctacagcaccctcgtcacccggtactcgccgatgatcgagggatacgacatcaccggcgcggccccctccatgggcccgaaccggatgagctacttccttgacctgcacggtccgagcgagcccgtcgacacggcctgttcgagcgcgctcgtcgccctgcaccgggccgtccaggccatccgcgacggtcagtcggacctggccatcgccggcggcgtcaacaccatggtcagcgtcgacgggcacatcagcatctccaaggcgggcatgctcagccccgaaggccgctgcaagaccttctccgaccgcgcggacggttatgcccgtggtgagggcgtgggcatgctggtgctcaagagcctgtcggcggccgagcgcgacggcgaccacatctacggggtcatccgctcgacggccgagaaccacggcggccggggcagctccctgaccgcgcccaaccccaaggcccaggccgccctcctgcgggaggcctacgggaaggccgggatcgatcctcggacggtgggctacatcgaggcccacggcaccggcaccaaactcggcgacccggtcgagatcaacgggctcaaggccgcgttccgggacatgtacgaggagcacggcgcggtggtcgaggaggcccactgcggtatcggctcggtgaagaccaatatcggtcatctcgaactggccgcgggcgccgccggcgtgatcaaggtgttgctccagatgcggcaccgcaccctggtcaagagcctgcactgcgacaccgtcaacccctacatcgacctcgacggcagcccgttccacctcgtacgcgaacggcagccctggcccgccctgcgcgatgccgaaggccgtgagctgccgcgccgggccggagtcagctccttcggcttcggcggcgtcaacgcccatgtggttcttgaggagtaccggccgcgcaccgcacccgagccggaccgggcgcccaccgcaccggtccccgtcgtcctgtcggcgagccaccccgacgtgctgtgcgaactcgccgagcgctgggtggacgcactgcgccgcggcgactacgacgacaccgacatggcgtcgatcgcctacaccacgcagaccggacgcacgcccatgaccgagcgcctcgcctgcctggcccgcacggccggcgaactgcgggaggcactggagtcctggctgcgcggcgagcccgcggccgacgtcttccgcggcaaggtcgcgcgcggcgtcgacctgccggacgcaccagccgggttcggcccgcacgacgaccacgacagcgcgggccggcacgactgggcccgcctgctccaggcatgggtgaacggcgcccccttcgactgggaccgcctccacaccgggcgccgcccgcgccggatcgccctgccgacctacccgttccgcctccggcgctactgggtcgacacctcgcgccccgcgaacggcacacaaacggaggcactgcacccgctggtgcacacgaacacctcggacctgaacgagcaccgctacacctcgcacttcaccggccgcgagttcttcctcgccgaccaccgcgtacgcgcccaggtgatggagacggtctccggctggcggcccggccgccggcccaccgcctacgacgtccgcgcggacgccgtgccggtgctgccggcggtggcctacctggagatggcgcgcgccgccgcggtccaggcggccggcggcgacgagcgcgcctggtcactgaagttggcctcctggctgcgcccgctcaccgtcgagaaggcgaccgacgtgcacaccacgctgaccacccgggccggcggcggactgagctacgaggtgtacgcggtggacgaggacggcgaacgcgtcaccttcggccgcggccagctgcggcgcgcaacagcggtgcccgccgagcggctcgacctcgcggccctgcgcgcgcagtgcgacggccccgtgctcgacgccgagacctgctacgcacgcttcaccggcatcggcatggcctacggcccggcactgcgcggcatcgagcgcctgcacaccggctcgcggcagtcggtggcgcggctgaagctgcccgccgccgcgtcccgcgagcgcggctgggtactcaacccgggcatgctcgacgccgccctccaagccacggtcggcctcttcgtcgacgaccccggcacgccgcgcacggcactgccgttcgccctgggcgagctggaggtgctgcgggcggtcccgggcaccggctgggtcgtggtccgcttcgccgaggacgaccacgtgggcgccgtgcgccgcctcgacctcgacctctgcgacgacgacggcgaggtgtgcgtacgcctgcgcggcttcagcgtccgcacgctcggcggcagcgagcccaccggtgacagcgagcccacccggcccgccgaacaggcacccgagccgccgtccgggtccgacgacgcctacctgctggacctgatcgaagccattggccgacgcgagatgagcgcggacgaattcaagaggagcctggcatga

SEQ ID NO: 14 - protein, 1953 aa, Streptomyces platensis subsp. rosaceus
VGQDEALRLIRDWKQEQEQEQEQDQNQEQARARTQTARPADVADTEALTERVCAVVVEKVCELLKVTTDDLDVHVDLSEYGLDSLVITQLVNMVNDALGLELVPTVLFEHATIQAFGAHLTDEYGPALAARLGLRSPGAATEPPAVEPVGTPVPAAAVPARAVPVPLPADRHDDPIAVVGMSGRFPQAEDLDAFWRNLRDGRDCIAEVPADRWDWRALFGDPLQEPGRTNVKWGGFMEGVADFDPLFFGIAPKDAVHMDPQQRLLMLYVWKALEDAGYAADALAGSSFGLFVGTSDTGYGLLSDRSSGRGESVTPTGSVPSVGPNRMSYFLDVHGPSEPIETACSSSLVAMHRGVISIERGECDMAVVGGINTMVIPDGHVSFSKSGMLSAEGRCKTFSDRADGYARGEGVGMLVLKSLSAAERDGDHVYGIIRSTAENHGGRSNSLTAPNPKAQAALIRRAYSTAGIDPRTVGYIEAHGTGTKLGDPVEINGLKAAFRELYEEHGAVVDDAHCGIGTVKTNIGHLELAAGVAGVIKVLLQMRHRTLAKSLHCDTVNPYIDLDGSPFHLVREQQPWPALRDAEGRELPRRAGVSSFGFGGVNAHVVLEEYVPRPVPPVSTPDPVAVVLSAPEPEMLRARARQLADRIDSGGLGEADLPDLAHTLQVGRVAMDERLAFLTSSLADLRERLGAFLDGGTVQGLHTGRAQRPGPWNELAGDDDIALALDSWIAKGKLGRLLKLWVTGFDVDWRRLYAGRPMRRIPLPVYPFQLKRYWITDAKSTTRPPAPVAAAPDAQPSPYRRDLTGHEFYVSDHRVGDTPVLPGTAYLEFVRDALVRATSAGTATGVRLRDVTWLRPLEVTALRTLAVDVDPAGGTFEVYDHGSGDRVLHANGTAHVDPALLAADDTHDIDALRANLPFRRDGAECYALFARRGMGYGPAFRAVQELYHGADTALARLLLPEAAASSLTLNPVMLDAALQATLGLALGEHVDAPQGTALPFTVREVQVLAPTPAEGWALVRRAADDRHDTGIRRLDIDLCDTQGNVCVRLLGFSTRMKPSPAPRAAEPTTTPALLIQADWRESAAREHHGDDVKRHVVLCELPAADATALGAALGGATCETWQARGETGTRYTEYAERLLKLLRDKAPEAARQPCLIQVVTPAHAPWLGGLSGMLRTARMEHPKLLTQWIALDGDGALAPAELAGRLRCDGADTAEEAVRYRGGRRQVSQWHEVAPAAPERPWRDGGVYLLTGGAGGLGALFAQDIARRVETPALVLCGRSPVGPAQQELLTALRALGARADYRVLDVADRADVTRVVREVQAEYGALHGIVHAAGVLRDGFVAKKTADDLREVFAAKVAGLCHLDEATASVPLDCFIGFSSMAAFGNVGQADYAAANAFMDGYAAHRDSLVDQGSRSGRTLMVNWPLWEKGGMGADPSTVQLLESVGMRPMRASVGIDALDRVWATGLPSAIALDGDHARMRERFLPAHPEPEAPAEPAPAAATLPATAPVAEPAEPSSVGTVVADLMATLLEVDVETLRWDKSLGDYGFDSIFMMQFLAQAQTHLDASLTLDVIADCETLQDVVDAITGTASDTGTAAPKPASVAPVEEAPAAAAPAKAPVRRAAAASPNDFPELVRMNGVTSGRPVFWVHHGNGGVESYAPLAARCPRPFYGIQPKGWIDSTDILTGQYAMAEHYASLILAVQPEGPYDIGGFSLGGLFAYETVRQLQLTGADVRTLVMLDTLDAESTNKANALIVGGNFDADVVTKVSDFRAVNLILGNNRFDSHGGATPILRRDEVDTTLEPKEFLGSLIDAALARGVSKTETQLRSRVRQLSRYFEATQGETYTVDPLPRRDGLRCYYLRNRGGKFFGAFEEHMVLFPNPELPTVDGVAYWQEWADQIDDFFTIDVDTSMHAEVMTAPQSLDKLMRLCDRLYAAEDAAAPATSAQGGR

SEQ ID NO: 15 - DNA, 5862 bp, Streptomyces platensis subsp. rosaceus
gtgggacaggacgaagcgctccgcctgatccgcgactggaagcaggagcaggagcaggagcaggagcaggatcagaatcaggagcaggcgcgagcacggacgcagaccgcacggccggctgacgtcgccgacaccgaggccctgacggagcgggtctgcgccgtcgtggtggagaaggtctgcgagctgctcaaggtcaccacggacgacctggacgtgcatgtcgacctcagcgaatacgggctcgactccctcgtcatcactcagctggtgaacatggtgaacgacgctctgggtctggaactcgtgcccaccgtgctgttcgagcacgcgacgatccaggccttcggcgcccacctgaccgacgagtacggccctgcgctggccgcccgcctggggctgcggtcgcccggcgccgccacggagccccctgccgtcgagcccgtcggtacgcctgtgccggccgcagccgtccccgcacgggccgtacccgtcccgctgcccgccgaccggcacgacgacccgatcgcggtggtcggcatgagcggccggttcccccaggccgaggacctcgacgccttctggcgcaacctgcgcgacggccgcgactgcatcgcggaagtccccgccgaccggtgggactggcgcgccctcttcggcgacccccttcaggaaccgggccgcaccaacgtgaagtggggcgggttcatggagggcgtcgccgacttcgatccgctgttcttcggcatcgctccgaaggacgccgtccacatggacccgcagcagcgcctgctgatgctgtacgtgtggaaggcgctggaggacgccggctacgccgccgacgccctggccgggagcagcttcggcctgttcgtcggcaccagcgacaccggctacggcctgctctccgaccgcagcagcggcaggggcgagagcgtcacgcccacgggcagcgtcccctccgtcggcccgaaccggatgagctacttcctggacgtacacgggccgagcgagccgatcgagacggcctgttcgagttccctggtcgccatgcaccgcggcgtcatctcgatcgaacgcggcgagtgcgacatggccgtcgtcggcggtatcaacaccatggtgatccccgatggccacgtcagcttcagcaagtccgggatgctcagcgccgaggggcgctgcaagaccttctccgaccgcgcggacggttatgcccgtggtgagggcgtgggcatgctggtgctcaagagcctgtcggcggccgagcgcgacggcgaccacgtctacggcatcatccgctcgacggccgagaaccacggcggccgctccaactccctgaccgcgcccaaccccaaggcccaggccgccctgatccggcgcgcctacagcaccgcgggcatcgaccctcggacggtgggctacatcgaggcccacggcaccggcaccaagctcggcgacccggtcgagatcaacgggctcaaggccgcgttccgggaactgtacgaggagcacggcgcggtggtcgacgacgcccactgcggtatcggcacggtgaagaccaacatcggccacctcgaactcgcggcgggcgtcgccggcgtgatcaaggtgctgctgcagatgcggcaccgcacgctcgccaagagcctgcactgcgacaccgtcaacccctacatcgacctcgacggcagcccgttccacctcgtacgcgagcagcagccctggcccgccctgcgcgatgcggagggccgtgagctgccgcgccgggccggagtgagctccttcggcttcggcggcgtcaacgcccatgtggtgcttgaggagtacgtgccgcgccccgtaccgccggtgagcacaccggaccccgtggccgtcgtcctgtcggcccccgagcccgagatgctgcgcgcccgggcccggcagctggccgaccggatcgactcgggcgggctcggcgaggccgacctgccggacctcgcccacacgctgcaggtgggccgcgtcgcgatggacgagcgcctcgccttcctgacctcctcgctcgccgacctgcgcgagcggctgggcgccttcctcgacggcggcaccgtacagggcctccacaccggacgggcacagcgcccggggccgtggaacgagctcgccggagacgacgacatcgccctcgccctcgacagctggatagccaagggcaagctcggacgcctgctcaaactctgggtcaccggcttcgacgtggactggcggcgcctgtacgccggccggccgatgcggcgcatcccgctgcccgtctacccgttccagctgaagcgctactggatcaccgacgcgaagagcacgacccggcccccggcaccggtggccgcggcgccggacgcacaaccgtcgccctaccgccgcgacctgaccgggcacgagttctacgtgagcgaccaccgcgtgggggacacgcccgtcctgcccggcaccgcctacctcgagttcgtgcgcgacgcgctcgtccgggccacgtccgcaggcaccgccacgggcgtgcgcctgcgcgacgtgacctggctgcgtcccctggaggtgacggcactgcgcaccctcgccgtcgacgtggacccggccggtgggacattcgaggtgtacgaccacggctccggcgaccgcgtcctgcacgcgaacggcaccgcacacgtcgaccccgcactcctcgccgccgacgacacccacgacatcgacgcactgcgcgcgaacctcccgttccggcgtgacggcgccgagtgctacgcgctgttcgcgcgcaggggcatgggatacggccccgcgttccgggccgtgcaggagctgtaccacggtgcggacaccgcccttgcccgcctcctcctccccgaggcggcggcatcctcgctgacgctcaacccggtcatgctcgacgccgccctgcaggcgaccctgggactggcgctcggcgagcacgtcgacgccccgcaggggacggcacttccgttcaccgtgcgcgaggtacaggtcctggcccccaccccggccgagggctgggcgctcgtgcgccgtgccgcggacgaccgccacgacaccggcatacgccgcctggacatcgacctctgtgacacgcagggcaacgtctgcgtgcgtctgctgggcttttccacccgcatgaagccgagcccggcgccccgcgccgccgagccgaccaccacgcccgcactgctgatccaggcggactggcgcgagagcgcggcacgggagcaccacggcgacgacgtcaagcggcacgtcgtcctgtgcgaactcccggcggcggacgccaccgcgctcggcgcagcgctgggcggcgccacctgcgaaacctggcaggcccgcggcgagaccggcacccgctacaccgagtacgccgagcggttgctgaagctgctgcgcgacaaggcaccggaggccgcccggcagccgtgcctgatccaggtcgtcacccccgcgcacgcgccctggctgggcgggttgagcggcatgctccgcacggcgcgcatggagcaccccaagctgctgacgcagtggatcgctctggacggtgacggcgccctggccccggccgagctggccggacggctgcggtgcgacggcgccgacacggccgaggaggccgtgcgctaccgcggaggacgccggcaggtgtcccagtggcacgaagtcgcaccggccgcacccgaacggccctggcgcgacggcggcgtctacctgctgaccggcggcgccggaggactcggcgccctgttcgcgcaggacatcgcccggcgcgtcgagacgcccgccctggtcctgtgcggtcgcagcccggtcggcccggcacagcaggaactgcttaccgccctgcgcgcgctgggtgcccgtgccgactaccgcgtgctcgatgtcgccgaccgcgccgacgtgacccgggtcgtgcgcgaggtccaggccgagtacggcgcgctgcatggcatcgtgcacgccgccggagtgctgcgcgacggcttcgtggccaagaagaccgcggacgacctccgcgaggtgttcgcggccaaggtggccgggctgtgccacctcgacgaggcgactgcctccgtcccgctcgactgcttcatcggcttctcctccatggccgccttcggcaacgtcggacaggccgactacgccgccgccaacgccttcatggacggatacgccgcccaccgcgactccctggtggaccagggcagccggtcgggccgcaccctgatggtgaactggccgctgtgggagaagggcggcatgggcgccgacccgtcgaccgtccagctcctggagtccgtgggcatgcggccgatgcgcgcatccgtgggcatcgacgccctcgaccgcgtctgggcgaccggcctgcccagcgccatcgccctcgacggcgaccacgcccggatgcgggagcgcttcctgccggcgcaccccgagccggaggcccctgccgaacccgcgccggccgccgcgacgttgccggccaccgcaccggtcgccgagccggccgagccgtcgagcgtgggaaccgtcgtcgcggacctcatggcgacactgctggaggtcgacgtcgagaccctgcggtgggacaagtccctgggtgactacggcttcgactccatcttcatgatgcagttcctcgcccaggcgcagacgcacctcgacgcgtccctcaccctcgacgtcatcgccgactgcgaaacgctgcaggacgtcgtcgacgcgatcaccggcaccgcctctgacaccggcacggccgccccaaagcccgcttcggtggctcccgttgaggaggccccggcggccgccgcaccggcaaaggccccggtacggcgcgccgcggccgcatcgccgaacgacttccccgaactggtccgcatgaacggtgtcacctccggccggcccgtgttctgggtccaccacggcaacggcggcgtggagtcctacgccccgctggccgcacgctgcccgcgtcccttctacggcatccagccgaagggctggatcgactccaccgacatcctcaccggtcagtacgccatggccgagcactacgcgtccctcatcctcgccgtacagccggaaggcccgtacgacatcggcgggttctccctgggcggcctgttcgcgtacgagaccgtgcggcagctccagctgacgggcgccgacgtgcgcacgctggtcatgctggacacgctggacgccgaatccaccaacaaggccaacgccctcatcgtcggcgggaacttcgacgccgacgtggtcaccaaggtgagcgacttccgcgccgtcaacctgatcctcggcaacaaccgcttcgactcgcacggcggcgccaccccgatcctgcgccgcgacgaggtcgacaccaccctggaacccaaggagttcctcggctccctgatcgacgccgccctcgcccgcggcgtcagcaagaccgagacgcagctgcgctcccgcgtccggcaactgtcccgctacttcgaggccacgcagggcgagacgtacacggtggacccgctgccgcggcgcgacggactgcgctgctactacctgcgcaaccggggcggcaagttcttcggcgccttcgaggagcacatggtgctcttcccgaaccccgagctccccaccgtggacggcgtcgcgtactggcaggagtgggccgaccagatcgacgacttcttcaccatcgacgtcgacacctcgatgcatgccgaggtcatgacggccccgcagtcgctcgacaagctgatgcgcctctgcgaccggctctacgccgccgaggacgccgccgccccggcgacctccgcgcagggaggccgctga

SEQ ID NO: 16 - protein, 751 aa, Streptomyces platensis subsp. rosaceus
MKAVVFPGQGAQRRGMGRELFDAYPELADEASEVLGYSLRTLCLDDPHQQLGRTEYTQPALFVVGALAHRQWRESTGDEPAFLAGHSLGEYCALHAAGAFDFATGLRLVQRRGALMAQARGGGMAAVVGVDAARLRELLDEGGFCRLTVANDNAPQQKVVSGDTATVDALVAYLEARDVRCVRLNVSGAFHSPLMRQAQQDFARFADGFALGDPATPVIANATARPYVPGRTARTLVDQIVQPVRWTESVHHLLDQGVTDFVELGGRVLVRLIDQIRSAPRPVAQHDAPAARPDTPAAALGSPLFRRRMGVRHAYAVGGMYRGIASAQMVVRLGRHRILGFLGTGGLPLPEIEQGVKEVQHGLADGQPYGVNVLADHDDPAAERALVDLLMRHGVPVIEASAFMQMTPALVLYRARGLRRGADGRTVCDHRIVAKVSRPEVAEQFMAPAPGPVLDRLRREHALTDEQAELARTVPMSHDITVEADSGGHTDGGVATVMMPAMLKLRQQAQDRYGYDEPICMGLAGGLGTPAAVAAAFMLGADYVLTGSINQCTVESGMSTEVKDMLQDVGIADTAYAPAGDMFEFGAKVQVLRKGVFFPTRANRLFSLYSHYDGLDDIPQKTRSLLERTYFGKSIEEVWDEVRAYLRSQGRDADIDRADAEPKQKMALVFRWYFFHTTRLAMDGDGSGKVNYQVQTGPALGAFNQWVEGTELASWRHRHVDRIGLMLLDGAAEHIATACRHWRDTLGVPSA

SEQ ID NO: 17 - DNA, 2256 bp, Streptomyces platensis subsp. rosaceus
atgaaggcagtcgtctttcccggccagggcgcccagcggcgcggcatgggacgcgagctgttcgacgcgtatcccgaactcgccgacgaagcctccgaagtcctcggctactccctccgcacgctgtgcctggacgatccgcaccagcaactgggccgcaccgagtacacgcagcccgcgctgttcgtggtcggggcgctcgcccaccggcagtggcgcgagagcaccggcgacgagccggccttcctcgccgggcacagcctgggggagtactgcgccctgcacgccgccggcgccttcgacttcgcgaccggactgcgcctcgtccagcggcgcggcgcactgatggcgcaggcacggggcggtggcatggcggccgtggtcggggtcgacgcggcacggctgcgcgaactcctcgacgaaggcggcttctgccgcctgaccgtcgccaacgacaacgcaccccagcagaaggtcgtgtccggcgacaccgcgaccgtcgacgcgctggtggcatacctcgaggcgcgcgacgtccgctgcgtgagactgaacgtgtccggcgccttccactcgcccctgatgcggcaggcacagcaggacttcgcccgcttcgccgacggattcgccctcggggacccggcgacgcccgtgatcgccaacgccacggcgcgcccgtacgtccccggccggaccgcgcgcacactcgtagaccagatcgtgcagcccgtgcgctggaccgagagcgtgcaccacctcctcgaccagggcgtcaccgacttcgtcgaactgggaggccgggtgctcgtcaggctgatcgaccagatccgctccgccccgcggccggtcgcccagcacgatgcaccggctgcccggcccgacacaccggccgccgcgctcggcagccccttgttccggcggcgcatgggcgtgcgccacgcctacgccgtgggcggcatgtaccggggaatcgcctcggcccagatggtcgtcaggctcggccgtcaccgcattctcggcttcctgggaaccggcggcctgccgctcccggagatcgagcagggggtgaaggaggtccagcacggcctggccgacgggcagccctacggcgtcaacgtactggccgaccacgacgatcccgcggccgagcgcgcgctggtcgacttgctgatgcgccacggcgtccccgtcatcgaggcgtcagccttcatgcagatgacccccgcgctcgttctctaccgggcacggggactccgccgcggtgccgacggccggacggtgtgcgaccaccgcatcgtggccaaggtctcccggccggaggtcgccgagcagttcatggcacccgcccccgggccggtcctggacaggctccgccgggagcacgccctcaccgacgaacaggcggaactcgcccggacggtgccgatgagccacgacatcacggtcgaggcggactccggagggcacacggacggcggcgtcgccacggtcatgatgcccgccatgctcaagctccggcagcaggcccaggaccggtacggctacgacgaaccgatctgcatggggctcgccggcggcctcggcacccccgcggcggtcgcggcggccttcatgctgggcgccgactacgtactcaccggatccatcaaccagtgcacggtggaatccggcatgagcaccgaggtcaaggacatgctgcaggacgtcggcatcgccgacaccgcctacgcgcccgcaggcgacatgttcgaattcggtgccaaggtgcaggtgctccgcaaaggcgtcttcttccccacgcgggccaacagactgttctccctctactcccactacgacggcctcgacgatattccgcagaaaacgcgctccctcctggagagaacctacttcggaaagagcatcgaagaggtctgggacgaagtacgcgcctatctccgttcgcaggggcgcgacgccgacatcgaccgggccgacgccgagcccaaacagaagatggcgctggtattccgctggtacttcttccacaccacgcgcctcgccatggacggcgacggctccggaaaagtgaactaccaggtccagaccggtccggcgctgggtgccttcaaccagtgggtcgaaggcaccgaactcgcctcatggcggcaccgccacgtagaccggatcggcctcatgctgctcgacggtgccgccgaacacatcgccaccgcatgccggcactggcgcgacaccctcggggtgcccagtgcgtga

SEQ ID NO: 18 - protein, 338 aa, Streptomyces platensis subsp. rosaceus
MTVPSTGTEVRLATRPEGWPTTENFSVVQAEPPAVRTGQVLIRNLVMSVDPYMRGRMNKTRSYVPPFAVGKALDGGAVGEVVVSKSSQLAVGDLVLHGLGWREYAVVGAAGAVRIDPALAPPGAYLGVLGMPGHAAYTGLLKAAEFRPGDTVFVSGAAGAVGSLVGQIARLCGAERVIGSAGSAEKVAYLTGELGFDAAFDYKDGPVLEQLAKAAPTGIDVYFDNVGGDHLDAALVLARMGARFALCGNISQANEKDPPAGPRNLTQAIAKGITLRGVLVGGHADLPDEFTARMGGWLADGRISYRETVVRGLENAPAAFIDMLRGANTGKMLVRIAE

SEQ ID NO: 19 - DNA, 1017 bp, Streptomyces platensis subsp. rosaceus
atgaccgttccgtccacgggcaccgaagtccggctcgccacccgccccgaggggtggccgaccactgagaacttctcggtcgtgcaggcggaaccgcccgcggtcaggaccggccaggtgctgatccgcaacctggtgatgagcgtcgacccgtacatgcgcgggcggatgaacaaaaccaggtcctacgttccgccgttcgccgtcggcaaggcgctcgacgggggcgccgtcggcgaggtcgtcgtctcgaagtcatcgcaactcgccgtcggtgacctggtcctgcacggcctcggctggcgggagtacgccgtcgtgggcgctgccggcgcggtcaggatcgacccggcgctcgcgccgcccggcgcgtatctcggagtgctcggcatgccggggcacgccgcctacacggggttgctcaaggccgccgaattccggcccggcgacaccgtgttcgtctccggggccgcgggcgcggtgggctccctcgtcggtcagatcgcccggctctgcggcgcggaacgcgtgatcggatcggcgggcagcgccgagaaagtcgcctatctgaccggggagctcggcttcgacgcggcattcgactacaaggacgggccggttctcgaacagctggcgaaggccgcgccgacgggcatcgacgtgtacttcgacaacgtgggcggcgaccacctggacgccgccctggtcctggccaggatgggcgcgcggttcgccctctgcggcaacatctcgcaggccaacgagaaggacccgccggccggcccacggaacctgacgcaggccatcgccaagggcatcaccctgcgcggcgtcctcgtcggaggccacgccgacctcccggacgagttcaccgcccgcatgggtggctggctggcagacgggagaatctcctaccgggagaccgtcgtcaggggactggagaacgcacccgccgccttcatcgacatgctgcgcggcgccaacaccggcaaaatgctcgtgagaatcgccgaatga

SEQ ID NO: 20 - protein, 281 aa, Streptomyces platensis subsp. rosaceus
MSSTSTTAPVSVPVSAPVPEEVGHLYDRLTALDTEAAGGSLHLGYWDVDDNDTPLVEAADRLTDTMTDRLRIDQGQRVLDVGCGVGQPAMRIARRTGAHVTGIAISKDQIARATALAEGAGLSDRVEFRHADAMELPFPDDSFDAAIAIESIFHMPDRGRVLAEIRRVLRPGGRLVLTDFFERGPVPAEKQPAVDRLLRDFIMTLARPEDYVPMLRDAGLRFVELLDITEQSVRQTFEQMSQGSQEMQTVFDDEAEEKFSPASMIDVDEFGSVLLTAQKPL

SEQ ID NO: 21 - DNA, 846 bp, Streptomyces platensis subsp. rosaceus
atgtccagcacgtccacgaccgcccccgtctccgtccccgtctccgcccccgtccccgaagaggtcggacacctctacgaccgcctcaccgcactggacaccgaagcggccggcggcagcctccacctcggctactgggacgtcgacgacaacgacaccccgctcgtggaagcggccgaccggctcaccgacacgatgaccgaccgcctgcggatcgaccagggacagcgggtcctcgacgtcggctgcggagtcggccagccggccatgcggatcgcccggcgcaccggcgcccatgtcacgggcatcgcgatcagcaaggaccagatcgcccgcgccaccgccctcgccgagggcgccggcctgagcgaccgcgtggagttccggcacgccgacgccatggaactgcccttccccgacgactccttcgacgccgccatcgccatcgagtcgatcttccacatgcccgaccgcggacgggtcctcgccgagatccgccgcgtactgcgccccggcggccgcctggtcctcaccgacttcttcgagcgcggccccgtccccgccgagaagcagcccgcggtggaccggctcctccgcgacttcatcatgacgctggcccggcccgaggactacgtgcccatgctgcgcgacgcaggcctgcgcttcgtcgagctcctcgacatcaccgagcagagcgtgcgtcagaccttcgagcagatgagccagggctcccaggagatgcagaccgtcttcgacgacgaggcagaggaaaagttcagccccgcctccatgatcgacgtcgacgaattcggctccgttctgctgaccgcccaaaagcccctctga

SEQ ID NO: 22 - DNA, 1700 bp, Streptomyces platensis subsp. rosaceus
tgccggcagaagtacaacggcctccccaaaaagcgggccccgtttagaaggtgatcctgccgaccggtaagccgggcgtggatcctgacccgctacgacgacgttcgcaaggcgctgctcgacccgcatctcagctccgaccgggccgatccgaacttcccgatgctggtcgagctcgcgcggagcgacaaggactccacactgtccctggcgggcatggacgcgcccgaacacacccgcgcgcggcgcgccgtggtcggcgagttcaccttccgccggatggaagccctgcgcccgcgcgtccaggagatcgtcgacgagtgcgtcgacgcgatgctcgccggccccaacccggccgacctggtgaagacgatgtccttcccggtgccctccctggtcatctgcgagctgctcggcgtgtccgtcgccgaccgcgacttcttcgaggaccgcacctcccgcatgctcagcatgaccctcccgccgctgacccggcagcaggcgttcttcgacctgcagacctacttggacgagctcgtgacggccaaggagaaagtcccgggggacgacctgctcagccgccagatcgccaagggccgaaaggacagcgcgtacgaccacgacgcactcgtcgacctggcgttcctgctgctgaccgccgggcacgacacgaccggaaacatgatctcgctgggcatgctcgccctgatggagacgcccgagctgcgggcgcgcatcaccgacgaccccggcaccacgccgcaggtcgtcgaggaactgctgcgctacttcaccatcaccgacatcatcaccacgcgcgtggccaaggaggacgtcgagatcggcgggcagaccatccgcgccggcgaaggcgtcttcgcgctgggcggctcggccaaccacgacccggacgtcttcgagaaccccggaaagctggacgtcgaccgcggcgcacgccagcacctcgccttcggccacggaccgcaccagtgcctcgggcagagcctcgcccgcatggaactggagatcgtctacgacaccctgctccgccgcatccccggactccggccggccggccccgctgaagacctgccgctgaagaacgacgcggccatcttcggcctgcacgaactcccggtcatctggtaggcggagcctgtgcgcgccaggcggtcatgacgtggctgtccgtgcggtgacgcgggccggggcgtcgtgcatcggtcgtactcaggggcttggtggtgatgccgatcagctgtaggacccgcggccgccggtgtggagactgatcaccatgcgtactgactttgatcccgctgccatgtcccgtaacgacttctaccggctcctgaccgcgacggtggtgccccggccgatcgcctgggtgtcgaccacgtcggcagacggaacgcacaacctcgcacccggacaccccgatggcgaggttgtcatcggtcccggaggccagggtcgcggcaacgccttcgggtgagcggcggtgctgctcagcccagtcttcggcaacggatcgggaccacctgctgcgcccgttgacagtggattgtggagggggaacgtcgccttcaccgcgggagatgtaagcgcgcagggtggaagcggcaatggcgccgatcttcgccatgtccggcaccggctgctggtgggacggcaggggagttgcgggccccaccagttgg

SEQ ID NO: 23 - protein, 328 aa, Streptomyces platensis subsp. rosaceus
MLVELARSDKDSTLSLAGMDAPEHTRARRAVVGEFTFRRMEALRPRVQEIVDECVDAMLAGPNPADLVKTMSFPVPSLVICELLGVSVADRDFFEDRTSRMLSMTLPPLTRQQAFFDLQTYLDELVTAKEKVPGDDLLSRQIAKGRKDSAYDHDALVDLAFLLLTAGHDTTGNMISLGMLALMETPELRARITDDPGTTPQVVEELLRYFTITDIITTRVAKEDVEIGGQTIRAGEGVFALGGSANHDPDVFENPGKLDVDRGARQHLAFGHGPHQCLGQSLARMELEIVYDTLLRRIPGLRPAGPAEDLPLKNDAAIFGLHELPVIW

SEQ ID NO: 24 - DNA, 987 bp, Streptomyces platensis subsp. rosaceus
atgctggtcgagctcgcgcggagcgacaaggactccacactgtccctggcgggcatggacgcgcccgaacacacccgcgcgcggcgcgccgtggtcggcgagttcaccttccgccggatggaagccctgcgcccgcgcgtccaggagatcgtcgacgagtgcgtcgacgcgatgctcgccggccccaacccggccgacctggtgaagacgatgtccttcccggtgccctccctggtcatctgcgagctgctcggcgtgtccgtcgccgaccgcgacttcttcgaggaccgcacctcccgcatgctcagcatgaccctcccgccgctgacccggcagcaggcgttcttcgacctgcagacctacttggacgagctcgtgacggccaaggagaaagtcccgggggacgacctgctcagccgccagatcgccaagggccgaaaggacagcgcgtacgaccacgacgcactcgtcgacctggcgttcctgctgctgaccgccgggcacgacacgaccggaaacatgatctcgctgggcatgctcgccctgatggagacgcccgagctgcgggcgcgcatcaccgacgaccccggcaccacgccgcaggtcgtcgaggaactgctgcgctacttcaccatcaccgacatcatcaccacgcgcgtggccaaggaggacgtcgagatcggcgggcagaccatccgcgccggcgaaggcgtcttcgcgctgggcggctcggccaaccacgacccggacgtcttcgagaaccccggaaagctggacgtcgaccgcggcgcacgccagcacctcgccttcggccacggaccgcaccagtgcctcgggcagagcctcgcccgcatggaactggagatcgtctacgacaccctgctccgccgcatccccggactccggccggccggccccgctgaagacctgccgctgaagaacgacgcggccatcttcggcctgcacgaactcccggtcatctggtag

SEQ ID NO: 25 - DNA, 50543 bp, Streptomyces amphibiosporus
gtgctcttcccgggacagggctctcagtcgaagggaatgggaagagaactcttcgaccgttttcccgagaccaccgcgtcggcctgcgacgtcctcggatacgacctgcgcgagctgtgcctggagaacccggagggccggctcgacgacacccggtacacccagtccgccctctacaccgtcaacgcccttgagtatctggggtcgttggaggacggggcgccggagggcgactacctgctggggcacagcctcggcgagtacaacgcgctgctcgcggcgggcgtcttcgacttcgagaccgggctgcggctcgtcctcaagcgcggtgagctgatggcgcgggcgcgccacggcgggatgctggccgtactggggcccggcgaggaggagttgcgccggacgctggccgaggagggcatggagcgtctcgacgtcgccaacgtcaacacccccgcgcagaccgtgctttcggggccggtggaggagatcgagcgcgcccagcggcacttcgacgagcgccgggtgcgtacggcgcggctgaaggtctccgccgccttccactcacggctgatgagacccgcgcgggaggaattccgtgcgtttctccggggattccgcttcgcgtcgccgcgtgccacggtgatcgccaatgtgtcggcacggccctacggcgatgtcgcggagatgctgagcgagcagatcgccgggccggtgcggtggctggagagcgtccggtacgtgctggagcggacctccgccgagcgcggcagggaggcgggacccgggaccgtactgacgcggatgctgcggcagatcgacggtgtgtccggggcggggaattccccctccgtctctgcgtccggctccgtgcccgctgcctccgccccggccgagtctccgtccgctcccgcagcgtcaacgtccggcagcgcgcggcccgttgggcgcgccaccgccgccaccgccgatgccgtacgggagcccacccggcccctgctgatctgcgcgccctacgcgggcggcgacgagcgctcctacgcggggctcgccgagcaactgcccgaggcggacgtcgtcaccctggagcggccgggccgcgggcggcgggtctccgagccgctgctgaccgaaccggggcccgtcgtcgaggacatgctgtcccggatacgggaccgggtgagccggccgtacgcgctctacgggcacagcctcggcgcgcggctcgtccatctcctcgcccgccggctgcgcgaggagggcctgcccggcccccgccatctgttcgtctccggcgagtgcggcccctcgcggcccagccgggagcgctacaccagcgatctgccgaccgacgccttctggaagcacctgagagaactcggcggcgtgccggacgagttgttcgagtacgaggacctcaccacgttctacgagcgcgttctgcgcgccgacttcaccgtcctcggggcctgcgcgtacacccccgccgcacccctggactgccccgtcaccgccatgaccggcgacgaggagggcctgaccgaggccgacgtcggggcctggcagcgggagaccaccgcgccgctcacggcccgggtcttcaccggagaccacttcttcatccgggcgcactggcccggcgtcgcacgcgtcgtcgccgccgggctgggcgcccgccgacccgcagggacccgctgacccggaggaacgcgacaggaccgtacgaggacccaccgcggcagcgcgtcgacggcgacgcgcggcgagtcggccccggcacgaccggttccgtccgcaccgccgtgccggctcccgggtaccgcccgggccccggctccgccccaacccgcccgcatcccgggcccgaccacacccggaaccagatccggaacgagatgaggtacgccatggagcaggaactcaagcagtacatggaagagcagttcatgttcgagttcgattcggagatcaccgaggacaccgacctgttcaaggcgggcgtgctcgactcgttcggctacatctcgctcatcgggcacatcgagggggagtacggcgtcaagttcggcgaggaggcgctgctcggcaacgtcgccgtcaccttcgccggcctcgtcgagtccgtggcgtccgcccgtcggcagaccgccgagagcaagtaaccggtgtgcggcatagcgggcttccacgcgagccccctgcacccggagagctaccgggacatcgccggtgccatgctcgcgcagatcgagcaccggggccccgacgaggcgggctgcttcctggacgaccgtacggccatgggcacggtgcggctgagcatcatcgacctcgcctccggctcgcagcccgtcggcagccccgacggccggtactggctctgctacaacggcgagctgtacaactaccgggaactgcgcgccgagttggcgggccgcggggtgtccttccgtacggagtccgacaccgaggtcgtcctgatggcctgggcgcactgggggcgctcgtgcctcgaacgcttcaacggtgccttcgcgttcgccctgaaggacaccgtcaccggtgaactgcacctggcccgcgaccggttcggcaagcggccgctgtatgtggcgcggcacggcgacgcatggctgttcgcctccgagatgaaggccttcctggcctaccccggcttcgagttcgccttcgacgaagagcatctcgcctcgacgttcgccacctggacgccgctgcccgcgcagagcggataccgcggcgtcgaacagctccccatgggcgagtatctgacggtccgcgggacggagaccgaacgcggccgctgggcgtcgctcgacctgaccggcggcgagccgcccgccaccgaggacgaggccgtcgacctcgtgcgcgccgatctggaggccgccgtcgacctgcggctgcgcagcgacgtcgaggtcggcgtctacgcctccggcggtctggactcctcgatcctcgcccatctcaccaaggagcgggccgggctgccgccgcgtacgttctccatccagttcgaggacgccgagttcgacgagaccgccgagcaggaggagctgaccaagcacctcgggacgcaccactccaccgtccgcgtctccgactccgacgtcgtggagaccttccccgaggccgtacgccacgccgaagtccccgtcttccgcacggcgttcgtgccgatgtacctgctggcgcagcatgtgcgcagcgaaggcgtcaaggtcgtgctcagcggcgagggcgccgacgaggcattcctcggctacggcatcttcaaggacgcccggctgctctccgagtggcacgagctggacgaggcgacccgcatgcggcgcatggcgcagctctacccgtatctgcgccacttcagcggcgaggacgggcaccgccggatgctgggcctctaccggcagttcacggaggagaccatgcccggtctcttctcccaccagatgcggttccagaacggccggttcgccgtgcggctgctcaaggacgcgggcgaccccttcgccgcggtacggcggctcgtcgcggaggagcccggatacgcggagctgtccgcggtgcagaaggcacagtggctggagttccgtacgctgctcagcggctatctgctcgccacccagggcgagcggatggcgctcgcgcacggcgtggagaaccgctgcccgttcctcgacccggcggtggtgcgccgcgcggcgtccgtcaacggccgcttcggcgacccgtacgacgagaagtacctgctcaagcgcgcgtacggggacgtgctgcccgaacgcatcgtcagcaagggcaagttcccctaccgggcgccggacagcgccgcgttcgtacggtcccgtcccgactaccgcgacctgctggccgaccccggcaccctcggcgacatcggcgtgctcgacgagcgcttcgtgcggcgcttcaccgaccgggtcttcgacaggccgcccgagcggatcggcaccaaggagaaccaggcgttcgtcctgctggcgtccacggtctggctgcaccactggtacgtgcgcggcaacgcccgccgcgacacccccctcgctgtccccctgtacgtcgtcgaccggcgcagcagcgcgctgccggcctaggacggagatcctgcgatgaaggaagaatccggcgccctccccgaggaaggccccgtcggcaccgctgtcggcaccgccgccgacggcgcggccggcggcccggtggacgggcaggacatcgctgtcgtgggcctgtccctgcggctgccgggcgcacggaacccggaggagttctgggagcacctggccgcgggccgctcgctgatcagcgaggtgcccgagcggcgctggcgcaaggaggaccatctcggcaacccgcgccgggagttcaacaagaccaacagcgtgtggggcggcttcgtcgacgacgccgactgcttcgacgccgagttcttccatgtctcgccgcgcgaggcccgctccatggacccgcagcagcggatggcgctggagatgagctggcaggcgctggaggacgccggataccgggccgaccgggtcgccggctcccgtacgggcgtcttcatgggggtgtgccactgggactacgccgagctcatcgagaaggaggtctccgaggtcgacgcctactacccgacgggcgccgcgtacgcgatcatcgccaaccgcgtatcgcaccacttcgatttccgcgggcccagcgtcgtcaacgacaccgcgtgcgccagttcgctggtggcggtgcagcaggcggtgcaggcgctccagtccggggactgcgaccacgcgctggccggaggcgtcaacctgacctggtcgccacggcacttcatcgccttcgccaaggcgggcatgctctcgccggacgggctctgccgcgccttcgacgcggacgccaacggctacgtacggggtgagggcggcggcgtcgtcctgctgaagcgggcggcggacgcccgccgggacggcgaccccgtacacgcggtgatcaagggcatcggcagcaaccacggcgggcgcaccagttcgctcaccgtcaccaaccccgccgcgcaggccgagctgatcgccgggatctaccggcgggcggggatcgccccggagtccgtctcctacatcgagacccacgggccgggcaccccggtcggcgaccccatcgaagtcagcggcctcaagcgggcgttcgcgcagctcggcgagggacgggaggccgagccgtccgggcaccgctgcggcatcggctcggtgaagaccaacatcgggcatctggagggcgccgcgggcatcgccgggatgctcaaggtgatcctggcgatgcgtcaccgcaagctgcccgcgacggtgaacttccgccgtctcaacccgctgatcacgctggacggcagcccgctgtacgtcgtggaccggctcaccgactgggagacggacggcgacgggacgctgcgggcgggtgtcagctcgttcggcttcggcggcaccaacgcgcatgtggtgctggaggcgcccggcggtcacgcggcggaggtcacggacgcggaggcggtcacggacgcggaggcggacgccgacgtggacggcgggccggacgaggggcccgacgagggcgccgaaccgcgtgctctgcggctccccgtctccgccgacgacgaggagcggctgcgtgagctgtgccgctcgctcgccgagtgggcccgtgcccgcgaagccgaaggcacggcgccgccgctggccgacatcgcccgcaccctgcgcgaagggcgggtgccgatgcgggagcgtgcggtcttccgcgcgcggagcgtcgccgagtgggcggaacagctcacggccctcgccgaggggaccggcggggagccgcccgctggctgtctgcgcggacgcgcggaggacggcgccggggacggcctggacgccgacgatgtcgcggccctgaccgcgcgctggcgggagcgggacgaggaggagaagttcgccgcggcctggacgaggggcctgcccgtggactgggcgcagtggcccgccgagggccgccgcgtccatctgcccggacaggtcttccagcggacgccgcactggttccgtccggacgaacagccgcgcggcgaggccgagtcggcgggcggtgcggcggctcagcgggacaccgctccagagcgggacgcggcgtccgggtcggaacgcgggcccggcgcaccggagccggcgggaccggtgggagggccggggctgccgggcgagggcgtccaggacgggcggggctggcacttcccgctgcgcttcgccgccaccgaccccttcgtacgcgaccatctggtcttgggcgcccgtatcgtccccggcgtcgtggcgctggaggccgtgaccgccgccgcggcacgtcccgctgtggccggtgcccgtgccggtgcggcgccgcacatccgcaacgcggtgtgggtgcggccgctgcgcgtggacggccaagtcctcgaaacgagcctgcggttgacgcccgccggcccggagtccggcggcggctacgactgggccgtcaccgacgccgcgggcacgccgtacagcagcggtcgcgtcgagtacgccgacgggcccgcgcccgccgccacggatctggacgcgctgcaccggcggcacacccgtcccgtggaggtggccgggggatacgcggcgctgtacgccagcggcatcgagcacggcccggcgctgcgcgccctgcacacgctgcgcgccgggcccgaagggctgctggccgagctgcggttgcccgccgagccggctgcgggcgcggctctccagcccgccgttctggacagcgccctgctggccgtgctcgcgctcggtacgggcggtggcgacggcacagagggcaccggcggcacagacggcgcgggctggcgccgaccggacgcgcccgccgtgccgttcgcgctggacggcctgaccgcgtacgccccgaccacggccacgacatgggcctggctgcggcccgcgggcggacgccgtcccggcgccgccgacatcgatctgttcgacgagcgggggcggttgtgcgcccgtctgacgggctacacctcgcgtgaactgtccaccgggagcccggcgttgagggaagcgcgcgtttctgcaccggcaccgggcgaaggggcggcgggcgaggaggctccggggaaggacgctccgggcgagctgctggaggtcaccgggcgctggacgcccgcgcccctcggcctccccgccgccgaggcgggaccggccgcggcacagtcgggcgccgccgctcccgtcacggtgctgaacgccgccctggacgccgacctcgtcgccgcgagcgccgcccgtctcggcatggacgtcgagcacctggccgtaccgcgcgacgcgggcgacgcggacgccatgaaggcggcgttcgcggcctgttacccccatgtccggcggctcgtcggacagtcgcggcgcgttctgctcgtggcccccggcgcacccgactccccggtcttcgcgccgctggcggccctgctgaagacggcacaccaggagaacccgtccttccacggcaccaccgtgctcctggagggcttcgacccgcgtgactccgcacgcttcgagcaggtcgtccgtacggaggcggcagcgacggcggacgccgcgggcggcgcggcggacgaggaggtcgcccacaccgccgacggccgccggctgcgccatgagacggccgaactgccgcacggcacaacgggcgagagcctgctggcggagggcggcgtctactggatcaccggcggcgcgggcggcatcggcctgctgctggccgagcggctgtgcctgcggtacggggcgacggtcgtcctcagcggacggtcgcccgctgcgcccgcggccgacgcgctcgcctcccggctcacccgcggcacgctggcctaccgcggcgcggacgtcaccgaccaggacagcgtggacgcgctggtggccgccgtgctggccgaacacggccggatcgacggtgtgttccacgccgccggggtgctcgacgacggctatctgacggccaagccgctcgccgggaccgaggccgtgctcgcgccgaaggtggacggcgtcacctgcgtcgaccgcgccacgcgcgcgggcgctccgggcttcctgctggtcttcgggtcggtcgcgggtgccttcggcaacgcggcgcaggccggctacgccgccgcgaacgcctacctcgacgcgttcgccgcccggcggcaggccgccgggctgaccacccgggccgtcgactggccgctgtgggccgagggcggcatgcgcgtggacgacgcgagcctgaagtatctgcgcaagcgcaccggcaccgtgccgctgccctccggcaccggcctcgacgcgctggagcgtgcgctgcacaccggttcgccggtgcggcgcgtggtcctctacggcgaccgtccggcgctgcgggtgtacgcgggtctggaccgtccgcaggtcacgggcgcacggtccggctccgcgtccgcgtccgcgccgggctcctcgtccgggtccgtgtccgccgtgcgcggcgaggggaccgggacggcaccggccgcgctcaccgacgccgaactcctcgtccgcacacaggacttcctgcgggagcagttcgccgaggtcaccctccaggacgccgagcagatccaccccgaggagaagctggagacgtacgggatcgagtcgatctcgatcgtcgatctgacgagcaggctggaggacgtcttcggctcgctgcccaagaccctcttcttcgagtacgtcgatctgaagggcgtggccgagtacttcgtggccgagcaccgtgcgcggctgaccgaactcttcgcgccggaggagccgcaggcgtccgaggcggcggagcccgcaccggaagaacccgtggcgcccgcccccgtaccggtggagccggccgccgcggcacccgcacccgcacccgcacctgtaccggcgcctccggcgccaaccgccgcaccgggtacgtccgttgaggccgtccccgcgcccgttcccgcttccgtacccacgccgcgccccgcccccgccgggaacggcgacatcgctgtcgtcggcatggcgggccgctacccgggcgccgacaccctggaggagttctgggagctgctcagcgagggacggcacagcttcgagcccgtgccgtcctcgcggtggccgcacggcgacctgtacttcgacgagcgggacgtgctgggcaagaccacggtgcgcaccggcaccttcctgcgtgacgtcgacgccttcgacccccgctacttcagcatctcccagcgcgacgccgaactcctctcgcccgaggtgcgcctcttcctccaggcgggcgtgacggctctggaggacgccgggtactccaaggagacgctgcgccgccgctacgacggcgacgtgggcgtgctcgtcggctcgatgaacaacagctacgcctactacggcttcgagaacatgctgatgcgcggcaccgcgatgagcggcagcgaggtcggcgtgatggccaacatgctctcgtactactacgggttcacgggcccgtcgatgttcgtcgacaccatgtgctcgtcgtcctcggcctgtgtgcaccaggcgctgagcatgctgcgcggcggcgagtgccgcatggtcgtcgtcggcggcatcaacctgatgctccacccgtacgacctgatcgccacctcgcaggcgcacttcaccaccaagtcggcggaggtcgtgcgcagttacggactcggcgcggacggcacgatcctcggcgagggcgtggggaccctggtgctcaagccgctcgccgaggccgtcgcggacggcgaccacgtctacggcgtcatcaagggcagcggcatgaccaacgccggtgtgcgcaacggcttcacggtgcccagcccgcagcagcaggcgagggcgatcgagagggcgctcgacgacgccgccgtggacgcgcgcaccgtcagctacctggagggccacggctcggcgacctcgctgggcgacccgatcgagatcaagggcgcgagcctcgccttcggccgggacacccgggacgtgggctactgcgcgatcgggtcggtcaagtccaacgtggcgcatctgctgtccggttcgggcctcgtcggcctgacgaaggtgctgcttcagctacggcaccggacgctggcgccgtcgctgcactccgaaaccctcagccccgccatcgacttcggctcgacgccgttcgtggtgcagcgtgagcgcgccgagtggcggcgtcccgtcgtacacggcgcggaggtgccgcgccgcgcgggcgtcacctcgatcggcgcgggcggcatcaacgtgcacctgatcgtcgaggagttcgacggcacggtgaactccgcgcccgacgacggcggttcacagcttctggtgttctccgcgatgacgccgcaggccctgggcaccgtgctgcgcgacgcgcaccgccatgtcgccgacgaggcgcccgcgctcaacgccctcgcgtacaccctccagaccggcaagaacgaactcccctgccggctggccttcgtcgcgcacggcacggccgacgccgaggcccggctcgccgcgctggcggcggtggactggacgtccggcgcacccgcgctgcccgacgctgtgcgcttcaccgagagcacgctgcggaagcggcgcagcgtggcggccgccgacgtcgaacgggccctggcgcaggccgacttggcggagctggccggatactggatctccggcgcggccgtggactgggacctgctgtggccctcgggtacccgtccggcgaagctggcgctgcccgcgtacccgttcgagaaggtgcgctgctggtacccgggtttcgacgacgcccccagcgtgctgcggcccctggccttcacccggcgcgggcacccctgggtcggcgtcaaccgctccgatctgcacggcgtgcgcttcgcgctggagctgacgggcgacgaactcctcgactacgtccacacggtgggccgcacccgccgcttcacgagcgtcgccctgctggacggggcgctggcgttcgcgcggctcgcgggcctggacggcgcgctgcggctgcgcgacgcgcggtgggcggagctgccctcgcccggcgacgccacggaggtcttcgagtggcggctcgcgctctccggcgagggagcgtccggtgacgcggcgtccggcggaggagcgtccggtgcgggcggccatcgtgtcgagctgtggcaggccgagcgcggcacgctgcacttctcggcggaggtcgtaccggcaactgccgtggcggcgggggccgttgacgcgcggcctgccgacgccgcggcgctgctcgccgcacccgtgacgctggacggcgacgcgttctactccgcgctcggcgaggcgggcgtggacgcccgcccgtacgcgcgcgccgtcaccggcgtcaccgaggccggcggacggcggctgctcgtccgtgtcgccgaaccggcgatgtgccaggacccgcacaagcagcacgtcagcattcccgcgtgggtgctggcggggctggcacagggcgtgcagcacgccacgggccggccgcgtacgacggcactgcgcgccgccgcgctgtacggcgccgacctgacggacacccgcgccctgctgctggagcccgtcgcggaggccaccttccggatcaccttcctggacggggacgggcgggcgctgggcgcggtggaggacgcggagttcaccgccgggacgttgccgccgtcgctggagggcggcgccgtaccggtacgggccggactgccgggcgcggcacgcccgtcggcgacggcctcggcaccggcctcggcctcggtaccggtaccggcgctcgcggccgcacccgtcgcacctgccgtgcccgtcgagcccgtcgagcccgccgcggaggcggacgccgacgcgggggacgcgctcgtcgccgtgctgcgggagacggtcgccgacctgctcaagttcgagctggacgagatcgacctcgacacccacttccacgcgtacggcttcgagtccatcgccctcgcgcggctggcctccgaactcaacggcctgctcggaacggacctcagccctgtcgtgttcttcgagtgccccgacatccgcagcctcgccgcccatctgcgcgagcgctacgacgcggagacggcggcccgcgccgtccgcggcaccggcggcggcaccgggacggacgcggcccgggcgccgactcccgctccggcaccggcagtcggggcggcgtccgcggccaccgcgcccgctcccctctcgtccgccgagcccgtgtccgaccacgaggccgactacccgggcgccgtcgccgtcgtcggtgtggcgggccgcttccccggcgcgccggacgccgacaccttctggcagcggctgcgcgcgggggacgacctgatcggggagtacccgggcgaccggttcgacgagcgctacaccggcgtcgtcgcacggtcggacttcccgaagttcgcgggcgtcctcgacgacgtcgaccgcttcgacgccggcttcttcaacctctcccggctcgaagccgaactgatggacccccagcaccggttggccctggagacggtgtgggcggcgctggaggacggcggctacgcgcccggccggctgccggagaacaccggcgtctacgtcggcgtctccggcagcgactaccaccacctgctgaacgccagcggcgtggcgccggacggcttcaccgccaccggcaacgcccactcgatgctggccaaccggatctccttcgtcctggacgtgcacggcccgagcgagccggtggacaccgcgtgctccagctcgctcgtcgccctgcaccgcgcggtggagagcatcaggtcgggccgctgcgacatggccctggcgggaggcgtcaacctgctgctgagcatcgacacgttcgccgcgacgcagatggcgggcatgctcagcccggacggccgctgcaagaccttctccgcggacgccgacggctacgtacgtgccgagggcgtcgccgcggtgctactcaagccgctggagcgggcgttggcggacggcgacccggtctggggcgtcgtacgcggcagcgccgagaaccacggcggccgtgccggttcgctcaccgcacccaacgccgtcgcgcagaccgcgctcatccgcgaggccatgcgcggcaccgaccccgacagcgtcggctatgtcgaggcgcacggcacgggcaccggtctcggcgaccccgtcgaggtcggcgccctcgacagcgcctaccgcgcgctgcgttcggaccgcgggcgtgtcgagagcggcaccgcaccggtggcgctgggctcggtgaagaccaacatcgggcacgccgagtcggcggccggactcgccggggtgctgaaggtgctgctggccatgcgccacggcgaactgccgccgaccctccactgcgaccggctcaatccccatctgccgctgtccggcggcgggttcgaggtggtgcgcgaggtacgccgctgggagccgcggctcgacgcggacgggcggccgtggccgctgcgcgccggggtgagcagcttcggcttcggcggcgccaacgcgcacgtcgtgctggaggccgcacccgcggcggcacgggaacgggccgtacgggaaacggcttcgcggagtgcgtccgtacggtccgcgcacgggacgcagggcgctccgcaggccgtcgctccgcaggccgtcggtccgcagatcgtcgccgtctcggcgcgtgacggcgagcggctgcggatcgtggccgagcggctgcgggacttcctgcggcgggagcacggcgcgggccgggcgcccgcgacggccgacctcgcccgcacgttgcagacgggacgggaggcgatggaggcgcgtctcgccttcgtcgccgaggagaccggggacgtgctcgacgtactggaccggttcctcaagggcgaggagcccgacggctggcacaccggcgcactgcggcgctcgcgcggcgcgggagtgcggcgcgaccgggcgcaggacccgcgggtgacccgcgcgctgcgggacggggacctcgatgcggccgccgcgctgtggtgcgagggggccctcgtcgactggcagtcgctgcacccgccgggggagcgccgcaccgtgcggctgccctcgtacccgttcgcccgcgaacgctactgggtgcccacggacggggcggcaccgccaccggagaccggcgggcccggcggcgtcgaggacggcggcgtcgagtacggcaccgggtccggcgccgcccaactcggcgacagcgggagcgcgttcgacgccggagcccttgccgcggtgctcgacgcggtcctcgacggacgggccgatcccgacgacctcgcccgtacctgacgccgtacgcgcccgccccgccccctcttctcttcggacgagcggccccgcgccgcaccgtgacctgacctggaacggatcccacagtgagtcgaaacatcctgcgtgtgccggcatggcgagacgagccgtcgcgcgggcaggcggcaccggccggagtccgccggctggccgtcctgtgcgacgtcccggacgcggaggcggcgctgctgcggcagcactcgccccgcctgcccgtcgtcctggtggagagccgggacgacgggcccgccgccgcgtacgagcacgcagccacccggctgctcgccgagctccagaggctgctgggccgcccggcggcgggtccgtgccgggtgcaggtggtgtgccgggagagcacgccgcagggctgggcgggactgctcggcatgctgcgtacggcggcgcaggaagacccccgactccggggccagctcatcgagttcgaccgactgccgggcggcgccgagctggcgcgcgtgctggacgaggaggccgccgaggaggcggatcatgtgcggcgggccgccggtgcagccggtacgggtaccggaaccggagccgtacggcaggtgcgccactggagcgcggcccggtcggcgggtcgcgcgtcgtccgccgggaacccggcgccggtgtggcggcccggcggcgtctatctcgtcagcggcggtgccggcggcctcgggcggctgctcgccgccgacgtgcggcggcacgcgcccggcgcggtcacggtcgtgtgcggtcgtggcccggcgccgtggcagggggcggaaccgcccgccgacggcgtcgagtaccacagcgtggacgtcaccgaccgggccgcggtggccgccctggtcgatcgtgtgctgagtgcacacggcaggctcgacggtgtcgtgcacgcggcggggctgctcgccgacgactacgtggtccgcgcgtcgcaccgcgagacccagcgcgtactggcgcccaaggtcgccggtctggtccacctcgacgaggccacccgcgaactgccgctggacttcctggcggccttctcctccgccgccgggacgctcggcaacgcgggccaggccggttacgccgcggccaacggcttcctcgacgcctaccagacccaccgcgccgcgctggccgaggcgggcgagcggcacggccgttcgctctcggtcggctggccgctgtggcgggacggcgggatgaccgtgccggacgagcaactgcccgaactcaccgagcggttcgggcgtccgctgacgaccggcacggcgctgacggcactgcacgccgcgctcgccctcggcacaccgcacgtcctggtacgggacggcgcggaggcggacgaaaccggagccgtcaacgcaaccggggccgggaccgcgaccgggatcgcgaccgaggtcgaggtcccggctgtgaacgaagccgtcggcacggccgtcgacgacgccctggaggacgacgccccggagggggacaggaagggaactccggctgtggaaccgcgcctccgcgtactgcccgcgctgaagcaactcgtcgccgagaccgtgcggttggacccggccgcgctggacgccgccgcgccgctggacggcttcggcatcgactcgctggccgtcacccggctcaaccgccgcttcgcgcagtggttcggggcgctccccaagacgctgctgtaccagtacccgacgctgaacgagctggccggatatctcgccgagcaccatccggagggctgccgccgctggctcgccgacacggcgtccccgtccctgtccccttccgcgtccgcgtccgcttccccgtccccgtccccggcaacgtccacgtccgtgtccgctccctccgctcaggagcggcggccgtcaactcccgtcgccgccggggccgttcgcacggccgggacgaacggcacgagcggtgctgccgccccggtttccgccgaggcccccgttcccgcccgtacgtcacctgtcgacgagccgatcgccgtcatcggtctgcacgggcgctaccccggtgcccccaccctggacgccttctgggagaacctgcgctccggccgggacggcgtcaccgagatccccgccgaacgctggccgctggagggcttctgggagcccgacgtcgagcgcgcggtgcgcgagggcgcgagctacagcaagtggggcggattcctcgacgggttcgcgcagttcgacgcgctgtttttcgggatcgcgccgcgcgaggccgccgacatggacccgcaggagcggctgttcgtggagagcgcgtggtccgtgctggaggacgcgggctacacccggcggcgcctcgccgagcaacaccgctcccgcgtcggggtgttcgcgggcatcaccaagaccggcttcgaccggcaccgcccggccgcccccgcggagacggacgcttcctccgccacgggcggcgtgccgcccgcctccccgcgtacgtccttcggctcgctcgccaaccgcgtctcgtacctgctcgatctgcgcgggcccagcatgcccgtcgacaccatgtgctcggcgtccctcacggccgtgcacgaggcgtgtgagcatctgcggcacggcgcctgcgagttggccgtcgccggcggcgtcaacctgtatctgcacccctcgacgtacgtggagctgtgccgttcgcggatgctcgcccgcggcggcgagtgccgcagcttcggcaccggcggcgacggcttcgtgcccggcgagggcgtcggcacggtgctgctgaagccgctgtcgaaggcggaggccgacggcgaccccgtacacgcggtgatcctcggctcggccatcaaccacggcggccgcaccaacggttacaccgtgcccaatccgcgcgcgcaggcggagctgatccgcgaggcgatggaccgcgcgggcgtctccgccgacgaggtcggctgtgtcgaggcgcacggcaccggaacggcgctcggcgaccccgtcgagatcgagggcctggcgcaggcgttcgccgaccgtacggacacggcggcgccgtgcgccctcagttcggtgaagtccaacatcgggcatctggaggccgcggcgggcatcgcgggcctgacgaagctcgtgctccagctccggcacggcgagctggcgcccacgctgcacgccgaagtgcccaaccccgacatcgacttcggctccgtaccgttcgcgctccagaccgccgcggcgccctggccgcggaccggagggaacagcggacggcggatcgcggggctgtcgtcgttcggcgcgggcggggcgaacgcgcatgtggtcgtcgcggagtacacgggcgccccggccgcccgcacctccgcacctgccgtggccgacgggtccgccgcgaccgccgggtccggacgcccggtgctgctgccgctgtccgccaggacgcccgaggatctgcgggcccgcgccgtacagctcgccgactggctcgactcccgcgacgcggtcgatctgacgtcggtcgcggcgacgctccagacgggccgcgaggccatggacgagcggctgtgctgtgtggcgtccacgcccggcgaatggcgcgaacagctccgtgcgttcgccgacgacccggagcgcgagggcccctggcaccgtggccgggtgcgggcgaccggcgaggcgctggccgcgctggcggagaaggacgaactccgggcgctcgtcggacgctggaccgcccgcggcgagtgggcggaactggccgcgttctgggccaagggcatgccgctggactggagccgcctgtacgcggacggccgggtcccggcccggctccatctgcccgcctatcccttcgccgggcgccgctactggcccggacccgcggacgtacggaacacggcggacgcgcaagcgccccgcacctccacgcccagcccgtccacgctcagcacgtcaactcccggcgcgtccaggcccgttgccgtcgcgcccgttgccgccgcgccgtcggcggagtcgtacatcgaacgcgtgctgctcgacgcgctcggcgaggccctccagatgacgcccgcggagatcgacccgcgccgcccgttcgcggactacgggctggactccatcctcggcgtccacctggtcaacgtcctcaacgagacgctgggcaccggcctggagaccaccgacctcttcgaccacggcaccgccgagcggctgcgcgcgttcctcaccgagacctacggcggcacggtgaccgtccccgacggcacgggcgccgctgccgagttcgtccccgacgctcccgtcccggcccgcgaggcggacgacccggtcgccgtcgtcggcatggccgcgcgctacggcgacgccgaggacccccgcgccctgtgggaccgcctgctggccggtgacgacctcgtcgagccggtcacccgctgggacctggggcccgaagtgacgtgccgcgcgggcagtttcgtacgcggcatggaccggttcgacccggtcttcttcgcgatctccggtgtcgaggcggcccatatggacccgcagcagcggatcttcctggagcagtgctggaacgccctggaggacgccggatacaccggcgagcggctgcgcgagcgcaactgcggcgtgtacgtgggctgttacgcgggcgactactacgacagcatcggcgaccgcgccccggcccaggcgctgtggggcaccatgggctcggtcgtcgcctcgcgcatcgcctatcaactcgacctgaagggcccggcgctcaccaccgacacttcctgctccagctcgctcgtctccctccatctggcctgccgcgatctgcgcacgggcgccgccgacatggcgatcgcgggcggtgtcttcctccagacgacgccgcggctgtacgaggccgcgacccgtgctggcatgctctcgcccaccggccgctgccacagcttcgactcccgcgccgacggcttcgtccccggtgagggcgcgggcgccgtcgtactgaagcggctgtcggacgcgttgcgcgacggcgaccacgtctatggcctcgtccgcgccaccggcgtcaaccaggacggcaccaccaacggcatcaccgcgcccagcgcggcctcgcaggaggcgctgctgcgcgaggtgcacgcgggcgtcgcgcccggcggcgtccagttggtcgaggcgcacggcaccggtacgcagctcggcgacccgatcgaattccgcgccctcagccgggtgttcggggacgcgcccgccggcagcgtcgtcctgggctcggtgaagaccaacctgggacacacccagttcgcggcgggcatcgccggtgtcctcaaggcgctgctggcgttgcaggagcagcgcgttccgccgtcgctgcacttcgcggaggccaacgcgcgcgtaccgctggacggcagtcccttcaccgtcgcgacgacggcacagccgtggcccgagcccgccgagggaccgcgccgggcagccgtcagctccttcggggccagcggcaccaacgcccatgtcgtactggaggagcacccgcccgtacgggcgaccacggggccggagtccgccggaggggacggcgaggccgccttcctgctgtcggcccgcacccccgccgcgctgcgggccgtcgcggagcggctgctcgcgcggatcgagcgtgaaccgggcctgcccgcccggcaggtggcctacagcctcgccgccgggcgtcgccacttcccgcaccggctggccgtcgtcgccaccgggctgcctgctctcgccgcccggctgcgtgcctggctggcggacgaacagccgggcggcgaggggacgttgctgcacggcgtcgcccacgccggaacgcggcaggccgcgctgggcgggctcgcacccgccgagctggcggcggcgtatgtgggcggcgccgaggggccgttcgccgagagcttcccggccggggcgcggcgtcaagtgccgctgcccacctatccgttcgagcggcagcgctactgggcggaggggacggacggacacgctgtcccggccgccgccggtacgtccgccgtggagccgggcggccggcgcaccgcgtaccgcacgcggctcacgggtgaggagttcttcctcgccgatcaccgggtgggcggccgtacggtgctgccgggcgtgctgacgctggaggcggtacggcgcgcggtcaccgccggagacggcggcgacgggaccggaatcggcaccggcggcggtacgggcgtcccgactcccctgcggttgcgtgacgtcgtgtggcccgcgcccttccccgtcggcgcggacggggccgaactgcgcgtcgatctcgacggcgacgccttcgccgtacggcaggacggctcgtccgtgcacgcgcagggccgctggacgatggtgcccgcccccgccgcctccacgtcgctggagaccctgcgggagcgctgcgcacgccgcacgctgacgcgcgagcagtgccgtgcggcgctggaggccgtcggcatccggcacggtgagcggctccgcgcgatcgagcaactgtccgtcggggacggcgagttgctcgcccggctggtgctgcccgcgaccgtggagacggggacgcaggccacggagacgttcggactgcacccggcgatgctcgacagcgccatccaggccgtcgtcggactctacggcgacgagaccggagccctcggcgagcgtccggacgcccccgcactgcccttcgccctggacaccgccgacgtcctcgctcccaccaccgaccggatgtgggcccatctgcgctgggcggacggttacgcgcccggcgaggccggagaggtgacgaagaccgacatcgatctgtacgacgacgcggggcggctctgtgtgcgcctgcgcggctacgcctcccgccgcgtcacgcccgccgccaccggctccgcgaccgcggccccggccggtacggacgacgccgacgcgccacgggcgcagctcctcgcaccggtgtgggacgcacagccgcatgcggacggcccccgcagccccgagcccggtgcacacgtcgtcctgctcggcggcacaccggaggaacgggacgggctgcgccgtctcgtcgccgacgtcaccgtcgtggaaccggaacgccacgcgtccgccggggagttggccgcgctgctgccgacgggcgccgagcacgtcgtctggctcgcgccccgcgacgcgtcgcccgccgcctcctccgccgagggacccgacggggcgctcgccgtcttccggctcgtcaaggcgctgctcgcggacggcgcggacgccagggagctgagcttcaccgcggtcacccgccaggcccggctgctgcccggcgacgcggactgcgacccggcgcacgccggagtgcacggtctgctgggcacgttggccaaggagtacccgcactggcgggtgcggggcgccgacatcgagcgggacgtctccgtaccgtggccggagctgctgtcgctgcccgcggatccgcgcggcgaggtgtcggcccgccggcacggcgagtggtaccggcagcggctgctggaggtcgccctcgacgcgtccggcgccgcctccttctccgccggctcccaggcccccaacccccaggcccccaactctcaggcgagtggcgcccgttcggcactgcgcgagcccggcggcgtcgtcgtcgccatcggcggcgcgggcggcatcggcaccgtgtggaccgagcacatgatgcgacggcacggcgcccgcgtggtgtggatcggccgccgcccctacgacgaggagatcgccgcgcggcaggaccgcctcgcggcctgcggcccgcgcccggagtacgtacgggccgacgcgaccgacgcccgcgctctgcgccgcgccgtcgcggagatcgagcgccgccacggccccgtacgcggcgtcctgcacaccgcgatcgtcctcggcgaccagagcctggccaggatggacgaggccgccttccgcaccacgtacgaggccaaggccgccgtctcggtcaacatggccgacgcgttcgccggtcagccgctggagttcgtggccttcttctcctccatgcaggcgttcttcaaggcgccgggccaggccaactacgcggcgggctgcacgttttccgacgcctgggccgagcggctctccaccgcgctcgactgccccgtgaaggtcatgagctggggctactgggccggagtcggcatcgtgaccgccgacggctaccggcagcgcatggcgcagctcgggctcgggtccatcgaaccggacgagggcatggccgccttcgacgcgctgctggcctccccgtaccggcagctcgccctgctcaaggcgaccgacagccgcagcatcgacgggatctacggcgacgacgagctgcggcaactgccgcccgccgcgcccgcgctcgcggacaccctccgcacggaccgccccgaccggaacgcggagatccggcggctgcgggagcaggccgacggccacgccggagtcatgtacgacgctcttgtccgcgtcacctgggcgctgttgacgtcgctgggactcttccgcgacggccgcgcggccaccgccgccgagtggcgcgccgtcggcggcatcgaggagcgctaccagcgctggacggaacacacgctggaggtgctgaccgccgccggacgtctgcgccgcgcgggcgaggaccggtacgccgccgtcgcccccggagccgtacccgccgaggacgcctgggccgagtgggaccgggcgcgggaggtgtggctcgcggacgaggccaagcaggcgcaggccgtgctcgtcgacaccacgctgcgggagctgacggcgatcctcaccggccgccgcgccgccaccgacgtgatgttcccgggctcctcgctgcggctcgtcgaggccgtctacaagaacaaccccgtcgcggactacttcaacgaggtgctcgccgacaccctcgtcgcctacctcgaacaccggctgcgccaggacccgtccgcgcggctgcgcatcctggagatcggcgccgggaccgggggcaccagctccgtggtcttccggcggctccggccgctggccgggcacatcgagacctacacctacaccgacatctccaaggcgttcctgctgcacgcccggcgtgcgtacggggagatcgcgccgtacctggacgggcagctcttcgacgcggagaagccgctcgccggacagccggtcgccgtcggcggacacgacgtggtgatcgccaccaacgtgctgcacgcgacgggcaacatccgcaacaccctgcgcaacgcgaaggccgccgtacgcgccaacggcctgctgctgctcaacgagttgagcgacaacatcctcttcagccacctcaccttcggtctgctggacggctggtggctctacgacgacccggcgccgcgcatcccgggatcgccggggctgacgccgcagagctggcgccgcgtcctggacgaggtgggcttccgcgggtcgttcgtcgcagccgagggcgccgacgacctcggccagcaggtgatcgtcgccgagagcgacggagccgtgcggcagccgcggcccggcggggtctccgccttccggggcagcctgccggaggcgcggccggctcaacccacgggcggggcggggcacttggcggtgccggcggagcacggctccgcgcctgccgtgaccgtgccggtcaccgccgcgtccgcttcctccgcaccgggctccgcgcccgccgccgtcccgtccggtgatccgtccggcgacgggagcatggccgcacgtgtggccggaccggcacgggacctcttccgggggctcgtcgcggacgtcctgcaactgcccgtcggcgacatccgtgccgacgtgcccttcgagcggtacggcatcgactcgatcctcgtcgtccaactcaccgacgccgtacggaaggtgctcgacggcgtgggcagcacgctcttcttcgaggtgagcacggtcgacggcctcgtggagcacttcctgcgcacccggccggacgaactcgccgcgctcgtcggcgtatccgccgcggagcacccggaacccgcggcggaagccgccgcaccggaggcggtcaccgaggagccggcggcctctgtacccgcacccgcacccgtagccgctcctgtctccgtacccgtgcccgccgccccgggtgaggacgtccccgtcgccgtcgtcggcatggccgggcgctaccccggcgccgccgacctggacgccttctgggagaacctgctcgcgggccgcgactgcgtcaccgagatccccgacggccgctgggaccacggccgttactacgacgagcgccgcggcgtgcccggcaggacgtacagcaagtggggcggattcctcgacggcgtcgacgagttcgactcgctgttcttcggcatctcgccgaaggccgcgtccacgatggacccgcaggagcggctgttcctccagtgcgcgtggacggcgctggaggacgcgggccacacgcgggcctcgctgcgctccgcctcccgcgcccggctgcccgaagacgccggggacatcggggtgttcgtgggcgcgatgtactccgagtaccagctctacggcgcggagcagggcgtacggggcgagcccgtcgtcgtacccggcagcctcgcctcgatcgcgaaccggctgtcgtacttcctcgacgcgtccgggcccagcgtcgccgtcgacacgatgtgcgcctcggccctgtccgccgtgcatctggcgtgtgccgcgatccggcgcggtgagtgcgcctcggcggtggccggcggcgtcaatctgtcgctgcaccccagcaagtacctgatgatcggcgagggacagttcgcctcctcagacgggcgctgccgcagcttcggcgcggacggcgacggctatgtgcccggcgagggcgtgggcgcggtgctgctgcggccgctcgccgacgccgtggccgacggcgaccgcgtgctcggcgtgatccgcggcagcgccgtgaaccacggcgggcacacgcacggcttcaccgttcccaacccgctcgcccaggcgtccgtgatccgcggcgcgtggcgccgctccggcgtggacccgcgcgacatcggctgcatcgaggcacacggcacgggcaccgcgctgggcgaccccgtggagatcgccggactgaacgccgccttcggcgagttcacctccgagcgcaccttctgctccctcggctccgccaagtccaacatcggacatctggagtcggcggcgggcgtcgcgggcctggccaagatgctgctccagatgcggcacggcacgctggtgccgtcgctgcacgccgagcgcaccaaccccgaaatcgacttcgccgccacgccgttcgtgctccagcgggaggccgcgccctggccgcgccgcgaggggcgcccacggctcggcggcatctccgcgttcggcgcgggcggttcgaacgcgcatctgctcgtcgaggagtacgtaccgacggcggcaccgccgcggcgtgcggcgccgggcccggtcctcgcggtgctctccgcgcgggacggcgagcgcctgcgggagtacgccgggaagctgcgggacgcactgcgctccgggcagtggaccgacgaggacctgccggacatcgcctacaccctccaggtcggacgggaggcgatgagcgcacggttcgccgccgaggtgagcaccctggccggactcatggacgcgctggacgcgtgcgcacggggcgccgccctgccgcccggcgcccggctgcgtaccgacggcgggcggggcggaccggtccaggacctcgcggacgacgaggacttccgggagaccgtcgtgcgctggctgcgccgcgggaagctggcgccgctcgccgaggcgtggaccggcggcctcgacgtggactgggcccgcggccacggcaccggcgaggaccggccgcgcaaggtcggcctgcccggctacccgttcgcgcgggagaggtactggtggaacgacgggctggccgaggccggaggcgagggcgctgacggtctgggagacgagggcgccgccggcggcaccgccggttccggtaacggttccgggccccgttccgctcgtacggacgggacgcgccccggtgaactgcccccgggcgacctcacgttgcaccccgtctgggagcccgtacatgcggcgggcggcggcgcggacgcgcccttcccgcagcccgcggaccgcgtcgtcgcggtcggcctggcaccggaggcccgtgccgcgctggaggcgtacggcacccgtgtggtgacgctcccggcaccccgggacggcggccgttccgtggcggacgtccgccgcgaactggagaccgcgggccccttcgaccacgtcgtcgtggagtgccccacccccgccgcacagggcgcgcggcagcgcgtcgaggctcaacgcgcctccgtacgcggcctgttccggctgctccaggcgctctccgccctccgcgcggacgagccgcggaccggtctgaccctcgtcacccgcgacgcgttcgacccggatcgcacgggcggcgccgacccggcgcaggccgcgctgcacggcctcgtcggcggcctcgccaaggaacagccgtactggcgcgtgcgcgccgtcgacctggccgagggcgagcccttcgtgcccgaggagatctgcgccctgcccgccgaccgccgggcgcatccgctcgtccggcgcggcggccagtggctgagccgcaggctgctgcccgtcggcgacgtacggcccggcacgccggacgacggcccgcgtacggctgttgacgggacggacgccgcgccgcaggccgtctccgtcccgtccggttccgtctcgtccgtctccgtcccgtccggcggtttccgcggtgacggcgtctatgtgctgatcggcggcgcgggcgacctcggcaccgtcctcaccgagcatctgctgcgccgctacgacgcccgcgtggtgtgggtgggccgccgtgcggaggacgacgccgtacgcgccgcggcggcacgcgtcgccgcggccaccggcggcgaggcccccgtgtatctgtccgccgacgcccgcgacccgggcgcgctcgcccgcgtacgggacgaggtgctgcgccggtacggacgcatcgacggcctggtgcacctggcgatggtcttcagccacacgctcctcgcggagctgccggaggaggacctgaacgcgacgctcgccgccaaggccgacccgaccgagcacttcgccgacgtcttcgcgggacagcggctcgacttcgtgctgctggtctcctccctcgtcagcttcatccgtaactcccaccaggcgcactacgcggcggcctgcgcctacgaggacgcccgcgcgcccggtctgggccgggcactgggctgccccgtcaaggtcgtcaactggggctactggggcaacgtcagcgacgaagtgctgcgcggcgtcacggagatggggctcgcgcccatcgaacccgcctccgccatggcggccgtcgaagagctgctgaccggcccgctcgaccagatcggcttcatgcggctcggccgtccgctgcccgtcgagggcgtgctcgcgggggagacgctgagcgggcatccgtacgcggcggtctcccgtacggccgcggagcccgcccccgtgccggtgccggccgcgctggcggagcaccacgcggggcctgtgccgggtgagatcgacgccctgctgtgccgctgtgtcgcggccacgctgcggcgtgccgggctgcgccgcccggccgacggcttcgcctccggttccggttccggttccggggccgggagcgcgggcgtgcgcgtggacgagcggttcgacggctggttcgcggcgaccgtacgcaccctgcgcgagtacgggctcgtcgactcccgcggcgactggagcgagcgcgcaccgggcgcgggggacgccgccgcctgcctggccgagtgggagcgcgcggccgagcggtgggcctctgcgcacgccgatctgcgggcgccgacggggctgttgggccgtacgctgcccgcgctggccgacatcctgcgcggccggatcccggcgaccgacgtgctgttcccggaggggtcgttctccctggtggagggcgtctaccgggacaacgccgtggccgcgcacttcaacgccgtactcgccgcacaggtgacggcgttcctgcacggccgccgcgcggccgacccggcggcgcggctgcgcgtactggagatcggcgcgggcaccggcggcaccaccgcgcccgtgctggagcaactggagtgcgcgggcctggagttggccgagtactgcttcaccgacctctccctcgccttcctccagcgtgccgaggacgccttcggccccggccgcgcccacttcgcctgccgcaccctcgacgtgtcacgggcaccgcgcacgcagggcttcgacgcgggggcgtacgacgtggtgatcgccgccaacgtgctgcacgccacggacgacgtacggaccgcgctgcggcacgccaagtcgctgctgcgcggcggcggaatgctggcgctgaacgagatcagcggcttctacctcgtcaaccacctcaccttcggcctgctggacggctggtggctctacggcgacgccgaactgcgggcgccgggcagccccgcgctgcctccggagagctggcgccgggtgctgacgcaggagggcttcaccggcgtcgcggacccggcacgggacgcccgtgccctgggacagcaggtcgtgatcgcccacagcgacggactggcccgcggcccggtgacggacgcggcaccggcggcaccggcagcaccggcggccgtggcgcggccggagacgaacacggcggtgagcgcggcgcccaacatggcggtgagcgcggcgagttcggcggcgggcggtccgcagacctccggcggtccggacgtgcgcgtggtcgccgacgtcgtcgagacggagctggccgacgcgctgcggctgccggcggaacggatcgaccgggcgggcgcgttcgcggacgtgggcctggactccatcgtcggcgcgcgcttcgtacggcggctcaacgaggaactgggcctggacctgccgacgacggtgatcttcgattaccggagcgtcgacgaactggccgcgcacatcgtggaggaccaccgtccgacctcgcctgcgccgggcggtaccggggcggccaccgctcaggagcccccggccgagcgggagtcggggcgcgccccggagcgggagcacgggcccgttgtggcgcccgatgtcaccgtgcccgatgccaccgagcccggttccgccccgtacggccgggagcccatcgccgtcgtgggcgtcagcgggcgcttcgccggttccgacgatctcgacgccctgtggcggcatctggccgcgggcgacgacctcgtcgggccgatcgaccgctgggatctctcggcctacggcgaggacgaactgacctgccgcagcggcagtttcctcgacggcatcgaccggttcgacgcccgcttcttcaagctgtcgggccgcgaggccgcctacaccgacccgcagcagcgcctcttcctcgaacaggcatggacggccctggaggacgccgggcacggcggcgcctcgaccgacggcatgcgctgcggcgtctacgtcggctgcaccggcggcgactacaaggaccacttcgaggacgcgccgcccgcgcaggccgtctggggcaacgcgccctcgatcgtccccgcgcgcatcgcctaccacctcaacctccagggcccggccatcgcggtcgacacggcctgctccagctcgctggtcgccgtgcatctggcctgccagggactgtggagcggcgagaccgagatggccgtggcgggcggcgtcagcgtgcagaccactccggccacctatctctcggccagccgcgccgggatgctctcgccgacgggacgctgccacaccttcgacgccgccgcggacgggttcgtaccgggcgagggcgtcggcgtcgtggtgctgcgcaggctctcggacgcgctgcgcgacggcgaccacgtgcacgccgtcatccgcggttcgggcgtcaaccaggacggcgccaccaacgggatcaccgcgcccagcgccctgtcccaggaacggctgctgcgccaggtctacgaggacttcgcgatcgacccgtccgagatcggcatggtcgaggcccacggcaccggcacacagctcggagacccgatcgaatgccacgccctgcggcgggtgttcgagggcagcgacgtccccggcggctgcgcgctcggttcgatcaagacgaacctcggccacaccacgtccgcggcgggcgtcgcgggtctgctgaagatcgtcctgtcgctgcggcaccggcagatcccgccctccctgcactaccgcgaccgcaatcccgagatccggctggaaggcggccccctgtacgtgaacacctcactgcgcccctgggagccgaacgcgggcggcagccgtgccgccgccctcagctcgttcggcttcagcggcaccaacagccatctcgtcgtcgaggaggcaccggcgcgtcccgggcggtccccgctctccggcgccgccgccgtggaggagcccggactgccccgggtcttcccgctgtccgcgccccagccggcggcgctgcgcgagcgcgtccgcgatctggccgtccatctgcggagcacgccggacgccgtcctcgtcgacgtcagccacaccctggcgacgggccgcgcccacttcgcgcaccgcgccgccttcgtcgcccgcacccgcgaggagctgatcggtcaactcgacgactggctcgacggggaggccggagacgccgggaaggcggcgaagaccggggaggccgcgaagaccggagacgtcggcgaggccgggggcgccgggccggaggagctggcccgcgaccgctacctcgccggtgaacccgccgacttcgccgcgctgttcgccggttccggcgcccgtcgcacaccgctgccgacgtaccccttccagcgcaggagccactgggtgcgcggcggcgcaccggggagcgccccggacgcggccgggtccggtacgtccaccacgtccggcacgcccgccctccgtaccgacgcaagggagaagggccgcggagccgcccgcgcggaggacgacgccgtcgccgtcgtcggcctctccgcccgcttcgcgcagtcgccggacgccgaggccctgtgggcacatctcgccgcgggcgacgacctggtcggcgaggtgacccgctgggacctgtcgcagatcagcggcggacgcaccgaacacggcagcttcgtcgaggacatcgcccgtttcgacgccctgtacttcggcgtctcgggcaacgaggccacgtacgccgacccgcagcagcgcatctacctggaggagtgctggcacgccctcgaggacgccggttacgcgggggagcggctggacgggcggggctgcggcgtctacgtgggcgcctaccccggcgactaccacgagctgatcggcgccgaccgcccgccgcagacgatgtggggcaacatggcctcggtcatcgcctcgcgcatctcctacttcctcgacctggacggcccggcgatgtccgtcgactcggcctgctccagctcgctcgtcgccatccacaccgcgtgccaggacctgcgtctgggcacgacctccatggcgctggcgggcggtgtgttcatccaggcgacgccgcggctctaccagtactcgggcaaggcgcggatgctctcggccaccggacgctgccacgccttcgacgccgccgcggacgggttcgtccccggcgagggcgccggagtcgtcgtcctcaagcggctgtcggacgcgctgcgcgacggcgaccgcgtctacggcgtgatccgctcctcgggcgtcaaccaggacggcaccaccaacggcatcacggcacccagcggcgcggctcaggagaacctcgtccgcgacgtgtacgagcgcgcgggcgtcgccccgtccgggatccagctcatcgaggcgcacggcaccggcacaccgctcggcgacccgatcgaattcgaggcgctgcgcgccgtgttcgcggacgcgccgacgggcggctgcgcgctgggcacgatcaagagcaacgtgggccacacccagttcaccgccggagtcgcgggggtcctcaaggtgctgctcgcgctcgaccacgaacagctcccgccctccttgcacttcacccggcccaacccggccatcgacctcgcgaacagccccttccacgtcaacaccgaactgctgccctggcgcgcgcccgccgacgggccgcgccgcgcgggcgtcagctccttcggcgccgccggcaccaacgcgcacgtcctgatcgaacaggcaccgtccgacgccgccgcccgcgcacgccgacacgggcgcgcacagtggctgttggtgctctccggccaggacggcaccgcgctgcgcgcccaggccgagcggatgctggaccacgtcgaacgccacccggacctcgacctcggcgacaccgcctggacgctcgccacgggacgccgccacagcgctcaccgtctggcgtgcgtcgccgccgaccgcgagcagtggacggcggcactgcggggctggctgcgcgacggccgtgccgagggcgtgtggacgggcgaggccgacgagtcgccccgctccgggcacagcggcgagagcggcgagggcagcggcgaaccggcccgcgccgaggcgctgatggccgagcacgaccgtcccggaaacctcgccgcgctcgcggagctgtacgtacggggcgaggtcgcgcgcttcgcaccgctgtacgccgacggggacttccgcatcgtctccctgcccggctaccccttcggcggcgagcgctactggaccgggccgctgcccggggacacgcccgacgggacggacggaacggacgggacgtacggcacggacgggatcagcgaatccggtggcgaatcccggccgtccgccgaaccccggccgtacgccggggcgttggcgctgaccggggaggagttcttcctcgacgaccaccgggtcggcggcgtccccgtactgccgggcgtcgcgtatctggaactcgcgcacgcggcggcgaccgcacagggcggcctcgcccccggcggtgtgctgctgcgcaacgtcgtctggtcccgcccggcgcgcgtcaccgagccgctctccgtggagacggtgctcgaaccacgcgccgcggacggcacgttcggatacgagatcgccaccgtccgggacggcgcccggcggctggtgcacggccggggccggatcgagccgcgtcccggcggcgcgcccgcccggctcggcctcgccgcgctgcgtgagcggtgcgacgtgcggagcctggaccacgcggagtgctacgcgttgctcggcgccaccgggatgtcgtacggcgccgcgatgcgcggtctggaggagctgcacgtcggccgcgggctcgcgctcggccggctgcgcgttccgcgcgaggcacgcgacggacgcccctggacgctccatcccgccctgctggacgcggcgttgcaggcgacggtcgggctggccctggacggggagtccgacgggctgacggccgcactgcccttcgcggtggagcaggtgcaggtgctcgccgcgagcccggagagcggctgggccgtggcccgtcccgcggacggcgccgccgagggcccggtccggcggatggacgtggagatctgcgacgacgagggcacggtgtgcgtacggctcctcggcttcagcacccgcgaactcccgggcgccaccgcgtcggtgacgaccggagcgacgaccggggcggggtccggggcggggtccgccgccgcgtcccctgctccggccgccgccgatcccggcgcgcccgccgacggctccctggtcttcgcccggcccgtctggcgcgccgtgccctccgcagacgtacgggaggagcgcccggcgccgagtccggcaccgtaccgggagatcctgctggccggtccggagtccgtcgacgctgcggaggtgcggaagcgctcgggcgtcccgtgctcggcgctgcccggcggcgccgatctgcccgagcggtacacccggcaggcccaggcgctgctggcgaaggtgcagcaactcctgccgcgcgtacgggaggagcgcgtcctgctccaggtcgcggtccccgcgcacggagaaggccggctcttcgcggggctcgcgggtctgctgcgtacggcctgcgcggagcaccccggactggccgcgcagctcgtcgagaccgacgccgccgacgcggcgacgctctgcgcccacctcgacgccgaggccgcgcagcccggcgtggcgacggtgcgccgcacgggcggcgaacggctggtgcggcagtggcacggcttccgtccggagcgcggcgatcagccctggaagccgggcggggtccatctcgtcaccggcggcgccggcggcctcggagcgctgttcgcccgccggatcgcccgtaccgcgcccggatccgtactggtgctgtgcggccgctccccggagggcgcggcacagcgcgaactcctgggtgagctgcgggagtcgggcgccgcacacgcggagtaccacagcctggacgtcggcaggcgtgcggacgtcgtccggctcgtgcggcaggtcgtggaccggcacggacggctcgacggcgtgatccacagcgccggagtgctgcgggacggcttcgtcgcccacaagaccccggagtacctgggcgaggtcttcgccccgaaggccgggggagtggtgcacctcgacgaggccaccgccgcactggagttggacttcttcctcgtcttctcctcgatgtcggtgctcggcaaccccggccaggccgactacgcggcggccaacgcgttcctggacgcgtacgtcgcccaccgcgccggactggcggaccgcggtgagcgccacggccgctcgctctcggtcggctggcccctgtgggcggacggcggcatgcacgtggacgcggccacggagcgccgcatccaccagagctccggaatgcggccgttgcgtgcccgtgaggggttcgaggcgctggagcgcctgtacgggagcggactgccgcacgcgctgaccgcgttcggcgaccgcgagcgcatcgcgtcggtgctgctcgacggttccgagggctccgacggctcggctcgtccggacggtccggacgcggagcgggagacggacgagcggcgccggaccccggcggacgcgaacgacgaacggaacgaggccatgtcacacacggcgctggtcggccgactcgccgcccatctctcggagttgctggacgtaccggcggaggagatcgagggcggggtcgagctgagcgagtacggcttcgactcgatctcgctgacggagttcgtcacgctgctcaacggcgcgtacgggctgtcgctcgtgccgacggtgctcttcgagcactcgacgctcgacggggtcgcgggacatctgctggaggagtacgcggaccgcttcgcgccggagccggagccggagccggagccgcagccggtgcaggcgcagatgccggagccggtgccggtgccggagccggaacctgcaccggtgccggcgcgcgggcccgtggcaccgtcaaccgcccccgtggcggccgacgatgacgacgcgctgcgccgtgcgctggtcaagcggctgcgggagctgacgtcccgcatcctccgggtgcccgcggagaagatcagcgccacgcaggagatgagcaagtacggcgtggactccctgtcgctggcggagctggcggcggccgtgaacgcggagttctcgctgatgctggacccgacgctgttcttcgagcacccgacgctggaggccgtcgcccgctatctcctcgaccggcacgccgaccggctcaccggcctggtgaccgaggagacccccgaaccggccatgaccgaacaggccgtggctgaaccggtcgtggccgagccgcccgtcgtcgaatcgcccgctacgacgtcacccgccgcggagacgtccgtcaccgagacgtccgtacgtgaacccgccgcccccgcggcggctccggctccggctttcgccgcggccccggggccgggtgctgcggaggagcccgtcgccgtcatcgggatcagcgcgcgttttccgatggcggatgatctggcggagttctgggagaacttgcgtgagggccgggactgtatccgtgaggtgccctcggatcgctgggactggcgcgagtactacggcgaccccgtcaaggagcccaacaagaccaacgtgacgtccggtggattcatggacggcgtgggcgacttcgacccgctcttcttcgacatctcgcccaaggaagcggagttgatggacccgcagcagcggctgctgatgctccacacctggaaggccctggaggacgcgggctatgcgccggacagtctcgccggcaccggcacggccctcttcgtcggtacgacgaacaccggttacggaagcatggtcagccgctattcaccggtgatcgagggatacgacgcgaccggggccgcgccctgcatgggcccgaaccggatgagccatttcctcgatctgcacggccccagcgagcccgtggacaccgcctgttccagttcgctcatcgcaatgcaccgcgccattcaggcaattcacgacggccattccgacatggcgatcgcgggcggcgtcaacacgatggtgagcatcgacggccacatcagcatttcccgtgcggggatgttgagtgtggatggtcggtgtaagacgttttcggtgggggctgatggttatgggcgtggtgagggggtggggattttggtgttgaagcgtttgtcggctgcggtgcgtgatggtgatcatgtgtatggggtggtgcgtggttcggcggtgaatcatgggggtcgtgcgaattctttgacggcgccgaatcctcgtgctcaggcggatttggtggtgggtgcgtggtcgcgggcgggtgttgatccgcggtcggtgggttatgtggaggcgcatggtacggggacggggctgggtgatccggttgaggtgaacgggttgaaggctgcgtttgcggagttgtatgagcggtggggtgtttcgggggccggtgaggcgcattgtggtctgggttcggtgaagacgaatatcgggcatttggagttggcgtcgggtgtcgcgggtgtgatcaaggtgttgttgcagatgcggcatcggacgttggtggggagtttgcattgtgggtcggtgaatccgtatgtgcggttggaggggagtcctttccgtctggtgcgggagcgtgagccgtggcgggcggtacgggatgagaacgggcgggagttgccgcgccgtgcgggcgtgagttccttcggtttcggcggcgccaacgcccatatcgttctggaggaataccagcccccggccggcacgcagaccgacgcccacacccgcaccggcccctcaaccaccgtccacagcggccccgttgccgtcctgctctccgcccaccgtcccgacgtactgcgggagtcggcgacccgctgggtcgaggtcctgcggcgcggcgactaccgcgacgccgacctcccggcgctgtcgtacacctcgcagacgggacgcaccgccatggccgagcggctcgcggtcgtcgccgggacgctggaggagctgcgcgcgggactggagtcctggctccgcggcgagccgaccccggccgtgttcaccggacgcgccccgcgcgacggcgacgcaccggcggcaccggccgccctcaccgacgggttcgcctccgggggacgtacggaggcgcggcactgggcgccggtgctccaggcgtggacgacgggcgccgagtgcgactggcggacgctgtggggcgaacggcacccgcaacggatctccatgccgacgtaccccttccaactccggcgctactggctcgacatgaccaccccggcgcacggcccgcacgtctcccgcggactgcatccgctggtgcaccggaacacctccgacctgagcgagcagcgctacacctcgcacttcaccggccgggagttctacatcgccgaccaccgggtgcagggcgaacaggtcgtccccggcgcggcgttgctggagatggcacgcgccgccgccgtcctcgcggcgggcggtgcggagaccgactgggcgctgcgccaggtggtctggtcccggccgctgacggccggacggcccgtcgacgtgcacaccgccgtgtccgtgcgggcggacggcgaaccggccttcgagatctacacggagggccccggcggcgaacgcgtcgtgcactccaccggacggctgcaccgcaggaccgccgggaacgccgccgaactcctggacggacccgaactccccggcggcgccggacacttggacgtcgccgcgctgcgtgcccagtgcgacggcaccgtcctcgacgccgaggagtgctacgcccggttctccggcgtgggcctggagtacgggcccacgctgcgagccgtcgagacgctgagcggcggcacccggcaggcggtggcccggctgcggctgtcggccgccgcgtccgcgaggaccgggttcgccctgcacccgagcctgctcgacgccgcactccagtccacggcgggcctcttcaccggttccggtacgtcctcggcggccctgccgtttgccctggaccggctggaggtgctgcgcgcgacgccctcctcggggtgggcggtggcacgcttcgccgccgacgaccgcccaggcggggtgcgccgcctggacatcgacgtgtgcgacgacgacggcgaggtgtgcgtacggatccggggcttccaggtccgtacgtacggcggcgacgccgccccgtccgcctccggcgccgcaaacggcacccacgccgtgaccacggacggcaccggaaacggcacagacaccggcaacggaaacagcaccggcaccggcagcgaggcggacgcggacgcccggctgctgcacctcatccacgccatcggcgagggcgccctgagcgctgacgaattccagcggagcctcatatgaccactcacgccacgtcacttaccgaactgcacgagcagatccgtaccggacggatcggccaggacgaggccctccggctgatccgggcctggcagcagggccggcagaccgggagcgagggcgggccggcggagcggcaggccacgggcgacgacgccgcggcccgtggcgaggccctgcgcgagcgcgtgtgcgacatcgtgacgcacgcggtcagcgagttgctgaaggtcggcccggacgacctggacgccgacgtcgaactcagcgagtacgggctggactcgatcgtgatgagccagctcgtcaacgcggtgaacgacgaactcggcctggaactcgcccccacggtcctcttcgagcacccgaatctgcgggccttcagcgcccacctcgccgacacgtacgcggactcgctctccgtacggctgctcggcacgcccggcacggggcccgcgcccgccccctcgaccgcgacatcgcccgccccctcgaccgcgacatcggccgaacccgcggccgtcgccgctccgtcgacgtcaccgtcggaggcccgtacggaatcccgggtgcctacgcctccggcaaccggaggccgcttcttccccgccgccgtcccatccgccccatccgccgaaaccgaacccgtcaccgcacccgcacccgcgcccgcctctgaacaggcttcccctgcgcaggcttccgctgcggaggagcccgtcgccgtcgtcggcatgagcggacgttttccgatggcggatgatctggcggagttctgggagaacttgcgtgagggccgggactgtattcgtgaggtgccctcggatcgctgggactggcgcgagtactacggcgatcccgtcgaggagcccggccgcaccgatgtgaagtggggcggattcatcgacggcgtcgccgacttcgatccgctcttcttcggcatcgcgccgaaggaagccctccatatggacccgcagcagcggctgttgatgctctacgtctggaaggccctggaggacgcgggccattcggcggacagtctcgccgggagcgatctggcgatgttcgtcggtacgaacgacaccggttacggcacgctcgccgaacggtgcggaaaacgggacagcgtctcgcccaccggcggcgtcccctcactcggcccgaaccgcatgagcttctttctcgacgtgcacggtcccagcgagcccgtggaaacggcgtgttcgagttcgctggtcgccatgcaccgcggtgtcacggcgatcgcccgcgcggaatgtgagaccgccgtggtcggcggcatcaacaccatcgtcgttcccgacggtcacgtcagcttctcccgtgcggggatgttgagtgtggatggtcggtgtaagacgttttcggtgggggctgatggttatgggcgtggtgagggggtggggattttggtgttgaagcgtttgtcggctgcggtgcgtgatggtgatcatgtgtatggggtggtgcgtggttcggcggtgaatcatgggggtcgtgcgaattctttgacggcgccgaatcctcgtgctcaggcggatttggtggtgggtgcgtggtcgcgggcgggtgttgatccgcggtcggtgggttatgtggaggcgcatggtacggggacggggctgggtgatccggttgaggtgaacgggttgaaggctgcgtttgcggagttgtatgagcggtggggtgtttcgggggccggtgaggcgcattgtggtctgggttcggtgaagacgaatatcgggcatttggagttggcgtcgggtgtcgcgggtgtgatcaaggtgttgttgcagatgcggcatcggacgttggtggggagtttgcattgtgggtcggtgaatccgtatgtgcggttggaggggagtcctttccgtctggtgcgggagcgtgagccgtggcgggcggtacgggatgagaacgggcgggagttgccgcgccgtgcgggcgtgagttccttcggtttcggcggcgccaacgcccatatcgttctggaggaataccagcccccggccggcacgcagaccgacgcccacacccgcaccggcccctcaaccaccgtccacagcggccccgttgccgtcctgctctccgcgcgtgagcccgagacgctgcgcgcccgcgcacggcagctcgtcgactggctcgacaagggcgaggccaccgaggccgatctgccgcggatctcctacaccctccaggtcggccgggtcgcgatgccggaacgactggcctgtgtgacggagtcgctggccgaactgcgcgcccagctccaggagttcctcgacggcgaacggccccgtggcgtacggaccgggcgtgccgagcggcgcggcatctggaacgacctggccgacgacgaggacatcaccgccgcggtcgacaactggatggccaagggcaagctcgaccggctgctcaaactctgggtcgccggcgccgagttcgactggcggcggctgtggggcgaacaccccccgcggcgtattccgctgcccgcctaccccttccggctccagcgctactggatcgccgacggcacaagcggccggtccacacggcggccgtcgaccgcgcgcgagggaacgccgtacggaggcaccccgcgggacgcggagtaccgcgaactgctgcgcggcgacgagtacttcctgcgtgaccaccgcgtgggcggcgtgccgacgctgcccggtgccgcctgtctggagctggtccgcgcggcctggacccacgccgaccgtgcccccgacaccgccccgctgcggctgcgcgacgtgctgtggctgcgtccgctccaggtcaccgcgccccgtaccgtcgccgtcgccctcgacccggccgacggcacgtacgaggtgcgcgccgcggacggcgacgagcgcgaggtgtacgcacgcggcaccgtcaccgctgacggcccccacaccgtcgacggcccccacagcgccggcggcgaccgtccgggcgggaccgccgaaccggcggagcccgtaccggcacacgacatcgccgccctgcgcgaccgctgcccccaccgtctcgacgccgacggctgctacgaccggttcgcggacctgggcctcgcctacggcccggccctgcgcgccgtcgagacgctgcactacggcgcggacctggcactggcccgtctggtgctgccggaggcggcggccggggaacggacgctcaacccgagcatgctggacgccgcattccagaccaccctcggcgtgctcctcggcgagcaggcccaggcggcggacgccgaacgggccgctgccggcggttccgaggacgtcgcggcgctgccgttcgccgtgcgcgaggtacggatcctcgcccccacccccgccgagggctgggccgtggcacgcgccgcggagggcgaccggcccggcgggacggtacgcaccctggacatcgacctgtgcgacacctccgggcgggtctgcgtacgcctcacggggttcagcacccgtacggtccccgaggacggtgcgccccaactccccggggagccgcccgtgttgatgatcgagcccgcctggcgcgaggcggaggcggcctccgtggcggccatgccggaggaccaccgggtcgtgctgtgcgaactgcccggcgtggacgcggccgagctggccggctccctcggcggcgactgcgagacctggcaggccgagggggacgtcgccgcccgctacaccgagtacgcccggcggctgctggaactcctccaggaggaggcccgcagcccggccccggccccggcccccgcccctggcgggacctccggcggggttcccggcgggcggctcgtccagctcgtcaccccgtcctcggcaccctggctcggcggtctgagcggcatggtgcgcaccgcccgccaggagcaccccaagctcctcgtccagtggatcgaggccgaagacgactgctccgccgatgagttggcggtgctgctgcgcggcgacggcgccgaccccgccgaggtggcggtacggcacggcgacggacggcgcagggtctctcggtggcgcgagacgccgccgcccgcgccccgcgtcccctggcgcgacggcggggtctatctcgtcaccggcggctcgggcggcctcgccgcgctgttcgccaaggacatggcccgtcgcgtgcggcggccgtcgctggtcctctgcggacgcggcgcggccggtcccgaacagcgggagctggtcgcggagttggaggcgctgggcgcccgcgcggagtaccgcgtactggacgtctccgacgccggggccgtcagcgcggcggtccgggaggtggtcgccgcacacggcgccctgcacggtgtcgtccacgcggcgggcgtgctgcgggacggcttcctcgcgcgcaagagcgccgaggagctgcggcaggtcttcgcggggaaggtcgccgggctgcgccatctcgacgaggccacggcggacgtggagttggacttcctgatcgccttctcatcgatggccgccttcggcaacgccgggcaggccgactacgccgccgccaacgcgttcctcgacggctacgcccagcaccgcgaagcgctccgcgcgcgcggcgagcgccacgggcggaccctctcggtgaactggccgctgtgggagaagggcggtatgcgcggcggcgcgggcaccgaggccgtgctccagggcgtgggcatgcgcccgatgcgggcggagacggggctcgacgccctgtaccgggcgtgggcgtgcggtctgacgtccgtcctggtgctggagggcgaccacgagcggatgcgctcccggctgctgccggagcagcccccgctgcccgagcccccgcaccggccggacgcacagaagccgctggacgctcagaagcccctggacgcaccggagttgccggacggaccggaagcgacgaggacgggcggcggcgtgccggtgcgctccggctccgcggacgtcgcccgccgggtcaccgccgtcctggcggacctgctggagatcgacgcggagtccctgcggcccgacgtgccgctacgcgagtacggactcgactcgatcttcctcacccagttcctcggcacggcccgcaaggagttcgacccggcgctcacgctcgacgtcatagcgggctgcgagacgctgacggacttcatcgacgcgatcgagcgcgccgtcgccccgccggccgccacgcctccggcgcccgcatccgcgtccgcgccctcaccctcgtccggtacggaaggggaaccgtcaacgcgccccgccccggagccggatggcgtaccggtggtgcgtcccgtcgccaaggctcccgaggagttccccgagctgatccccatgaacgccgtacgggagggccgcccggtcttctgggtccaccacggcaacggcggagtcgagtcgtacgcggcggtggccgagtgctgcggacgccccttctacggcatccagccccgcggctggacgggctcggaggacatcctcaccggccaggaggccatggccgcctactacgtggacatcatccgcgccgtccagccggagggcccgtacgacgtcggcggcttctccctcggcggtctgttcgcctacgaggtcgtacgccaactccagctccaggacgccacggtggacaccctggtgatgctggacaccctcgacgccgcctcgaccaacctggccaactccctgatgacgggcggccgtcaggacgacgccgacgtggtggcgaaggtcagcgccttccgcgccgtcaacctgatgctgggcaacgacagcctggacgcgcgcgagggcacctcgtccgtcctgcaccgggacgaggtggacaccgcgctcgacccggacgccttcctggactccctcgtggaggccgccgtcgcccgcggcatccacaagaccccggcccaactgcggacgcgcgtacggcagttggcgcggtacttcgacgccgtgcacggcgagcggcacgtggtgcacccgctgccgcggcgcgaggaggtgcgctgctactacctgcgcaacgcgggcgggcagttcttcggccccttcgaggagtacatggtcctcttccccgaaccggacctcccggcggtggacggcaccccgtactggcgcgaatgggccgacgccgtcgacgacttcttcgtcatcgacgtcgacacggagacccacgcgcaggtgatgacggagcccgcggcgctgaagaaggtgctgcggctgtgcgaccggctgtacgcgccggagcagcacgcgcagggaggccggtgagatggaggcggtcgtctttcccgggcagggcgcgcagcgccgcggcatgggccgcgagctgttcgacgcctttccctcgctcaccgagcaggcgtccgacgtcctcgggtactccgtacgcgagttgtgcgtggcggaccccgagcgccggctgcgcagcaccgagtacacccagcccgcgctgttcgtcgtcggcaccctcgcgcacctcaagtggcaggaggagacggggcgttcggccgcgtacttcgccgggcacagcgtgggggagtacaccgcgctgcacgccgcgggcgccttcggcttcgagaccgggctgcgcctggtgcagcggcgcggtctcctcatgtcgcaggcggagggcggcggcatggcggccgtactcggcctcggcgccggggagttgaccgagctgctccgcgagggcggcttcgtctccctcgccctcgccaacgacaacacgcccgatcagcaggtcgtctcgggcgccgcgcacgagatcgacgtcctggaggcgtatctgtccgaacgcggcgtgcgcggtgtgcggctgaacgtctcgggcgccttccactcgccgctgatgctgcccgcacaggaggcgttcgccgcgtacgtacgggacttcacgctcggcgacccggagacgccggtgatctccaacgtcaccgcgcggccccatccgccgggcggtacggccgagttgctcgtacggcagatctccagccccgtgcggtggacggagagcgtgcgccatctgctcgacctcggcgtggaggagttcaccgaactcggcggcagcgtggtcgcgaagctcgtacggcagatccgcgaggcgcaccgcagggacgcggacgcctccggcccggcgcccgccgcgcccgcggctcccgtacggtccgaacccgccgcacggtccgcgctcggcagcgcggtcttccgcgagcggctggggctgcgccactcctacgcggcgggcggcatgtaccggggcatcgcctccccggccatggtggtgcgcctcgcccgcgccgggatgctcggcttcctcggcacgggcgggctgacgcccgaggccgtcgaggagcggatcctccaggtccggcgggagctgcgcgagggcgagcccttcggcgtcaacttcctcgccgaccacgacgatccggccgccgaacgccgcgtcgccgagatgctgatgcgccgcggcgtgaccgtcgtcgaggcggcggccttcatcggcatgaccccggcctcgtcctctaccgcgcgcgcgggcatgcaccgcggaccggacggggccccgcgctgcgcccaccgcatcgtcgccaaggtctcccgccccgaggtgggccggcgccttcatggcaccgcgcccgggaaggtggtcgacggcctgctccgggagggcgcgatcaccggcgagcaggcggagctcgtacggcaggtcccgatgagccacgacatcaccgtcgaggcggactccggcggccacaccgacgggggcatcgccaccgtcatgctgcccgcgatgctcggcctccggcggcaggcccagcgcagccacgactacgcggagccgctgtgcatggggctcgccggcggcctcggcactccggaggccgtcgccgcggccttcatgctgggcgccgactacgtcctgacgggctccgtcaaccagtgcaccgtcgaggcggacaccagcgacgccgtcaaggacatgctccagaccatcgacatccaggacacgggctacgcgcccgcgggcgacatgttcgagatgggagcccgggtccaagtgctgcgcaagggcgtcttcttcccgacccgggccaacaagctgtacgcgctctaccagcaccacgacggcctcgacgacctgcccgcgaagacccgtgccctgctggagaggtcgtacttccaccgcaccttcgacgagatctggacggaggtacgcgagcactaccgcgccaagggccagcccgaggtcaccgacaaggcggagcggcagccgaaggtgaagatggcgctggtcttccgctggtacttcgcctactcggcccggctggcactggccgggcaggacggcgacaaggtcaacttccagatccacacggggcccgcgctcggcgccttcaaccagtgggtcaagggcacggcctgcgagtcctggcgcgcccggcatgccgacgccatcgggctgatgctcatggagggcgcggcagaacacgtcgcggcggcctgcgagtcgtggggcggtacctcccgcttcgcccccgcggaggaacgcgccctggccgcccgcacctgaccccgcacctgaccccgtaccggcctcgacggcggaagccggctgccgcaccggaccggaccgtccggaccacgtaccccggccacgtaccacggcccacggcccacggcccacggcgtgaagtccgcgtcggcgcaggccggttcacgttcacgacggcccccgcacacacaccaccaccgcaccggataccgcgccgcacggcgccatgagaggacacgcagtgaccacccacctcaccaccgacatcgacgagatcgtcacggacgtcttccagtccaccgaggggaagaagaacccctaccccctgtaccggcgcctccaggagctgggccaggtgcaccgctccgagcaactgggctgggtcgccacgggatacgaggtgtgcagcgccgcgctgcgcgacccccgcgtcatcaagggccccgagcagatccagccgggccgccccgaccccgccgagcactccgccgaggcgttgctgcgcggcacgatgcaccggctcgacccgccggaccacacccggctgcgccgcctcgtcaacggcgccttcacgccgcgcagcgtcgccgcgctggagccggacatccaggaactgatcgacgacctgatcacaccggccgtcaagaaggcggaggcgggcgagcccgtcgacatgatgagcggcttcgccttcccgctctccgtcgccgtgatcggccgcatgctcggcgtgcccgcgtccgactggcaccgcttccacgacgtcgtgctcgacctgtcctccatggtcgaactcggcttcaccggcgacgagttgcccaaggccgacgccgccgcggacgaactgatcgcctacttccgcaagttgggcgccgagcgcatgcgcaaccccgccgacgacctgacctccacgctcgccaacgcgaccgaggccggtgaccgcctcaccgagcaggaactcgtcaccatgctgatcctgctgttcatggccggtttcgagacgacgacccactccatgggcaacggcatgttcgccctgctggagaacccggagcagacgcagtggctgcgccgcaacatggacgccatgcccgcggccgtcgaggagctgatccgctacgactccccggtgcagttcatcgccggctacaccaaggagcccgtggaactggcggacggcaccgccgtccccgcggacgagtatctgttcctgatgatcggcgcggccaaccgcgacccgcgcgtcttctccgaccccgaactgctgcgtctggaccgcggtgaggccgcgccgatgagcttcggcggcggcatccactactgcctcggcgcgggcctggcccggctggagatccggaagatcttcacctcgctgctcacccgcttctccgcgatcgaactggccgagcccgaaccggaacgccgcagcggactcgccctgcgcggctacgcccgcatcccgatgtggctcaccccggcgtaacccgccccgcccgtgaccgtacgaaggctccccgccccgttccgcctctcccgcggaacggggcggacccgtacgggaccggcaggcggaaggtgagcgggctcagcggagcgccgcgggccccgtctccgtccccgtctccgtctccgacttggaccccgcccctgctcccgtctcggtcctgctccccaagaggacggcggtggccagcagcccccgccgtgccgcccagcggccggtgaaaccgcgcaaccgctcgccgccgagcaccggcccctccacgaggagccgcgcggagaacgtgcccgcgtccgcgtcgatggtgatctcggcctcggagaagtcgagttcgcgtgaggtgagcggataccagaccttgaagacgctctccttcgcgctgaacagcaggcggtcccaggccacttggggatgcgccgccgccagcgtgcgcaggtgttcccgctcggcgggcagggagatcacgcccagaacgccgtccggcgtcggctctgcgggttcggcgtcgatgccgatgccgaccgcgtcgctgccgcgggcgacggccgccgcccggtagccgtcgcagtgcgtcatgctgccgacgacaccctccggccactgcggggcgccccggtgccccggcaccagcggcacgggcgccacacccagccgccccagtgcactccgcgcacacgcccgtacgtccgcgaactcccgctgccgcttgggcaccgcccgggccacggcggcccgctcctcggggaagagcccactcagcgtgtcctcctgcccaccgacgaagatctcctcggtgctcacccaccccggcaacatccgcccgatcac

SEQ ID NO: 26 - protein, 565 aa, Streptomyces amphibiosporus
VLFPGQGSQSKGMGRELFDRFPETTASACDVLGYDLRELCLENPEGRLDDTRYTQSALYTVNALEYLGSLEDGAPEGDYLLGHSLGEYNALLAAGVFDFETGLRLVLKRGELMARARHGGMLAVLGPGEEELRRTLAEEGMERLDVANVNTPAQTVLSGPVEEIERAQRHFDERRVRTARLKVSAAFHSRLMRPAREEFRAFLRGFRFASPRATVIANVSARPYGDVAEMLSEQIAGPVRWLESVRYVLERTSAERGREAGPGTVLTRMLRQIDGVSGAGNSPSVSASGSVPAASAPAESPSAPAASTSGSARPVGRATAATADAVREPTRPLLICAPYAGGDERSYAGLAEQLPEADVVTLERPGRGRRVSEPLLTEPGPVVEDMLSRIRDRVSRPYALYGHSLGARLVHLLARRLREEGLPGPRHLFVSGECGPSRPSRERYTSDLPTDAFWKHLRELGGVPDELFEYEDLTTFYERVLRADFTVLGACAYTPAAPLDCPVTAMTGDEEGLTEADVGAWQRETTAPLTARVFTGDHFFIRAHWPGVARVVAAGLGARRPAGTR

SEQ ID NO: 27 - DNA, 1698 bp, Streptomyces amphibiosporus
gtgctcttcccgggacagggctctcagtcgaagggaatgggaagagaactcttcgaccgttttcccgagaccaccgcgtcggcctgcgacgtcctcggatacgacctgcgcgagctgtgcctggagaacccggagggccggctcgacgacacccggtacacccagtccgccctctacaccgtcaacgcccttgagtatctggggtcgttggaggacggggcgccggagggcgactacctgctggggcacagcctcggcgagtacaacgcgctgctcgcggcgggcgtcttcgacttcgagaccgggctgcggctcgtcctcaagcgcggtgagctgatggcgcgggcgcgccacggcgggatgctggccgtactggggcccggcgaggaggagttgcgccggacgctggccgaggagggcatggagcgtctcgacgtcgccaacgtcaacacccccgcgcagaccgtgctttcggggccggtggaggagatcgagcgcgcccagcggcacttcgacgagcgccgggtgcgtacggcgcggctgaaggtctccgccgccttccactcacggctgatgagacccgcgcgggaggaattccgtgcgtttctccggggattccgcttcgcgtcgccgcgtgccacggtgatcgccaatgtgtcggcacggccctacggcgatgtcgcggagatgctgagcgagcagatcgccgggccggtgcggtggctggagagcgtccggtacgtgctggagcggacctccgccgagcgcggcagggaggcgggacccgggaccgtactgacgcggatgctgcggcagatcgacggtgtgtccggggcggggaattccccctccgtctctgcgtccggctccgtgcccgctgcctccgccccggccgagtctccgtccgctcccgcagcgtcaacgtccggcagcgcgcggcccgttgggcgcgccaccgccgccaccgccgatgccgtacgggagcccacccggcccctgctgatctgcgcgccctacgcgggcggcgacgagcgctcctacgcggggctcgccgagcaactgcccgaggcggacgtcgtcaccctggagcggccgggccgcgggcggcgggtctccgagccgctgctgaccgaaccggggcccgtcgtcgaggacatgctgtcccggatacgggaccgggtgagccggccgtacgcgctctacgggcacagcctcggcgcgcggctcgtccatctcctcgcccgccggctgcgcgaggagggcctgcccggcccccgccatctgttcgtctccggcgagtgcggcccctcgcggcccagccgggagcgctacaccagcgatctgccgaccgacgccttctggaagcacctgagagaactcggcggcgtgccggacgagttgttcgagtacgaggacctcaccacgttctacgagcgcgttctgcgcgccgacttcaccgtcctcggggcctgcgcgtacacccccgccgcacccctggactgccccgtcaccgccatgaccggcgacgaggagggcctgaccgaggccgacgtcggggcctggcagcgggagaccaccgcgccgctcacggcccgggtcttcaccggagaccacttcttcatccgggcgcactggcccggcgtcgcacgcgtcgtcgccgccgggctgggcgcccgccgacccgcagggacccgctga

SEQ ID NO: 28 - protein, 84 aa, Streptomyces amphibiosporus
MEQELKQYMEEQFMFEFDSEITEDTDLFKAGVLDSFGYISLIGHIEGEYGVKFGEEALLGNVAVTFAGLVESVASARRQTAESK

SEQ ID NO: 29 - DNA, 255 bp, Streptomyces amphibiosporus
atggagcaggaactcaagcagtacatggaagagcagttcatgttcgagttcgattcggagatcaccgaggacaccgacctgttcaaggcgggcgtgctcgactcgttcggctacatctcgctcatcgggcacatcgagggggagtacggcgtcaagttcggcgaggaggcgctgctcggcaacgtcgccgtcaccttcgccggcctcgtcgagtccgtggcgtccgcccgtcggcagaccgccgagagcaagtaa

SEQ ID NO: 30 - protein, 656 aa, Streptomyces amphibiosporus
VCGIAGFHASPLHPESYRDIAGAMLAQIEHRGPDEAGCFLDDRTAMGTVRLSIIDLASGSQPVGSPDGRYWLCYNGELYNYRELRAELAGRGVSFRTESDTEVVLMAWAHWGRSCLERFNGAFAFALKDTVTGELHLARDRFGKRPLYVARHGDAWLFASEMKAFLAYPGFEFAFDEEHLASTFATWTPLPAQSGYRGVEQLPMGEYLTVRGTETERGRWASLDLTGGEPPATEDEAVDLVRADLEAAVDLRLRSDVEVGVYASGGLDSSILAHLTKERAGLPPRTFSIQFEDAEFDETAEQEELTKHLGTHHSTVRVSDSDVVETFPEAVRHAEVPVFRTAFVPMYLLAQHVRSEGVKVVLSGEGADEAFLGYGIFKDARLLSEWHELDEATRMRRMAQLYPYLRHFSGEDGHRRMLGLYRQFTEETMPGLFSHQMRFQNGRFAVRLLKDAGDPFAAVRRLVAEEPGYAELSAVQKAQWLEFRTLLSGYLLATQGERMALAHGVENRCPFLDPAVVRRAASVNGRFGDPYDEKYLLKRAYGDVLPERIVSKGKFPYRAPDSAAFVRSRPDYRDLLADPGTLGDIGVLDERFVRRFTDRVFDRPPERIGTKENQAFVLLASTVWLHHWYVRGNARRDTPLAVPLYVVDRRSSALPA

SEQ ID NO: 31 - DNA, 1971 bp, Streptomyces amphibiosporus
gtgtgcggcatagcgggcttccacgcgagccccctgcacccggagagctaccgggacatcgccggtgccatgctcgcgcagatcgagcaccggggccccgacgaggcgggctgcttcctggacgaccgtacggccatgggcacggtgcggctgagcatcatcgacctcgcctccggctcgcagcccgtcggcagccccgacggccggtactggctctgctacaacggcgagctgtacaactaccgggaactgcgcgccgagttggcgggccgcggggtgtccttccgtacggagtccgacaccgaggtcgtcctgatggcctgggcgcactgggggcgctcgtgcctcgaacgcttcaacggtgccttcgcgttcgccctgaaggacaccgtcaccggtgaactgcacctggcccgcgaccggttcggcaagcggccgctgtatgtggcgcggcacggcgacgcatggctgttcgcctccgagatgaaggccttcctggcctaccccggcttcgagttcgccttcgacgaagagcatctcgcctcgacgttcgccacctggacgccgctgcccgcgcagagcggataccgcggcgtcgaacagctccccatgggcgagtatctgacggtccgcgggacggagaccgaacgcggccgctgggcgtcgctcgacctgaccggcggcgagccgcccgccaccgaggacgaggccgtcgacctcgtgcgcgccgatctggaggccgccgtcgacctgcggctgcgcagcgacgtcgaggtcggcgtctacgcctccggcggtctggactcctcgatcctcgcccatctcaccaaggagcgggccgggctgccgccgcgtacgttctccatccagttcgaggacgccgagttcgacgagaccgccgagcaggaggagctgaccaagcacctcgggacgcaccactccaccgtccgcgtctccgactccgacgtcgtggagaccttccccgaggccgtacgccacgccgaagtccccgtcttccgcacggcgttcgtgccgatgtacctgctggcgcagcatgtgcgcagcgaaggcgtcaaggtcgtgctcagcggcgagggcgccgacgaggcattcctcggctacggcatcttcaaggacgcccggctgctctccgagtggcacgagctggacgaggcgacccgcatgcggcgcatggcgcagctctacccgtatctgcgccacttcagcggcgaggacgggcaccgccggatgctgggcctctaccggcagttcacggaggagaccatgcccggtctcttctcccaccagatgcggttccagaacggccggttcgccgtgcggctgctcaaggacgcgggcgaccccttcgccgcggtacggcggctcgtcgcggaggagcccggatacgcggagctgtccgcggtgcagaaggcacagtggctggagttccgtacgctgctcagcggctatctgctcgccacccagggcgagcggatggcgctcgcgcacggcgtggagaaccgctgcccgttcctcgacccggcggtggtgcgccgcgcggcgtccgtcaacggccgcttcggcgacccgtacgacgagaagtacctgctcaagcgcgcgtacggggacgtgctgcccgaacgcatcgtcagcaagggcaagttcccctaccgggcgccggacagcgccgcgttcgtacggtcccgtcccgactaccgcgacctgctggccgaccccggcaccctcggcgacatcggcgtgctcgacgagcgcttcgtgcggcgcttcaccgaccgggtcttcgacaggccgcccgagcggatcggcaccaaggagaaccaggcgttcgtcctgctggcgtccacggtctggctgcaccactggtacgtgcgcggcaacgcccgccgcgacacccccctcgctgtccccctgtacgtcgtcgaccggcgcagcagcgcgctgccggcctag

SEQ ID NO: 32 - protein, 3436 aa, Streptomyces amphibiosporus
MKEESGALPEEGPVGTAVGTAADGAAGGPVDGQDIAVVGLSLRLPGARNPEEFWEHLAAGRSLISEVPERRWRKEDHLGNPRREFNKTNSVWGGFVDDADCFDAEFFHVSPREARSMDPQQRMALEMSWQALEDAGYRADRVAGSRTGVFMGVCHWDYAELIEKEVSEVDAYYPTGAAYAIIANRVSHHFDFRGPSVVNDTACASSLVAVQQAVQALQSGDCDHALAGGVNLTWSPRHFIAFAKAGMLSPDGLCRAFDADANGYVRGEGGGVVLLKRAADARRDGDPVHAVIKGIGSNHGGRTSSLTVTNPAAQAELIAGIYRRAGIAPESVSYIETHGPGTPVGDPIEVSGLKRAFAQLGEGREAEPSGHRCGIGSVKTNIGHLEGAAGIAGMLKVILAMRHRKLPATVNFRRLNPLITLDGSPLYVVDRLTDWETDGDGTLRAGVSSFGFGGTNAHVVLEAPGGHAAEVTDAEAVTDAEADADVDGGPDEGPDEGAEPRALRLPVSADDEERLRELCRSLAEWARAREAEGTAPPLADIARTLREGRVPMRERAVFRARSVAEWAEQLTALAEGTGGEPPAGCLRGRAEDGAGDGLDADDVAALTARWRERDEEEKFAAAWTRGLPVDWAQWPAEGRRVHLPGQVFQRTPHWFRPDEQPRGEAESAGGAAAQRDTAPERDAASGSERGPGAPEPAGPVGGPGLPGEGVQDGRGWHFPLRFAATDPFVRDHLVLGARIVPGVVALEAVTAAAARPAVAGARAGAAPHIRNAVWVRPLRVDGQVLETSLRLTPAGPESGGGYDWAVTDAAGTPYSSGRVEYADGPAPAATDLDALHRRHTRPVEVAGGYAALYASGIEHGPALRALHTLRAGPEGLLAELRLPAEPAAGAALQPAVLDSALLAVLALGTGGGDGTEGTGGTDGAGWRRPDAPAVPFALDGLTAYAPTTATTWAWLRPAGGRRPGAADIDLFDERGRLCARLTGYTSRELSTGSPALREARVSAPAPGEGAAGEEAPGKDAPGELLEVTGRWTPAPLGLPAAEAGPAAAQSGAAAPVTVLNAALDADLVAASAARLGMDVEHLAVPRDAGDADAMKAAFAACYPHVRRLVGQSRRVLLVAPGAPDSPVFAPLAALLKTAHQENPSFHGTTVLLEGFDPRDSARFEQVVRTEAAATADAAGGAADEEVAHTADGRRLRHETAELPHGTTGESLLAEGGVYWITGGAGGIGLLLAERLCLRYGATVVLSGRSPAAPAADALASRLTRGTLAYRGADVTDQDSVDALVAAVLAEHGRIDGVFHAAGVLDDGYLTAKPLAGTEAVLAPKVDGVTCVDRATRAGAPGFLLVFGSVAGAFGNAAQAGYAAANAYLDAFAARRQAAGLTTRAVDWPLWAEGGMRVDDASLKYLRKRTGTVPLPSGTGLDALERALHTGSPVRRVVLYGDRPALRVYAGLDRPQVTGARSGSASASAPGSSSGSVSAVRGEGTGTAPAALTDAELLVRTQDFLREQFAEVTLQDAEQIHPEEKLETYGIESISIVDLTSRLEDVFGSLPKTLFFEYVDLKGVAEYFVAEHRARLTELFAPEEPQASEAAEPAPEEPVAPAPVPVEPAAAAPAPAPAPVPAPPAPTAAPGTSVEAVPAPVPASVPTPRPAPAGNGDIAVVGMAGRYPGADTLEEFWELLSEGRHSFEPVPSSRWPHGDLYFDERDVLGKTTVRTGTFLRDVDAFDPRYFSISQRDAELLSPEVRLFLQAGVTALEDAGYSKETLRRRYDGDVGVLVGSMNNSYAYYGFENMLMRGTAMSGSEVGVMANMLSYYYGFTGPSMFVDTMCSSSSACVHQALSMLRGGECRMVVVGGINLMLHPYDLIATSQAHFTTKSAEVVRSYGLGADGTILGEGVGTLVLKPLAEAVADGDHVYGVIKGSGMTNAGVRNGFTVPSPQQQARAIERALDDAAVDARTVSYLEGHGSATSLGDPIEIKGASLAFGRDTRDVGYCAIGSVKSNVAHLLSGSGLVGLTKVLLQLRHRTLAPSLHSETLSPAIDFGSTPFVVQRERAEWRRPVVHGAEVPRRAGVTSIGAGGINVHLIVEEFDGTVNSAPDDGGSQLLVFSAMTPQALGTVLRDAHRHVADEAPALNALAYTLQTGKNELPCRLAFVAHGTADAEARLAALAAVDWTSGAPALPDAVRFTESTLRKRRSVAAADVERALAQADLAELAGYWISGAAVDWDLLWPSGTRPAKLALPAYPFEKVRCWYPGFDDAPSVLRPLAFTRRGHPWVGVNRSDLHGVRFALELTGDELLDYVHTVGRTRRFTSVALLDGALAFARLAGLDGALRLRDARWAELPSPGDATEVFEWRLALSGEGASGDAASGGGASGAGGHRVELWQAERGTLHFSAEVVPATAVAAGAVDARPADAAALLAAPVTLDGDAFYSALGEAGVDARPYARAVTGVTEAGGRRLLVRVAEPAMCQDPHKQHVSIPAWVLAGLAQGVQHATGRPRTTALRAAALYGADLTDTRALLLEPVAEATFRITFLDGDGRALGAVEDAEFTAGTLPPSLEGGAVPVRAGLPGAARPSATASAPASASVPVPALAAAPVAPAVPVEPVEPAAEADADAGDALVAVLRETVADLLKFELDEIDLDTHFHAYGFESIALARLASELNGLLGTDLSPVVFFECPDIRSLAAHLRERYDAETAARAVRGTGGGTGTDAARAPTPAPAPAVGAASAATAPAPLSSAEPVSDHEADYPGAVAVVGVAGRFPGAPDADTFWQRLRAGDDLIGEYPGDRFDERYTGVVARSDFPKFAGVLDDVDRFDAGFFNLSRLEAELMDPQHRLALETVWAALEDGGYAPGRLPENTGVYVGVSGSDYHHLLNASGVAPDGFTATGNAHSMLANRISFVLDVHGPSEPVDTACSSSLVALHRAVESIRSGRCDMALAGGVNLLLSIDTFAATQMAGMLSPDGRCKTFSADADGYVRAEGVAAVLLKPLERALADGDPVWGVVRGSAENHGGRAGSLTAPNAVAQTALIREAMRGTDPDSVGYVEAHGTGTGLGDPVEVGALDSAYRALRSDRGRVESGTAPVALGSVKTNIGHAESAAGLAGVLKVLLAMRHGELPPTLHCDRLNPHLPLSGGGFEVVREVRRWEPRLDADGRPWPLRAGVSSFGFGGANAHVVLEAAPAAARERAVRETASRSASVRSAHGTQGAPQAVAPQAVGPQIVAVSARDGERLRIVAERLRDFLRREHGAGRAPATADLARTLQTGREAMEARLAFVAEETGDVLDVLDRFLKGEEPDGWHTGALRRSRGAGVRRDRAQDPRVTRALRDGDLDAAAALWCEGALVDWQSLHPPGERRTVRLPSYPFARERYWVPTDGAAPPPETGGPGGVEDGGVEYGTGSGAAQLGDSGSAFDAGALAAVLDAVLDGRADPDDLART

SEQ ID NO: 33 - DNA, 10311 bp, Streptomyces amphibiosporus
atgaaggaagaatccggcgccctccccgaggaaggccccgtcggcaccgctgtcggcaccgccgccgacggcgcggccggcggcccggtggacgggcaggacatcgctgtcgtgggcctgtccctgcggctgccgggcgcacggaacccggaggagttctgggagcacctggccgcgggccgctcgctgatcagcgaggtgcccgagcggcgctggcgcaaggaggaccatctcggcaacccgcgccgggagttcaacaagaccaacagcgtgtggggcggcttcgtcgacgacgccgactgcttcgacgccgagttcttccatgtctcgccgcgcgaggcccgctccatggacccgcagcagcggatggcgctggagatgagctggcaggcgctggaggacgccggataccgggccgaccgggtcgccggctcccgtacgggcgtcttcatgggggtgtgccactgggactacgccgagctcatcgagaaggaggtctccgaggtcgacgcctactacccgacgggcgccgcgtacgcgatcatcgccaaccgcgtatcgcaccacttcgatttccgcgggcccagcgtcgtcaacgacaccgcgtgcgccagttcgctggtggcggtgcagcaggcggtgcaggcgctccagtccggggactgcgaccacgcgctggccggaggcgtcaacctgacctggtcgccacggcacttcatcgccttcgccaaggcgggcatgctctcgccggacgggctctgccgcgccttcgacgcggacgccaacggctacgtacggggtgagggcggcggcgtcgtcctgctgaagcgggcggcggacgcccgccgggacggcgaccccgtacacgcggtgatcaagggcatcggcagcaaccacggcgggcgcaccagttcgctcaccgtcaccaaccccgccgcgcaggccgagctgatcgccgggatctaccggcgggcggggatcgccccggagtccgtctcctacatcgagacccacgggccgggcaccccggtcggcgaccccatcgaagtcagcggcctcaagcgggcgttcgcgcagctcggcgagggacgggaggccgagccgtccgggcaccgctgcggcatcggctcggtgaagaccaacatcgggcatctggagggcgccgcgggcatcgccgggatgctcaaggtgatcctggcgatgcgtcaccgcaagctgcccgcgacggtgaacttccgccgtctcaacccgctgatcacgctggacggcagcccgctgtacgtcgtggaccggctcaccgactgggagacggacggcgacgggacgctgcgggcgggtgtcagctcgttcggcttcggcggcaccaacgcgcatgtggtgctggaggcgcccggcggtcacgcggcggaggtcacggacgcggaggcggtcacggacgcggaggcggacgccgacgtggacggcgggccggacgaggggcccgacgagggcgccgaaccgcgtgctctgcggctccccgtctccgccgacgacgaggagcggctgcgtgagctgtgccgctcgctcgccgagtgggcccgtgcccgcgaagccgaaggcacggcgccgccgctggccgacatcgcccgcaccctgcgcgaagggcgggtgccgatgcgggagcgtgcggtcttccgcgcgcggagcgtcgccgagtgggcggaacagctcacggccctcgccgaggggaccggcggggagccgcccgctggctgtctgcgcggacgcgcggaggacggcgccggggacggcctggacgccgacgatgtcgcggccctgaccgcgcgctggcgggagcgggacgaggaggagaagttcgccgcggcctggacgaggggcctgcccgtggactgggcgcagtggcccgccgagggccgccgcgtccatctgcccggacaggtcttccagcggacgccgcactggttccgtccggacgaacagccgcgcggcgaggccgagtcggcgggcggtgcggcggctcagcgggacaccgctccagagcgggacgcggcgtccgggtcggaacgcgggcccggcgcaccggagccggcgggaccggtgggagggccggggctgccgggcgagggcgtccaggacgggcggggctggcacttcccgctgcgcttcgccgccaccgaccccttcgtacgcgaccatctggtcttgggcgcccgtatcgtccccggcgtcgtggcgctggaggccgtgaccgccgccgcggcacgtcccgctgtggccggtgcccgtgccggtgcggcgccgcacatccgcaacgcggtgtgggtgcggccgctgcgcgtggacggccaagtcctcgaaacgagcctgcggttgacgcccgccggcccggagtccggcggcggctacgactgggccgtcaccgacgccgcgggcacgccgtacagcagcggtcgcgtcgagtacgccgacgggcccgcgcccgccgccacggatctggacgcgctgcaccggcggcacacccgtcccgtggaggtggccgggggatacgcggcgctgtacgccagcggcatcgagcacggcccggcgctgcgcgccctgcacacgctgcgcgccgggcccgaagggctgctggccgagctgcggttgcccgccgagccggctgcgggcgcggctctccagcccgccgttctggacagcgccctgctggccgtgctcgcgctcggtacgggcggtggcgacggcacagagggcaccggcggcacagacggcgcgggctggcgccgaccggacgcgcccgccgtgccgttcgcgctggacggcctgaccgcgtacgccccgaccacggccacgacatgggcctggctgcggcccgcgggcggacgccgtcccggcgccgccgacatcgatctgttcgacgagcgggggcggttgtgcgcccgtctgacgggctacacctcgcgtgaactgtccaccgggagcccggcgttgagggaagcgcgcgtttctgcaccggcaccgggcgaaggggcggcgggcgaggaggctccggggaaggacgctccgggcgagctgctggaggtcaccgggcgctggacgcccgcgcccctcggcctccccgccgccgaggcgggaccggccgcggcacagtcgggcgccgccgctcccgtcacggtgctgaacgccgccctggacgccgacctcgtcgccgcgagcgccgcccgtctcggcatggacgtcgagcacctggccgtaccgcgcgacgcgggcgacgcggacgccatgaaggcggcgttcgcggcctgttacccccatgtccggcggctcgtcggacagtcgcggcgcgttctgctcgtggcccccggcgcacccgactccccggtcttcgcgccgctggcggccctgctgaagacggcacaccaggagaacccgtccttccacggcaccaccgtgctcctggagggcttcgacccgcgtgactccgcacgcttcgagcaggtcgtccgtacggaggcggcagcgacggcggacgccgcgggcggcgcggcggacgaggaggtcgcccacaccgccgacggccgccggctgcgccatgagacggccgaactgccgcacggcacaacgggcgagagcctgctggcggagggcggcgtctactggatcaccggcggcgcgggcggcatcggcctgctgctggccgagcggctgtgcctgcggtacggggcgacggtcgtcctcagcggacggtcgcccgctgcgcccgcggccgacgcgctcgcctcccggctcacccgcggcacgctggcctaccgcggcgcggacgtcaccgaccaggacagcgtggacgcgctggtggccgccgtgctggccgaacacggccggatcgacggtgtgttccacgccgccggggtgctcgacgacggctatctgacggccaagccgctcgccgggaccgaggccgtgctcgcgccgaaggtggacggcgtcacctgcgtcgaccgcgccacgcgcgcgggcgctccgggcttcctgctggtcttcgggtcggtcgcgggtgccttcggcaacgcggcgcaggccggctacgccgccgcgaacgcctacctcgacgcgttcgccgcccggcggcaggccgccgggctgaccacccgggccgtcgactggccgctgtgggccgagggcggcatgcgcgtggacgacgcgagcctgaagtatctgcgcaagcgcaccggcaccgtgccgctgccctccggcaccggcctcgacgcgctggagcgtgcgctgcacaccggttcgccggtgcggcgcgtggtcctctacggcgaccgtccggcgctgcgggtgtacgcgggtctggaccgtccgcaggtcacgggcgcacggtccggctccgcgtccgcgtccgcgccgggctcctcgtccgggtccgtgtccgccgtgcgcggcgaggggaccgggacggcaccggccgcgctcaccgacgccgaactcctcgtccgcacacaggacttcctgcgggagcagttcgccgaggtcaccctccaggacgccgagcagatccaccccgaggagaagctggagacgtacgggatcgagtcgatctcgatcgtcgatctgacgagcaggctggaggacgtcttcggctcgctgcccaagaccctcttcttcgagtacgtcgatctgaagggcgtggccgagtacttcgtggccgagcaccgtgcgcggctgaccgaactcttcgcgccggaggagccgcaggcgtccgaggcggcggagcccgcaccggaagaacccgtggcgcccgcccccgtaccggtggagccggccgccgcggcacccgcacccgcacccgcacctgtaccggcgcctccggcgccaaccgccgcaccgggtacgtccgttgaggccgtccccgcgcccgttcccgcttccgtacccacgccgcgccccgcccccgccgggaacggcgacatcgctgtcgtcggcatggcgggccgctacccgggcgccgacaccctggaggagttctgggagctgctcagcgagggacggcacagcttcgagcccgtgccgtcctcgcggtggccgcacggcgacctgtacttcgacgagcgggacgtgctgggcaagaccacggtgcgcaccggcaccttcctgcgtgacgtcgacgccttcgacccccgctacttcagcatctcccagcgcgacgccgaactcctctcgcccgaggtgcgcctcttcctccaggcgggcgtgacggctctggaggacgccgggtactccaaggagacgctgcgccgccgctacgacggcgacgtgggcgtgctcgtcggctcgatgaacaacagctacgcctactacggcttcgagaacatgctgatgcgcggcaccgcgatgagcggcagcgaggtcggcgtgatggccaacatgctctcgtactactacgggttcacgggcccgtcgatgttcgtcgacaccatgtgctcgtcgtcctcggcctgtgtgcaccaggcgctgagcatgctgcgcggcggcgagtgccgcatggtcgtcgtcggcggcatcaacctgatgctccacccgtacgacctgatcgccacctcgcaggcgcacttcaccaccaagtcggcggaggtcgtgcgcagttacggactcggcgcggacggcacgatcctcggcgagggcgtggggaccctggtgctcaagccgctcgccgaggccgtcgcggacggcgaccacgtctacggcgtcatcaagggcagcggcatgaccaacgccggtgtgcgcaacggcttcacggtgcccagcccgcagcagcaggcgagggcgatcgagagggcgctcgacgacgccgccgtggacgcgcgcaccgtcagctacctggagggccacggctcggcgacctcgctgggcgacccgatcgagatcaagggcgcgagcctcgccttcggccgggacacccgggacgtgggctactgcgcgatcgggtcggtcaagtccaacgtggcgcatctgctgtccggttcgggcctcgtcggcctgacgaaggtgctgcttcagctacggcaccggacgctggcgccgtcgctgcactccgaaaccctcagccccgccatcgacttcggctcgacgccgttcgtggtgcagcgtgagcgcgccgagtggcggcgtcccgtcgtacacggcgcggaggtgccgcgccgcgcgggcgtcacctcgatcggcgcgggcggcatcaacgtgcacctgatcgtcgaggagttcgacggcacggtgaactccgcgcccgacgacggcggttcacagcttctggtgttctccgcgatgacgccgcaggccctgggcaccgtgctgcgcgacgcgcaccgccatgtcgccgacgaggcgcccgcgctcaacgccctcgcgtacaccctccagaccggcaagaacgaactcccctgccggctggccttcgtcgcgcacggcacggccgacgccgaggcccggctcgccgcgctggcggcggtggactggacgtccggcgcacccgcgctgcccgacgctgtgcgcttcaccgagagcacgctgcggaagcggcgcagcgtggcggccgccgacgtcgaacgggccctggcgcaggccgacttggcggagctggccggatactggatctccggcgcggccgtggactgggacctgctgtggccctcgggtacccgtccggcgaagctggcgctgcccgcgtacccgttcgagaaggtgcgctgctggtacccgggtttcgacgacgcccccagcgtgctgcggcccctggccttcacccggcgcgggcacccctgggtcggcgtcaaccgctccgatctgcacggcgtgcgcttcgcgctggagctgacgggcgacgaactcctcgactacgtccacacggtgggccgcacccgccgcttcacgagcgtcgccctgctggacggggcgctggcgttcgcgcggctcgcgggcctggacggcgcgctgcggctgcgcgacgcgcggtgggcggagctgccctcgcccggcgacgccacggaggtcttcgagtggcggctcgcgctctccggcgagggagcgtccggtgacgcggcgtccggcggaggagcgtccggtgcgggcggccatcgtgtcgagctgtggcaggccgagcgcggcacgctgcacttctcggcggaggtcgtaccggcaactgccgtggcggcgggggccgttgacgcgcggcctgccgacgccgcggcgctgctcgccgcacccgtgacgctggacggcgacgcgttctactccgcgctcggcgaggcgggcgtggacgcccgcccgtacgcgcgcgccgtcaccggcgtcaccgaggccggcggacggcggctgctcgtccgtgtcgccgaaccggcgatgtgccaggacccgcacaagcagcacgtcagcattcccgcgtgggtgctggcggggctggcacagggcgtgcagcacgccacgggccggccgcgtacgacggcactgcgcgccgccgcgctgtacggcgccgacctgacggacacccgcgccctgctgctggagcccgtcgcggaggccaccttccggatcaccttcctggacggggacgggcgggcgctgggcgcggtggaggacgcggagttcaccgccgggacgttgccgccgtcgctggagggcggcgccgtaccggtacgggccggactgccgggcgcggcacgcccgtcggcgacggcctcggcaccggcctcggcctcggtaccggtaccggcgctcgcggccgcacccgtcgcacctgccgtgcccgtcgagcccgtcgagcccgccgcggaggcggacgccgacgcgggggacgcgctcgtcgccgtgctgcgggagacggtcgccgacctgctcaagttcgagctggacgagatcgacctcgacacccacttccacgcgtacggcttcgagtccatcgccctcgcgcggctggcctccgaactcaacggcctgctcggaacggacctcagccctgtcgtgttcttcgagtgccccgacatccgcagcctcgccgcccatctgcgcgagcgctacgacgcggagacggcggcccgcgccgtccgcggcaccggcggcggcaccgggacggacgcggcccgggcgccgactcccgctccggcaccggcagtcggggcggcgtccgcggccaccgcgcccgctcccctctcgtccgccgagcccgtgtccgaccacgaggccgactacccgggcgccgtcgccgtcgtcggtgtggcgggccgcttccccggcgcgccggacgccgacaccttctggcagcggctgcgcgcgggggacgacctgatcggggagtacccgggcgaccggttcgacgagcgctacaccggcgtcgtcgcacggtcggacttcccgaagttcgcgggcgtcctcgacgacgtcgaccgcttcgacgccggcttcttcaacctctcccggctcgaagccgaactgatggacccccagcaccggttggccctggagacggtgtgggcggcgctggaggacggcggctacgcgcccggccggctgccggagaacaccggcgtctacgtcggcgtctccggcagcgactaccaccacctgctgaacgccagcggcgtggcgccggacggcttcaccgccaccggcaacgcccactcgatgctggccaaccggatctccttcgtcctggacgtgcacggcccgagcgagccggtggacaccgcgtgctccagctcgctcgtcgccctgcaccgcgcggtggagagcatcaggtcgggccgctgcgacatggccctggcgggaggcgtcaacctgctgctgagcatcgacacgttcgccgcgacgcagatggcgggcatgctcagcccggacggccgctgcaagaccttctccgcggacgccgacggctacgtacgtgccgagggcgtcgccgcggtgctactcaagccgctggagcgggcgttggcggacggcgacccggtctggggcgtcgtacgcggcagcgccgagaaccacggcggccgtgccggttcgctcaccgcacccaacgccgtcgcgcagaccgcgctcatccgcgaggccatgcgcggcaccgaccccgacagcgtcggctatgtcgaggcgcacggcacgggcaccggtctcggcgaccccgtcgaggtcggcgccctcgacagcgcctaccgcgcgctgcgttcggaccgcgggcgtgtcgagagcggcaccgcaccggtggcgctgggctcggtgaagaccaacatcgggcacgccgagtcggcggccggactcgccggggtgctgaaggtgctgctggccatgcgccacggcgaactgccgccgaccctccactgcgaccggctcaatccccatctgccgctgtccggcggcgggttcgaggtggtgcgcgaggtacgccgctgggagccgcggctcgacgcggacgggcggccgtggccgctgcgcgccggggtgagcagcttcggcttcggcggcgccaacgcgcacgtcgtgctggaggccgcacccgcggcggcacgggaacgggccgtacgggaaacggcttcgcggagtgcgtccgtacggtccgcgcacgggacgcagggcgctccgcaggccgtcgctccgcaggccgtcggtccgcagatcgtcgccgtctcggcgcgtgacggcgagcggctgcggatcgtggccgagcggctgcgggacttcctgcggcgggagcacggcgcgggccgggcgcccgcgacggccgacctcgcccgcacgttgcagacgggacgggaggcgatggaggcgcgtctcgccttcgtcgccgaggagaccggggacgtgctcgacgtactggaccggttcctcaagggcgaggagcccgacggctggcacaccggcgcactgcggcgctcgcgcggcgcgggagtgcggcgcgaccgggcgcaggacccgcgggtgacccgcgcgctgcgggacggggacctcgatgcggccgccgcgctgtggtgcgagggggccctcgtcgactggcagtcgctgcacccgccgggggagcgccgcaccgtgcggctgccctcgtacccgttcgcccgcgaacgctactgggtgcccacggacggggcggcaccgccaccggagaccggcgggcccggcggcgtcgaggacggcggcgtcgagtacggcaccgggtccggcgccgcccaactcggcgacagcgggagcgcgttcgacgccggagcccttgccgcggtgctcgacgcggtcctcgacggacgggccgatcccgacgacctcgcccgtacctga

SEQ ID NO: 34 - protein, 8360 aa, Streptomyces amphibiosporus
VSRNILRVPAWRDEPSRGQAAPAGVRRLAVLCDVPDAEAALLRQHSPRLPVVLVESRDDGPAAAYEHAATRLLAELQRLLGRPAAGPCRVQVVCRESTPQGWAGLLGMLRTAAQEDPRLRGQLIEFDRLPGGAELARVLDEEAAEEADHVRRAAGAAGTGTGTGAVRQVRHWSAARSAGRASSAGNPAPVWRPGGVYLVSGGAGGLGRLLAADVRRHAPGAVTVVCGRGPAPWQGAEPPADGVEYHSVDVTDRAAVAALVDRVLSAHGRLDGVVHAAGLLADDYVVRASHRETQRVLAPKVAGLVHLDEATRELPLDFLAAFSSAAGTLGNAGQAGYAAANGFLDAYQTHRAALAEAGERHGRSLSVGWPLWRDGGMTVPDEQLPELTERFGRPLTTGTALTALHAALALGTPHVLVRDGAEADETGAVNATGAGTATGIATEVEVPAVNEAVGTAVDDALEDDAPEGDRKGTPAVEPRLRVLPALKQLVAETVRLDPAALDAAAPLDGFGIDSLAVTRLNRRFAQWFGALPKTLLYQYPTLNELAGYLAEHHPEGCRRWLADTASPSLSPSASASASPSPSPATSTSVSAPSAQERRPSTPVAAGAVRTAGTNGTSGAAAPVSAEAPVPARTSPVDEPIAVIGLHGRYPGAPTLDAFWENLRSGRDGVTEIPAERWPLEGFWEPDVERAVREGASYSKWGGFLDGFAQFDALFFGIAPREAADMDPQERLFVESAWSVLEDAGYTRRRLAEQHRSRVGVFAGITKTGFDRHRPAAPAETDASSATGGVPPASPRTSFGSLANRVSYLLDLRGPSMPVDTMCSASLTAVHEACEHLRHGACELAVAGGVNLYLHPSTYVELCRSRMLARGGECRSFGTGGDGFVPGEGVGTVLLKPLSKAEADGDPVHAVILGSAINHGGRTNGYTVPNPRAQAELIREAMDRAGVSADEVGCVEAHGTGTALGDPVEIEGLAQAFADRTDTAAPCALSSVKSNIGHLEAAAGIAGLTKLVLQLRHGELAPTLHAEVPNPDIDFGSVPFALQTAAAPWPRTGGNSGRRIAGLSSFGAGGANAHVVVAEYTGAPAARTSAPAVADGSAATAGSGRPVLLPLSARTPEDLRARAVQLADWLDSRDAVDLTSVAATLQTGREAMDERLCCVASTPGEWREQLRAFADDPEREGPWHRGRVRATGEALAALAEKDELRALVGRWTARGEWAELAAFWAKGMPLDWSRLYADGRVPARLHLPAYPFAGRRYWPGPADVRNTADAQAPRTSTPSPSTLSTSTPGASRPVAVAPVAAAPSAESYIERVLLDALGEALQMTPAEIDPRRPFADYGLDSILGVHLVNVLNETLGTGLETTDLFDHGTAERLRAFLTETYGGTVTVPDGTGAAAEFVPDAPVPAREADDPVAVVGMAARYGDAEDPRALWDRLLAGDDLVEPVTRWDLGPEVTCRAGSFVRGMDRFDPVFFAISGVEAAHMDPQQRIFLEQCWNALEDAGYTGERLRERNCGVYVGCYAGDYYDSIGDRAPAQALWGTMGSVVASRIAYQLDLKGPALTTDTSCSSSLVSLHLACRDLRTGAADMAIAGGVFLQTTPRLYEAATRAGMLSPTGRCHSFDSRADGFVPGEGAGAVVLKRLSDALRDGDHVYGLVRATGVNQDGTTNGITAPSAASQEALLREVHAGVAPGGVQLVEAHGTGTQLGDPIEFRALSRVFGDAPAGSVVLGSVKTNLGHTQFAAGIAGVLKALLALQEQRVPPSLHFAEANARVPLDGSPFTVATTAQPWPEPAEGPRRAAVSSFGASGTNAHVVLEEHPPVRATTGPESAGGDGEAAFLLSARTPAALRAVAERLLARIEREPGLPARQVAYSLAAGRRHFPHRLAVVATGLPALAARLRAWLADEQPGGEGTLLHGVAHAGTRQAALGGLAPAELAAAYVGGAEGPFAESFPAGARRQVPLPTYPFERQRYWAEGTDGHAVPAAAGTSAVEPGGRRTAYRTRLTGEEFFLADHRVGGRTVLPGVLTLEAVRRAVTAGDGGDGTGIGTGGGTGVPTPLRLRDVVWPAPFPVGADGAELRVDLDGDAFAVRQDGSSVHAQGRWTMVPAPAASTSLETLRERCARRTLTREQCRAALEAVGIRHGERLRAIEQLSVGDGELLARLVLPATVETGTQATETFGLHPAMLDSAIQAVVGLYGDETGALGERPDAPALPFALDTADVLAPTTDRMWAHLRWADGYAPGEAGEVTKTDIDLYDDAGRLCVRLRGYASRRVTPAATGSATAAPAGTDDADAPRAQLLAPVWDAQPHADGPRSPEPGAHVVLLGGTPEERDGLRRLVADVTVVEPERHASAGELAALLPTGAEHVVWLAPRDASPAASSAEGPDGALAVFRLVKALLADGADARELSFTAVTRQARLLPGDADCDPAHAGVHGLLGTLAKEYPHWRVRGADIERDVSVPWPELLSLPADPRGEVSARRHGEWYRQRLLEVALDASGAASFSAGSQAPNPQAPNSQASGARSALREPGGVVVAIGGAGGIGTVWTEHMMRRHGARVVWIGRRPYDEEIAARQDRLAACGPRPEYVRADATDARALRRAVAEIERRHGPVRGVLHTAIVLGDQSLARMDEAAFRTTYEAKAAVSVNMADAFAGQPLEFVAFFSSMQAFFKAPGQANYAAGCTFSDAWAERLSTALDCPVKVMSWGYWAGVGIVTADGYRQRMAQLGLGSIEPDEGMAAFDALLASPYRQLALLKATDSRSIDGIYGDDELRQLPPAAPALADTLRTDRPDRNAEIRRLREQADGHAGVMYDALVRVTWALLTSLGLFRDGRAATAAEWRAVGGIEERYQRWTEHTLEVLTAAGRLRRAGEDRYAAVAPGAVPAEDAWAEWDRAREVWLADEAKQAQAVLVDTTLRELTAILTGRRAATDVMFPGSSLRLVEAVYKNNPVADYFNEVLADTLVAYLEHRLRQDPSARLRILEIGAGTGGTSSVVFRRLRPLAGHIETYTYTDISKAFLLHARRAYGEIAPYLDGQLFDAEKPLAGQPVAVGGHDVVIATNVLHATGNIRNTLRNAKAAVRANGLLLLNELSDNILFSHLTFGLLDGWWLYDDPAPRIPGSPGLTPQSWRRVLDEVGFRGSFVAAEGADDLGQQVIVAESDGAVRQPRPGGVSAFRGSLPEARPAQPTGGAGHLAVPAEHGSAPAVTVPVTAASASSAPGSAPAAVPSGDPSGDGSMAARVAGPARDLFRGLVADVLQLPVGDIRADVPFERYGIDSILVVQLTDAVRKVLDGVGSTLFFEVSTVDGLVEHFLRTRPDELAALVGVSAAEHPEPAAEAAAPEAVTEEPAASVPAPAPVAAPVSVPVPAAPGEDVPVAVVGMAGRYPGAADLDAFWENLLAGRDCVTEIPDGRWDHGRYYDERRGVPGRTYSKWGGFLDGVDEFDSLFFGISPKAASTMDPQERLFLQCAWTALEDAGHTRASLRSASRARLPEDAGDIGVFVGAMYSEYQLYGAEQGVRGEPVVVPGSLASIANRLSYFLDASGPSVAVDTMCASALSAVHLACAAIRRGECASAVAGGVNLSLHPSKYLMIGEGQFASSDGRCRSFGADGDGYVPGEGVGAVLLRPLADAVADGDRVLGVIRGSAVNHGGHTHGFTVPNPLAQASVIRGAWRRSGVDPRDIGCIEAHGTGTALGDPVEIAGLNAAFGEFTSERTFCSLGSAKSNIGHLESAAGVAGLAKMLLQMRHGTLVPSLHAERTNPEIDFAATPFVLQREAAPWPRREGRPRLGGISAFGAGGSNAHLLVEEYVPTAAPPRRAAPGPVLAVLSARDGERLREYAGKLRDALRSGQWTDEDLPDIAYTLQVGREAMSARFAAEVSTLAGLMDALDACARGAALPPGARLRTDGGRGGPVQDLADDEDFRETVVRWLRRGKLAPLAEAWTGGLDVDWARGHGTGEDRPRKVGLPGYPFARERYWWNDGLAEAGGEGADGLGDEGAAGGTAGSGNGSGPRSARTDGTRPGELPPGDLTLHPVWEPVHAAGGGADAPFPQPADRVVAVGLAPEARAALEAYGTRVVTLPAPRDGGRSVADVRRELETAGPFDHVVVECPTPAAQGARQRVEAQRASVRGLFRLLQALSALRADEPRTGLTLVTRDAFDPDRTGGADPAQAALHGLVGGLAKEQPYWRVRAVDLAEGEPFVPEEICALPADRRAHPLVRRGGQWLSRRLLPVGDVRPGTPDDGPRTAVDGTDAAPQAVSVPSGSVSSVSVPSGGFRGDGVYVLIGGAGDLGTVLTEHLLRRYDARVVWVGRRAEDDAVRAAAARVAAATGGEAPVYLSADARDPGALARVRDEVLRRYGRIDGLVHLAMVFSHTLLAELPEEDLNATLAAKADPTEHFADVFAGQRLDFVLLVSSLVSFIRNSHQAHYAAACAYEDARAPGLGRALGCPVKVVNWGYWGNVSDEVLRGVTEMGLAPIEPASAMAAVEELLTGPLDQIGFMRLGRPLPVEGVLAGETLSGHPYAAVSRTAAEPAPVPVPAALAEHHAGPVPGEIDALLCRCVAATLRRAGLRRPADGFASGSGSGSGAGSAGVRVDERFDGWFAATVRTLREYGLVDSRGDWSERAPGAGDAAACLAEWERAAERWASAHADLRAPTGLLGRTLPALADILRGRIPATDVLFPEGSFSLVEGVYRDNAVAAHFNAVLAAQVTAFLHGRRAADPAARLRVLEIGAGTGGTTAPVLEQLECAGLELAEYCFTDLSLAFLQRAEDAFGPGRAHFACRTLDVSRAPRTQGFDAGAYDVVIAANVLHATDDVRTALRHAKSLLRGGGMLALNEISGFYLVNHLTFGLLDGWWLYGDAELRAPGSPALPPESWRRVLTQEGFTGVADPARDARALGQQVVIAHSDGLARGPVTDAAPAAPAAPAAVARPETNTAVSAAPNMAVSAASSAAGGPQTSGGPDVRVVADVVETELADALRLPAERIDRAGAFADVGLDSIVGARFVRRLNEELGLDLPTTVIFDYRSVDELAAHIVEDHRPTSPAPGGTGAATAQEPPAERESGRAPEREHGPVVAPDVTVPDATEPGSAPYGREPIAVVGVSGRFAGSDDLDALWRHLAAGDDLVGPIDRWDLSAYGEDELTCRSGSFLDGIDRFDARFFKLSGREAAYTDPQQRLFLEQAWTALEDAGHGGASTDGMRCGVYVGCTGGDYKDHFEDAPPAQAVWGNAPSIVPARIAYHLNLQGPAIAVDTACSSSLVAVHLACQGLWSGETEMAVAGGVSVQTTPATYLSASRAGMLSPTGRCHTFDAAADGFVPGEGVGVVVLRRLSDALRDGDHVHAVIRGSGVNQDGATNGITAPSALSQERLLRQVYEDFAIDPSEIGMVEAHGTGTQLGDPIECHALRRVFEGSDVPGGCALGSIKTNLGHTTSAAGVAGLLKIVLSLRHRQIPPSLHYRDRNPEIRLEGGPLYVNTSLRPWEPNAGGSRAAALSSFGFSGTNSHLVVEEAPARPGRSPLSGAAAVEEPGLPRVFPLSAPQPAALRERVRDLAVHLRSTPDAVLVDVSHTLATGRAHFAHRAAFVARTREELIGQLDDWLDGEAGDAGKAAKTGEAAKTGDVGEAGGAGPEELARDRYLAGEPADFAALFAGSGARRTPLPTYPFQRRSHWVRGGAPGSAPDAAGSGTSTTSGTPALRTDAREKGRGAARAEDDAVAVVGLSARFAQSPDAEALWAHLAAGDDLVGEVTRWDLSQISGGRTEHGSFVEDIARFDALYFGVSGNEATYADPQQRIYLEECWHALEDAGYAGERLDGRGCGVYVGAYPGDYHELIGADRPPQTMWGNMASVIASRISYFLDLDGPAMSVDSACSSSLVAIHTACQDLRLGTTSMALAGGVFIQATPRLYQYSGKARMLSATGRCHAFDAAADGFVPGEGAGVVVLKRLSDALRDGDRVYGVIRSSGVNQDGTTNGITAPSGAAQENLVRDVYERAGVAPSGIQLIEAHGTGTPLGDPIEFEALRAVFADAPTGGCALGTIKSNVGHTQFTAGVAGVLKVLLALDHEQLPPSLHFTRPNPAIDLANSPFHVNTELLPWRAPADGPRRAGVSSFGAAGTNAHVLIEQAPSDAAARARRHGRAQWLLVLSGQDGTALRAQAERMLDHVERHPDLDLGDTAWTLATGRRHSAHRLACVAADREQWTAALRGWLRDGRAEGVWTGEADESPRSGHSGESGEGSGEPARAEALMAEHDRPGNLAALAELYVRGEVARFAPLYADGDFRIVSLPGYPFGGERYWTGPLPGDTPDGTDGTDGTYGTDGISESGGESRPSAEPRPYAGALALTGEEFFLDDHRVGGVPVLPGVAYLELAHAAATAQGGLAPGGVLLRNVVWSRPARVTEPLSVETVLEPRAADGTFGYEIATVRDGARRLVHGRGRIEPRPGGAPARLGLAALRERCDVRSLDHAECYALLGATGMSYGAAMRGLEELHVGRGLALGRLRVPREARDGRPWTLHPALLDAALQATVGLALDGESDGLTAALPFAVEQVQVLAASPESGWAVARPADGAAEGPVRRMDVEICDDEGTVCVRLLGFSTRELPGATASVTTGATTGAGSGAGSAAASPAPAAADPGAPADGSLVFARPVWRAVPSADVREERPAPSPAPYREILLAGPESVDAAEVRKRSGVPCSALPGGADLPERYTRQAQALLAKVQQLLPRVREERVLLQVAVPAHGEGRLFAGLAGLLRTACAEHPGLAAQLVETDAADAATLCAHLDAEAAQPGVATVRRTGGERLVRQWHGFRPERGDQPWKPGGVHLVTGGAGGLGALFARRIARTAPGSVLVLCGRSPEGAAQRELLGELRESGAAHAEYHSLDVGRRADVVRLVRQVVDRHGRLDGVIHSAGVLRDGFVAHKTPEYLGEVFAPKAGGVVHLDEATAALELDFFLVFSSMSVLGNPGQADYAAANAFLDAYVAHRAGLADRGERHGRSLSVGWPLWADGGMHVDAATERRIHQSSGMRPLRAREGFEALERLYGSGLPHALTAFGDRERIASVLLDGSEGSDGSARPDGPDAERETDERRRTPADANDERNEAMSHTALVGRLAAHLSELLDVPAEEIEGGVELSEYGFDSISLTEFVTLLNGAYGLSLVPTVLFEHSTLDGVAGHLLEEYADRFAPEPEPEPEPQPVQAQMPEPVPVPEPEPAPVPARGPVAPSTAPVAADDDDALRRALVKRLRELTSRILRVPAEKISATQEMSKYGVDSLSLAELAAAVNAEFSLMLDPTLFFEHPTLEAVARYLLDRHADRLTGLVTEETPEPAMTEQAVAEPVVAEPPVVESPATTSPAAETSVTETSVREPAAPAAAPAPAFAAAPGPGAAEEPVAVIGISARFPMADDLAEFWENLREGRDCIREVPSDRWDWREYYGDPVKEPNKTNVTSGGFMDGVGDFDPLFFDISPKEAELMDPQQRLLMLHTWKALEDAGYAPDSLAGTGTALFVGTTNTGYGSMVSRYSPVIEGYDATGAAPCMGPNRMSHFLDLHGPSEPVDTACSSSLIAMHRAIQAIHDGHSDMAIAGGVNTMVSIDGHISISRAGMLSVDGRCKTFSVGADGYGRGEGVGILVLKRLSAAVRDGDHVYGVVRGSAVNHGGRANSLTAPNPRAQADLVVGAWSRAGVDPRSVGYVEAHGTGTGLGDPVEVNGLKAAFAELYERWGVSGAGEAHCGLGSVKTNIGHLELASGVAGVIKVLLQMRHRTLVGSLHCGSVNPYVRLEGSPFRLVREREPWRAVRDENGRELPRRAGVSSFGFGGANAHIVLEEYQPPAGTQTDAHTRTGPSTTVHSGPVAVLLSAHRPDVLRESATRWVEVLRRGDYRDADLPALSYTSQTGRTAMAERLAVVAGTLEELRAGLESWLRGEPTPAVFTGRAPRDGDAPAAPAALTDGFASGGRTEARHWAPVLQAWTTGAECDWRTLWGERHPQRISMPTYPFQLRRYWLDMTTPAHGPHVSRGLHPLVHRNTSDLSEQRYTSHFTGREFYIADHRVQGEQVVPGAALLEMARAAAVLAAGGAETDWALRQVVWSRPLTAGRPVDVHTAVSVRADGEPAFEIYTEGPGGERVVHSTGRLHRRTAGNAAELLDGPELPGGAGHLDVAALRAQCDGTVLDAEECYARFSGVGLEYGPTLRAVETLSGGTRQAVARLRLSAAASARTGFALHPSLLDAALQSTAGLFTGSGTSSAALPFALDRLEVLRATPSSGWAVARFAADDRPGGVRRLDIDVCDDDGEVCVRIRGFQVRTYGGDAAPSASGAANGTHAVTTDGTGNGTDTGNGNSTGTGSEADADARLLHLIHAIGEGALSADEFQRSLI

SEQ ID NO: 35 - DNA, 25085 bp, Streptomyces amphibiosporus
gtgagtcgaaacatcctgcgtgtgccggcatggcgagacgagccgtcgcgcgggcaggcggcaccggccggagtccgccggctggccgtcctgtgcgacgtcccggacgcggaggcggcgctgctgcggcagcactcgccccgcctgcccgtcgtcctggtggagagccgggacgacgggcccgccgccgcgtacgagcacgcagccacccggctgctcgccgagctccagaggctgctgggccgcccggcggcgggtccgtgccgggtgcaggtggtgtgccgggagagcacgccgcagggctgggcgggactgctcggcatgctgcgtacggcggcgcaggaagacccccgactccggggccagctcatcgagttcgaccgactgccgggcggcgccgagctggcgcgcgtgctggacgaggaggccgccgaggaggcggatcatgtgcggcgggccgccggtgcagccggtacgggtaccggaaccggagccgtacggcaggtgcgccactggagcgcggcccggtcggcgggtcgcgcgtcgtccgccgggaacccggcgccggtgtggcggcccggcggcgtctatctcgtcagcggcggtgccggcggcctcgggcggctgctcgccgccgacgtgcggcggcacgcgcccggcgcggtcacggtcgtgtgcggtcgtggcccggcgccgtggcagggggcggaaccgcccgccgacggcgtcgagtaccacagcgtggacgtcaccgaccgggccgcggtggccgccctggtcgatcgtgtgctgagtgcacacggcaggctcgacggtgtcgtgcacgcggcggggctgctcgccgacgactacgtggtccgcgcgtcgcaccgcgagacccagcgcgtactggcgcccaaggtcgccggtctggtccacctcgacgaggccacccgcgaactgccgctggacttcctggcggccttctcctccgccgccgggacgctcggcaacgcgggccaggccggttacgccgcggccaacggcttcctcgacgcctaccagacccaccgcgccgcgctggccgaggcgggcgagcggcacggccgttcgctctcggtcggctggccgctgtggcgggacggcgggatgaccgtgccggacgagcaactgcccgaactcaccgagcggttcgggcgtccgctgacgaccggcacggcgctgacggcactgcacgccgcgctcgccctcggcacaccgcacgtcctggtacgggacggcgcggaggcggacgaaaccggagccgtcaacgcaaccggggccgggaccgcgaccgggatcgcgaccgaggtcgaggtcccggctgtgaacgaagccgtcggcacggccgtcgacgacgccctggaggacgacgccccggagggggacaggaagggaactccggctgtggaaccgcgcctccgcgtactgcccgcgctgaagcaactcgtcgccgagaccgtgcggttggacccggccgcgctggacgccgccgcgccgctggacggcttcggcatcgactcgctggccgtcacccggctcaaccgccgcttcgcgcagtggttcggggcgctccccaagacgctgctgtaccagtacccgacgctgaacgagctggccggatatctcgccgagcaccatccggagggctgccgccgctggctcgccgacacggcgtccccgtccctgtccccttccgcgtccgcgtccgcttccccgtccccgtccccggcaacgtccacgtccgtgtccgctccctccgctcaggagcggcggccgtcaactcccgtcgccgccggggccgttcgcacggccgggacgaacggcacgagcggtgctgccgccccggtttccgccgaggcccccgttcccgcccgtacgtcacctgtcgacgagccgatcgccgtcatcggtctgcacgggcgctaccccggtgcccccaccctggacgccttctgggagaacctgcgctccggccgggacggcgtcaccgagatccccgccgaacgctggccgctggagggcttctgggagcccgacgtcgagcgcgcggtgcgcgagggcgcgagctacagcaagtggggcggattcctcgacgggttcgcgcagttcgacgcgctgtttttcgggatcgcgccgcgcgaggccgccgacatggacccgcaggagcggctgttcgtggagagcgcgtggtccgtgctggaggacgcgggctacacccggcggcgcctcgccgagcaacaccgctcccgcgtcggggtgttcgcgggcatcaccaagaccggcttcgaccggcaccgcccggccgcccccgcggagacggacgcttcctccgccacgggcggcgtgccgcccgcctccccgcgtacgtccttcggctcgctcgccaaccgcgtctcgtacctgctcgatctgcgcgggcccagcatgcccgtcgacaccatgtgctcggcgtccctcacggccgtgcacgaggcgtgtgagcatctgcggcacggcgcctgcgagttggccgtcgccggcggcgtcaacctgtatctgcacccctcgacgtacgtggagctgtgccgttcgcggatgctcgcccgcggcggcgagtgccgcagcttcggcaccggcggcgacggcttcgtgcccggcgagggcgtcggcacggtgctgctgaagccgctgtcgaaggcggaggccgacggcgaccccgtacacgcggtgatcctcggctcggccatcaaccacggcggccgcaccaacggttacaccgtgcccaatccgcgcgcgcaggcggagctgatccgcgaggcgatggaccgcgcgggcgtctccgccgacgaggtcggctgtgtcgaggcgcacggcaccggaacggcgctcggcgaccccgtcgagatcgagggcctggcgcaggcgttcgccgaccgtacggacacggcggcgccgtgcgccctcagttcggtgaagtccaacatcgggcatctggaggccgcggcgggcatcgcgggcctgacgaagctcgtgctccagctccggcacggcgagctggcgcccacgctgcacgccgaagtgcccaaccccgacatcgacttcggctccgtaccgttcgcgctccagaccgccgcggcgccctggccgcggaccggagggaacagcggacggcggatcgcggggctgtcgtcgttcggcgcgggcggggcgaacgcgcatgtggtcgtcgcggagtacacgggcgccccggccgcccgcacctccgcacctgccgtggccgacgggtccgccgcgaccgccgggtccggacgcccggtgctgctgccgctgtccgccaggacgcccgaggatctgcgggcccgcgccgtacagctcgccgactggctcgactcccgcgacgcggtcgatctgacgtcggtcgcggcgacgctccagacgggccgcgaggccatggacgagcggctgtgctgtgtggcgtccacgcccggcgaatggcgcgaacagctccgtgcgttcgccgacgacccggagcgcgagggcccctggcaccgtggccgggtgcgggcgaccggcgaggcgctggccgcgctggcggagaaggacgaactccgggcgctcgtcggacgctggaccgcccgcggcgagtgggcggaactggccgcgttctgggccaagggcatgccgctggactggagccgcctgtacgcggacggccgggtcccggcccggctccatctgcccgcctatcccttcgccgggcgccgctactggcccggacccgcggacgtacggaacacggcggacgcgcaagcgccccgcacctccacgcccagcccgtccacgctcagcacgtcaactcccggcgcgtccaggcccgttgccgtcgcgcccgttgccgccgcgccgtcggcggagtcgtacatcgaacgcgtgctgctcgacgcgctcggcgaggccctccagatgacgcccgcggagatcgacccgcgccgcccgttcgcggactacgggctggactccatcctcggcgtccacctggtcaacgtcctcaacgagacgctgggcaccggcctggagaccaccgacctcttcgaccacggcaccgccgagcggctgcgcgcgttcctcaccgagacctacggcggcacggtgaccgtccccgacggcacgggcgccgctgccgagttcgtccccgacgctcccgtcccggcccgcgaggcggacgacccggtcgccgtcgtcggcatggccgcgcgctacggcgacgccgaggacccccgcgccctgtgggaccgcctgctggccggtgacgacctcgtcgagccggtcacccgctgggacctggggcccgaagtgacgtgccgcgcgggcagtttcgtacgcggcatggaccggttcgacccggtcttcttcgcgatctccggtgtcgaggcggcccatatggacccgcagcagcggatcttcctggagcagtgctggaacgccctggaggacgccggatacaccggcgagcggctgcgcgagcgcaactgcggcgtgtacgtgggctgttacgcgggcgactactacgacagcatcggcgaccgcgccccggcccaggcgctgtggggcaccatgggctcggtcgtcgcctcgcgcatcgcctatcaactcgacctgaagggcccggcgctcaccaccgacacttcctgctccagctcgctcgtctccctccatctggcctgccgcgatctgcgcacgggcgccgccgacatggcgatcgcgggcggtgtcttcctccagacgacgccgcggctgtacgaggccgcgacccgtgctggcatgctctcgcccaccggccgctgccacagcttcgactcccgcgccgacggcttcgtccccggtgagggcgcgggcgccgtcgtactgaagcggctgtcggacgcgttgcgcgacggcgaccacgtctatggcctcgtccgcgccaccggcgtcaaccaggacggcaccaccaacggcatcaccgcgcccagcgcggcctcgcaggaggcgctgctgcgcgaggtgcacgcgggcgtcgcgcccggcggcgtccagttggtcgaggcgcacggcaccggtacgcagctcggcgacccgatcgaattccgcgccctcagccgggtgttcggggacgcgcccgccggcagcgtcgtcctgggctcggtgaagaccaacctgggacacacccagttcgcggcgggcatcgccggtgtcctcaaggcgctgctggcgttgcaggagcagcgcgttccgccgtcgctgcacttcgcggaggccaacgcgcgcgtaccgctggacggcagtcccttcaccgtcgcgacgacggcacagccgtggcccgagcccgccgagggaccgcgccgggcagccgtcagctccttcggggccagcggcaccaacgcccatgtcgtactggaggagcacccgcccgtacgggcgaccacggggccggagtccgccggaggggacggcgaggccgccttcctgctgtcggcccgcacccccgccgcgctgcgggccgtcgcggagcggctgctcgcgcggatcgagcgtgaaccgggcctgcccgcccggcaggtggcctacagcctcgccgccgggcgtcgccacttcccgcaccggctggccgtcgtcgccaccgggctgcctgctctcgccgcccggctgcgtgcctggctggcggacgaacagccgggcggcgaggggacgttgctgcacggcgtcgcccacgccggaacgcggcaggccgcgctgggcgggctcgcacccgccgagctggcggcggcgtatgtgggcggcgccgaggggccgttcgccgagagcttcccggccggggcgcggcgtcaagtgccgctgcccacctatccgttcgagcggcagcgctactgggcggaggggacggacggacacgctgtcccggccgccgccggtacgtccgccgtggagccgggcggccggcgcaccgcgtaccccgcacgcggctcacgggtgaggagttcttcctcgccgatcaccgggtgggcggccgtacggtgctgccgggcgtgctgacgctggaggcggtacggcgcgcggtcaccgccggagacggcggcgacgggaccggaatcggcaccggcggcggtacgggcgtcccgactcccctgcggttgcgtgacgtcgtgtggcccgcgcccttccccgtcggcgcggacggggccgaactgcgcgtcgatctcgacggcgacgccttcgccgtacggcaggacggctcgtccgtgcacgcgcagggccgctggacgatggtgcccgcccccgccgcctccacgtcgctggagaccctgcgggagcgctgcgcacgccgcacgctgacgcgcgagcagtgccgtgcggcgctggaggccgtcggcatccggcacggtgagcggctccgcgcgatcgagcaactgtccgtcggggacggcgagttgctcgcccggctggtgctgcccgcgaccgtggagacggggacgcaggccacggagacgttcggactgcacccggcgatgctcgacagcgccatccaggccgtcgtcggactctacggcgacgagaccggagccctcggcgagcgtccggacgcccccgcactgcccttcgccctggacaccgccgacgtcctcgctcccaccaccgaccggatgtgggcccatctgcgctgggcggacggttacgcgcccggcgaggccggagaggtgacgaagaccgacatcgatctgtacgacgacgcggggcggctctgtgtgcgcctgcgcggctacgcctcccgccgcgtcacgcccgccgccaccggctccgcgaccgcggccccggccggtacggacgacgccgacgcgccacgggcgcagctcctcgcaccggtgtgggacgcacagccgcatgcggacggcccccgcagccccgagcccggtgcacacgtcgtcctgctcggcggcacaccggaggaacgggacgggctgcgccgtctcgtcgccgacgtcaccgtcgtggaaccggaacgccacgcgtccgccggggagttggccgcgctgctgccgacgggcgccgagcacgtcgtctggctcgcgccccgcgacgcgtcgcccgccgcctcctccgccgagggacccgacggggcgctcgccgtcttccggctcgtcaaggcgctgctcgcggacggcgcggacgccagggagctgagcttcaccgcggtcacccgccaggcccggctgctgcccggcgacgcggactgcgacccggcgcacgccggagtgcacggtctgctgggcacgttggccaaggagtacccgcactggcgggtgcggggcgccgacatcgagcgggacgtctccgtaccgtggccggagctgctgtcgctgcccgcggatccgcgcggcgaggtgtcggcccgccggcacggcgagtggtaccggcagcggctgctggaggtcgccctcgacgcgtccggcgccgcctccttctccgccggctcccaggcccccaacccccaggcccccaactctcaggcgagtggcgcccgttcggcactgcgcgagcccggcggcgtcgtcgtcgccatcggcggcgcgggcggcatcggcaccgtgtggaccgagcacatgatgcgacggcacggcgcccgcgtggtgtggatcggccgccgcccctacgacgaggagatcgccgcgcggcaggaccgcctcgcggcctgcggcccgcgcccggagtacgtacgggccgacgcgaccgacgcccgcgctctgcgccgcgccgtcgcggagatcgagcgccgccacggccccgtacgcggcgtcctgcacaccgcgatcgtcctcggcgaccagagcctggccaggatggacgaggccgccttccgcaccacgtacgaggccaaggccgccgtctcggtcaacatggccgacgcgttcgccggtcagccgctggagttcgtggccttcttctcctccatgcaggcgttcttcaaggcgccgggccaggccaactacgcggcgggctgcacgttttccgacgcctgggccgagcggctctccaccgcgctcgactgccccgtgaaggtcatgagctggggctactgggccggagtcggcatcgtgaccgccgacggctaccggcagcgcatggcgcagctcgggctcgggtccatcgaaccggacgagggcatggccgccttcgacgcgctgctggcctccccgtaccggcagctcgccctgctcaaggcgaccgacagccgcagcatcgacgggatctacggcgacgacgagctgcggcaactgccgcccgccgcgcccgcgctcgcggacaccctccgcacggaccgccccgaccggaacgcggagatccggcggctgcgggagcaggccgacggccacgccggagtcatgtacgacgctcttgtccgcgtcacctgggcgctgttgacgtcgctgggactcttccgcgacggccgcgcggccaccgccgccgagtggcgcgccgtcggcggcatcgaggagcgctaccagcgctggacggaacacacgctggaggtgctgaccgccgccggacgtctgcgccgcgcgggcgaggaccggtacgccgccgtcgcccccggagccgtacccgccgaggacgcctgggccgagtgggaccgggcgcgggaggtgtggctcgcggacgaggccaagcaggcgcaggccgtgctcgtcgacaccacgctgcgggagctgacggcgatcctcaccggccgccgcgccgccaccgacgtgatgttcccgggctcctcgctgcggctcgtcgaggccgtctacaagaacaaccccgtcgcggactacttcaacgaggtgctcgccgacaccctcgtcgcctacctcgaacaccggctgcgccaggacccgtccgcgcggctgcgcatcctggagatcggcgccgggaccgggggcaccagctccgtggtcttccggcggctccggccgctggccgggcacatcgagacctacacctacaccgacatctccaaggcgttcctgctgcacgcccggcgtgcgtacggggagatcgcgccgtacctggacgggcagctcttcgacgcggagaagccgctcgccggacagccggtcgccgtcggcggacacgacgtggtgatcgccaccaacgtgctgcacgcgacgggcaacatccgcaacaccctgcgcaacgcgaaggccgccgtacgcgccaacggcctgctgctgctcaacgagttgagcgacaacatcctcttcagccacctcaccttcggtctgctggacggctggtggctctacgacgacccggcgccgcgcatcccgggatcgccggggctgacgccgcagagctggcgccgcgtcctggacgaggtgggcttccgcgggtcgttcgtcgcagccgagggcgccgacgacctcggccagcaggtgatcgtcgccgagagcgacggagccgtgcggcagccgcggcccggcggggtctccgccttccggggcagcctgccggaggcgcggccggctcaacccacgggcggggcggggcacttggcggtgccggcggagcacggctccgcgcctgccgtgaccgtgccggtcaccgccgcgtccgcttcctccgcaccgggctccgcgcccgccgccgtcccgtccggtgatccgtccggcgacgggagcatggccgcacgtgtggccggaccggcacgggacctcttccgggggctcgtcgcggacgtcctgcaactgcccgtcggcgacatccgtgccgacgtgcccttcgagcggtacggcatcgactcgatcctcgtcgtccaactcaccgacgccgtacggaaggtgctcgacggcgtgggcagcacgctcttcttcgaggtgagcacggtcgacggcctcgtggagcacttcctgcgcacccggccggacgaactcgccgcgctcgtcggcgtatccgccgcggagcacccggaacccgcggcggaagccgccgcaccggaggcggtcaccgaggagccggcggcctctgtacccgcacccgcacccgtagccgctcctgtctccgtacccgtgcccgccgccccgggtgaggacgtccccgtcgccgtcgtcggcatggccgggcgctaccccggcgccgccgacctggacgccttctgggagaacctgctcgcgggccgcgactgcgtcaccgagatccccgacggccgctgggaccacggccgttactacgacgagcgccgcggcgtgcccggcaggacgtacagcaagtggggcggattcctcgacggcgtcgacgagttcgactcgctgttcttcggcatctcgccgaaggccgcgtccacgatggacccgcaggagcggctgttcctccagtgcgcgtggacggcgctggaggacgcgggccacacgcgggcctcgctgcgctccgcctcccgcgcccggctgcccgaagacgccggggacatcggggtgttcgtgggcgcgatgtactccgagtaccagctctacggcgcggagcagggcgtacggggcgagcccgtcgtcgtacccggcagcctcgcctcgatcgcgaaccggctgtcgtacttcctcgacgcgtccgggcccagcgtcgccgtcgacacgatgtgcgcctcggccctgtccgccgtgcatctggcgtgtgccgcgatccggcgcggtgagtgcgcctcggcggtggccggcggcgtcaatctgtcgctgcaccccagcaagtacctgatgatcggcgagggacagttcgcctcctcagacgggcgctgccgcagcttcggcgcggacggcgacggctatgtgcccggcgagggcgtgggcgcggtgctgctgcggccgctcgccgacgccgtggccgacggcgaccgcgtgctcggcgtgatccgcggcagcgccgtgaaccacggcgggcacacgcacggcttcaccgttcccaacccgctcgcccaggcgtccgtgatccgcggcgcgtggcgccgctccggcgtggacccgcgcgacatcggctgcatcgaggcacacggcacgggcaccgcgctgggcgaccccgtggagatcgccggactgaacgccgccttcggcgagttcacctccgagcgcaccttctgctccctcggctccgccaagtccaacatcggacatctggagtcggcggcgggcgtcgcgggcctggccaagatgctgctccagatgcggcacggcacgctggtgccgtcgctgcacgccgagcgcaccaaccccgaaatcgacttcgccgccacgccgttcgtgctccagcgggaggccgcgccctggccgcgccgcgaggggcgcccacggctcggcggcatctccgcgttcggcgcgggcggttcgaacgcgcatctgctcgtcgaggagtacgtaccgacggcggcaccgccgcggcgtgcggcgccgggcccggtcctcgcggtgctctccgcgcgggacggcgagcgcctgcgggagtacgccgggaagctgcgggacgcactgcgctccgggcagtggaccgacgaggacctgccggacatcgcctacaccctccaggtcggacgggaggcgatgagcgcacggttcgccgccgaggtgagcaccctggccggactcatggacgcgctggacgcgtgcgcacggggcgccgccctgccgcccggcgcccggctgcgtaccgacggcgggcggggcggaccggtccaggacctcgcggacgacgaggacttccgggagaccgtcgtgcgctggctgcgccgcgggaagctggcgccgctcgccgaggcgtggaccggcggcctcgacgtggactgggcccgcggccacggcaccggcgaggaccggccgcgcaaggtcggcctgcccggctacccgttcgcgcgggagaggtactggtggaacgacgggctggccgaggccggaggcgagggcgctgacggtctgggagacgagggcgccgccggcggcaccgccggttccggtaacggttccgggccccgttccgctcgtacggacgggacgcgccccggtgaactgcccccgggcgacctcacgttgcaccccgtctgggagcccgtacatgcggcgggcggcggcgcggacgcgcccttcccgcagcccgcggaccgcgtcgtcgcggtcggcctggcaccggaggcccgtgccgcgctggaggcgtacggcacccgtgtggtgacgctcccggcaccccgggacggcggccgttccgtggcggacgtccgccgcgaactggagaccgcgggccccttcgaccacgtcgtcgtggagtgccccacccccgccgcacagggcgcgcggcagcgcgtcgaggctcaacgcgcctccgtacgcggcctgttccggctgctccaggcgctctccgccctccgcgcggacgagccgcggaccggtctgaccctcgtcacccgcgacgcgttcgacccggatcgcacgggcggcgccgacccggcgcaggccgcgctgcacggcctcgtcggcggcctcgccaaggaacagccgtactggcgcgtgcgcgccgtcgacctggccgagggcgagcccttcgtgcccgaggagatctgcgccctgcccgccgaccgccgggcgcatccgctcgtccggcgcggcggccagtggctgagccgcaggctgctgcccgtcggcgacgtacggcccggcacgccggacgacggcccgcgtacggctgttgacgggacggacgccgcgccgcaggccgtctccgtcccgtccggttccgtctcgtccgtctccgtcccgtccggcggtttccgcggtgacggcgtctatgtgctgatcggcggcgcgggcgacctcggcaccgtcctcaccgagcatctgctgcgccgctacgacgcccgcgtggtgtgggtgggccgccgtgcggaggacgacgccgtacgcgccgcggcggcacgcgtcgccgcggccaccggcggcgaggcccccgtgtatctgtccgccgacgcccgcgacccgggcgcgctcgcccgcgtacgggacgaggtgctgcgccggtacggacgcatcgacggcctggtgcacctggcgatggtcttcagccacacgctcctcgcggagctgccggaggaggacctgaacgcgacgctcgccgccaaggccgacccgaccgagcacttcgccgacgtcttcgcgggacagcggctcgacttcgtgctgctggtctcctccctcgtcagcttcatccgtaactcccaccaggcgcactacgcggcggcctgcgcctacgaggacgcccgcgcgcccggtctgggccgggcactgggctgccccgtcaaggtcgtcaactggggctactggggcaacgtcagcgacgaagtgctgcgcggcgtcacggagatggggctcgcgcccatcgaacccgcctccgccatggcggccgtcgaagagctgctgaccggcccgctcgaccagatcggcttcatgcggctcggccgtccgctgcccgtcgagggcgtgctcgcgggggagacgctgagcgggcatccgtacgcggcggtctcccgtacggccgcggagcccgcccccgtgccggtgccggccgcgctggcggagcaccacgcggggcctgtgccgggtgagatcgacgccctgctgtgccgctgtgtcgcggccacgctgcggcgtgccgggctgcgccgcccggccgacggcttcgcctccggttccggttccggttccggggccgggagcgcgggcgtgcgcgtggacgagcggttcgacggctggttcgcggcgaccgtacgcaccctgcgcgagtacgggctcgtcgactcccgcggcgactggagcgagcgcgcaccgggcgcgggggacgccgccgcctgcctggccgagtgggagcgcgcggccgagcggtgggcctctgcgcacgccgatctgcgggcgccgacggggctgttgggccgtacgctgcccgcgctggccgacatcctgcgcggccggatcccggcgaccgacgtgctgttcccggaggggtcgttctccctggtggagggcgtctaccgggacaacgccgtggccgcgcacttcaacgccgtactcgccgcacaggtgacggcgttcctgcacggccgccgcgcggccgacccggcggcgcggctgcgcgtactggagatcggcgcgggcaccggcggcaccaccgcgcccgtgctggagcaactggagtgcgcgggcctggagttggccgagtactgcttcaccgacctctccctcgccttcctccagcgtgccgaggacgccttcggccccggccgcgcccacttcgcctgccgcaccctcgacgtgtcacgggcaccgcgcacgcagggcttcgacgcgggggcgtacgacgtggtgatcgccgccaacgtgctgcacgccacggacgacgtacggaccgcgctgcggcacgccaagtcgctgctgcgcggcggcggaatgctggcgctgaacgagatcagcggcttctacctcgtcaaccacctcaccttcggcctgctggacggctggtggctctacggcgacgccgaactgcgggcgccgggcagccccgcgctgcctccggagagctggcgccgggtgctgacgcaggagggcttcaccggcgtcgcggacccggcacgggacgcccgtgccctgggacagcaggtcgtgatcgcccacagcgacggactggcccgcggcccggtgacggacgcggcaccggcggcaccggcagcaccggcggccgtggcgcggccggagacgaacacggcggtgagcgcggcgcccaacatggcggtgagcgcggcgagttcggcggcgggcggtccgcagacctccggcggtccggacgtgcgcgtggtcgccgacgtcgtcgagacggagctggccgacgcgctgcggctgccggcggaacggatcgaccgggcgggcgcgttcgcggacgtgggcctggactccatcgtcggcgcgcgcttcgtacggcggctcaacgaggaactgggcctggacctgccgacgacggtgatcttcgattaccggagcgtcgacgaactggccgcgcacatcgtggaggaccaccgtccgacctcgcctgcgccgggcggtaccggggcggccaccgctcaggagcccccggccgagcgggagtcggggcgcgccccggagcgggagcacgggcccgttgtggcgcccgatgtcaccgtgcccgatgccaccgagcccggttccgccccgtacggccgggagcccatcgccgtcgtgggcgtcagcgggcgcttcgccggttccgacgatctcgacgccctgtggcggcatctggccgcgggcgacgacctcgtcgggccgatcgaccgctgggatctctcggcctacggcgaggacgaactgacctgccgcagcggcagtttcctcgacggcatcgaccggttcgacgcccgcttcttcaagctgtcgggccgcgaggccgcctacaccgacccgcagcagcgcctcttcctcgaacaggcatggacggccctggaggacgccgggcacggcggcgcctcgaccgacggcatgcgctgcggcgtctacgtcggctgcaccggcggcgactacaaggaccacttcgaggacgcgccgcccgcgcaggccgtctggggcaacgcgccctcgatcgtccccgcgcgcatcgcctaccacctcaacctccagggcccggccatcgcggtcgacacggcctgctccagctcgctggtcgccgtgcatctggcctgccagggactgtggagcggcgagaccgagatggccgtggcgggcggcgtcagcgtgcagaccactccggccacctatctctcggccagccgcgccgggatgctctcgccgacgggacgctgccacaccttcgacgccgccgcggacgggttcgtaccgggcgagggcgtcggcgtcgtggtgctgcgcaggctctcggacgcgctgcgcgacggcgaccacgtgcacgccgtcatccgcggttcgggcgtcaaccaggacggcgccaccaacgggatcaccgcgcccagcgccctgtcccaggaacggctgctgcgccaggtctacgaggacttcgcgatcgacccgtccgagatcggcatggtcgaggcccacggcaccggcacacagctcggagacccgatcgaatgccacgccctgcggcgggtgttcgagggcagcgacgtccccggcggctgcgcgctcggttcgatcaagacgaacctcggccacaccacgtccgcggcgggcgtcgcgggtctgctgaagatcgtcctgtcgctgcggcaccggcagatcccgccctccctgcactaccgcgaccgcaatcccgagatccggctggaaggcggccccctgtacgtgaacacctcactgcgcccctgggagccgaacgcgggcggcagccgtgccgccgccctcagctcgttcggcttcagcggcaccaacagccatctcgtcgtcgaggaggcaccggcgcgtcccgggcggtccccgctctccggcgccgccgccgtggaggagcccggactgccccgggtcttcccgctgtccgcgccccagccggcggcgctgcgcgagcgcgtccgcgatctggccgtccatctgcggagcacgccggacgccgtcctcgtcgacgtcagccacaccctggcgacgggccgcgcccacttcgcgcaccgcgccgccttcgtcgcccgcacccgcgaggagctgatcggtcaactcgacgactggctcgacggggaggccggagacgccgggaaggcggcgaagaccggggaggccgcgaagaccggagacgtcggcgaggccgggggcgccgggccggaggagctggcccgcgaccgctacctcgccggtgaacccgccgacttcgccgcgctgttcgccggttccggcgcccgtcgcacaccgctgccgacgtaccccttccagcgcaggagccactgggtgcgcggcggcgcaccggggagcgccccggacgcggccgggtccggtacgtccaccacgtccggcacgcccgccctccgtaccgacgcaagggagaagggccgcggagccgcccgcgcggaggacgacgccgtcgccgtcgtcggcctctccgcccgcttcgcgcagtcgccggacgccgaggccctgtgggcacatctcgccgcgggcgacgacctggtcggcgaggtgacccgctgggacctgtcgcagatcagcggcggacgcaccgaacacggcagcttcgtcgaggacatcgcccgtttcgacgccctgtacttcggcgtctcgggcaacgaggccacgtacgccgacccgcagcagcgcatctacctggaggagtgctggcacgccctcgaggacgccggttacgcgggggagcggctggacgggcggggctgcggcgtctacgtgggcgcctaccccggcgactaccacgagctgatcggcgccgaccgcccgccgcagacgatgtggggcaacatggcctcggtcatcgcctcgcgcatctcctacttcctcgacctggacggcccggcgatgtccgtcgactcggcctgctccagctcgctcgtcgccatccacaccgcgtgccaggacctgcgtctgggcacgacctccatggcgctggcgggcggtgtgttcatccaggcgacgccgcggctctaccagtactcgggcaaggcgcggatgctctcggccaccggacgctgccacgccttcgacgccgccgcggacgggttcgtccccggcgagggcgccggagtcgtcgtcctcaagcggctgtcggacgcgctgcgcgacggcgaccgcgtctacggcgtgatccgctcctcgggcgtcaaccaggacggcaccaccaacggcatcacggcacccagcggcgcggctcaggagaacctcgtccgcgacgtgtacgagcgcgcgggcgtcgccccgtccgggatccagctcatcgaggcgcacggcaccggcacaccgctcggcgacccgatcgaattcgaggcgctgcgcgccgtgttcgcggacgcgccgacgggcggctgcgcgctgggcacgatcaagagcaacgtgggccacacccagttcaccgccggagtcgcgggggtcctcaaggtgctgctcgcgctcgaccacgaacagctcccgccctccttgcacttcacccggcccaacccggccatcgacctcgcgaacagccccttccacgtcaacaccgaactgctgccctggcgcgcgcccgccgacgggccgcgccgcgcgggcgtcagctccttcggcgccgccggcaccaacgcgcacgtcctgatcgaacaggcaccgtccgacgccgccgcccgcgcacgccgacacgggcgcgcacagtggctgttggtgctctccggccaggacggcaccgcgctgcgcgcccaggccgagcggatgctggaccacgtcgaacgccacccggacctcgacctcggcgacaccgcctggacgctcgccacgggacgccgccacagcgctcaccgtctggcgtgcgtcgccgccgaccgcgagcagtggacggcggcactgcggggctggctgcgcgacggccgtgccgagggcgtgtggacgggcgaggccgacgagtcgccccgctccgggcacagcggcgagagcggcgagggcagcggcgaaccggcccgcgccgaggcgctgatggccgagcacgaccgtcccggaaacctcgccgcgctcgcggagctgtacgtacggggcgaggtcgcgcgcttcgcaccgctgtacgccgacggggacttccgcatcgtctccctgcccggctaccccttcggcggcgagcgctactggaccgggccgctgcccggggacacgcccgacgggacggacggaacggacgggacgtacggcacggacgggatcagcgaatccggtggcgaatcccggccgtccgccgaaccccggccgtacgccggggcgttggcgctgaccggggaggagttcttcctcgacgaccaccgggtcggcggcgtccccgtactgccgggcgtcgcgtatctggaactcgcgcacgcggcggcgaccgcacagggcggcctcgcccccggcggtgtgctgctgcgcaacgtcgtctggtcccgcccggcgcgcgtcaccgagccgctctccgtggagacggtgctcgaaccacgcgccgcggacggcacgttcggatacgagatcgccaccgtccgggacggcgcccggcggctggtgcacggccggggccggatcgagccgcgtcccggcggcgcgcccgcccggctcggcctcgccgcgctgcgtgagcggtgcgacgtgcggagcctggaccacgcggagtgctacgcgttgctcggcgccaccgggatgtcgtacggcgccgcgatgcgcggtctggaggagctgcacgtcggccgcgggctcgcgctcggccggctgcgcgttccgcgcgaggcacgcgacggacgcccctggacgctccatcccgccctgctggacgcggcgttgcaggcgacggtcgggctggccctggacggggagtccgacgggctgacggccgcactgcccttcgcggtggagcaggtgcaggtgctcgccgcgagcccggagagcggctgggccgtggcccgtcccgcggacggcgccgccgagggcccggtccggcggatggacgtggagatctgcgacgacgagggcacggtgtgcgtacggctcctcggcttcagcacccgcgaactcccgggcgccaccgcgtcggtgacgaccggagcgacgaccggggcggggtccggggcggggtccgccgccgcgtcccctgctccggccgccgccgatcccggcgcgcccgccgacggctccctggtcttcgcccggcccgtctggcgcgccgtgccctccgcagacgtacgggaggagcgcccggcgccgagtccggcaccgtaccgggagatcctgctggccggtccggagtccgtcgacgctgcggaggtgcggaagcgctcgggcgtcccgtgctcggcgctgcccggcggcgccgatctgcccgagcggtacacccggcaggcccaggcgctgctggcgaaggtgcagcaactcctgccgcgcgtacgggaggagcgcgtcctgctccaggtcgcggtccccgcgcacggagaaggccggctcttcgcggggctcgcgggtctgctgcgtacggcctgcgcggagcaccccggactggccgcgcagctcgtcgagaccgacgccgccgacgcggcgacgctctgcgcccacctcgacgccgaggccgcgcagcccggcgtggcgacggtgcgccgcacgggcggcgaacggctggtgcggcagtggcacggcttccgtccggagcgcggcgatcagccctggaagccgggcggggtccatctcgtcaccggcggcgccggcggcctcggagcgctgttcgcccgccggatcgcccgtaccgcgcccggatccgtactggtgctgtgcggccgctccccggagggcgcggcacagcgcgaactcctgggtgagctgcgggagtcgggcgccgcacacgcggagtaccacagcctggacgtcggcaggcgtgcggacgtcgtccggctcgtgcggcaggtcgtggaccggcacggacggctcgacggcgtgatccacagcgccggagtgctgcgggacggcttcgtcgcccacaagaccccggagtacctgggcgaggtcttcgccccgaaggccgggggagtggtgcacctcgacgaggccaccgccgcactggagttggacttcttcctcgtcttctcctcgatgtcggtgctcggcaaccccggccaggccgactacgcggcggccaacgcgttcctggacgcgtacgtcgcccaccgcgccggactggcggaccgcggtgagcgccacggccgctcgctctcggtcggctggcccctgtgggcggacggcggcatgcacgtggacgcggccacggagcgccgcatccaccagagctccggaatgcggccgttgcgtgcccgtgaggggttcgaggcgctggagcgcctgtacgggagcggactgccgcacgcgctgaccgcgttcggcgaccgcgagcgcatcgcgtcggtgctgctcgacggttccgagggctccgacggctcggctcgtccggacggtccggacgcggagcgggagacggacgagcggcgccggaccccggcggacgcgaacgacgaacggaacgaggccatgtcacacacggcgctggtcggccgactcgccgcccatctctcggagttgctggacgtaccggcggaggagatcgagggcggggtcgagctgagcgagtacggcttcgactcgatctcgctgacggagttcgtcacgctgctcaacggcgcgtacgggctgtcgctcgtgccgacggtgctcttcgagcactcgacgctcgacggggtcgcgggacatctgctggaggagtacgcggaccgcttcgcgccggagccggagccggagccggagccgcagccggtgcaggcgcagatgccggagccggtgccggtgccggagccggaacctgcaccggtgccggcgcgcgggcccgtggcaccgtcaaccgcccccgtggcggccgacgatgacgacgcgctgcgccgtgcgctggtcaagcggctgcgggagctgacgtcccgcatcctccgggtgcccgcggagaagatcagcgccacgcaggagatgagcaagtacggcgtggactccctgtcgctggcggagctggcggcggccgtgaacgcggagttctcgctgatgctggacccgacgctgttcttcgagcacccgacgctggaggccgtcgcccgctatctcctcgaccggcacgccgaccggctcaccggcctggtgaccgaggagacccccgaaccggccatgaccgaacaggccgtggctgaaccggtcgtggccgagccgcccgtcgtcgaatcgcccgctacgacgtcacccgccgcggagacgtccgtcaccgagacgtccgtacgtgaacccgccgcccccgcggcggctccggctccggctttcgccgcggccccggggccgggtgctgcggaggagcccgtcgccgtcatcgggatcagcgcgcgttttccgatggcggatgatctggcggagttctgggagaacttgcgtgagggccgggactgtatccgtgaggtgccctcggatcgctgggactggcgcgagtactacggcgaccccgtcaaggagcccaacaagaccaacgtgacgtccggtggattcatggacggcgtgggcgacttcgacccgctcttcttcgacatctcgcccaaggaagcggagttgatggacccgcagcagcggctgctgatgctccacacctggaaggccctggaggacgcgggctatgcgccggacagtctcgccggcaccggcacggccctcttcgtcggtacgacgaacaccggttacggaagcatggtcagccgctattcaccggtgatcgagggatacgacgcgaccggggccgcgccctgcatgggcccgaaccggatgagccatttcctcgatctgcacggccccagcgagcccgtggacaccgcctgttccagttcgctcatcgcaatgcaccgcgccattcaggcaattcacgacggccattccgacatggcgatcgcgggcggcgtcaacacgatggtgagcatcgacggccacatcagcatttcccgtgcggggatgttgagtgtggatggtcggtgtaagacgttttcggtgggggctgatggttatgggcgtggtgagggggtggggattttggtgttgaagcgtttgtcggctgcggtgcgtgatggtgatcatgtgtatggggtggtgcgtggttcggcggtgaatcatgggggtcgtgcgaattctttgacggcgccgaatcctcgtgctcaggcggatttggtggtgggtgcgtggtcgcgggcgggtgttgatccgcggtcggtgggttatgtggaggcgcatggtacggggacggggctgggtgatccggttgaggtgaacgggttgaaggctgcgtttgcggagttgtatgagcggtggggtgtttcgggggccggtgaggcgcattgtggtctgggttcggtgaagacgaatatcgggcatttggagttggcgtcgggtgtcgcgggtgtgatcaaggtgttgttgcagatgcggcatcggacgttggtggggagtttgcattgtgggtcggtgaatccgtatgtgcggttggaggggagtcctttccgtctggtgcgggagcgtgagccgtggcgggcggtacgggatgagaacgggcgggagttgccgcgccgtgcgggcgtgagttccttcggtttcggcggcgccaacgcccatatcgttctggaggaataccagcccccggccggcacgcagaccgacgcccacacccgcaccggcccctcaaccaccgtccacagcggccccgttgccgtcctgctctccgcccaccgtcccgacgtactgcgggagtcggcgacccgctgggtcgaggtcctgcggcgcggcgactaccgcgacgccgacctcccggcgctgtcgtacacctcgcagacgggacgcaccgccatggccgagcggctcgcggtcgtcgccgggacgctggaggagctgcgcgcgggactggagtcctggctccgcggcgagccgaccccggccgtgttcaccggacgcgccccgcgcgacggcgacgcaccggcggcaccggccgccctcaccgacgggttcgcctccgggggacgtacggaggcgcggcactgggcgccggtgctccaggcgtggacgacgggcgccgagtgcgactggcggacgctgtggggcgaacggcacccgcaacggatctccatgccgacgtaccccttccaactccggcgctactggctcgacatgaccaccccggcgcacggcccgcacgtctcccgcggactgcatccgctggtgcaccggaacacctccgacctgagcgagcagcgctacacctcgcacttcaccggccgggagttctacatcgccgaccaccgggtgcagggcgaacaggtcgtccccggcgcggcgttgctggagatggcacgcgccgccgccgtcctcgcggcgggcggtgcggagaccgactgggcgctgcgccaggtggtctggtcccggccgctgacggccggacggcccgtcgacgtgcacaccgccgtgtccgtgcgggcggacggcgaaccggccttcgagatctacacggagggccccggcggcgaacgcgtcgtgcactccaccggacggctgcaccgcaggaccgccgggaacgccgccgaactcctggacggacccgaactccccggcggcgccggacacttggacgtcgccgcgctgcgtgcccagtgcgacggcaccgtcctcgacgccgaggagtgctacgcccggttctccggcgtgggcctggagtacgggcccacgctgcgagccgtcgagacgctgagcggcggcacccggcaggcggtggcccggctgcggctgtcggccgccgcgtccgcgaggaccgggttcgccctgcacccgagcctgctcgacgccgcactccagtccacggcgggcctcttcaccggttccggtacgtcctcggcggccctgccgtttgccctggaccggctggaggtgctgcgcgcgacgccctcctcggggtgggcggtggcacgcttcgccgccgacgaccgcccaggcggggtgcgccgcctggacatcgacgtgtgcgacgacgacggcgaggtgtgcgtacggatccggggcttccaggtccgtacgtacggcggcgacgccgccccgtccgcctccggcgccgcaaacggcacccacgccgtgaccacggacggcaccggaaacggcacagacaccggcaacggaaacagcaccggcaccggcagcgaggcggacgcggacgcccggctgctgcacctcatccacgccatcggcgagggcgccctgagcgctgacgaattccagcggagcctcatatga

SEQ ID NO: 36 - protein, 2098 aa, Streptomyces amphibiosporus
MTTHATSLTELHEQIRTGRIGQDEALRLIRAWQQGRQTGSEGGPAERQATGDDAAARGEALRERVCDIVTHAVSELLKVGPDDLDADVELSEYGLDSIVMSQLVNAVNDELGLELAPTVLFEHPNLRAFSAHLADTYADSLSVRLLGTPGTGPAPAPSTATSPAPSTATSAEPAAVAAPSTSPSEARTESRVPTPPATGGRFFPAAVPSAPSAETEPVTAPAPAPASEQASPAQASAAEEPVAVVGMSGRFPMADDLAEFWENLREGRDCIREVPSDRWDWREYYGDPVEEPGRTDVKWGGFIDGVADFDPLFFGIAPKEALHMDPQQRLLMLYVWKALEDAGHSADSLAGSDLAMFVGTNDTGYGTLAERCGKRDSVSPTGGVPSLGPNRMSFFLDVHGPSEPVETACSSSLVAMHRGVTAIARAECETAVVGGINTIVVPDGHVSFSRAGMLSVDGRCKTFSVGADGYGRGEGVGILVLKRLSAAVRDGDHVYGVVRGSAVNHGGRANSLTAPNPRAQADLVVGAWSRAGVDPRSVGYVEAHGTGTGLGDPVEVNGLKAAFAELYERWGVSGAGEAHCGLGSVKTNIGHLELASGVAGVIKVLLQMRHRTLVGSLHCGSVNPYVRLEGSPFRLVREREPWRAVRDENGRELPRRAGVSSFGFGGANAHIVLEEYQPPAGTQTDAHTRTGPSTTVHSGPVAVLLSAREPETLRARARQLVDWLDKGEATEADLPRISYTLQVGRVAMPERLACVTESLAELRAQLQEFLDGERPRGVRTGRAERRGIWNDLADDEDITAAVDNWMAKGKLDRLLKLWVAGAEFDWRRLWGEHPPRRIPLPAYPFRLQRYWIADGTSGRSTRRPSTAREGTPYGGTPRDAEYRELLRGDEYFLRDHRVGGVPTLPGAACLELVRAAWTHADRAPDTAPLRLRDVLWLRPLQVTAPRTVAVALDPADGTYEVRAADGDEREVYARGTVTADGPHTVDGPHSAGGDRPGGTAEPAEPVPAHDIAALRDRCPHRLDADGCYDRFADLGLAYGPALRAVETLHYGADLALARLVLPEAAAGERTLNPSMLDAAFQTTLGVLLGEQAQAADAERAAAGGSEDVAALPFAVREVRILAPTPAEGWAVARAAEGDRPGGTVRTLDIDLCDTSGRVCVRLTGFSTRTVPEDGAPQLPGEPPVLMIEPAWREAEAASVAAMPEDHRVVLCELPGVDAAELAGSLGGDCETWQAEGDVAARYTEYARRLLELLQEEARSPAPAPAPAPGGTSGGVPGGRLVQLVTPSSAPWLGGLSGMVRTARQEHPKLLVQWIEAEDDCSADELAVLLRGDGADPAEVAVRHGDGRRRVSRWRETPPPAPRVPWRDGGVYLVTGGSGGLAALFAKDMARRVRRPSLVLCGRGAAGPEQRELVAELEALGARAEYRVLDVSDAGAVSAAVREVVAAHGALHGVVHAAGVLRDGFLARKSAEELRQVFAGKVAGLRHLDEATADVELDFLIAFSSMAAFGNAGQADYAAANAFLDGYAQHREALRARGERHGRTLSVNWPLWEKGGMRGGAGTEAVLQGVGMRPMRAETGLDALYRAWACGLTSVLVLEGDHERMRSRLLPEQPPLPEPPHRPDAQKPLDAQKPLDAPELPDGPEATRTGGGVPVRSGSADVARRVTAVLADLLEIDAESLRPDVPLREYGLDSIFLTQFLGTARKEFDPALTLDVIAGCETLTDFIDAIERAVAPPAATPPAPASASAPSPSSGTEGEPSTRPAPEPDGVPVVRPVAKAPEEFPELIPMNAVREGRPVFWVHHGNGGVESYAAVAECCGRPFYGIQPRGWTGSEDILTGQEAMAAYYVDIIRAVQPEGPYDVGGFSLGGLFAYEVVRQLQLQDATVDTLVMLDTLDAASTNLANSLMTGGRQDDADVVAKVSAFRAVNLMLGNDSLDAREGTSSVLHRDEVDTALDPDAFLDSLVEAAVARGIHKTPAQLRTRVRQLARYFDAVHGERHVVHPLPRREEVRCYYLRNAGGQFFGPFEEYMVLFPEPDLPAVDGTPYWREWADAVDDFFVIDVDTETHAQVMTEPAALKKVLRLCDRLYAPEQHAQGGR

SEQ ID NO: 37 - DNA, 6297 bp, Streptomyces amphibiosporus
atgaccactcacgccacgtcacttaccgaactgcacgagcagatccgtaccggacggatcggccaggacgaggccctccggctgatccgggcctggcagcagggccggcagaccgggagcgagggcgggccggcggagcggcaggccacgggcgacgacgccgcggcccgtggcgaggccctgcgcgagcgcgtgtgcgacatcgtgacgcacgcggtcagcgagttgctgaaggtcggcccggacgacctggacgccgacgtcgaactcagcgagtacgggctggactcgatcgtgatgagccagctcgtcaacgcggtgaacgacgaactcggcctggaactcgcccccacggtcctcttcgagcacccgaatctgcgggccttcagcgcccacctcgccgacacgtacgcggactcgctctccgtacggctgctcggcacgcccggcacggggcccgcgcccgccccctcgaccgcgacatcgcccgccccctcgaccgcgacatcggccgaacccgcggccgtcgccgctccgtcgacgtcaccgtcggaggcccgtacggaatcccgggtgcctacgcctccggcaaccggaggccgcttcttccccgccgccgtcccatccgccccatccgccgaaaccgaacccgtcaccgcacccgcacccgcgcccgcctctgaacaggcttcccctgcgcaggcttccgctgcggaggagcccgtcgccgtcgtcggcatgagcggacgttttccgatggcggatgatctggcggagttctgggagaacttgcgtgagggccgggactgtattcgtgaggtgccctcggatcgctgggactggcgcgagtactacggcgatcccgtcgaggagcccggccgcaccgatgtgaagtggggcggattcatcgacggcgtcgccgacttcgatccgctcttcttcggcatcgcgccgaaggaagccctccatatggacccgcagcagcggctgttgatgctctacgtctggaaggccctggaggacgcgggccattcggcggacagtctcgccgggagcgatctggcgatgttcgtcggtacgaacgacaccggttacggcacgctcgccgaacggtgcggaaaacgggacagcgtctcgcccaccggcggcgtcccctcactcggcccgaaccgcatgagcttctttctcgacgtgcacggtcccagcgagcccgtggaaacggcgtgttcgagttcgctggtcgccatgcaccgcggtgtcacggcgatcgcccgcgcggaatgtgagaccgccgtggtcggcggcatcaacaccatcgtcgttcccgacggtcacgtcagcttctcccgtgcggggatgttgagtgtggatggtcggtgtaagacgttttcggtgggggctgatggttatgggcgtggtgagggggtggggattttggtgttgaagcgtttgtcggctgcggtgcgtgatggtgatcatgtgtatggggtggtgcgtggttcggcggtgaatcatgggggtcgtgcgaattctttgacggcgccgaatcctcgtgctcaggcggatttggtggtgggtgcgtggtcgcgggcgggtgttgatccgcggtcggtgggttatgtggaggcgcatggtacggggacggggctgggtgatccggttgaggtgaacgggttgaaggctgcgtttgcggagttgtatgagcggtggggtgtttcgggggccggtgaggcgcattgtggtctgggttcggtgaagacgaatatcgggcatttggagttggcgtcgggtgtcgcgggtgtgatcaaggtgttgttgcagatgcggcatcggacgttggtggggagtttgcattgtgggtcggtgaatccgtatgtgcggttggaggggagtcctttccgtctggtgcgggagcgtgagccgtggcgggcggtacgggatgagaacgggcgggagttgccgcgccgtgcgggcgtgagttccttcggtttcggcggcgccaacgcccatatcgttctggaggaataccagcccccggccggcacgcagaccgacgcccacacccgcaccggcccctcaaccaccgtccacagcggccccgttgccgtcctgctctccgcgcgtgagcccgagacgctgcgcgcccgcgcacggcagctcgtcgactggctcgacaagggcgaggccaccgaggccgatctgccgcggatctcctacaccctccaggtcggccgggtcgcgatgccggaacgactggcctgtgtgacggagtcgctggccgaactgcgcgcccagctccaggagttcctcgacggcgaacggccccgtggcgtacggaccgggcgtgccgagcggcgcggcatctggaacgacctggccgacgacgaggacatcaccgccgcggtcgacaactggatggccaagggcaagctcgaccggctgctcaaactctgggtcgccggcgccgagttcgactggcggcggctgtggggcgaacaccccccgcggcgtattccgctgcccgcctaccccttccggctccagcgctactggatcgccgacggcacaagcggccggtccacacggcggccgtcgaccgcgcgcgagggaacgccgtacggaggcaccccgcgggacgcggagtaccgcgaactgctgcgcggcgacgagtacttcctgcgtgaccaccgcgtgggcggcgtgccgacgctgcccggtgccgcctgtctggagctggtccgcgcggcctggacccacgccgaccgtgcccccgacaccgccccgctgcggctgcgcgacgtgctgtggctgcgtccgctccaggtcaccgcgccccgtaccgtcgccgtcgccctcgacccggccgacggcacgtacgaggtgcgcgccgcggacggcgacgagcgcgaggtgtacgcacgcggcaccgtcaccgctgacggcccccacaccgtcgacggcccccacagcgccggcggcgaccgtccgggcgggaccgccgaaccggcggagcccgtaccggcacacgacatcgccgccctgcgcgaccgctgcccccaccgtctcgacgccgacggctgctacgaccggttcgcggacctgggcctcgcctacggcccggccctgcgcgccgtcgagacgctgcactacggcgcggacctggcactggcccgtctggtgctgccggaggcggcggccggggaacggacgctcaacccgagcatgctggacgccgcattccagaccaccctcggcgtgctcctcggcgagcaggcccaggcggcggacgccgaacgggccgctgccggcggttccgaggacgtcgcggcgctgccgttcgccgtgcgcgaggtacggatcctcgcccccacccccgccgagggctgggccgtggcacgcgccgcggagggcgaccggcccggcgggacggtacgcaccctggacatcgacctgtgcgacacctccgggcgggtctgcgtacgcctcacggggttcagcacccgtacggtccccgaggacggtgcgccccaactccccggggagccgcccgtgttgatgatcgagcccgcctggcgcgaggcggaggcggcctccgtggcggccatgccggaggaccaccgggtcgtgctgtgcgaactgcccggcgtggacgcggccgagctggccggctccctcggcggcgactgcgagacctggcaggccgagggggacgtcgccgcccgctacaccgagtacgcccggcggctgctggaactcctccaggaggaggcccgcagcccggccccggccccggcccccgcccctggcgggacctccggcggggttcccggcgggcggctcgtccagctcgtcaccccgtcctcggcaccctggctcggcggtctgagcggcatggtgcgcaccgcccgccaggagcaccccaagctcctcgtccagtggatcgaggccgaagacgactgctccgccgatgagttggcggtgctgctgcgcggcgacggcgccgaccccgccgaggtggcggtacggcacggcgacggacggcgcagggtctctcggtggcgcgagacgccgccgcccgcgccccgcgtcccctggcgcgacggcggggtctatctcgtcaccggcggctcgggcggcctcgccgcgctgttcgccaaggacatggcccgtcgcgtgcggcggccgtcgctggtcctctgcggacgcggcgcggccggtcccgaacagcgggagctggtcgcggagttggaggcgctgggcgcccgcgcggagtaccgcgtactggacgtctccgacgccggggccgtcagcgcggcggtccgggaggtggtcgccgcacacggcgccctgcacggtgtcgtccacgcggcgggcgtgctgcgggacggcttcctcgcgcgcaagagcgccgaggagctgcggcaggtcttcgcggggaaggtcgccgggctgcgccatctcgacgaggccacggcggacgtggagttggacttcctgatcgccttctcatcgatggccgccttcggcaacgccgggcaggccgactacgccgccgccaacgcgttcctcgacggctacgcccagcaccgcgaagcgctccgcgcgcgcggcgagcgccacgggcggaccctctcggtgaactggccgctgtgggagaagggcggtatgcgcggcggcgcgggcaccgaggccgtgctccagggcgtgggcatgcgcccgatgcgggcggagacggggctcgacgccctgtaccgggcgtgggcgtgcggtctgacgtccgtcctggtgctggagggcgaccacgagcggatgcgctcccggctgctgccggagcagcccccgctgcccgagcccccgcaccggccggacgcacagaagccgctggacgctcagaagcccctggacgcaccggagttgccggacggaccggaagcgacgaggacgggcggcggcgtgccggtgcgctccggctccgcggacgtcgcccgccgggtcaccgccgtcctggcggacctgctggagatcgacgcggagtccctgcggcccgacgtgccgctacgcgagtacggactcgactcgatcttcctcacccagttcctcggcacggcccgcaaggagttcgacccggcgctcacgctcgacgtcatagcgggctgcgagacgctgacggacttcatcgacgcgatcgagcgcgccgtcgccccgccggccgccacgcctccggcgcccgcatccgcgtccgcgccctcaccctcgtccggtacggaaggggaaccgtcaacgcgccccgccccggagccggatggcgtaccggtggtgcgtcccgtcgccaaggctcccgaggagttccccgagctgatccccatgaacgccgtacgggagggccgcccggtcttctgggtccaccacggcaacggcggagtcgagtcgtacgcggcggtggccgagtgctgcggacgccccttctacggcatccagccccgcggctggacgggctcggaggacatcctcaccggccaggaggccatggccgcctactacgtggacatcatccgcgccgtccagccggagggcccgtacgacgtcggcggcttctccctcggcggtctgttcgcctacgaggtcgtacgccaactccagctccaggacgccacggtggacaccctggtgatgctggacaccctcgacgccgcctcgaccaacctggccaactccctgatgacgggcggccgtcaggacgacgccgacgtggtggcgaaggtcagcgccttccgcgccgtcaacctgatgctgggcaacgacagcctggacgcgcgcgagggcacctcgtccgtcctgcaccgggacgaggtggacaccgcgctcgacccggacgccttcctggactccctcgtggaggccgccgtcgcccgcggcatccacaagaccccggcccaactgcggacgcgcgtacggcagttggcgcggtacttcgacgccgtgcacggcgagcggcacgtggtgcacccgctgccgcggcgcgaggaggtgcgctgctactacctgcgcaacgcgggcgggcagttcttcggccccttcgaggagtacatggtcctcttccccgaaccggacctcccggcggtggacggcaccccgtactggcgcgaatgggccgacgccgtcgacgacttcttcgtcatcgacgtcgacacggagacccacgcgcaggtgatgacggagcccgcggcgctgaagaaggtgctgcggctgtgcgaccggctgtacgcgccggagcagcacgcgcagggaggccggtga

SEQ ID NO: 38 - protein, 768 aa, Streptomyces amphibiosporus
MEAVVFPGQGAQRRGMGRELFDAFPSLTEQASDVLGYSVRELCVADPERRLRSTEYTQPALFVVGTLAHLKWQEETGRSAAYFAGHSVGEYTALHAAGAFGFETGLRLVQRRGLLMSQAEGGGMAAVLGLGAGELTELLREGGFVSLALANDNTPDQQVVSGAAHEIDVLEAYLSERGVRGVRLNVSGAFHSPLMLPAQEAFAAYVRDFTLGDPETPVISNVTARPHPPGGTAELLVRQISSPVRWTESVRHLLDLGVEEFTELGGSVVAKLVRQIREAHRRDADASGPAPAAPAAPVRSEPAARSALGSAVFRERLGLRHSYAAGGMYRGIASPAMVVRLARAGMLGFLGTGGLTPEAVEERILQVRRELREGEPFGVNFLADHDDPAAERRVAEMLMRRGVTVVEAAAFIGMTPASSSTARAGMHRGPDGAPRCAHRIVAKVSRPEVGRRLHGTAPGKVVDGLLREGAITGEQAELVRQVPMSHDITVEADSGGHTDGGIATVMLPAMLGLRRQAQRSHDYAEPLCMGLAGGLGTPEAVAAAFMLGADYVLTGSVNQCTVEADTSDAVKDMLQTIDIQDTGYAPAGDMFEMGARVQVLRKGVFFPTRANKLYALYQHHDGLDDLPAKTRALLERSYFHRTFDEIWTEVREHYRAKGQPEVTDKAERQPKVKMALVFRWYFAYSARLALAGQDGDKVNFQIHTGPALGAFNQWVKGTACESWRARHADAIGLMLMEGAAEHVAAACESWGGTSRFAPAEERALAART

SEQ ID NO: 39 - DNA, 2307 bp, Streptomyces amphibiosporus
atggaggcggtcgtctttcccgggcagggcgcgcagcgccgcggcatgggccgcgagctgttcgacgcctttccctcgctcaccgagcaggcgtccgacgtcctcgggtactccgtacgcgagttgtgcgtggcggaccccgagcgccggctgcgcagcaccgagtacacccagcccgcgctgttcgtcgtcggcaccctcgcgcacctcaagtggcaggaggagacggggcgttcggccgcgtacttcgccgggcacagcgtgggggagtacaccgcgctgcacgccgcgggcgccttcggcttcgagaccgggctgcgcctggtgcagcggcgcggtctcctcatgtcgcaggcggagggcggcggcatggcggccgtactcggcctcggcgccggggagttgaccgagctgctccgcgagggcggcttcgtctccctcgccctcgccaacgacaacacgcccgatcagcaggtcgtctcgggcgccgcgcacgagatcgacgtcctggaggcgtatctgtccgaacgcggcgtgcgcggtgtgcggctgaacgtctcgggcgccttccactcgccgctgatgctgcccgcacaggaggcgttcgccgcgtacgtacgggacttcacgctcggcgacccggagacgccggtgatctccaacgtcaccgcgcggccccatccgccgggcggtacggccgagttgctcgtacggcagatctccagccccgtgcggtggacggagagcgtgcgccatctgctcgacctcggcgtggaggagttcaccgaactcggcggcagcgtggtcgcgaagctcgtacggcagatccgcgaggcgcaccgcagggacgcggacgcctccggcccggcgcccgccgcgcccgcggctcccgtacggtccgaacccgccgcacggtccgcgctcggcagcgcggtcttccgcgagcggctggggctgcgccactcctacgcggcgggcggcatgtaccggggcatcgcctccccggccatggtggtgcgcctcgcccgcgccgggatgctcggcttcctcggcacgggcgggctgacgcccgaggccgtcgaggagcggatcctccaggtccggcgggagctgcgcgagggcgagcccttcggcgtcaacttcctcgccgaccacgacgatccggccgccgaacgccgcgtcgccgagatgctgatgcgccgcggcgtgaccgtcgtcgaggcggcggccttcatcggcatgaccccggcctcgtcctctaccgcgcgcgcgggcatgcaccgcggaccggacggggccccgcgctgcgcccaccgcatcgtcgccaaggtctcccgccccgaggtgggccggcgccttcatggcaccgcgcccgggaaggtggtcgacggcctgctccgggagggcgcgatcaccggcgagcaggcggagctcgtacggcaggtcccgatgagccacgacatcaccgtcgaggcggactccggcggccacaccgacgggggcatcgccaccgtcatgctgcccgcgatgctcggcctccggcggcaggcccagcgcagccacgactacgcggagccgctgtgcatggggctcgccggcggcctcggcactccggaggccgtcgccgcggccttcatgctgggcgccgactacgtcctgacgggctccgtcaaccagtgcaccgtcgaggcggacaccagcgacgccgtcaaggacatgctccagaccatcgacatccaggacacgggctacgcgcccgcgggcgacatgttcgagatgggagcccgggtccaagtgctgcgcaagggcgtcttcttcccgacccgggccaacaagctgtacgcgctctaccagcaccacgacggcctcgacgacctgcccgcgaagacccgtgccctgctggagaggtcgtacttccaccgcaccttcgacgagatctggacggaggtacgcgagcactaccgcgccaagggccagcccgaggtcaccgacaaggcggagcggcagccgaaggtgaagatggcgctggtcttccgctggtacttcgcctactcggcccggctggcactggccgggcaggacggcgacaaggtcaacttccagatccacacggggcccgcgctcggcgccttcaaccagtgggtcaagggcacggcctgcgagtcctggcgcgcccggcatgccgacgccatcgggctgatgctcatggagggcgcggcagaacacgtcgcggcggcctgcgagtcgtggggcggtacctcccgcttcgcccccgcggaggaacgcgccctggccgcccgcacctga

SEQ ID NO: 40 - protein, 418 aa, Streptomyces amphibiosporus
MRGHAVTTHLTTDIDEIVTDVFQSTEGKKNPYPLYRRLQELGQVHRSEQLGWVATGYEVCSAALRDPRVIKGPEQIQPGRPDPAEHSAEALLRGTMHRLDPPDHTRLRRLVNGAFTPRSVAALEPDIQELIDDLITPAVKKAEAGEPVDMMSGFAFPLSVAVIGRMLGVPASDWHRFHDVVLDLSSMVELGFTGDELPKADAAADELIAYFRKLGAERMRNPADDLTSTLANATEAGDRLTEQELVTMLILLFMAGFETTTHSMGNGMFALLENPEQTQWLRRNMDAMPAAVEELIRYDSPVQFIAGYTKEPVELADGTAVPADEYLFLMIGAANRDPRVFSDPELLRLDRGEAAPMSFGGGIHYCLGAGLARLEIRKIFTSLLTRFSAIELAEPEPERRSGLALRGYARIPMWLTPA

SEQ ID NO: 41 - DNA, 1257 bp, Streptomyces amphibiosporus
atgagaggacacgcagtgaccacccacctcaccaccgacatcgacgagatcgtcacggacgtcttccagtccaccgaggggaagaagaacccctaccccctgtaccggcgcctccaggagctgggccaggtgcaccgctccgagcaactgggctgggtcgccacgggatacgaggtgtgcagcgccgcgctgcgcgacccccgcgtcatcaagggccccgagcagatccagccgggccgccccgaccccgccgagcactccgccgaggcgttgctgcgcggcacgatgcaccggctcgacccgccggaccacacccggctgcgccgcctcgtcaacggcgccttcacgccgcgcagcgtcgccgcgctggagccggacatccaggaactgatcgacgacctgatcacaccggccgtcaagaaggcggaggcgggcgagcccgtcgacatgatgagcggcttcgccttcccgctctccgtcgccgtgatcggccgcatgctcggcgtgcccgcgtccgactggcaccgcttccacgacgtcgtgctcgacctgtcctccatggtcgaactcggcttcaccggcgacgagttgcccaaggccgacgccgccgcggacgaactgatcgcctacttccgcaagttgggcgccgagcgcatgcgcaaccccgccgacgacctgacctccacgctcgccaacgcgaccgaggccggtgaccgcctcaccgagcaggaactcgtcaccatgctgatcctgctgttcatggccggtttcgagacgacgacccactccatgggcaacggcatgttcgccctgctggagaacccggagcagacgcagtggctgcgccgcaacatggacgccatgcccgcggccgtcgaggagctgatccgctacgactccccggtgcagttcatcgccggctacaccaaggagcccgtggaactggcggacggcaccgccgtccccgcggacgagtatctgttcctgatgatcggcgcggccaaccgcgacccgcgcgtcttctccgaccccgaactgctgcgtctggaccgcggtgaggccgcgccgatgagcttcggcggcggcatccactactgcctcggcgcgggcctggcccggctggagatccggaagatcttcacctcgctgctcacccgcttctccgcgatcgaactggccgagcccgaaccggaacgccgcagcggactcgccctgcgcggctacgcccgcatcccgatgtggctcaccccggcgtaa

SEQ ID NO: 42 - protein, 247 aa, Streptomyces amphibiosporus
VIGRMLPGWVSTEEIFVGGQEDTLSGLFPEERAAVARAVPKRQREFADVRACARSALGRLGVAPVPLVPGHRGAPQWPEGVVGSMTHCDGYRAAAVARGSDAVGIGIDAEPAEPTPDGVLGVISLPAEREHLRTLAAAHPQVAWDRLLFSAKESVFKVWYPLTSRELDFSEAEITIDADAGTFSARLLVEGPVLGGERLRGFTGRWAARRGLLATAVLLGSRTETGAGAGSKSETETGTETGPAALR

SEQ ID NO: 43 - DNA, 744 bp, Streptomyces amphibiosporus
gtgatcgggcggatgttgccggggtgggtgagcaccgaggagatcttcgtcggtgggcaggaggacacgctgagtgggctcttccccgaggagcgggccgccgtggcccgggcggtgcccaagcggcagcgggagttcgcggacgtacgggcgtgtgcgcggagtgcactggggcggctgggtgtggcgcccgtgccgctggtgccggggcaccggggcgccccgcagtggccggagggtgtcgtcggcagcatgacgcactgcgacggctaccgggcggcggccgtcgcccgcggcagcgacgcggtcggcatcggcatcgacgccgaacccgcagagccgacgccggacggcgttctgggcgtgatctccctgcccgccgagcgggaacacctgcgcacgctggcggcggcgcatccccaagtggcctgggaccgcctgctgttcagcgcgaaggagagcgtcttcaaggtctggtatccgctcacctcacgcgaactcgacttctccgaggccgagatcaccatcgacgcggacgcgggcacgttctccgcgcggctcctcgtggaggggccggtgctcggcggcgagcggttgcgcggtttcaccggccgctgggcggcacggcgggggctgctggccaccgccgtcctcttggggagcaggaccgagacgggagcaggggcggggtccaagtcggagacggagacggggacggagacggggcccgcggcgctccgctga